(19) United States
(12) Patent Application Publication
(10) Pub. No.: US 2009/0247519 A1
(43) Pub. Date: Oct. 1, 2009

(54) AMINO-ETHYL-AMINO-ARYL (AEAA) COMPOUNDS AND THEIR USE

(76) Inventors: Tony Michael Raynham, Greater London (GB); Timothy Robin Hammonds, Greater London (GB); Julia Helen Gilliatt, Greater London (GB); Mark David Charles, Greater London (GB); Gregoire Alexandre Pave, Greater London (GB); Caroline Heather Foxton, Greater London (GB); James Lindsay Carr, Greater London (GB); Neela Sumit Mistry, Greater London (GB)

Correspondence Address: MICHAEL BEST & FRIEDRICH LLP, ONE SOUTH PINCKNEY STREET, P O BOX 1806, MADISON, WI, 53701, US

(21) Appl. No.: 12/298,311

(22) PCT Filed: Apr. 26, 2007
(86) PCT No.: PCT/GB2007/001537

§ 371 Date: Oct. 24, 2008

Related U.S. Application Data
(60) Provisional application No. 60/745,630, filed on Apr. 26, 2006.

(30) Foreign Application Priority Data
Apr. 26, 2006 (GB) 0608269.7

(51) Int. Cl. A61K 31/5377; C07D 403/06; A61K 31/506; A61K 31/505; A61K 31/517; C07D 239/72; C07D 413/12

(52) U.S. Cl. 514/234.5; 544/328; 514/256; 514/266.4; 544/293; 544/119

(57) ABSTRACT

The present invention pertains generally to the field of therapeutic compounds, and more specifically to certain amino-ethyl-amino-aryl (AEAA) compounds which, inter alia, inhibit protein kinase D (PKD) (e.g., PKD1, PKD2, PKD3). The present invention also pertains to pharmaceutical compositions comprising such compounds, and the use of such compounds and compositions, both in vitro and in vivo, to inhibit PKD, and in the treatment of diseases and conditions that are mediated by PKD, that are ameliorated by the inhibition of PKD, etc., including proliferative conditions such as cancer, etc.

### RELATED APPLICATIONS

This application is related to: United Kingdom patent application number 0608269.7 filed 26 Apr. 2006 and U.S. patent application No. 60/745,630 filed 26 Apr. 2006; the contents of each of which are incorporated herein by reference in their entirety.

### TECHNICAL FIELD

The present invention pertains generally to the field of therapeutic compounds, and more specifically to certain amino-ethyl-amino-aryl (AEAA) compounds which, inter alia, inhibit protein kinase D (PKD) (e.g., PKD1, PKD2, PKD3). The present invention also pertains to pharmaceutical compositions comprising such compounds, and the use of such compounds and compositions, both in vitro and in vivo, to inhibit PKD, and in the treatment of diseases and conditions that are mediated by PKD, that are ameliorated by the inhibition of PKD, etc., including proliferative conditions such as cancer, etc.

### BACKGROUND

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### Protein Kinase D

Protein Kinase D1 (PKD), also known as Protein Kinase C mu (PKCμ), is the prototypical member of a family of three highly related serine/threonine kinase isoforms, PKD1, PKD2 and PKD3 (formally PKDv). These were intitially classified as members of the PKC superfamily by way of C1 domains (see, e.g., Van Lint, 2002). The many related PKC isoforms are classified into distinct groups: classical PKCs (α, βI, βII, and γ), regulated by calcium, DAG and phospholipids; novel PKCs (δ, ε, η, and θ), regulated by DAG and phospholipids; and the atypical PKCs (ξ and λ) which lack calcium or DAG binding domains. More recently, based on sequence similarities, the PKDs are now grouped into the calcium calmodulin-dependent kinase (CAMK) family of kinases (see, e.g., Doppler, 2005). Except where otherwise indicated, a reference to PKD is intended to be a reference to one or more or all of PKD1, PKD2, and PKD3.

The activity of the PKD family is regulated by at least three different means. Firstly, the PKDs are targets for the actions of the phorbol esters that are known tumour promoters (see, e.g., Van Lint et al., 1995). Phorbol esters regulate the cell localisation and activity of proteins containing conserved DAG-binding cysteine-rich domain (C1 domains). Secondly, the PKDs are activated in a PKC and/or tyrosine kinase dependent manner in response to multiple mitogenic signals including bombesin and PDGF (see, e.g., Zugaza et al., 1996; Matthews et al., 2000b; Storz, et al. 2004a). Thirdly, the activity of the PKDs can also be regulated by their interaction with lipids and/or proteins that also regulate their sub-cellular localisation (see, e.g., Wood et al, 2005).

Recent findings have shown that PKD1 is phosphorylated on multiple sites during in vivo activation. Five phosphorylation sites have been identified in PKD1: two sites in the regulatory domain, two in the catalytic domain, and one at the C-terminus. Ser744 and Ser748 (both in the activation loop) play a crucial role in the activation of PKD1. Substitution of these amino acids with alanine completely blocks PKD activation, while substitution with glutamic acid (mimicking phosphorylation) causes a constitutive activation. Ser916 (C-terminus) is an autophosphorylation site, not required for activation but rather regulating the conformation of PKD1. Ser203 (regulatory domain) is an autophosphorylation site and is located in the region that interacts with 14-3-3 proteins. Ser255 (in the regulatory domain) is a transphosphorylation site, targeted by PKC or a PKC-activated kinase.

The PKD family is an integral part of a number of signalling cascades that are aberrantly activated during a number of pathological conditions. Activated PKDs are known to be required for a number of cellular processes that have been demonstrated to be suitable points of therapeutic intervention:

### Cancer

The PKDs play a key role in promotion of cell proliferation, invasion, and inhibition of apoptosis, indicating that they are suitable targets for anti-cancer therapeutics. Evidence for these activities comes from the following observations:
Proliferation associated expression of PKD1 and PKD2 has been observed in CML, prostate cancer, small cell lung cancer, and pancreatic carcinoma lines (see, e.g., Mihailovic et al., 2004; Stewart and O'Brian, 2004; Paolucci and Rozengurt, 1999; Guha et al., 2002, 2003).
PKD1 is activated by growth stimuli in both small cell lung cancer (see, e.g., Paolucci & Rozengurt, 1999) and pancreatic cancer cell lines (see, e.g., Guha et al., 2002) contributing to increased colony formation, activation of the MEK/ERK pathway (see, e.g., Guha et al., 2003) and apoptotic blockade (see, e.g., Trauzold et al., 2003).
Inhibition of PKD1 and PKD2 activation by known pharmacological agents (e.g., GF 109203X, U0126) blocks proliferation and colony formation in pancreatic and small cell carcinoma cell lines (see, e.g., Guha et al., 2002, 2003).
The interaction of PKD1 signalling with other transduction pathways (e.g., c-JUN, EGF stimulation of proliferation) is altered in cancer-derived cell lines (see, e.g., Hurd, 2002; Hurd and Rozengurt, 2003).
Mouse skin carcinomas display increased PKD expression and over-expression of PKD1 potentiates DNA synthesis and cell proliferation induced by bombesin, vasopressin. and phorbol esters (see, e.g., Zugaza et al., 1997).
In breast cancer, PKD1 is recruited to the leading edge of the cells invading the surrounding tissue forming a complex with actin-binding protein contactin and the focal adhesion protein paxillin (see, e.g., Bowden et al., 1999).
Activation of PKD1 is required for increased adhesion of breast cancer cells to collagen in response to arachidoic acid (see, e.g., Kennett et al., 2004).
Expression of PKD1 correlates with keratinocyte proliferation (see, e.g., Rennecke et al., 1999) and is high in basal dividing cells but low in differentiating cells.
Over-expression of PKD1 reduces the sensitivity of several cell types (human and murine) to TNF induced apoptosis (see, e.g., Johannes et al., 1998).

PKD1 phosphorylation of RIN1 increases RAS/RAF interactions in Cos7 cells; the authors postulate this to be an important inhibition of a negative regulator of a tumourigenic pathway (see, e.g., Wang, 2002).

PKD1 and PKD2 have been shown to selectively phosphorylate HSP27 at serine 82, an event which modulates HSP27 oligomerization and activity. Inhibiting this reaction would potentially be of therapeutic benefit because HSP27 is reported as a survival factor and/or indicator of poor prognosis in prostate, breast and colon cancers. (see, e.g., Doppler, 2005, Gamido, 2003).

Results from an siRNA screen of human kinases has identified PKD2 as a survival kinase (see, e.g., Mackeigan et al., 2005).

Additionally, PKD1 and PKD2 activity is required for cell survival mediated by NF-κB in response to oxidative stress which can be relevant in malignancy especially where DNA damaging agents are being used (see, e.g., Storz & Toker, 2003; Storz et al., 2004a; Storz et al., 2004b). Therefore inhibitors of PKD1 and PKD2 may also be useful as chemo- or radio-potentiating agents.

### Hyperproliferative Skin Disorders

Keratinocytes undergo a distinct pattern of proliferation and differentiation that is essential for the function of the skin as a protective barrier. Defects in the equilibrium between proliferation and differentiation compromise the skin's barrier function and give rise to human diseases such as psoriasis and non-melanoma skin cancer. The identification of protein kinase C (PKC) as a major cellular target for tumor-promoting phorbol esters suggested the involvement of this enzyme in the regulation of keratinocyte proliferation and tumorigenesis; however, results have demonstrated the existence in keratinocytes and other cell types of another diacylglycerol/phorbol ester-responsive protein kinase: protein kinase D 1 (PKD1).

Current treatment strategies for hyperproliferative skin disorders are often suboptimal, either because of lack of efficacy or because of contraindications due to deleterious side effects or aesthetic considerations. Thus, small molecule PKD1 inhibitors could be useful for treatment of hyperproliferative skin disorders such as psoriasis, actinic keratosis and nonmelanoma skin cancers (see, e.g., Bollag et al 2004; Ristich, 2006).

### Angiogenesis

Activity of PKD1 is known to be required for Vascular Endothelial Growth Factor (VEGF) stimulated endothelial cell proliferation (see, e.g., Wong and Jin, 2005). VEGF is essential for many angiogenic processes both in normal conditions and in pathological conditions. VEGF rapidly and strongly stimulated PKD1 phosphorylation and activation in endothelial cells via VEGF receptor 2 (VEGFR2). Small interfering RNA knockdown of PKD1 and PKCalpha expression significantly attenuated ERK activation and DNA synthesis in endothelial cells by VEGF. Small interfering RNA knockdown of PKD1 expression significantly attenuates angiogenesis in a matrigel in vivo study (Qin, 2006). Taken together, this demonstrates that VEGF activates PKD1 via the VEGFR2/PLCgamma/PKCalpha pathway and reveals a critical role of PKD1 in angiogenesis, VEGF-induced ERK signaling and endothelial cell proliferation.

### Inflammation

PKD1 is highly expressed in both T and B lymphocytes, and antigen receptor engagement rapidly stimulates PKD1 activity (see, e.g., Matthews et al., 2000a, 2000b). In T-cells, PKD1 is rapidly activated and recruited to the plasma membrane (see, e.g., Matthews et al., 2000a). PKD1 residence at the membrane is relatively short, and during the prolonged phase of antigen-receptor activation PKD1 relocates to the cytosol where it remains active for several hours. PKD1 is thus able to transduce a transient signal generated by antigen receptors at the plasma membrane into a sustained signal in the cell interior. As a result, inhibitors of PKD1 could be useful for treatment of inflammatory diseases involving pathological activation of T- and B-cell lymphocytes, neutrophils and Mast cells.

### Heart Failure

In response to acute and chronic stresses, the heart frequently undergoes a remodeling process that is accompanied by myocyte hypertrophy, impaired contractility, and pump failure, often culminating in sudden death. The existence of redundant signaling pathways that trigger heart failure poses challenges for therapeutic intervention. Cardiac remodeling is associated with the activation of a pathological gene program that weakens cardiac performance. Thus, targeting the disease process at the level of gene expression represents a potentially powerful therapeutic approach (see, e.g., Vega et al., 2004; McKinsey and Olson 2005; WO04112763).

PKD1, PKD2, and PKD3 phosphorylate HDAC5 (Huynk Q K, 2006) which results in HDAC nuclear export. Importantly, small molecule inhibitors that target PKC and PKD1, PKD2, and PKD3, but not CaMK, abolish agonist-mediated nuclear export of HDAC5 cardiac myocytes, which suggests a predominant role for this pathway in the control of HDAC5 in the heart. One point on intervention in this process is via inhibition of Histone DeAcetylases (HDACs). Therefore small molecule PKD inhibitors could be used to block pathologic cardiac hypertrophy or heart failure.

WO 2004/078733 (Vertex Pharmaceuticals Incorporated) describes a large number of compounds that apparently are useful as inhibitors of voltage-gated sodium channels and calcium channels, and in the treatment of pain. It appears that some of these compounds may be the following:

| # | Structure | Name | Registry No. |
|---|---|---|---|
| A1 | 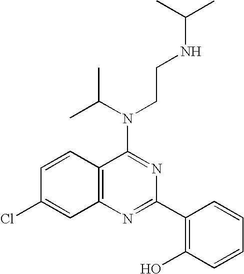 | 2-{7-chloro-4-[isopropyl- (2-isopropylamino-ethyl)- amino]-quinazolin-2-yl}-phenol | 757990-69-5 |
| A2 | 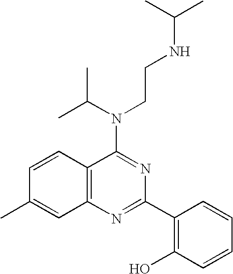 | 2-{7-methyl-4-[isopropyl-(2- isopropylamino-ethyl)-amino]- quinazolin-2-yl}-phenol | 757989-06-3 |
| A3 | 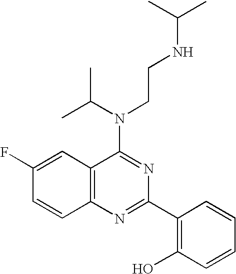 | 2-{6-Fluoro-4-[isopropyl-(2- isopropylamino-ethyl)-amino]- quinazolin-2-yl}-phenol | 757987-57-8 |
| A4 | 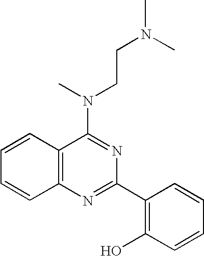 | 2-{4-[(2-Dimethylamino-ethyl)- methyl-amino]-quinazolin-2-yl}- phenol | 757984-15-9 (XX-110) |
| A5 | 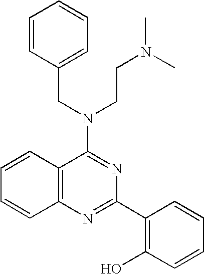 | 2-{4-[Benzyl-(2-dimethylamino- ethyl)-amino]-quinazolin-2-yl}- phenol | 757983-73-6 (XX-111) |
| A6 | 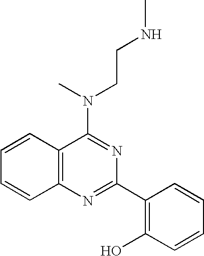 | 2-{4-[methyl-(2-methylamino- ethyl)-amino]-quinazolin-2-yl}- phenol | 757983-72-5 (XX-113) |

The following compounds may also be known (e.g., available from commercial sources):

| # | Structure | Name | Registry No. |
|---|---|---|---|
| B1 | 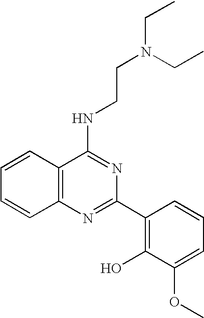 | 2-[4-(2-diethylamino- ethylamino)-quinazolin-2-yl]- 6-methoxy-phenol | 799791-56-3 |
| B2 | 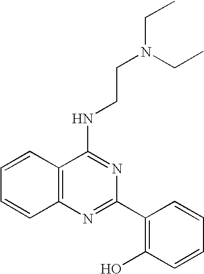 | 2-[4-(2-diethylamino- ethylamino)-quinazolin-2-yl]- phenol | 575460-22-9 |
| B3 | 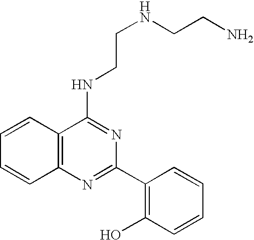 | 2-{4-[2-(2-amino-ethylamino)- ethylamino]-quinazolin-2-yl}- phenol | 406180-86-7 |
| B4 | 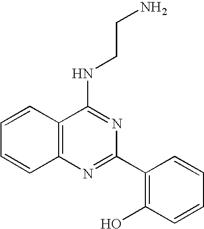 | 2-[4-(2-amino-ethylamino)- quinazolin-2-yl]-phenol | 406180-74-3 (XX-100) |

The following compounds may also be known (e.g., available from commercial sources):

| # | Structure | Name | Registry No. |
|---|---|---|---|
| C1 | 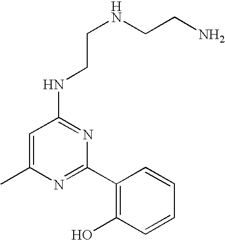 | 2-{4-[2-(2-amino-ethylamino)- ethylamino]-6-methyl-pyrimidin- 2-yl}-phenol | 406181-01-9 |
| C2 | 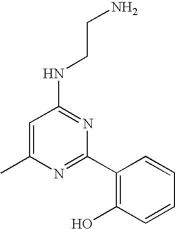 | 2-[4-(2-amino-ethylamino)-6- methyl-pyrimidin-2-yl]-phenol | 381704-89-8 |

The following compound may also be known (e.g., available from commercial sources):

| # | Structure | Name | Registry No. |
|---|---|---|---|
| D1 | 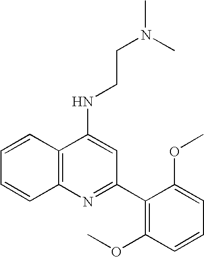 | N′-[2-(2,6- dimethoxy- phenyl)- quinolin-4-yl]- N,N-dimethyl- ethane-1,2- diamine | 790197-49-8 |

WO 2000/076982 (University of Iowa Research Foundation) describes a large number of compounds that apparently are useful as inhibitors of the immune system. It appears that one of these compounds may be the following (see Compound 7.26 in FIG. 1J therein):

| | | | |
|---|---|---|---|
| E1 | 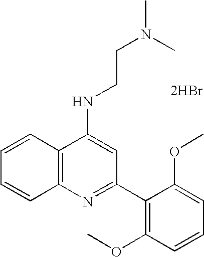 | N′-[2-(2,6- dimethoxy- phenyl)- quinolin-4-yl]- N,N-dimethyl- ethane-1,2- diamine bis (hydrobromide) | 313829-12-8 |

### BRIEF DESCRIPTION OF THE DRAWINGS

FIG. 1 shows the DNA sequence corresponding to murine PKD1.

FIG. 2 shows the amino acid sequence for the murine PKD1 protein used in the biological studies.

FIG. 3 shows the alignment of the kinase domain of murine PKD1 (mPKD1) with those of human PKD1, PKD2, and PKD3 (hPKD1, hPKD2, hPKD3, respectively). Those residues within the ATP binding site are shown in bold, and are completely conserved across the sequences. The kinase domain of murine PKD1 is 99.6%, 91.8% and 93.8% identical to, and 99.7%, 95.4% and 96.5% similar to, human PKD1, PKD2, and PKD3 respectively. The biological data generated in respect of compounds using murine PKD1 are predictive of their activity in respect of any of the human PKD isoforms.

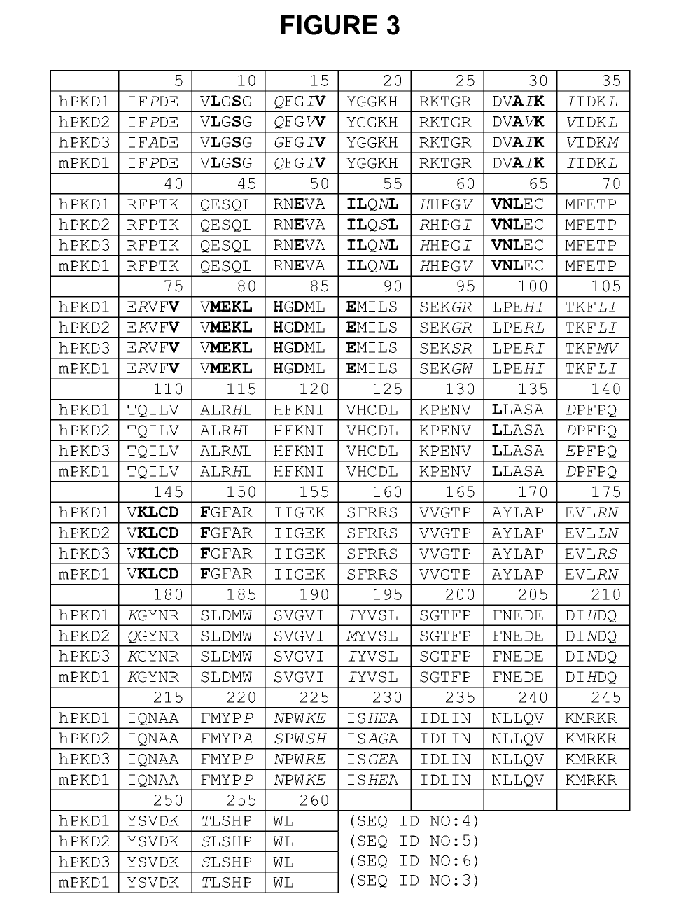

FIG. 4 is a photographic depiction of the western blot analysis of cell lysates of PANC-1 cells which were treated with increasing amounts (2, 5, 10, 30 μM) of an amino-ethyl-amino-aryl (AEAA) compound (XX-032), as described below for the Western Blot 916 (Phospho-Ser916 PKD1) Assay. Cell lysates were analysed using an anti-human PKD1 Antibody (middle panel), anti-phospho-human PKD1 (Ser916) Antibody (top panel) and anti-tubulin antibody (lower panel).

FIG. 5 is a depiction of the quantification of the western blot as shown in FIG. 4. The shown columns represent the percentage phosphorylation as measured by densitometry of phospho-human PKD1 (Ser916) levels, as described below for the Western Blot 916 Assay. The results were normalised to the measured PKD levels and expressed as a percentage of the level of phosphorylation in the PDBu-stimulated control.

FIG. 6 is a graphic representation of the results of the proliferation assay, as described below. The columns in the graph represent the mean percentage of BrdU incorporation into PANC-1 cells as a measure for cell proliferation. The two left-hand columns represent the controls of DMSO (basal level of non-stimulated cell proliferation) and DMSO plus 50 nM neurotensin (stimulated cell proliferation). The two right-hand columns represent the effect of two different concentrations (5 μM and 2 μM) of an amino-ethyl-amino-aryl (AEAA) compound (XX-032) on the neurotensin-stimulated cell proliferation. The graph illustrates that an increasing the amount of the amino-ethyl-amino-aryl (AEAA) compound inhibited stimulated cell proliferation.

FIG. 7 shows a graphic representation of the results obtained in the apoptosis assay, as described below. The depicted columns show the change in viability or induction of apotosis in the presence of an amino-ethyl-amino-aryl (AEAA) compound (XX-032). Cell viability was measured by the MTT assay at two different time points (24 and 48 hours) and induction of apoptosis was measured by the caspase assay at two different time points (24 and 48 hours). The data are expressed as a percentage of the level in the corresponding control.

### SUMMARY OF THE INVENTION

One aspect of the invention pertains to certain amino-ethyl-amino-aryl (AEAA) compounds, as described herein.

Another aspect of the invention pertains to a composition (e.g., a pharmaceutical composition) comprising an AEAA compound, as described herein, and a pharmaceutically acceptable carrier or diluent.

Another aspect of the invention pertains to method of preparing a composition (e.g., a pharmaceutical composition) comprising the step of admixing an AEAA compound, as described herein, and a pharmaceutically acceptable carrier or diluent.

Another aspect of the present invention pertains to a method of inhibiting PKD (e.g., PKD1, PKD2, PKD3) in a cell, in vitro or in vivo, comprising contacting the cell with an effective amount of an AEAA compound, as described herein.

Another aspect of the present invention pertains to a method of regulating (e.g., inhibiting) cell proliferation (e.g., proliferation of a cell), inhibiting cell cycle progression, promoting apoptosis, or a combination of one or more these, in vitro or in vivo, comprising contacting cells (or the cell) with an effective amount of an AEAA compound, as described herein.

Another aspect of the present invention pertains to a method for treatment comprising administering to a subject in need of treatment a therapeutically-effective amount of an AEAA compound, as described herein, preferably in the form of a pharmaceutical composition.

Another aspect of the present invention pertains to an AEAA compound as described herein for use in a method of treatment of the human or animal body by therapy.

Another aspect of the present invention pertains to use of an AEAA compound, as described herein, in the manufacture of a medicament for use in treatment.

In one embodiment, the treatment is treatment of a disease or condition that is mediated by PKD (e.g., PKD1, PKD2, PKD3).

In one embodiment, the treatment is treatment of a disease or condition that is ameliorated by the inhibition of PKD (e.g., PKD1, PKD2, PKD3).

In one embodiment, the treatment is treatment of a proliferative condition.

In one embodiment, the treatment is treatment of cancer.

In one embodiment, the treatment is treatment of a hyperproliferative skin disorder, for example, psoriasis, actinic keratosis, and/or non-melanoma skin cancer.

In one embodiment, the treatment is treatment of a disease or condition that is characterised by inappropriate, excessive, and/or undesirable angiogenesis, for example, macular degeneration, cancer (solid tumours), psoriasis, and obesity.

In one embodiment, the treatment is treatment of an inflammatory disease.

In one embodiment, the treatment is treatment a disease or disorder associated with heart remodelling, myocyte hypertrophy of the heart, impaired contractility of the heart, pump failure of the heart, pathologic cardiac hypertrophy, and/or heart failure.

Another aspect of the present invention pertains to a kit comprising (a) an AEAA compound, as described herein, preferably provided as a pharmaceutical composition and in a suitable container and/or with suitable packaging; and (b) instructions for use, for example, written instructions on how to administer the compound.

Another aspect of the present invention pertains to an AEAA compound obtainable by a method of synthesis as described herein, or a method comprising a method of synthesis as described herein.

Another aspect of the present invention pertains to an AEAA compound obtained by a method of synthesis as described herein, or a method comprising a method of synthesis as described herein.

Another aspect of the present invention pertains to novel intermediates, as described herein, which are suitable for use in the methods of synthesis described herein.

Another aspect of the present invention pertains to the use of such novel intermediates, as described herein, in the methods of synthesis described herein.

As will be appreciated by one of skill in the art, features and preferred embodiments of one aspect of the invention will also pertain to other aspect of the invention.

### DETAILED DESCRIPTION OF THE INVENTION

### Compounds

One aspect of the present invention pertains to compound selected from compounds of the following formula and pharmaceutically acceptable salts, solvates, hydrates, ethers, esters, chemically protected forms, and prodrugs thereof (collectively denoted “amino-ethyl-amino-aryl (AEAA) compounds”):

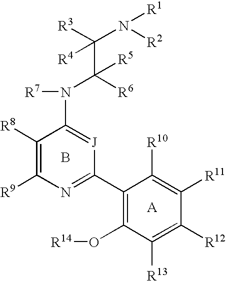

wherein:
J is independently N or CH;
and wherein:
(1) each of R⁸ and R⁹ is independently —H or a Ring B substituent;
or:
(2) R⁸ and R⁹, taken together with the atoms to which they are attached, form an aromatic Ring C having exactly 5 ring atoms or exactly 6 ring atoms, wherein each ring atom is a carbon ring atom or a nitrogen ring atom, wherein Ring C has exactly 0, exactly 1, or exactly 2 ring nitrogen atoms, and wherein Ring C is fused to Ring B;
and wherein:
(1) each of R¹⁰, R¹¹, R¹², and R¹³ is independently —H or a Ring A substituent;
or:
(2) each of R¹² and R¹³ is independently a —H or Ring A substituent; and R¹⁰ and R¹¹, taken together with the atoms to which they are attached, form an aromatic Ring D having exactly 6 ring atoms, wherein each ring atom is a carbon ring atom, and wherein Ring D is fused to Ring A;
or:
(3) each of R¹⁰ and R¹³ is independently —H or a Ring A substituent; and R¹¹ and R¹², taken together with the atoms to which they are attached, form an aromatic Ring E having exactly 6 ring atoms, wherein each ring atom is a carbon ring atom, and wherein Ring E is fused to Ring A;
or:
(4) each of R¹⁰ and R¹¹ is independently —H or a Ring A substituent; and R¹² and R¹³, taken together with the atoms to which they are attached, form an aromatic Ring F having exactly 6 ring atoms, wherein each ring atom is a carbon ring atom, and wherein Ring F is fused to Ring A;
and wherein:
each of R¹, R², R³, R⁴, R⁵, R⁶, and R⁷ is independently —H or a group G;
and additionally wherein:
each of R³, R⁴, R⁵, and R⁶ may be a group Y;
each of R¹, R², and R⁷ may be a group Z;
R³ and R⁴, taken together, may form a group ═O;
R⁵ and R⁶, taken together, may form a group ═O;
and wherein:
R¹⁴ is independently —H or a group W.

### Optional Provisos

In one or more aspects of the present invention (e.g., compounds, compositions, methods of treatment, compounds for use in therapy, use of compounds in the manufacture of a medicament, etc.), the compounds are optionally as defined herein, but with one or more optional provisios, as defined herein.

In one embodiment, the proviso is that the compound is not:
(A1) 2-{7-chloro-4-[isopropyl-(2-isopropylamino-ethyl)-amino]-quinazolin-2-yl}-phenol;
(A2) 2-{7-methyl-4-[isopropyl-(2-isopropylamino-ethyl)-amino]-quinazolin-2-yl}-phenol;
(A3) 2-{6-Fluoro-4-[isopropyl-(2-isopropylamino-ethyl)-amino]-quinazolin-2-yl}-phenol;
(A4) 2-{4-[(2-Dimethylamino-ethyl)-methyl-amino]-quinazolin-2-yl}-phenol (XX-110);
(A5) 2-{4-[Benzyl-(2-dimethylamino-ethyl)-amino]-quinazolin-2-yl}-phenol (XX-111);
(A6) 2-{4-[methyl-(2-methylamino-ethyl)-amino]-quinazolin-2-yl}-phenol (XX-113);
(B1) 2-[4-(2-diethylamino-ethylamino)-quinazolin-2-yl]-6-methoxy-phenol;
(B2) 2-[4-(2-diethylamino-ethylamino)-quinazolin-2-yl]-phenol;
(B3) 2-{4-[2-(2-amino-ethylamino)-ethylamino]-quinazolin-2-yl}-phenol;
(B4) 2-[4-(2-amino-ethylamino)-quinazolin-2-yl]-phenol (XX-100);
(C1) 2-{4-[2-(2-amino-ethylamino)-ethylamino]-6-methyl-pyrimidin-2-yl}-phenol;
(C2) 2-[4-(2-amino-ethylamino)-6-methyl-pyrimidin-2-yl]-phenol;
(D1) N′-[2-(2,6-dimethoxy-phenyl)-quinolin-4-yl]-N,N-dimethyl-ethane-1,2-diamine; or
(E1) N′-[2-(2,6-dimethoxy-phenyl)-quinolin-4-yl]-N,N-dimethyl-ethane-1,2-diamine bis(hydrobromide).

In one or more aspects of the present invention (e.g., compounds for use in therapy, use of compounds in the manufacture of a medicament, methods of treatment, etc.), the compounds are optionally as defined herein, but without the above proviso.

For example, a reference to a particular group of compounds “without the recited proviso” (e.g., for use in therapy) is intended to be a reference to the compounds as defined, but wherein the definition no longer includes the indicated proviso. In such cases, it is as if the indicated proviso has been deleted from the definition of compounds, and the definition has been expanded to encompass those compounds which otherwise would have been excluded by the indicated proviso.

### Structural Subdivision

For convenience, the structure of the compounds may be subdivided into three moieties:

(1) the amino-ethylene-amino group (M),
(2) the heteroaromatic core (Q), and
(3) the carboaromatic core (T),
as illustrated below:

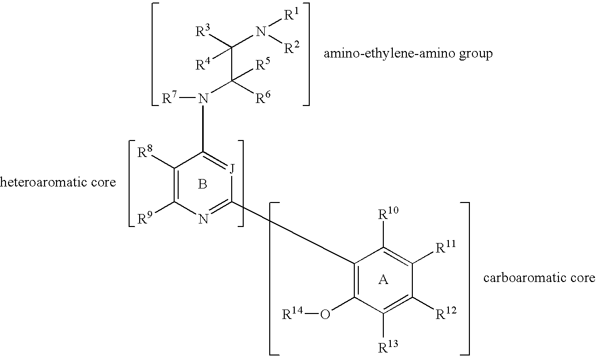

For convenience, the structure of the compounds may be represented as M-Q-T:

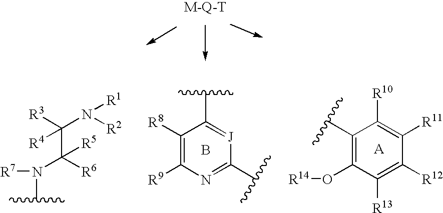

### Stereoisomerism

For the avoidance of doubt, it is intended that these three moieties are only linked as shown. For example, it is not intended that R⁷ and R⁸ together form one group. Similarly, it is not intended that R⁹ and R¹⁴ form one group.

Many of the chemical structures shown herein indicate one or more specific stereoisomeric configurations. Similarly, many of the chemical structures shown herein are silent in this respect, and do not indicate any stereoisomeric configuration. Similarly, many of the chemical structures shown herein indicate the specific stereoisomeric configurations at one or more positions, but are silent with respect to one or more other positions. Where a chemical structure herein is silent with respect to the stereoisomeric configuration at a position, that structure is intended to depict all possible stereoisomeric configuration at that position, both individually, as if each possible stereoisomeric configuration was individually recited, and also as a mixture (e.g., a racemic mixture) of stereoisomers. For example, SI denotes each of S4, S5, S6, and S7. Similarly, S2 denotes both S4 and S5; and S3 denotes both S6 and S7.

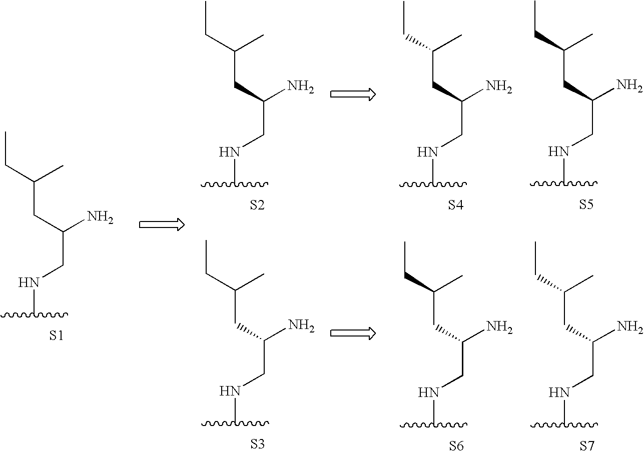

### The Group J

In one embodiment, J is independently N.

In one embodiment, J is independently CH.

### The Groups R⁸ and R⁹: Ring C is Absent

In one embodiment, each of R⁸ and R⁹ is independently —H or a Ring B substituent.

In one embodiment, each of R⁸ and R⁹ is independently a Ring B substituent.

In one embodiment, R⁸ is independently a Ring B substituent; and R⁹ is independently —H.

In one embodiment, R⁹ is independently a Ring B substituent; and R⁸ is independently —H.

In one embodiment, each of R⁸ and R⁹ is independently H, as in, for example:

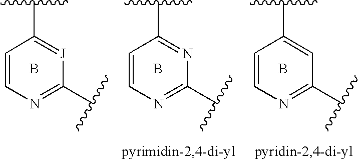

### The Groups R⁸ and R⁹: Ring C is Present

In one embodiment, R⁸ and R⁹, taken together with the atoms to which they are attached, form an aromatic Ring C having exactly 5 ring atoms or exactly 6 ring atoms, wherein each ring atom is a carbon ring atom or a nitrogen ring atom, wherein Ring C has exactly 0, exactly 1, or exactly 2 ring nitrogen atoms, and wherein Ring C is fused to Ring B.

In one embodiment, Ring C, if present, has exactly 5 ring atoms.

In one embodiment, Ring C, if present, has exactly 6 ring atoms.

In one embodiment, Ring C, if present, has exactly 0 ring nitrogen atoms.

In one embodiment, Ring C, if present, has exactly 1 ring nitrogen atom.

In one embodiment, Ring C, if present, has exactly 2 ring nitrogen atoms.

In one embodiment, R⁸ and R⁹, taken together with the atoms to which they are attached, form an aromatic Ring C having exactly 5 ring atoms, wherein each ring atom is a carbon ring atom or a nitrogen ring atom, wherein Ring C has exactly 0, exactly 1, or exactly 2 ring nitrogen atoms, and wherein Ring C is fused to Ring B.

In one embodiment, R⁸ and R⁹, taken together with the atoms to which they are attached, form an aromatic Ring C having exactly 6 ring atoms, wherein each ring atom is a carbon ring atom or a nitrogen ring atom, wherein Ring C has exactly 0, exactly 1, or exactly 2 ring nitrogen atoms, and wherein Ring C is fused to Ring B.

In one embodiment, R⁸ and R⁹, taken together with the atoms to which they are attached, form an aromatic Ring C having exactly 6 ring atoms, wherein each ring atom is a carbon ring atom, and wherein Ring C is fused to Ring B, for example, as in the following groups:

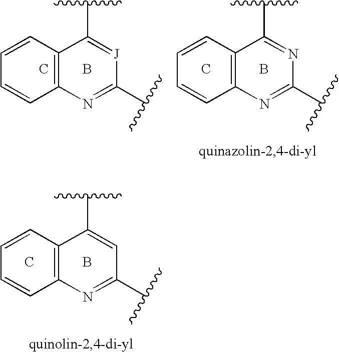

In one embodiment, Ring C, if present, independently is unsubstituted, or is substituted with one or more (e.g., 1, 2, 3, 4) Ring C substituents.

For the avoidance of doubt, it is not intended that Ring C substituents, if present, form a fused ring with Ring C and/or Ring C and Ring B.

In one embodiment, Ring C, if present, independently is unsubstituted.

### The Heteroaromatic Core, Q

In one embodiment, the “heteroaromatic core”, Q, shown below:

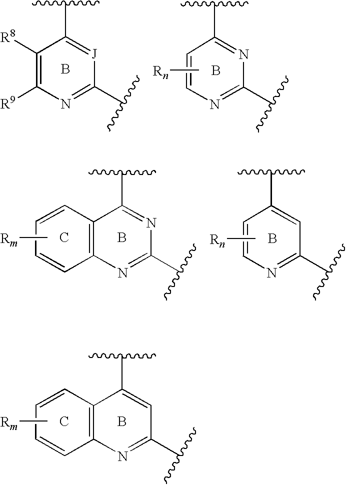

is independently selected from:
wherein:
each n is independently 0, 1, or 2;
each m is independently 0, 1, 2, 3, or 4; and
each R, if present, is independently 1° a carbo-substituent or a 1° hetero-substituent.

In one embodiment, each n is independently 0.

In one embodiment, each n is independently 1.

In one embodiment, each n is independently 2.

In one embodiment, each m is independently 0.

In one embodiment, each m is independently 1.

In one embodiment, each m is independently 2.

In one embodiment, each m is independently 3.

In one embodiment, each m is independently 4.

In one embodiment, the “heteroaromatic core” is independently selected from:

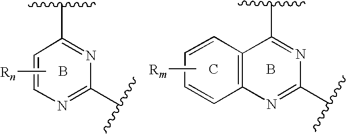

In one embodiment, the “heteroaromatic core” is:

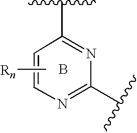

In one embodiment, the “heteroaromatic core” is:

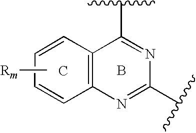

In one embodiment, each of R⁸ and R⁹ is independently selected from: —H, —F, —Cl, —Br, —I, C₁₋₇alkyl, pyrazole, or phenyl; wherein each pyrazole and phenyl, if present, is optionally substituted, for example, with one or more substituents selected from: —F, —Cl, —Br, —I, —OH, C₁₋₇alkyl, and —O—C₁₋₄alkyl.

In one embodiment, R⁸ is independently selected from: —H, —F, —Cl, —Br, —I, C₁₋₇alkyl, pyrazole, or phenyl; wherein each pyrazole and phenyl, if present, is optionally substituted, for example, with one or more substituents selected from: —F, —Cl, —Br, —I, —OH, C₁₋₇alkyl, and —O—C₁₋₄alkyl; and R⁹ is independently selected from: —H and C₁₋₄alkyl.

### The Groups R¹⁰, R¹¹, R¹² and R¹³: Ring D, Ring E, and Ring F are Absent

In one embodiment, each of R¹⁰, R¹¹, R¹², and R¹³ is independently —H or a Ring A substituent.

In one embodiment, each of R¹⁰, R¹¹, R¹², and R¹³ is independently a Ring A substituent.

In one embodiment, each of R¹⁰, R¹², and R¹³ is —H, and R¹¹ is independently a Ring A substituent.

In one embodiment, each of R¹⁰, R¹¹, R¹², and R¹³ is independently —H.

### The Groups R¹⁰, R¹¹, R¹², and R¹³: Ring D is Present

In one embodiment, each of R¹² and R¹³ is independently —H or a Ring A substituent; and R¹⁰ and R¹¹, taken together with the atoms to which they are attached, form an aromatic Ring D having exactly 6 ring atoms, wherein each ring atom is a carbon ring atom, and wherein Ring D is fused to Ring A, for example, as in the following group:

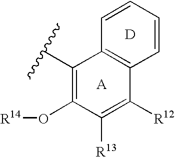

In one embodiment, each of R¹² and R¹³ is independently —H.

In one embodiment, Ring D, if present, independently is unsubstituted, or is substituted with one or more (e.g., 1, 2, 3, 4) Ring D substituents.

For the avoidance of doubt, it is not intended that Ring D substituents, if present, form a fused ring with Ring D and/or Ring D and Ring A.

In one embodiment, Ring D, if present, independently is unsubstituted.

### The Groups R¹⁰, R¹¹, R¹², and R¹³: Ring E is Present

In one embodiment, each of R¹⁰ and R¹³ is independently —H or a Ring A substituent; and R¹¹ and R¹², taken together with the atoms to which they are attached, form an aromatic Ring E having exactly 6 ring atoms, wherein each ring atom is a carbon ring atom, and wherein Ring E is fused to Ring A, for example, as in the following group:

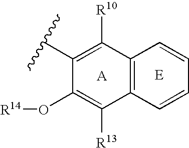

In one embodiment, each of R¹⁰ and R¹³ is independently —H.

In one embodiment, Ring E, if present, independently is unsubstituted, or is substituted with one or more (e.g., 1, 2, 3, 4) Ring E substituents.

For the avoidance of doubt, it is not intended that Ring E substituents, if present, form a fused ring with Ring E and/or Ring E and Ring A.

In one embodiment, Ring E, if present, independently is unsubstituted.

### The Groups R¹⁰, R¹¹, R¹², and R¹³: Ring F is Present

In one embodiment, each of R¹⁰ and R¹¹ is independently —H or a Ring A substituent; and R¹² and R¹³, taken together with the atoms to which they are attached, form an aromatic Ring F having exactly 6 ring atoms, wherein each ring atom is a carbon ring atom, and wherein Ring F is fused to Ring A, for example, as in the following group:

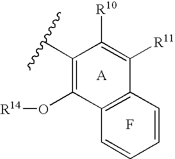

In one embodiment, each of R¹⁰ and R¹¹ is independently —H.

In one embodiment, Ring F, if present, independently is unsubstituted, or is substituted with one or more (e.g., 1, 2, 3, 4) Ring F substituents.

For the avoidance of doubt, it is not intended that Ring F substituents, if present, form a fused ring with Ring F and/or Ring F and Ring A.

In one embodiment, Ring F, if present, independently is unsubstituted.

### The Carboaromatic Core, T

In one embodiment, the “carboaromtic core”, shown below:

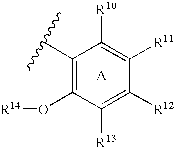

is independently selected from:

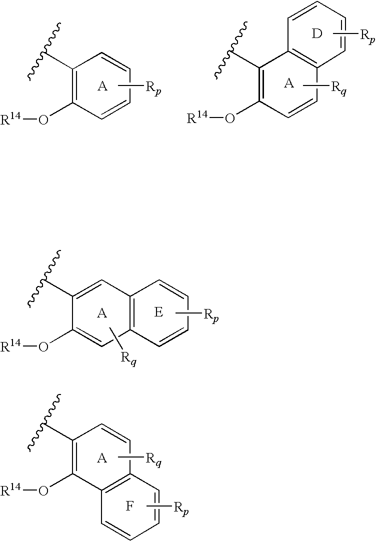

wherein:
each p is independently 0, 1, 2, 3, or 4;
each q is independently 0, 1, or 2; and
each R, if present, is independently a 1° carbo-substituent or a 1° hetero-substituent.

In one embodiment, each p is independently 0.

In one embodiment, each p is independently 1.

In one embodiment, each p is independently 2.

In one embodiment, each p is independently 3.

In one embodiment, each p is independently 4.

In one embodiment, each q is independently 0.

In one embodiment, each q is independently 1.

In one embodiment, each q is independently 2.

In one embodiment, the “carboaromatic core” is independently selected from:

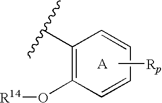

In one embodiment, the “carboaromatic core” is independently selected from:

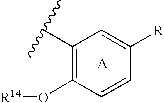

In one embodiment, the “carboaromatic core” is independently selected from:

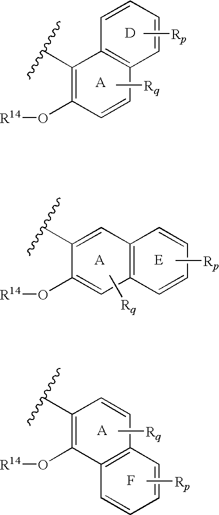

### Combinations of the Heteroaromatic Core (Q) and Carboaromatic Core (T)

In one embodiment, the combined heteroaromatic core and carboaromatic core (-Q-T) is a moiety independently selected from the following moieties:

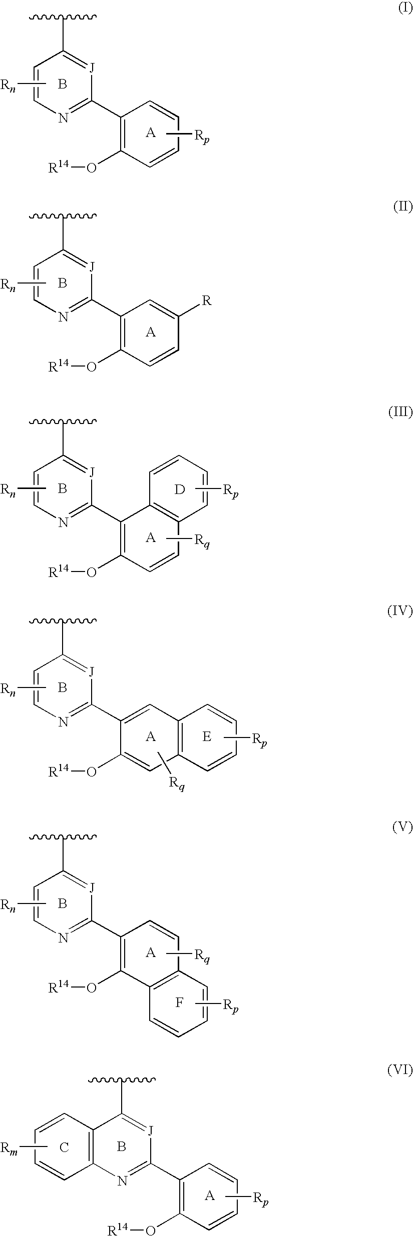

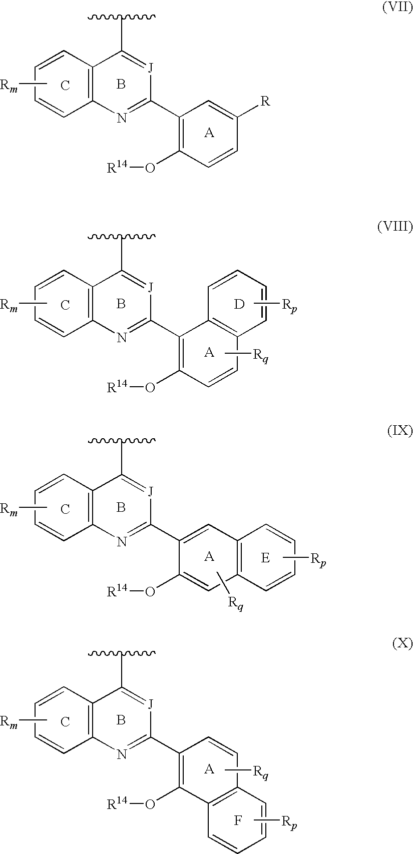

In one embodiment, the combined heteroaromatic core and carboaromatic core (-Q-T) is a moiety independently selected from the following moieties: (I), (II), (VI), and (VII).

In one embodiment, the combined heteroaromatic core and carboaromatic core (-Q-T) is a moiety independently selected from the following moieties: (II) and (VII).

In one embodiment, the combined heteroaromatic core and carboaromatic core (-Q-T) is a moiety independently the following moiety: (II).

In one embodiment, the combined heteroaromatic core and carboaromatic core (-Q-T) is a moiety independently the following moiety: (VII).

In one embodiment, the combined heteroaromatic core and carboaromatic core (-Q-T) is selected from the above, where J is N.

In one embodiment, the combined heteroaromatic core and carboaromatic core (-Q-T) is selected from the above, where J is CH.

In one embodiment, each R, if present, is independently a 1° carbo-substituent or a 1° hetero-substituent.

In one embodiment, each R, if present, is independently a 1° carbo-substituent selected from: (C-1), (C-3), (C-7), (C-8), (C-9) and (C-10), as defined herein, or a 1° hetero-substituent selected from: (H-1), (H-2), (H-3), (H-5), (H-6), (H-10), (H-11), (H-12), (H-13), (H-14), (H-21) and (H-22), as defined herein.

In one embodiment, each R, if present, is independently:
—F, —Cl, —Br, —I,
R^{D1},
—CF₃,
—OH,
-L¹-OH,
—OR^{D1},
-L¹-OR^{D1},
—OCF₃,
—SH,
SR^{D1},
—SCF₃,
—CN,
—NO₂,
—NH₂, —NHR^{D1}, NR^{D1}₂, NR^{N1}R^{N2},
-L¹-NH₂, -L¹NHR^{D1}, -L¹-NR^{D1}₂, -L¹-NR^{N1}R^{N2},
—C(═O)OH,
—C(═O)OR^{D1},
—C(═O)NH₂, —C(═O)NHR^{D1}, —C(═O)NR^{D1}₂, —C(═O)NR^{N1}R^{N2},
—NHC(═O)R^{D1}, —NR^{D1}C(═O)R^{D1},
—OC(═O)R^{D1},
—C(═O)R^{D},
—NHS(═O)₂R^{D1}, —NR^{D1}S(═O)₂R^{D1},
—S(═O)₂NH₂, —S(═O)₂NHR^{D1}, —S(═O)₂NR^{D1}₂, —S(═O)₂NR^{N1}R^{N2},
—S(═O)₂R^{D1},
—OS(═O)₂R^{D1}, or
—S(═O)₂OR^{D1},
and additionally, two adjacent R groups, if present, may together form a group —O-L²-O—;
   wherein:
each -L¹- is independently saturated aliphatic C₂₋₅alkylene;
each -L²- is independently saturated aliphatic C₁₋₃alkylene;
in each group —NR^{N1}R^{N2}, R^{N1} and R^{N2}, taken together with the nitrogen atom to which they are attached, form a 5-, 6-, or 7-membered non-aromatic ring having exactly 1 ring heteroatom or exactly 2 ring heteroatoms, wherein one of said exactly 2 ring heteroatoms is N, and the other of said exactly 2 ring heteroatoms is independently N or O;
each —R^{D1} is independently:
   —R^{E1}, —R^{E2}, —R^{E3}, —R^{E4}, —R^{E5}, —R^{E6}, —R^{E7}, —R^{E8},
   -L³-R^{E4}, -L³-R^{E5}, -L³-R^{E6}, -L³-R^{E7}, or -L³-R^{E8};
      wherein:
each —R^{E1} is independently saturated aliphatic C₁₋₆alkyl;
each —R^{E2} is independently aliphatic C₂₋₆alkenyl;
each —R^{E3} is independently aliphatic C₂₋₆alkynyl;
each —R^{E4} is independently saturated C₃₋₆cycloalkyl;
each —R^{E5} is independently C₃₋₆cycloalkenyl;
each —R^{E6} is independently non-aromatic C₃₋₇heterocyclyl;
each —R^{E7} is independently C₆₋₁₄carboaryl;
each —R^{E8} is independently C₅₋₁₄heteroaryl;
each -L³- is independently saturated aliphatic C₁₋₃alkylene;
   and wherein:
each C₁₋₆alkyl, C₂₋₆alkenyl, C₂₋₆alkynyl, C₃₋₆cycloalkyl, C₃₋₆cycloalkenyl, non-aromatic C₃₋₇heterocyclyl, C₆₋₁₄carboaryl, C₅₋₁₄heteroaryl, and C₁₋₃alkylene is optionally substituted with one or more substituents selected from:
—F, —Cl, —Br, —I,
R^{F1},
—CF₃,
—OH,
—OR^{F1},
—OCF₃,
—SH,
—SR^{F1},
—SCF₃,
—CN,
—NO₂,
—NH₂, —NHR^{F1}, NR^{F1}₂, —NR^{N3}R^{N4},
—C(═O)OH,
—C(═O)OR^{F1},
—C(═O)NH₂, —C(═O)NHR^{F1}, —C(═O)NR^{F1}₂, —C(═O)NR^{N3}R^{N4},
-L⁴-OH, -L⁴-OR^{F1},
-L⁴-NH₂, -L⁴-NHR^{F1}, -L⁴-NR^{F1}₂, or -L⁴ NR_{N3}R^{N4};
   wherein:
each —R^{F1} is independently saturated aliphatic C₁₋₄alkyl;
each -L⁴- is independently saturated aliphatic C₂₋₅alkylene; and
in each group —NR^{N3}R^{N4}, R^{N3} and R^{N4}, taken together with the nitrogen atom to which they are attached, form a 5-, 6-, or 7-membered non-aromatic ring having exactly 1 ring heteroatom or exactly 2 ring heteroatoms, wherein one of said exactly 2 ring heteroatoms is N, and the other of said exactly 2 ring heteroatoms is independently N or O.

In one embodiment, each —NR^{N1}R^{N2}, if present, is independently pyrrolidino, imidazolidino, pyrazolidino, piperidino, piperizino, morpholino, azepino, or diazepino, and is independently unsubstituted or substituted with one or more groups selected from C₁₋₃alkyl and —CF₃.

In one embodiment, each —NR^{N1}R^{N2}, if present, is independently pyrrolidino, piperidino, piperizino, or morpholino, and is independently unsubstituted or substituted with one or more groups selected from C₁₋₃alkyl and —CF₃.

In one embodiment, each -L²-, if present, is independently —CH₂—.

In one embodiment, each —R^{D1}, if present, is independently:

—R^{E1}, —R^{E4}, —R^{E7}, —R^{E8}, -L³-R^{E4}, -L³-R^{E7}, or -L³-R^{E8}.

In one embodiment, each —R^{E7}, if present, is independently phenyl, and is optionally substituted.

In one embodiment, each —R^{E8}, if present, is independently C₅₋₆heteroaryl, and is optionally substituted.

In one embodiment, each —R^{E8}, if present, is independently furanyl, thienyl, pyrrolyl, imidazolyl, pyrazolyl, oxazole, isoxazole, thiazole, isothiazole, pyridyl, pyrimidinyl, and pyridazinyl, and is optionally substituted.

In one embodiment, each —NR^{N3}R^{N4}, if present, is independently pyrrolidino, imidazolidino, pyrazolidino, piperidino, piperizino, morpholino, azepino, or diazepino, and is independently unsubstituted or substituted with one or more groups selected from C₁₋₃alkyl and —CF₃.

In one embodiment, each —NR^{N3}R^{N4}, if present, is independently pyrrolidino, piperidino, piperizino, or morpholino, and is independently unsubstituted or substituted with one or more groups selected from C₁₋₃alkyl and —CF₃.

In one embodiment, each R, if present, is independently selected from: —F, —Cl, —Br, —I, —OH, —O—C₁₋₇alkyl, —O—C₁₋₇haloalkyl, —S—C₁₋₇alkyl, —NH₂, —NH—C₁₋₇alkyl, —N(C₁₋₇alkyl)₂, —C(═O)OH, —C(═O)O—C₁₋₇alkyl, —C(═O)NH₂, —OC(═O)—C₁₋₇alkyl, —NO₂, C₁₋₇alkyl, —C₁₋₇haloalkyl, —CH₂-Ph, -Ph, -Ph-C₁₋₇haloalkyl.

In one embodiment, each R, if present, is independently selected from: —F, —Cl, —Br, —I, —OH, —OMe, —OCF₃, —SMe, —NH₂, —NHMe, —NMe₂, —C(═O)OH, —C(═O)OMe, —C(═O)NH₂, —OC(═O)Me, —NO₂, -Me, —CF₃, —CH₂-Ph, -Ph, -Ph-CF₃.

In one embodiment, the substituent on Ring A at the position para to the group —O—R¹⁴ (as in, for example, —R in moieties (II) and (VII), and also —R¹¹ in the formulae herein), if present, is independently —R^{G1}, wherein —R^{G1} is independently —R^{H7} or —R^{H8}, and wherein —R^{H7}, if present, is independently phenyl and —R^{H8}, if present, is independently pyrazolyl or pyridyl; and wherein said phenyl, pyrazolyl, or pyridyl is optionally substituted with one or more substituents selected from:
—F, —Cl, —Br, —I,
—R^{J1},
—CF₃,
—OH,
—OR^{J1},
—OCF₃,
—SH,
—SR^{J1},
—SCF₃,
—CN,
—NO₂,
—NH₂, —NHR^{J1}, —NR^{J1}₂, —NR^{N5}R^{N6},
—C(═O)OH,
—C(═O)OR^{J1},
—C(═O)NH₂, —C(═O)NHR^{J1}, —C(═O)NR^{J1}₂, —C(═O)NR^{N5}R^{N6},
-L⁵-OH, -L⁵-OR^{J1},
-L⁵-NH₂, -L⁵-NHR^{J1}, -L⁵-NR^{J1}₂, or -L⁵-NR^{N5}R^{N6};
   wherein:
each —R^{J1} is independently saturated aliphatic C₁₋₄alkyl;
each -L⁵- is independently saturated aliphatic C₂₋₅alkylene; and
in each group —NR^{N5}R^{N6}, R^{N5} and R^{N6}, taken together with the nitrogen atom to which they are attached, form a 5-, 6-, or 7-membered non-aromatic ring having exactly 1 ring heteroatom or exactly 2 ring heteroatoms, wherein one of said exactly 2 ring heteroatoms is N, and the other of said exactly 2 ring heteroatoms is independently N or O.

In one embodiment, each —NR^{N5}R^{N6}, if present, is independently pyrrolidino, imidazolidino, pyrazolidino, piperidino, piperizino, morpholino, azepino, or diazepino, and is independently unsubstituted or substituted with one or more groups selected from C₁₋₃alkyl and —CF₃.

In one embodiment, each —NR^{N5}R^{N6}, if present, is independently pyrrolidino, piperidino, piperizino, or morpholino, and is independently unsubstituted or substituted with one or more groups selected from C₁₋₃alkyl and —CF₃.

In one embodiment, the substituent on Ring A at the position para to the group —O—R¹⁴ (as in, for example, —R in moieties (II) and (VII), and also —R¹¹ in the formulae herein), if present, is independently —F, —Cl, —Br, —I, phenyl, pyrazolyl, or pyridyl; wherein said phenyl, pyrazolyl, or pyridyl is optionally substituted, for example, with one or more substituents independently selected from: —F, —Cl, —Br, —I, C₁₋₆alkyl, —CF₃, —OH, —O—C₁₋₆alkyl, and —OCF₃.

In one embodiment, the substituent on Ring A at the position para to the group —O—R¹⁴ (as in, for example, —R in moieties (II) and (VII), and also —R¹¹ in the formulae herein), if present, is independently pyrazolyl, wherein said pyrazolyl is optionally substituted, for example, with one or more C₁₋₆alkyl groups.

### Ring A Substituents

In one embodiment, each Ring A substituent, if present, is independently a 1° carbo-substituent or a 1° hetero-substituent.

In one embodiment, each Ring A substituent, if present, is independently a 1° carbo-substituent selected from: (C-1), (C-7), (C-8), (C-9) and (C-10), as defined herein, or a 1° hetero-substituent selected from: (H-1), (H-2), (H-3), (H-5), (H-6), (H-11), (H-12), (H-13), (H-14), and (H-21), as defined herein.

In one embodiment, each Ring A substituent, if present, is independently as defined above for R.

In one embodiment, each Ring A substituent, if present, is independently selected from: —F, —Cl, —Br, —I, —OH, —O—C₁₋₇alkyl, —O—C₁₋₇haloalkyl, —S—C₁₋₇alkyl, —NH₂, —NH—C₁₋₇alkyl, —N(C₁₋₇alkyl)₂, —C(═O)OH, —C(═O)O—C₁₋₇alkyl, —C(═O)NH₂, —OC(═O)—C₁₋₇alkyl, —NO₂, C₁₋₇alkyl, —C₁₋₇haloalkyl, —CH₂-Ph, -Ph, -Ph-C₁₋₇haloalkyl.

In one embodiment, each Ring A substituent, if present, is independently selected from: —F, —Cl, —Br, —I, —OH, —OMe, —OCF₃, —SMe, —NH₂, —NHMe, —NMe₂, —C(═O)OH, —C(═O)OMe, —C(═O)NH₂, —OC(═O)Me, —NO₂, -Me, —CF₃, —CH₂-Ph, -Ph, -Ph-CF₃.

In one embodiment, each Ring A substituent, if present, is independently a 1° carbo-substituent selected from: (C-7) and (C-8), as defined herein, or a 1° hetero-substituent selected from: (H-3), (H-5), (H-6), and (H-12), as defined herein.

In one embodiment, R¹¹, or, the group R at the position of R¹¹ (including, e.g., the group R in Formula (II) and Formula (VII)), if present, is independently a 1° carbo-substituent selected from: (C-7) and (C-8), as defined herein, or a 1° hetero-substituent selected from: (H-3), (H-5), (H-6), and (H-12), as defined herein.

In one embodiment, each Ring A substituent, if present, is independently a 1° carbo-substituent selected from: (C-7) and (C-8), as defined herein.

In one embodiment, R¹¹, or, the group R at the position of R¹¹ (including, e.g., the group R in Formula (II) and Formula (VII)), if present, is independently a 1° carbo-substituent

selected from: (C-7) and (C-8), as defined herein.

In one embodiment, each Ring A substituent, if present, is independently selected from those substituents exemplified under the heading “Some Preferred Embodiments.”

### Ring B Substituents

In one embodiment, each Ring B substituent, if present, is independently a 1° carbo-substituent or a 1° hetero-substituent.

In one embodiment, each Ring B substituent, if present, is independently a 1° carbo-substituent selected from: (C-1), (C-7), (C-8), (C-9) and (C-10), as defined herein, or a 1° hetero-substituent selected from: (H-1), (H-2), (H-3), (H-5), (H-6), (H-11), (H-12), (H-13), (H-14), and (H-21), as defined herein.

In one embodiment, each Ring B substituent, if present, is independently as defined above for R.

In one embodiment, each Ring B substituent, if present, is independently selected from: —F, —Cl, —Br, —I, —OH, —O—C₁₋₇alkyl, —O—C₁₋₇haloalkyl, —S—C₁₋₇alkyl, —NH₂, —NH—C₁₋₇alkyl, —N(C₁₋₇alkyl)₂, —C(═O)OH, —C(═O)O—C₁₋₇alkyl, —C(═O)NH₂, —OC(═O)—C₁₋₇alkyl, —NO₂, C₁₋₇alkyl, —C₁₋₇haloalkyl, —CH₂-Ph, -Ph, -Ph-C₁₋₇haloalkyl.

In one embodiment, each Ring B substituent, if present, is independently selected from: —F, —Cl, —Br, —I, —OH, —OMe, —OCF₃, —SMe, —NH₂, —NHMe, —NMe₂, —C(═O)OH, —C(═O)OMe, —C(═O)NH₂, —OC(═O)Me, —NO₂, -Me, —CF₃, —CH₂-Ph, -Ph, -Ph-CF₃.

In one embodiment, each Ring B substituent, if present, is independently a 1° carbo-substituent selected from: (C-7) and (C-8), as defined herein, or a 1° hetero-substituent selected from: (H-3), (H-5), (H-6), and (H-12), as defined herein.

In one embodiment, each Ring B substituent, if present, is independently a 1° hetero-substituent selected from: (H-7), (H-12), (H-13), and (H-14), as defined herein.

In one embodiment, each Ring B substituent, if present, is independently selected from: —F, —Cl, —Br, —I, C₁₋₇alkyl, pyrazole, or phenyl; wherein each pyrazole and phenyl, if present, is optionally substituted, for example, with one or more substituents selected from: —F, —Cl, —Br, —I, —OH, C₁₋₇alkyl, and —O—C₁₋₄alkyl.

In one embodiment, each Ring B substituent, if present, is independently selected from those substituents exemplified under the heading “Some Preferred Embodiments.”

### Ring C Substituents

In one embodiment, each Ring C substituent, if present, is independently a 1° carbo-substituent or a 1° hetero-substituent.

In one embodiment, each Ring C substituent, if present, is independently a 10 carbo-substituent selected from: (C-1), (C-7), (C-8), (C-9) and (C-10), as defined herein, or a 1° hetero-substituent selected from: (H-1), (H-2), (H-3), (H-5), (H-6), (H-11), (H-12), (H-13), (H-14), and (H-21), as defined herein.

In one embodiment, each Ring C substituent, if present, is independently as defined above for R.

In one embodiment, each Ring C substituent, if present, is independently selected from: —F, —Cl, —Br, —I, —OH, —O—C₁₋₇alkyl, —O—C₁₋₇haloalkyl, —S—C₁₋₇alkyl, —NH₂, —NH—C₁₋₇alkyl, —N(C₁₋₇alkyl)₂, —C(═O)OH, —C(═O)O—C₁₋₇alkyl, —C(═O)NH₂, —OC(═O)—C₁₋₇alkyl, —NO₂, C₁₋₇alkyl, —C₁₋₇haloalkyl, —CH₂-Ph, -Ph, -Ph-C₁₋₇haloalkyl.

In one embodiment, each Ring C substituent, if present, is independently selected from: —F, —Cl, —Br, —I, —OH, —OMe, —OCF₃, —SMe, —NH₂, —NHMe, —NMe₂, —C(═O)OH, —C(═O)OMe, —C(═O)NH₂, —OC(═O)Me, —NO₂, -Me, —CF₃, —CH₂-Ph, -Ph, -Ph-CF₃.

In one embodiment, each Ring C substituent, if present, is independently selected from those substituents exemplified under the heading “Some Preferred Embodiments.”

### Ring D, Ring E, and Ring F Substituents

In one embodiment, each Ring D substituent, if present, and each Ring E substituent, if present, and each Ring F substituent, if present, is independently a 1° carbo-substituent or a 1° hetero-substituent.

In one embodiment, each Ring D substituent, if present, and each Ring E substituent, if present, and each Ring F substituent, if present, is independently a 1° carbo-substituent selected from: (C-1), (C-7), (C-8), (C-9) and (C-10), as defined herein, or a 1° hetero-substituent selected from: (H-1), (H-2), (H-3), (H-5), (H-6), (H-11), (H-12), (H-13), (H-14), and (H-21), as defined herein.

In one embodiment, each Ring D substituent, if present, and each Ring E substituent, if present, and each Ring F substituent, if present, is independently as defined above for R.

In one embodiment, each Ring D substituent, if present, and each Ring E substituent, if present, and each Ring F substituent, if present, is independently selected from: —F, —Cl, —Br, —I, —OH, —O—C₁₋₇alkyl, —O—C₁₋₇haloalkyl, —S—C₁₋₇alkyl, —NH₂, —NH—C₁₋₇alkyl, —N(C₁₋₇alkyl)₂, —C(═O)OH, —C(═O)O—C₁₋₇alkyl, —C(═O)NH₂, —OC(═O)—C₁₋₇alkyl, —NO₂, C₁₋₇alkyl, —C₁₋₇haloalkyl, —CH₂-Ph, -Ph, -Ph-C₁₋₇haloalkyl.

In one embodiment, each Ring D substituent, if present, and each Ring E substituent, if present, and each Ring F substituent, if present, is independently selected from: —F, —Cl, —Br, —I, —OH, —OMe, —OCF₃, —SMe, —NH₂, —NHMe, —NMe₂, —C(═O)OH, —C(═O)OMe, —C(═O)NH₂, —OC(═O)Me, —NO₂, -Me, —CF₃, —CH₂-Ph, -Ph, -Ph-CF₃.

In one embodiment, each Ring D substituent, if present, and each Ring E substituent, if present, and each Ring F substituent, if present, is independently selected from those substituents exemplified under the heading “Some Preferred Embodiments.”

### The Group R¹⁴

In one embodiment, R¹⁴ is independently —H or a group W.

In one embodiment, R¹⁴ is independently —H.

In one embodiment, R¹⁴ is independently a group W.

### The Group W

In one embodiment, the group W, if present, is independently a 1° carbo-substituent.

In one embodiment, the group W, if present, is independently selected from:

-Me, -Et, -nPr, -iPr, -tBu, -Ph, —CH₂-Ph.

In one embodiment, the group W, if present, is independently selected from those groups exemplified under the heading “Some Preferred Embodiments.”

### The Amino-Ethylene-Amino Group: Stereoisomerism

In one embodiment, the “amino-ethylene-amino” group, M, shown below,

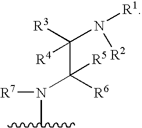

is the following group:

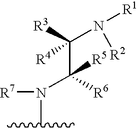

Note that when R³ and R⁴, taken together, form a group ═O, there is no chirality at the carbon to which they are attached. Similarly, when R⁵ and R⁶, taken together, form a group ═O, there is no chirality at the carbon to which they are attached. This is illustrated in the followed formulae:

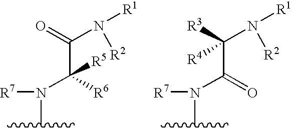

### The Groups R¹, R², R³, R⁴, R⁵, R⁶, and R⁷

For the avoidance of doubt, it is not intended that any two or more of R¹, R², R³, R⁴, R⁵, R⁶, and R⁷, taken together with the atoms they are attached to, form part of a ring. For example, it is not intended that R⁷ and R³, taken together with the —N—C—C— backbone to which they are attached, form a ring. Similarly, it is not intended that R¹ and R², taken together with the N atom to which they are attached form a ring.

In one embodiment:

each of R¹, R², R³, R⁴, R⁵, R⁶, and R⁷ is independently —H or a group G;
and additionally:
each of R³, R⁴, R⁵, and R⁶ may be a group Y;
each of R¹, R², and R⁷ may be a group Z;
R³ and R⁴, taken together, may form a group ═O;
R⁵ and R⁶, taken together, may form a group ═O.

In one embodiment:

each of R¹, R², R³, R⁴, R⁵, R⁶, and R⁷ is independently —H or a group G;
and additionally:
each of R³, R⁴, R⁵, and R⁶ may be a group Y;
each of R¹, R², and R⁷ may be a group Z.

(That is, R³ and R⁴, taken together, do not form a group ═O; and R⁵ and R⁶, taken together, do not form a group ═O.)

In one embodiment:

each of R¹, R², R³, R⁴, R⁵, R⁶, and R⁷ is independently —H or a group G;
and additionally:
R³ and R⁴, taken together, may form a group ═O;
R⁵ and R⁶, taken together, may form a group ═O.

(That is, none of R³, R⁴, R⁵, and R⁶ is a group Y; and none of R¹, R², and R⁷ is a group Z.)

In one embodiment, each of R¹, R², R³, R⁴, R⁵, R⁶, and R⁷ is independently —H or a group G.

(That is, none of R³, R⁴, R⁵, and R⁶ is a group Y; and none of R¹, R², and R⁷ is a group Z; and R³ and R⁴, taken together, do not form a group ═O; and R⁵ and R⁶, taken together, do not form a group ═O.)

In one embodiment:

one of R³ and R⁴ is independently C₁₋₆alkyl or C₃₋₆cycloalkyl;
the other of R³ and R⁴ is independently —H;
R⁷ is independently —H or C₁₋₆alkyl; and
each of R¹, R², R⁵, and R⁶ is independently —H.

In one embodiment:

one of R³ and R⁴ is independently C₁₋₄alkyl or C₃₋₄cycloalkyl;
the other of R³ and R⁴ is independently —H;
R⁷ is independently —H or C₁₋₄alkyl; and
each of R¹, R², R⁵, and R⁶ is independently —H.

### The Amino-Ethylene-Amino Group: Combinations of Substituents

In one embodiment, exactly one or exactly two or exactly three of R¹, R², R³, R⁴, R⁵, R⁶, and R⁷ is other than —H, and each of the others is —H.

In one embodiment, exactly one of R¹, R², R³, R⁴, R⁵, R⁶, and R⁷ is other than —H, and each of the others is —H, as in, for example:

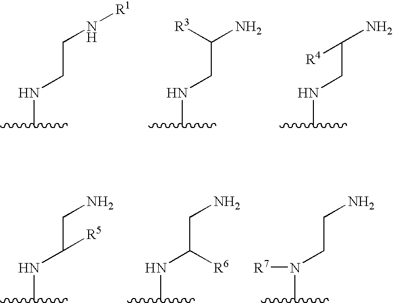

In one embodiment, exactly two of R¹, R², R³, R⁴, R⁵, R⁶, and R⁷ is other than —H, and each of the others is —H, as in, for example:

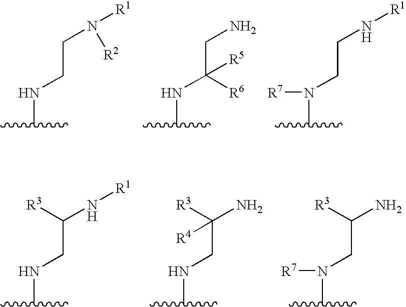

In one embodiment, exactly three of R¹, R², R³, R⁴, R⁵, R⁶, and R⁷ is other than —H, and each of the others are —H, as in, for example:

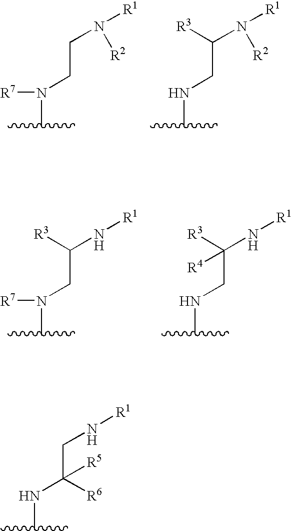

In one embodiment, R³ is other than —H, or R⁴ is other than —H.

In one embodiment, R³ is other than —H.

In one embodiment, R⁴ is other than —H.

In one embodiment, R³ is other than —H, and each of R¹, R², R⁴, R⁵, R⁶, and R⁷ is —H; or R⁴ is other than —H, and each of R¹, R², R³, R⁵, R⁶, and R⁷ is —H, as in, for example,

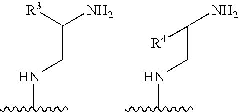

In one embodiment, R³ is other than —H, and each of R¹, R², R⁴, R⁵, R⁶, and R⁷ is —H, as in, for example,

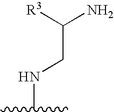

In one embodiment, R⁴ is other than —H, and each of R¹, R², R³, R⁴, R⁵, R⁶, and R⁷ is —H, as in, for example,

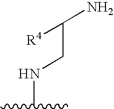

In one embodiment, either: R³ and R⁴, taken together, form a group ═O, or: R⁵ and R⁶, taken together, form a group ═O.

In one embodiment, R³ and R⁴, taken together, may form a group ═O, as in, for example:

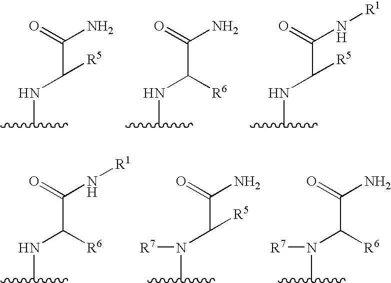

In one embodiment, R³ and R⁴ may not be taken together to form a group ═O (i.e., the case where R³ and R⁴, taken together, form a group ═O, is excluded).

In one embodiment, R⁵ and R⁶, taken together, may form a group ═O, as in, for example:

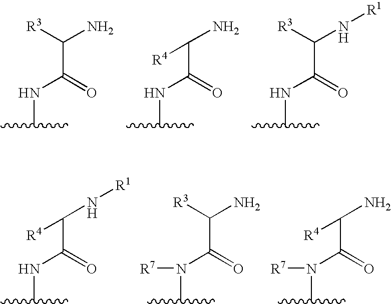

In one embodiment, R⁵ and R⁶ may not be taken together to form a group ═O (i.e., the case where R⁵ and R⁶, taken together, form a group ═O, is excluded).

In one embodiment, either: R³ and R⁴, taken together, form a group ═O, or: R⁵ and R⁶, taken together, form a group ═O.

### The Group G

In one embodiment, each group G, if present, is independently a 1° carbo-substituent.

In one embodiment, each group G, if present, is independently a 1° carbo-substituent selected from (C-1), (C-4), (C-7), (C-8), (C-9), and (C-10), as defined herein.

In one embodiment, each group G, if present, is independently a 1° carbo-substituent selected from (C-1), (C-7), and (C-9), as defined herein.

In one embodiment, each group G, if present, is independently C₁₋₇alkyl, and is independently unsubstituted or substituted with one or more (e.g., 1, 2, 3, 4) substituents selected from 1° hetero-substituents.

In one embodiment, each group G, if present, is independently C₁₋₇alkyl, and is unsubstituted.

In alternative narrower embodiments of the above, C₁₋₇alkyl is C₁₋₆alkyl.

In alternative narrower embodiments of the above, C₁₋₇alkyl is C₁₋₅alkyl.

In alternative narrower embodiments of the above, C₁₋₇alkyl is C₁₋₄alkyl.

In alternative narrower embodiments of the above, C₁₋₇alkyl is C₁₋₃alkyl.

In alternative narrower embodiments of the above, C₁₋₇alkyl is C₂₋₇alkyl.

In alternative narrower embodiments of the above, C₁₋₇alkyl is C₂₋₆alkyl.

In alternative narrower embodiments of the above, C₁₋₇alkyl is C₂₋₅alkyl.

In alternative narrower embodiments of the above, C₁₋₇alkyl is C₂₋₄alkyl.

In alternative narrower embodiments of the above, C₁₋₇alkyl is C₁alkyl.

In alternative narrower embodiments of the above, C₁₋₇alkyl is C₂alkyl.

In alternative narrower embodiments of the above, C₁₋₇alkyl is C₃alkyl.

In alternative narrower embodiments of the above, C₁₋₇alkyl is C₄alkyl.

In alternative narrower embodiments of the above, C₁₋₇alkyl is C₅alkyl.

In alternative narrower embodiments of the above, C₁₋₇alkyl is C₆alkyl.

In alternative narrower embodiments of the above, C₁₋₇alkyl is C₇alkyl.

In one embodiment, each group G is independently selected from the following, and is independently unsubstituted or substituted with one or more (e.g., 1, 2, 3, 4) substituents selected from 1° hetero-substituents:

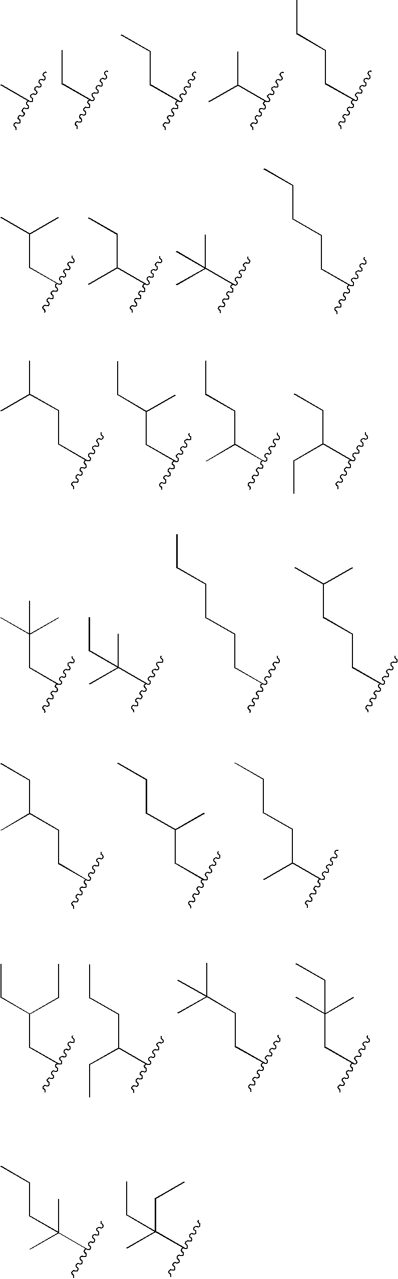

In one embodiment, each group G is independently selected from the following, and is independently unsubstituted or substituted with one or more (e.g., 1, 2, 3, 4) substituents selected from 1° hetero-substituents:

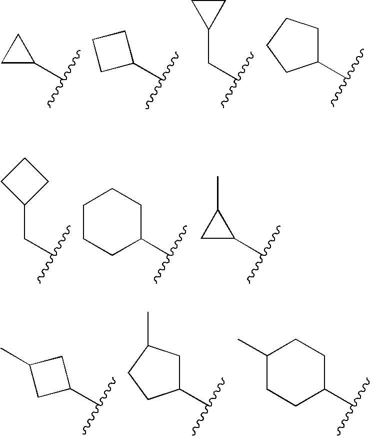

In one embodiment, each group G is independently selected from the following, and is independently unsubstituted or substituted with one or more (e.g., 1, 2, 3, 4) substituents selected from 1° hetero-substituents:

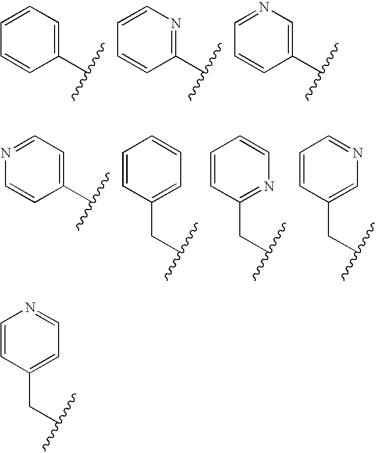

In one embodiment, 1° hetero-substituents on G, if present, are independently selected from: (H-1), (H-2), (H-3), (H-5), (H-6), (H-11), (H-12), (H-13), (H-14), and (H-21), as defined herein.

In one embodiment, 1° hetero-substituents on G, if present, are independently selected from: —F, —Cl, —Br, —I, —OH, —O—C₁₋₇alkyl, —O—C₁₋₇haloalkyl, —S—C₁₋₇alkyl, —NH₂, —NH—C₁₋₁₇alkyl, —N(C₁₋₇alkyl)₂, —C(═O)OH, —C(═O)O—C₁₋₁₇alkyl, —C(═O)NH₂, —OC(═O)—C₁₋₇alkyl, —NO₂.

In one embodiment, 1° hetero-substituents on G, if present, are independently selected from: —F, —Cl, —Br, —I, —OH, —OMe, —OCF₃, —SMe, —NH₂, —NHMe, —NMe₂, —C(═O)OH, —C(═O)OMe, —C(═O)NH₂, —OC(═O)Me, —NO₂.

In one embodiment, 2° carbo-substituents on G, if present, are independently selected from: C₁₋₇alkyl, C₁₋₇haloalkyl, —CH₂-Ph, -Ph, -Ph-C₁₋₇haloalkyl.

In one embodiment, 2° carbo-substituents on G, if present, are independently selected from: -Me, —CF₃, —CH₂-Ph, -Ph, -Ph-CF₃.

In one embodiment, each group G is independently as defined above, and is unsubstituted.

### The Group Y

As described herein, each of R³, R⁴, R⁵, and R⁶ may be a group Y.

In one embodiment, each group Y, if present, is independently a 1° hetero-substituent.

In one embodiment, each group Y, if present, is independently a 1° hetero-substituent selected from: (H-11), (H-12), and (H-13), as defined herein.

In one embodiment, each group Y, if present, is independently selected from: —C(═O)OH, —C(═O)OMe, —C(═O)OEt, —C(═O)OPh, —C(═O)OCH₂Ph, —C(═O)NH₂, —C(═O)NHMe, —C(═O)NHEt, —C(═O)NMe₂, —C(═O)NEt₂.

### The Group Z

As described herein, each of R¹, R², and R⁷ may be a group Z.

In one embodiment, each group Z, if present, is independently a 1° hetero-substituent selected from: (H-10), (H-12), (H-13), and (H-18), as defined herein.

In one embodiment, each group Y, if present, is independently selected from: —C(═O)Me, —C(═O)Et, —C(═O)OMe, —C(═O)OEt, —C(═O)OPh, —C(═O)OCH₂Ph, —C(═O)NH₂, —C(═O)NHMe, —C(═O)NHEt, —C(═O)NMe₂, —C(═O)NEt₂, —S(═O)₂Me, —S(═O)₂Et, —S(═O)₂Ph, —S(═O)₂Ph-Me.

### The Amino-Ethylene-Amino Group: Some Preferred Groups: R³ and R⁴

In one embodiment, the “amino-ethylene-amino” group, M, is one of the following groups:

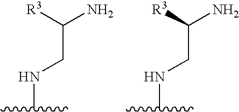

In one embodiment, the “amino-ethylene-amino” group, M, is one of the following groups:

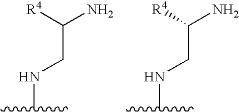

In one embodiment, the “amino-ethylene-amino” group, M, is selected from the following groups, and is independently unsubstituted or substituted with one or more (e.g., 1, 2, 3, 4) substituents selected from 1° hetero-substituents:

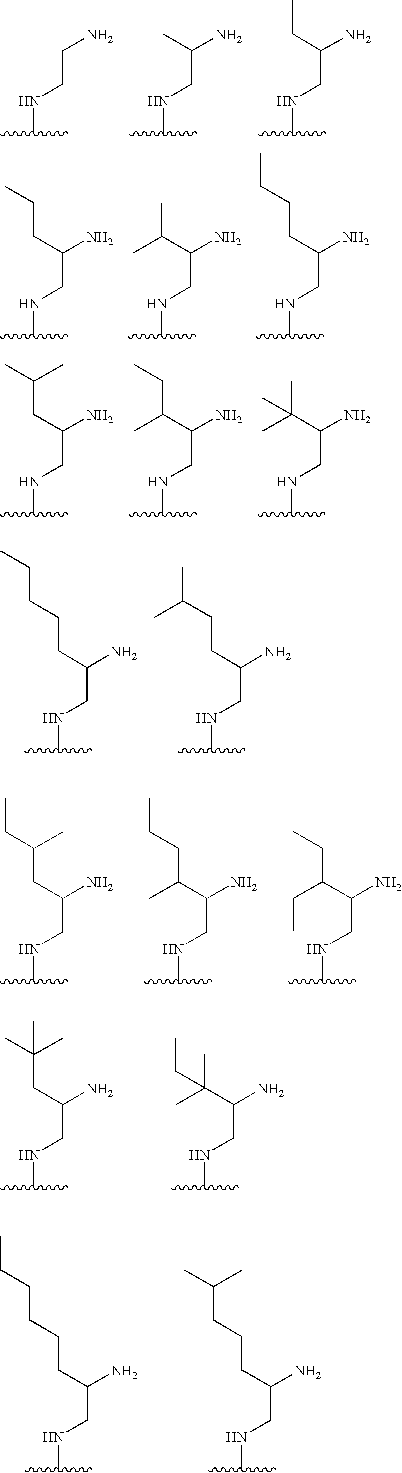

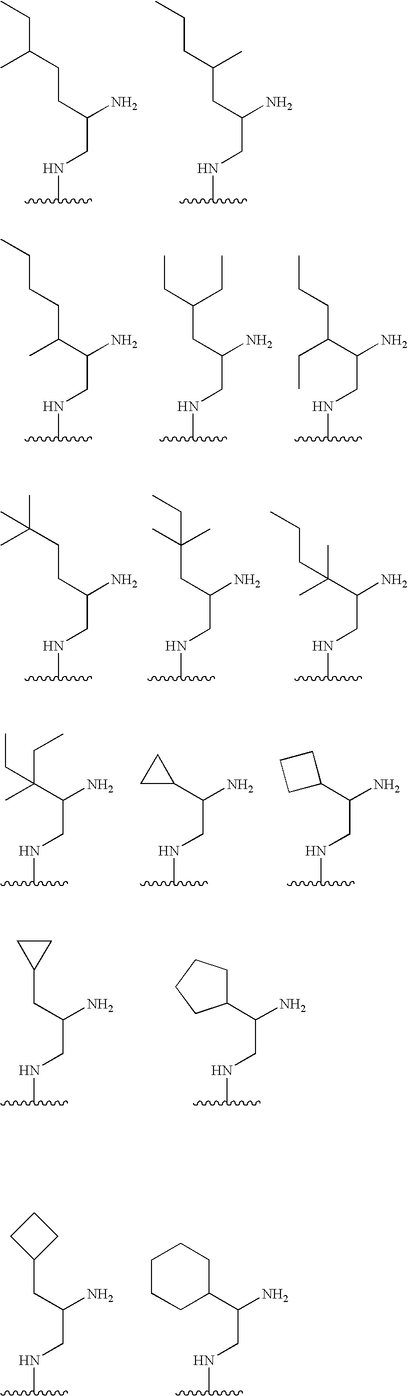

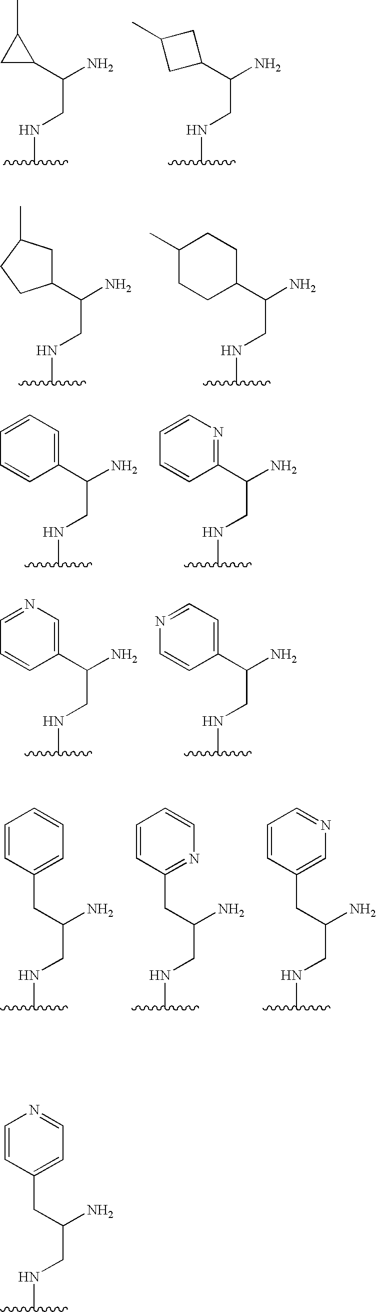

In one embodiment, the “amino-ethylene-amino” group, M, is as defined above, except that the bond marked a (alpha), if present, is “up”, as in, for example:

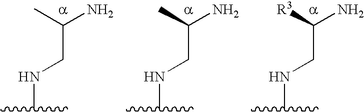

In one embodiment, the “amino-ethylene-amino” group, M, is as defined above, except that the bond marked a (alpha), if present, is “down”, as in, for example:

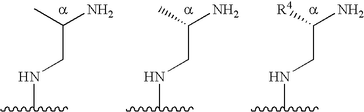

### The Amino-Ethylene-Amino Group: Some Other Preferred Groups

In one embodiment, the “amino-ethylene-amino” group, M, is selected from the following groups, and is independently unsubstituted or substituted with one or more (e.g., 1, 2, 3, 4) substituents selected from 1° hetero-substituents:

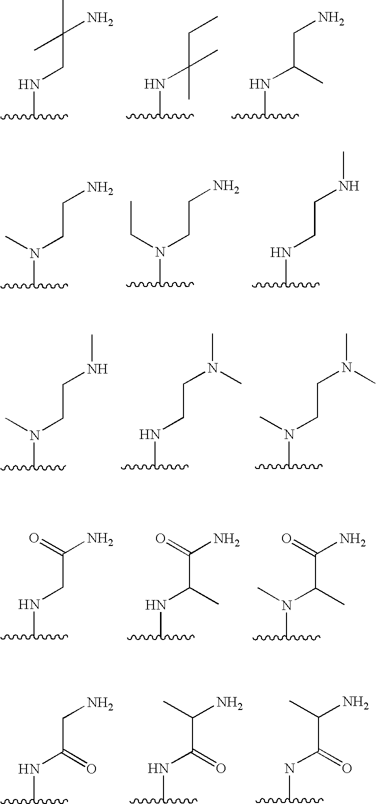

Examples of some substituted “amino-ethylene-amino” groups include the following:

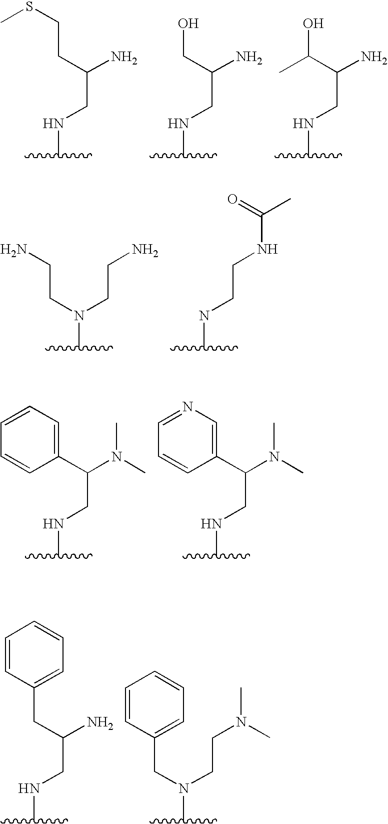

Additional examples of “amino-ethylene-amino” groups include the following:

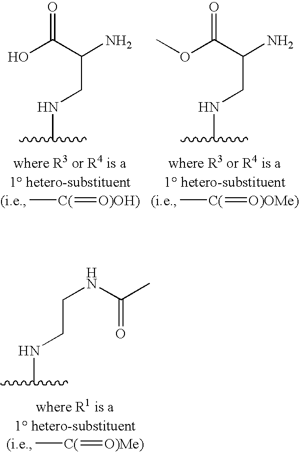

### Carbo-Substituents and Hetero-Substituents: Generally

Each of 1° carbo-substituent, 2° carbo-substituent, 3° carbo-substituent, 1° hetero-substituent, and 2° hetero-substituent are defined herein.

A 1° carbo-substituent may bear one or more (e.g., 1, 2, 3, 4) 1° hetero-substituents and/or one or more (e.g., 1, 2, 3, 4) 2° carbo-substituents (e.g., on C₃₋₁₄heterocyclyl, C₆₋₁₄carboaryl, and C₅₋₁₄heteroaryl). The (or each) 1° hetero-substituent may include one or more (e.g., 1, 2, 3, 4) 2° carbo-substituents. The (or each) 2° carbo-substituent may further bear one or more (e.g., 1, 2, 3, 4) 3° carbo-substituents (e.g., on C₃₋₁₄heterocyclyl, C₆₋₁₄carboaryl, and C₅₋₁₄heteroaryl) and/or one or more (e.g., 1, 2, 3, 4) 2° hetero-substituents. The (or each) 2° hetero-substituent may include one or more (e.g., 1, 2, 3, 4) 3° carbo-substituents. The (or each) 3° carbo-substituent is unsubstituted. This is illustrated by the following example:

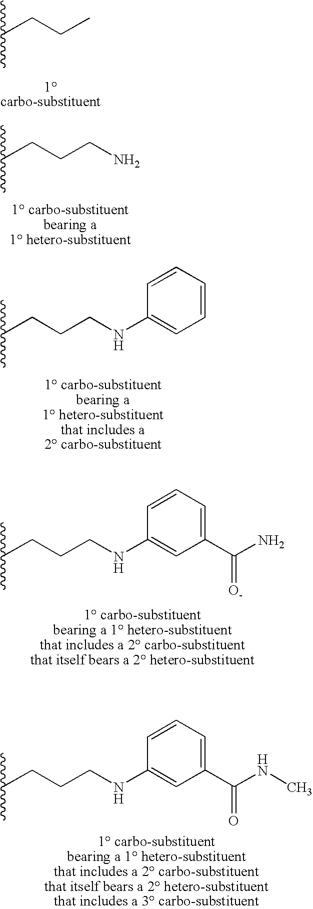

### Carbo-Substituents

The term “1° carbo-substituent,” as used herein, refers to a substituent independently selected from:
(C-1) C₁₋₇alkyl,
(C-2) C₂₋₇alkenyl,
(C-3) C₂₋₇alkynyl,
(C-4) C₃₋₇cycloalkyl,
(C-5) C₃₋₇cycloalkenyl,
(C-6) C₃₋₁₄heterocyclyl,
(C-7) C₆₋₁₄carboaryl,
(C-8) C₅₋₁₄heteroaryl,
(C-9) C₆₋₁₄carboaryl-C₁₋₇alkyl, and
(C-10) C₆₋₁₄heteroaryl-C₁₋₇alkyl;
wherein each C₁₋₇alkyl, C₂₋₇alkenyl, C₂₋₇alkynyl, C₃₋₇cycloalkyl, and C₃₋₇cycloalkenyl, is independently unsubstituted or substituted with one or more (e.g., 1, 2, 3, 4) substituents selected from 1° hetero-substituents; and
wherein each C₃₋₁₄heterocyclyl, C₆₋₁₄carboaryl, and C₅₋₁₄heteroaryl is independently unsubstituted or substituted with one or more (e.g., 1, 2, 3, 4) substituents selected from 1° hetero-substituents and 2° carbo-substituents.

The term “2° carbo-substituent,” as used herein, refers to a substituent as defined herein for “1° carbo-substituent,” except that:
each C₁₋₇alkyl, C₂₋₇alkenyl, C₂₋₇alkynyl, C₃₋₇cycloalkyl, and C₃₋₇cycloalkenyl, is independently unsubstituted or substituted with one or more (e.g., 1, 2, 3, 4) substituents selected from 2° hetero-substituents; and
each C₃₋₁₄heterocyclyl, C₆₋₁₄carboaryl, and C₅₋₁₄heteroaryl is independently unsubstituted or substituted with one or more (e.g., 1, 2, 3, 4) substituents selected from 2° hetero-substituents and 3° carbo-substituents.

The term “3° carbo-substituent,” as used herein, refers to a substituent as defined herein for “1° carbo-substituent,” except that:
each C₁₋₇alkyl, C₂₋₇alkenyl, C₂₋₇alkynyl, C₃₋₇cycloalkyl, and C₃₋₇cycloalkenyl, is unsubstituted; and
each C₃₋₁₄heterocyclyl, C₆₋₁₄carboaryl, and C₅₋₁₄heteroaryl is unsubstituted.

In one embodiment, the or each 1° carbo-substituent is independently selected from:

(C-1) C₁₋₇alkyl,
(C-4) C₃₋₇cycloalkyl,
(C-7) C₆₋₁₄carboaryl,
(C-8) C₅₋₁₄heteroaryl,
(C-9) C₆₋₁₄carboaryl-C₁₋₇alkyl, and
(C-10) C₅₋₁₄heteroaryl-C₁₋₇alkyl;
wherein each C₁₋₇alkyl and C₃₋₇cycloalkyl is independently unsubstituted or substituted with one or more (e.g., 1, 2, 3, 4) substituents selected from 1° hetero-substituents; and
wherein each C₆₋₁₄carboaryl and C₅₋₁₄heteroaryl is independently unsubstituted or substituted with one or more (e.g., 1, 2, 3, 4) substituents selected from 1° hetero-substituents and 2° carbo-substituents.

In one embodiment, the or each 2° carbo-substituent is correspondingly defined.

In one embodiment, the or each 3° carbo-substituent is correspondingly defined.

In one embodiment, the or each 1° carbo-substituent is independently selected from:

(C-1) C₁₋₇alkyl,
(C-4) C₃₋₇cycloalkyl,
(C-7) C₆carboaryl,
(C-8) C₅₋₆heteroaryl,
(C-9) C₆carboaryl-C₁₋₇alkyl, and
(C-10) C₅₋₆heteroaryl-C₁₋₇alkyl;
wherein each C₁₋₇alkyl and C₃₋₇cycloalkyl is independently unsubstituted or substituted with one or more (e.g., 1, 2, 3, 4) substituents selected from 1° hetero-substituents; and
wherein each C₆carboaryl and C₅₋₆heteroaryl is independently unsubstituted or substituted with one or more (e.g., 1, 2, 3, 4) substituents selected from 1° hetero-substituents and 2° carbo-substituents.

In one embodiment, the or each 2° carbo-substituent is correspondingly defined.

In one embodiment, the or each 3° carbo-substituent is correspondingly defined.

In one embodiment, the or each 1° carbo-substituent is independently selected from:

(C-1) C₁₋₇alkyl,
(C-4) C₃₋₇cycloalkyl,
(C-7) C₆carboaryl,
(C-8) C₅₋₆heteroaryl,
(C-9) C₆carboaryl-C₁₋₇alkyl, and
(C-10) C₅₋₆heteroaryl-C₁₋₇alkyl;
and is unsubstituted.

In one embodiment, the or each 2° carbo-substituent is correspondingly defined.

In one embodiment, the or each 3° carbo-substituent is correspondingly defined.

In one embodiment, the or each 1° carbo-substituent is independently (C-1) C₁₋₇alkyl, and is independently unsubstituted or substituted with one or more (e.g., 1, 2, 3, 4) substituents selected from 1° hetero-substituents.

In one embodiment, the or each 2° carbo-substituent is correspondingly defined.

In one embodiment, the or each 3° carbo-substituent is correspondingly defined.

In one embodiment, the or each 1° carbo-substituent is unsubstituted.

In one embodiment, the or each 2° carbo-substituent is unsubstituted.

In one embodiment, the or each 3° carbo-substituent is unsubstituted.

In one embodiment, each C₁₋₇alkyl group, if present, is independently selected from: methyl, ethyl, n-propyl, i-propyl, n-butyl, i-butyl, s-butyl, t-butyl, n-pentyl, i-pentyl, and n-hexyl.

In one embodiment, each C₃₋₇cycloalkyl group, if present, is independently selected from: cyclopropyl, cyclopropyl-methyl, cyclobutyl, cyclopentyl, and cyclohexyl.

In one embodiment, each C₆₋₁₄carboaryl group (or each C₆carboaryl group), if present, is independently phenyl.

In one embodiment, each C₅₋₁₄heteroaryl group (or each C₅₋₆heteroaryl group), if present, is independently pyridyl.

In one embodiment, each C₆₋₁₄carboaryl-C₁₋₇alkyl group (or each C₆carboaryl-C₁₋₇alkyl group), if present, is independently benzyl.

In one embodiment, each C₅₋₁₄heteroaryl-C₁₋₇alkyl group (or each C₅₋₆heteroaryl-C₁₋₇alkyl group), if present, is independently pyridyl-methyl.

Some examples of 1° carbo-substituents that are C₁₋₇alkyl groups substituted with one or more (e.g., 1, 2, 3, 4) 1° hetero-substituents include the following:

halo-C₁₋₇alkyl;
(e.g., —CF₃, —CH₂CF₃);
   amino-C₁₋₇alkyl
(e.g., —(CH₂)_{w}—NH₂, —(CH₂)_{w}—NHR^{A3}, —(CH₂)_{w}—NR^{A4}R^{A5}, wherein w is 1, 2, 3, or 4);
   hydroxy-C₁₋₇alkyl
(e.g., —(CH₂)_{w}—OH, w is 1, 2, 3, or 4);
   carboxy-C₁₋₇alkyl
(e.g., —(CH₂)_{w}—C(═O)OH, w is 1, 2, 3, or 4);
   C₁₋₇alkoxy-C₁₋₇alkyl
(e.g., —(CH₂)_{w}—O—C₁₋₇alkyl, w is 1, 2, 3, or 4);
   acyl-C₁₋₇alkyl
(e.g., —(CH₂)_{w}—C(═O)R^{A13}, w is 1, 2, 3, or 4);

### Hetero-Substituents

The term “1° hetero-substituent,” as used herein, refers to a substituent independently selected from:
(H-1) —F, —Cl, —Br, —I;
(H-2) —OH;
(H-3) —OR^{A1}, wherein R^{A1} is independently a 2° carbo-substituent;
(H-4) —SH;
(H-5) —SR^{A2}, wherein R^{A2} is independently a 2° carbo-substituent;
(H-6) —NH₂, —NHR^{A3}, —NR^{A4}R^{A5}, wherein each of R^{A3}, R^{A4}, and R^{A5} is independently a 2° carbo-substituent; or R^{A4} and R^{A5} taken together with the nitrogen atom to which they are attached form a ring having from 3 to 7 ring atoms;
(H-7) —NHC(═O)R^{A6}, —NR^{A7}C(═O)R^{A6}, wherein each of R^{A6} and R^{A7} is independently a 2° carbo-substituent;
(H-8) —NHC(═O)OR^{A9}, —NR^{A10}C(═O)OR^{A9}, wherein each of R^{A9} and R^{A10} is independently a 2° carbo-substituent;
(H-9) —NHC(═O)NH₂, —NR OC(═O)NH₂, —NHC(═O)NHR^{A11}, —NR^{A10}C(═O)NHR^{A11}, NHC(═O)NR^{A11}R^{A12}, NR^{A10}C(═O)NHR^{A11}R^{A12}, wherein each of R^{A10}, R^{A11}, and R^{A12} is independently a 2° carbo-substituent; or R^{A11} and R^{A12} taken together with the nitrogen atom to which they are attached form a ring having from 3 to 7 ring atoms;
(H-10) —C(═O)R^{A13}, wherein R^{A13} is independently a 2° carbo-substituent;
(H-11) —C(═O)OH;
(H-12) —C(═O)OR^{A14}, wherein R^{A14} is independently a 2° carbo-substituent;
(H-13) —C(═O)NH₂, —C(═O)NHR^{A15}, —C(═O)NR^{A15}R^{A16}, wherein each of R^{A15} and R^{A16} is independently a 2° carbo-substituent; or R^{A15} and R^{A16} taken together with the nitrogen atom to which they are attached form a ring having from 3 to 7 ring atoms;
(H-14) —OC(═O)R^{A17}, wherein R^{A17} is independently a 2° carbo-substituent;
(H-15) —OC(═O)NH₂, —OC(═O)NHR^{A18}, —OC(═O)NR^{A18}R^{A19}, wherein each of R^{A18} and R^{A19} is independently a 2° carbo-substituent; or R^{A13} and R^{A19} taken together with the nitrogen atom to which they are attached form a ring having from 3 to 7 ring atoms;
(H-16) —S(═O)₂NH₂, —S(═O)₂NHR^{A20}, —S(═O)₂NR^{A20}R^{A21}, wherein each of R^{A20} and R^{A21} is independently a 2° carbo-substituent; or R^{A20} and R^{A21} taken together with the nitrogen atom to which they are attached form a ring having from 3 to 7 ring atoms;
(H-17) —NHS(═O)₂R^{A22}, —NR^{A23}S(═O)₂R^{A22}, wherein each of R^{A22} and R^{A23} is independently a 2° carbo-substituent;
(H-18) —S(═O)₂R^{A24}, wherein R^{A24} is independently a 2° carbo-substituent;
(H-19) —S(═O)₂OH;
(H-20) —S(═O)₂OR^{A25}, —OS(═O)₂R^{A26}, wherein each of R^{A25} and R^{A26} is independently a 2° carbo-substituent;
(H-21) —NO₂; and
(H-22) —C≡N.

In one embodiment, the or each 1° hetero-substituent is as defined above, but is not (H-22) —C≡N.

The term “2° hetero-substituent,” as used herein, refers to a substituent as defined for “1° hetero-substituent,” except that: each 2° carbo-substituent is a 3° carbo-substituent.

In one embodiment, the or each 2° hetero-substituent is as defined above, but is not (H-22) —C≡N.

Examples of groups —NR^{a}R^{b}, where R^{a} and R^{b} taken together with the nitrogen atom to which they are attached form a ring having from 3 to 7 ring atoms include: piperidino, piperizino, and morpholino.

In one embodiment, the or each 1° hetero-substituent (and/or the or each 1° hetero-substituent) is independently selected from:
(H-1) —F, —Cl, —Br, —I;
(H-2) —OH;
(H-3) —OMe, —OEt, —O(iPr), —O(tBu), —OPh, —OCH₂Ph; —OCF₃, —OCH₂CF₃; —OCH₂CH₂OH, —OCH₂CH₂OMe, —OCH₂CH₂OEt; —OCH₂CH₂NH₂, —OCH₂CH₂NMe₂, —OCH₂CH₂N(iPr)₂; —OPh-Me, —OPh-OH, —OPh-OMe, —OPh-F, —OPh-Cl, —OPh-Br, —OPh-1;
(H-4) —SH;
(H-5) —SMe, —SEt, —SPh, —SCH₂Ph;
(H-6) —NH₂, —NHMe, —NHEt, —NH(iPr), —NMe₂, —NEt₂, —N(iPr)₂, —N(CH₂CH₂OH)₂; —NHPh, —NHCH₂Ph; piperidino, piperazino, morpholino;
(H-7) —NH(C═O)Me, —NH(C═O)Et, —NH(C═O)(nPr), —NH(C═O)Ph, —NHC(═O)CH₂Ph; —NMe(C═O)Me, —NMe(C═O)Et, —NMe(C═O)Ph, —NMeC(═O)CH₂Ph;
(H-8) —NH(C═O)OMe, —NH(C═O)OEt, —NH(C═O)O(nPr), —NH(C═O)OPh, —NHC(═O)OCH₂Ph; —NMe(C═O)OMe, —NMe(C═O)OEt, —NMe(C═O)OPh, —NMeC(═O)CH₂OPh;
(H-9) —NH(C═O)NH₂, —NH(C═O)NHMe, —NH(C═O)NHEt, —NH(C═O)NPh, —NH(C═O)NHCH₂Ph;
(H-10) —(C═O)Me, —(C═O)Et, —(C═O)(tBu), —(C═O)-cHex, —(C═O)Ph, —(C═O)CH₂Ph;
(H-11) —C(═O)OH;
(H-12) —OC(═O)Me, —OC(═O)Et, —OC(═O)(iPr), —OC(═O)(tBu); —OC(═O)(cPr); —OC(═O)CH₂CH₂OH, —OC(═O)CH₂CH₂OMe, —OC(═O)CH₂CH₂OEt; —OC(═O)Ph, —OC(═O)CH₂Ph;
(H-13) —(C═O)NH₂, —(C═O)NMe₂, —(C═O)NEt₂, —(C═O)N(iPr)₂, —(C═O)N(CH₂CH₂OH)₂; —(C═O)-piperidino, —(C═O)-morpholino, —(C═O)NHPh, —(C═O)NHCH₂Ph; (H-14) —OC(═O)Me, —OC(═O)Et, —OC(═O)(iPr), —OC(═O)(tBu); —OC(═O)(cPr); —OC(═O)CH₂CH₂OH, —OC(═O)CH₂CH₂OMe, —OC(═O)CH₂CH₂OEt; —OC(═O)Ph, —OC(═O)CH₂Ph;
(H-15) —OC(═O)NH₂, —OC(═O)NHMe, —OC(═O)NMe₂, —OC(═O)NHEt, —OC(═O)NEt₂, —OC(═O)NHPh, —OC(═O)NCH₂Ph;
(H-16) —S(═O)₂NH₂, —S(═O)₂NHMe, —S(═O)₂NHEt, —S(═O)₂NMe₂, —S(═O)₂NEt₂, —S(═O)₂-piperidino, —S(═O)₂-morpholino, —S(═O)₂NHPh, —S(═O)₂NHCH₂Ph;
(H-17) —NHS(═O)₂Me, —NHS(═O)₂Et, —NHS(═O)₂Ph, —NHS(═O)₂PhMe, —NHS(═O)₂CH₂Ph, —NMeS(═O)₂Me, —NMeS(═O)₂Et, —NMeS(═O)₂Ph, —NMeS(═O)₂PhMe, —NMeS(═O)₂CH₂Ph;
(H-18) —S(═O)₂Me, —S(═O)₂CF₃, —S(═O)₂Et, —S(═O)₂Ph, —S(═O)₂PhMe, —S(═O)₂CH₂Ph;
(H-19) —S(═O)₂OH;
(H-20) —OS(═O)₂OMe, —OS(═O)₂OCF₃, —OS(═O)₂OEt, —OS(═O)₂OPh, —OS(═O)₂OPh-Me, —OS(═O)₂OCH₂Ph;
(H-21) —NO₂; and
(H-22) —C≡N.

In one embodiment, the or each 1° hetero-substituent (and/or the or each 2° hetero-substituent) is as defined above, but is not (H-22) —C≡N.

In one embodiment, the or each 1° hetero-substituent (and/or the or each 2° hetero-substituent) is independently selected from: (H-1), (H-2), (H-3), (H-5), (H-6), (H-11), (H-12), (H-13), (H-14), (H-21), as defined herein.

In one embodiment, the or each 1° hetero-substituent (and/or the or each 2° hetero-substituent) is independently selected from: —F, —Cl, —Br, —I, —OH, —OMe, —OCF₃, —SMe, —NH₂, —NHMe, —NMe₂, —C(═O)OH, —C(═O)OMe, —C(═O)NH₂, —OC(═O)Me, —NO₂.

### DEFINITIONS AND EXAMPLES

The term “alkyl,” as used herein, pertains to a monovalent moiety obtained by removing a hydrogen atom from a carbon atom of a saturated aliphatic hydrocarbon compound having from 1 to 20 carbon atoms (unless otherwise specified).

Examples of (unsubstituted) alkyl groups include, but are not limited to, methyl (C₁), ethyl (C₂), propyl (C₃), butyl (C₄), pentyl (C₅), hexyl (C₆), heptyl (C₇).

Examples of (unsubstituted) linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇).

Examples of (unsubstituted) branched alkyl groups include iso-propyl (C₃), iso-butyl (C₄), sec-butyl (C₄), tert-butyl (C₄), iso-pentyl (C₅), and neo-pentyl (C₅).

The term “alkenyl,” as used herein, pertains to a monovalent moiety obtained by removing a hydrogen atom from a carbon atom of an unsaturated aliphatic hydrocarbon compound having from 1 to 20 carbon atoms (unless otherwise specified) and having one or more carbon-carbon double bonds.

Examples of (unsubstituted) alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

The term “alkenyl,” as used herein, pertains to a monovalent moiety obtained by removing a hydrogen atom from a carbon atom of an unsaturated aliphatic hydrocarbon compound having from 1 to 20 carbon atoms (unless otherwise specified) and having one or more carbon-carbon triple bonds.

Examples of (unsubstituted) alkynyl groups include, but are not limited to, ethynyl (ethinyl, —C≡CH) and 2-propynyl (propargyl, —CH₂—C≡CH).

The term “cycloalkyl,” as used herein, pertains to a monovalent moiety obtained by removing a hydrogen atom from a carbon atom of a saturated hydrocarbon compound having at least one carbocyclic ring, and having from 3 to 20 carbon atoms (unless otherwise specified), including from 3 to 20 ring atoms (unless otherwise specified).

Examples of (unsubstituted) cycloalkyl groups include, but are not limited to, cyclopropyl (C₃), cyclobutyl (C₄), cyclopentyl (C₅), cyclohexyl (C₆), cycloheptyl (C₇), methylcyclopropyl (C₄), dimethylcyclopropyl (C₅), methylcyclobutyl (C₅), dimethylcyclobutyl (C₆), methylcyclopentyl (C₆), dimethylcyclopentyl (C₇), methylcyclohexyl (C₇).

Additional examples of (unsubstituted) cycloalkyl groups include, but are not limited to, cyclopropylmethyl (C₄), cyclobutylmethyl (C₅), cyclopentylmethyl (C₆), cyclohexylmethyl (C₇), cyclopropylethyl (C₅), cyclobutylethyl (C₆), cyclopentylethyl (C₇).

The term “cycloalkenyl,” as used herein, pertains to a monovalent moiety obtained by removing a hydrogen atom from a carbon atom of an unsaturated hydrocarbon compound having at least one carbocyclic ring that has at least one carbon-carbon double bond as part of that ring, and having from 3 to 20 carbon atoms (unless otherwise specified), including from 3 to 20 ring atoms (unless otherwise specified).

Examples of (unsubstituted) cycloalkenyl groups include, but are not limited to, cyclopropenyl (C₃), cyclobutenyl (C₄), cyclopentenyl (C₅), cyclohexenyl (C₆), methylcyclopropenyl (C₄), dimethylcyclopropenyl (C₅), methylcyclobutenyl (C₅), dimethylcyclobutenyl (C₆), methylcyclopentenyl (C₆), dimethylcyclopentenyl (C₇), methylcyclohexenyl (C₇).

The term “heterocyclyl,” as used herein, pertains to a monovalent moiety obtained by removing a hydrogen atom from a non-aromatic ring atom of a compound having at least one non-aromatic heterocyclic ring, and having from 3 to 20 carbon atoms (unless otherwise specified), including from 3 to 20 ring atoms (unless otherwise specified), of which from 1 to 10 are ring heteroatoms (unless otherwise specified). Preferably, each ring of the compound has from 3 to 7 ring atoms, of which from 1 to 4 are ring heteroatoms.

In this context, the prefixes (e.g., C₃₋₂₀, C₃₋₇, C₅₋₆, etc.) denote the number of ring atoms, or range of number of ring atoms, whether carbon atoms or heteroatoms. For example, the term “C₆₋₆heterocyclyl,” as used herein, pertains to a heterocyclyl group having 5 or 6 ring atoms. Examples of groups of heterocyclyl groups include C₃₋₁₄heterocyclyl, C₅₋₁₄heterocyclyl, C₃₋₁₂heterocyclyl, C₅₋₁₂heterocyclyl, C₃₋₁₀heterocyclyl, C₅₋₁₀heterocyclyl, C₃₋₇heterocyclyl, C₅₋₇heterocyclyl, and C₅₋₆heterocyclyl.

Examples of monocyclic heterocyclyl groups include, but are not limited to:
N₁: aziridinyl (C₃), azetidinyl (C₄), pyrrolidinyl (tetrahydropyrrolyl) (C₅), pyrrolinyl (e.g., 3-pyrrolinyl, 2,5-dihydropyrrolyl) (C₅), 2H-pyrrolyl or 3H-pyrrolyl (isopyrrolyl, isoazolyl) (C₅), piperidinyl (C₆), dihydropyridinyl (C₆), tetrahydropyridinyl (C₆), azepinyl (C₇);
O₁: oxiranyl (C₃), oxetanyl (C₄), oxolanyl (tetrahydrofuranyl) (Ce), oxolyl (dihydrofuranyl) (C₅), oxanyl (tetrahydropyranyl) (C₆), dihydropyranyl (C₆), pyranyl (C₆), oxepinyl (C₇);
S₁: thiiranyl (C₃), thietanyl (C₄), thiolanyl (tetrahydrothienyl) (C₅), thianyl (tetrahydrothiopyranyl) (C₆), thiepanyl (C₇);
O₂: dioxolanyl (C₅), dioxanyl (C₆), and dioxepanyl (C₇);
O₃: trioxanyl (C₆);
N₂: imidazolidinyl (C₅), pyrazolidinyl (diazolidinyl) (C₅), imidazolinyl (C₅), pyrazolinyl (dihydropyrazolyl) (C₅), piperazinyl (C₆);
N₁O₁: tetrahydrooxazolyl (C₅), dihydrooxazolyl (C₅), tetrahydroisoxazolyl (C₅), dihydroisoxazolyl (C₅), morpholinyl (C₆), tetrahydrooxazinyl (C₆), dihydrooxazinyl (C₆), oxazinyl (C₆);
N₁S₁: thiazolinyl (C₅), thiazolidinyl (C₅), thiomorpholinyl (C₆);
N₂O₁: oxadiazinyl (C₆);
O₁S₁: oxathiolyl (C₆) and oxathianyl (thioxanyl) (C₆); and,
N₁O₁S₁: oxathiazinyl (C₆).

The term “aryl,” as used herein, pertains to a monovalent moiety obtained by removing a hydrogen atom from an aromatic ring atom of an aromatic compound, which moiety has from 3 to 20 ring atoms (unless otherwise specified). Preferably, each ring has from 5 to 7 ring atoms, of which from 0 to 4 are ring heteroatoms.

In this context, the prefixes (e.g., C₃₋₂₀, C₅₋₇, C₅₋₆, etc.) denote the number of ring atoms, or range of number of ring atoms, whether carbon atoms or heteroatoms. For example, the term “C₅₋₆aryl,” as used herein, pertains to an aryl group having 5 or 6 ring atoms. Examples of groups of aryl groups include C₅₋₁₄aryl, C₅₋₁₂aryl, C₅₋₁₀aryl, C₅₋₉aryl, C₅₋₆aryl, C₆aryl, and C₆aryl.

The ring atoms may be all carbon atoms, as in “carboaryl groups.” Examples of carboaryl groups include C₆₋₁₄carboaryl, C₆₋₁₂carboaryl, C₆₋₁₀carboaryl, C₆₋₉carboaryl, C₆₋₆carboaryl, and C₆carboaryl.

Examples of carboaryl groups include, but are not limited to, phenyl (C₆), naphthyl (C₁₀), azulenyl (C₁₀), anthracenyl (C₁₄), and phenanthrenyl (C₁₄).

Examples of carboaryl groups which comprise fused rings, include, but are not limited to, indanyl (C₉), indenyl (C₉), isoindenyl (C₉), tetralinyl (1,2,3,4-tetrahydronaphthalene (C₁₀), acenaphthenyl (C₁₂), fluorenyl (C₁₃), and phenalenyl (C₁₃).

Alternatively, the ring atoms may include one or more heteroatoms (for example, N, O, and/or S), as in “heteroaryl groups.” In this context, the prefixes (e.g., C₅₋₂₀, C₅₋₇, C₅₋₆, etc.) denote the number of ring atoms, or range of number of ring atoms, whether carbon atoms or heteroatoms. For example, the term “C₅₋₆heteroaryl,” as used herein, pertains to a heteroaryl group having 5 or 6 ring atoms. Examples of heteroaryl groups include C₅₋₁₄heteroaryl, C₅₋₁₂heteroaryl, C₅₋₁₀heteroaryl, C₅₋₉heteroaryl, C₅₋₆heteroaryl, C₅heteroaryl, and C₆heteroaryl.

Examples of heteroaryl groups include, but are not limited to,
N₁: pyrrolyl (azolyl) (C₅), pyridinyl (azinyl) (C₆);
01: furanyl (oxolyl) (C₅);
S₁: thiophenyl (thionyl) (C₅);
N₁O₁: oxazolyl (C₅), isoxazolyl (C₅), isoxazinyl (C₆);
N₂O₁: oxadiazolyl (furazanyl) (C₅);
N₃O₁: oxatriazolyl (C₅);
N₁₁S₁: thiazolyl (C₅), isothiazolyl (C₅);
N₂: imidazolyl (1,3-diazolyl) (C₅), pyrazolyl (1,2-diazolyl) (C₅), pyridazinyl (1,2-diazinyl) (C₆), pyrimidinyl (1,3-diazinyl) (C₆) (e.g., cytosinyl, thyminyl, uracilyl), pyrazinyl (1,4-diazinyl) (C₆);
N₃: triazolyl (C₅), triazinyl (C₆); and,
N₄: tetrazolyl (C₅).

Examples of heteroaryl groups which comprise fused rings, include, but are not limited to,
C₉heterocyclic groups (with 2 fused rings): benzofuranyl (O₁), isobenzofuranyl (O₁), indolyl (N₁), isoindolyl (N₁), indolizinyl (N₁), indolinyl (N₁), isoindolinyl (N₁), purinyl (N₄) (e.g., adeninyl, guaninyl), benzimidazolyl (N₂), indazolyl (N₂), benzoxazolyl (N₁O₁), benzisoxazolyl (N₁O₁), benzodioxolyl (O₂), benzofurazanyl (N₂O₁), benzotriazolyl (N₃), benzothiofuranyl (S₁), benzothiazolyl (N₁S₁), benzothiadiazolyl (N₂S);
C₁₀heterocyclic groups (with 2 fused rings): chromenyl (O₁), isochromenyl (O₁), chromanyl (O₁), isochromanyl (O₁), benzodioxanyl (O₂), quinolinyl (N₁), isoquinolinyl (N₁), quinolizinyl (N₁), benzoxazinyl (N₁O₁), benzodiazinyl (N₂), pyridopyridinyl (N₂), quinoxalinyl (N₂), quinazolinyl (N₂), cinnolinyl (N₂), phthalazinyl (N₂), naphthyridinyl (N₂), pteridinyl (N₄);
C₁₁heterocylic groups (with 2 fused rings): benzodiazepinyl (N₂);
C₁₋₃heterocyclic groups (with 3 fused rings): carbazolyl (N₁), dibenzofuranyl (O₁), dibenzothiophenyl (S₁), carbolinyl (N₂), pyridoindolyl (N₂); and,
C₁₄heterocyclic groups (with 3 fused rings): acridinyl (N₁), xanthenyl (O₁), thioxanthenyl (S₁), oxanthrenyl (O₂), phenoxathiin (O₁S₁), phenazinyl (N₂), phenoxazinyl (N₁O₁), phenothiazinyl (N₁S₁), thianthrene (S₂), phenanthridine (N₁), phenanthroline (N₂), phenazine (N₂).

Heteroaryl groups that have a nitrogen ring atom in the form of an —NH— group may be N-substituted, that is, as —NR—. For example, pyrrolyl may be N-methyl substituted, to give N-methylpyrrolyl. Examples of N-substitutents include, but are not limited to, C₁₋₇alkyl, C₅₋₁₄aryl, C₅₋₁₄aryl-C₁₋₇alkyl, C₁₋₇alkyl-acyl, and C₅₋₁₄aryl-C₁₋₇alkyl-acyl groups. Heteroaryl groups that have a nitrogen ring atom in the form of an —N═ group may be substituted in the form of an N-oxide, that is, as —N(→O)=(also denoted —N⁺(→O⁻)═). For example, quinoline may be substituted to give quinoline N-oxide; pyridine to give pyridine N-oxide; benzofurazan to give benzofurazan N-oxide (also known as benzofuroxan).

Cycloalkyl, cycloalkenyl, heterocyclic, carboaryl, and heteroaryl groups may additionally bear one or more oxo (═O) groups on ring carbon atoms (or ring sulfur atoms, if present).

Some monocyclic examples of such groups include, but are not limited to:

C₅: cyclopentanonyl, cyclopentenonyl, cyclopentadienonyl;
C₆: cyclohexanonyl, cyclohexenonyl, cyclohexadienonyl;
O₁: furanonyl (C₅), pyronyl (C₆);
N₁: pyrrolidonyl (pyrrolidinonyl) (C₅), piperidinonyl (piperidonyl) (C₆), piperidinedionyl (C₆);
N₂: imidazolidonyl (imidazolidinonyl) (C₅), pyrazolonyl (pyrazolinonyl) (C₅), piperazinonyl (C₆), piperazinedionyl (C₆), pyridazinonyl (C₆), pyrimidinonyl (C₆) (e.g., cytosinyl), pyrimidinedionyl (C₆) (e.g., thyminyl, uracilyl);
N₁S₁: thiazolonyl (C₅), isothiazolinyl (C₅);
N₁O₁: oxazolinonyl (C₅).

Some polycyclic examples of such groups include, but are not limited to:

C₉: indenedionyl;
C₁₀: tetralonyl, decalonyl;
C₁₄: anthronyl, phenanthronyl;
N₁: oxindolyl (C₉);
O₁: benzopyronyl (e.g., coumarinyl, isocoumarinyl, chromonyl) (C₁₀);
N₁O₁: benzoxazolinonyl (C₉), benzoxazolinonyl (C₁₀);
N₂: quinazolinedionyl (C₁₀); benzodiazepinonyl (C₁₁); benzodiazepinedionyl (C₁₁);
N₄: purinonyl (C₉) (e.g., guaninyl).

Still more examples of cyclic groups which bear one or more oxo (═O) groups on ring carbon atoms include, but are not limited to, those found in:
cyclic anhydrides (—C(═O)—O—C(═O)— in a ring), including but not limited to maleic anhydride (C₅), succinic anhydride (C₅), and glutaric anhydride (C₆); cyclic carbonates (—O—C(═O)—O— in a ring), such as ethylene carbonate (C₅) and 1,2-propylene carbonate (C₅);
imides (—C(═O)—NR—C(═O)— in a ring), including but not limited to, succinimide (C₅), maleimide (C₅), phthalimide, and glutarimide (C₆);
lactones (cyclic esters, —O—C(═O)— in a ring), including, but not limited to, β-propiolactone, γ-butyrolactone, δ-valerolactone (2-piperidone), and ε-caprolactone;
lactams (cyclic amides, —NR—C(═O)— in a ring), including, but not limited to, β-propiolactam (C₄), γ-butyrolactam (2-pyrrolidone) (C₅), 6-valerolactam (C₆), and ε-caprolactam (C₇);
cyclic carbamates (—O—C(═O)—NR— in a ring), such as 2-oxazolidone (C₅);
cyclic ureas (—NR—C(═O)—NR— in a ring), such as 2-imidazolidone (C₅) and pyrimidine-2,4-dione (e.g., thymine, uracil) (C₆).

### Molecular Weight

In one embodiment, the compound has a molecular weight of 229 to 1200.

In one embodiment, the bottom of range is 230; 250; 275; 325; 350; 375; 400; 425; 450.

In one embodiment, the top of range is 1100, 1000, 900; 800; 700; 600; 500.

In one embodiment, the range is 250 to 1100.

In one embodiment, the range is 250 to 1000.

In one embodiment, the range is 250 to 900.

In one embodiment, the range is 250 to 800.

In one embodiment, the range is 250 to 700.

In one embodiment, the range is 250 to 600.

In one embodiment, the range is 250 to 500.

### Some Preferred Embodiments

All compatible combinations of the embodiments described above are explicitly disclosed herein, as if each compatible combination was individually and explicitly recited.

Examples of some compounds (where J is N) are shown below.

| # | Structure | Name | |
|---|---|---|---|
| 1 | 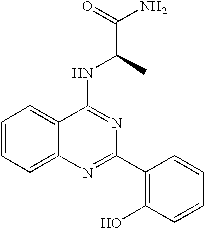 | (R)-2-[2-(2- Hydroxy- phenyl)- quinazolin-4- ylamino]- propionamide | XX-001 |
| 2 | 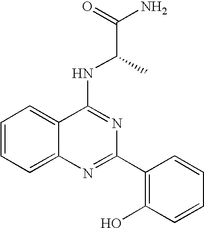 | (S)-2-[2-(2- Hydroxy- phenyl)- quinazolin-4- ylamino]- propionamide | XX-002 |
| 3 | 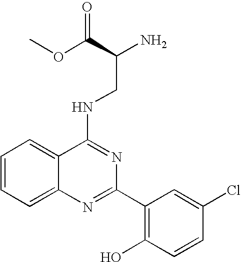 | (S)-2-Amino-3- [2-(5-chloro-2- hydroxy-phenyl)- quinazolin-4- ylamino]- propionic acid methyl ester | XX-003 |
| 4 | 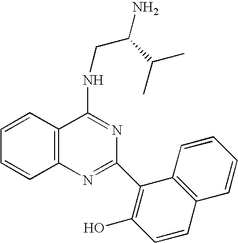 | 1-[4-((R)-2- Amino-3-methyl- butylamino)- quinazolin-2-yl]- naphthalen-2-ol | XX-004 |
| 5 | 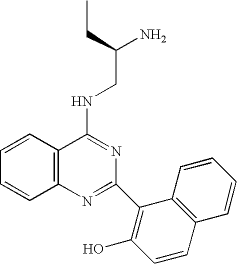 | 1-[4-((R)-2- Amino- butylamino)- quinazolin-2-yl]- naphthalen-2-ol | XX-005 |
| 6 | 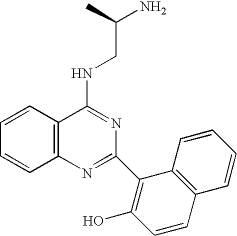 | 1-[4-((R)-2- Amino- propylamino)- quinazolin-2-yl]- naphthalen-2-ol | XX-006 |
| 7 | 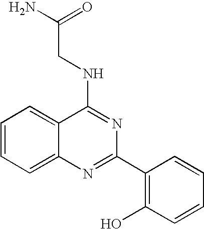 | 2-[2-(2-Hydroxy- phenyl)- quinazolin-4- ylamino]- acetamide | XX-007 |
| 8 | 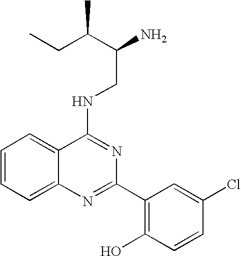 | 2-[4-((2R,3R)-2- Amino-3-methyl- pentylamino)- quinazolin-2-yl]- 4-chloro-phenol | XX-008 |
| 9 | 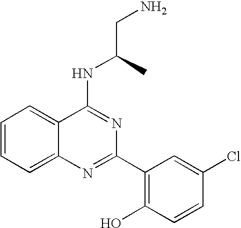 | 2-[4-((R)-2- Amino-1-methyl- ethylamino)- quinazolin-2-yl]- 4-chloro-phenol | XX-009 |
| 10 | 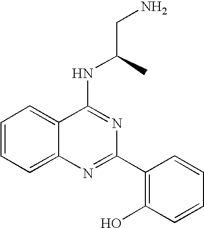 | 2-[4-((R)-2- Amino-1-methyl- ethylamino)- quinazolin-2-yl]- phenol | XX-010 |
| 11 | 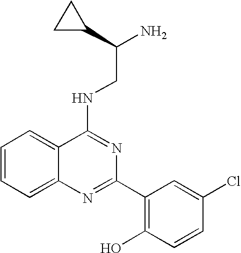 | 2-[4-((R)-2- Amino-2- cyclopropyl- ethylamino)- quinazolin-2-yl]- 4-chloro-phenol | XX-011 |
| 12 | 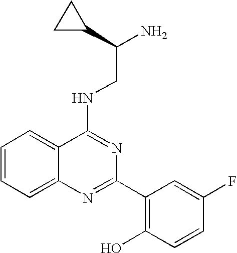 | 2-[4-((R)-2- Amino-2- cyclopropyl- ethylamino)- quinazolin-2-yl]- 4-fluoro-phenol | XX-012 |
| 13 | 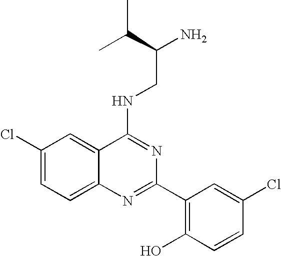 | 2-[4-((R)-2- Amino-3-methyl- butylamino)-6- chloro- quinazolin-2-yl]- 4-chloro-phenol | XX-013 |
| 14 | 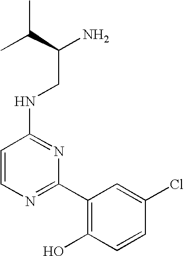 | 2-[4-((R)-2- Amino-3-methyl- butylamino)- pyrimidin-2-yl]-4- chloro-phenol | XX-014 |
| 15 | 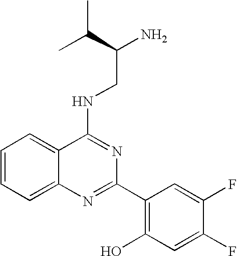 | 2-[4-((R)-2- Amino-3-methyl- butylamino)- quinazolin-2-yl]- 4,5-difluoro- phenol | XX-015 |
| 16 | 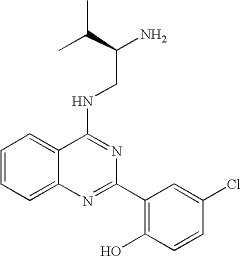 | 2-[4-((R)-2- Amino-3-methyl- butylamino)- quinazolin-2-yl]- 4-chloro-phenol | XX-016 |
| 17 | 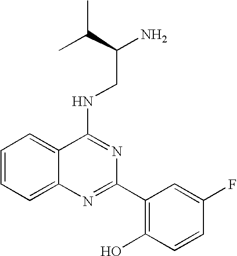 | 2-[4-((R)-2- Amino-3-methyl- butylamino)- quinazolin-2-yl]- 4-fluoro-phenol | XX-017 |
| 18 | 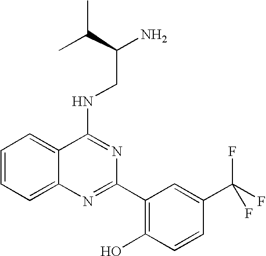 | 2-[4-((R)-2- Amino-3-methyl- butylamino)- quinazolin-2-yl]- 4-trifluoromethyl- phenol | XX-018 |
| 19 | 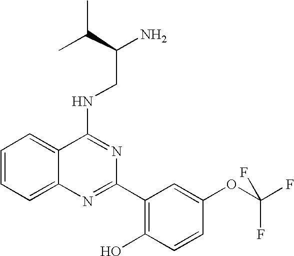 | 2-[4-((R)-2- Amino-3-methyl- butylamino)- quinazolin-2-yl]- 4- trifluoromethoxy- phenol | XX-019 |
| 20 | 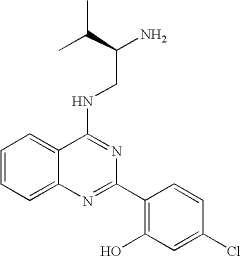 | 2-[4-((R)-2- Amino-3-methyl- butylamino)- quinazolin-2-yl]- 5-chloro-phenol | XX-020 |
| 21 | 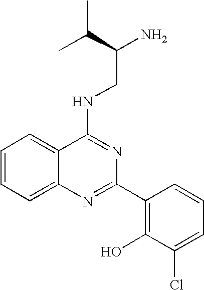 | 2-[4-((R)-2- Amino-3-methyl- butylamino)- quinazolin-2-yl]- 6-chloro-phenol | XX-021 |
| 22 | 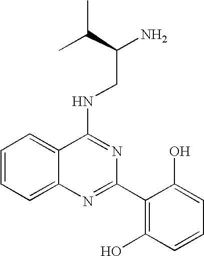 | 2-[4-((R)-2- Amino-3-methyl- butylamino)- quinazolin-2-yl]- benzene-1,3-diol | XX-022 |
| 23 | 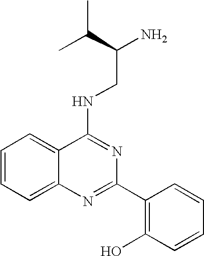 | 2-[4-((R)-2- Amino-3-methyl- butylamino)- quinazolin-2-yl]- phenol | XX-023 |
| 24 | 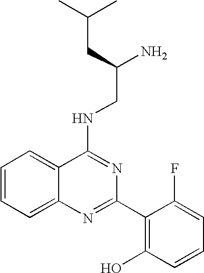 | 2-[4-((R)-2- Amino-4-methyl- pentylamino)- quinazolin-2-yl]- 3-fluoro-phenol | XX-024 |
| 25 | 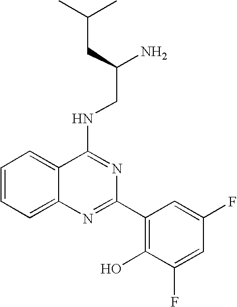 | 2-[4-((R)-2- Amino-4-methyl- pentylamino)- quinazolin-2-yl]- 4,6-difluoro- phenol | XX-025 |
| 26 | 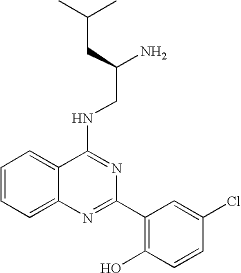 | 2-[4-((R)-2- Amino-4-methyl- pentylamino)- quinazolin-2-yl]- 4-chloro-phenol | XX-026 |
| 27 | 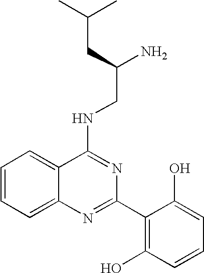 | 2-[4-((R)-2- Amino-4-methyl- pentylamino)- quinazolin-2-yl]- benzene-1,3-diol | XX-027 |
| 28 | 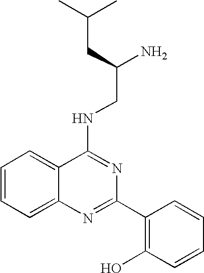 | 2-[4-((R)-2- Amino-4-methyl- pentylamino)- quinazolin-2-yl]- phenol | XX-028 |
| 29 | 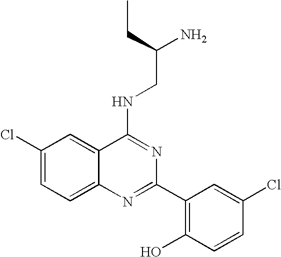 | 2-[4-((R)-2- Amino- butylamino)-6- chloro- quinazolin-2-yl]- 4-chloro-phenol | XX-029 |
| 30 | 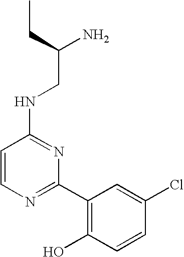 | 2-[4-((R)-2- Amino- butylamino)- pyrimidin-2-yl]-4- chloro-phenol | XX-030 |
| 31 | 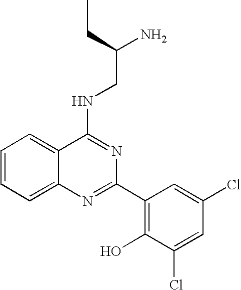 | 2-[4-((R)-2- Amino- butylamino)- quinazolin-2-yl]- 4,6-dichloro- phenol | XX-031 |
| 32 | 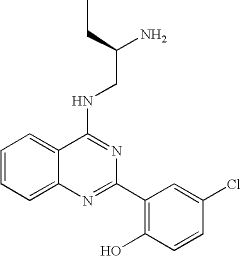 | 2-[4-((R)-2- Amino- butylamino)- quinazolin-2-yl]- 4-chloro-phenol | XX-032 |
| 33 | 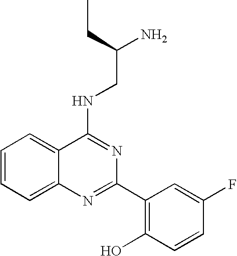 | 2-[4-((R)-2- Amino- butylamino)- quinazolin-2-yl]- 4-fluoro-phenol | XX-033 |
| 34 | 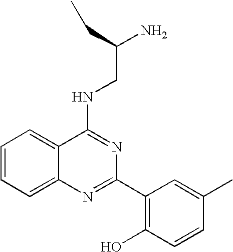 | 2-[4-((R)-2- Amino- butylamino)- quinazolin-2-yl]- 4-methyl-phenol | XX-034 |
| 35 | 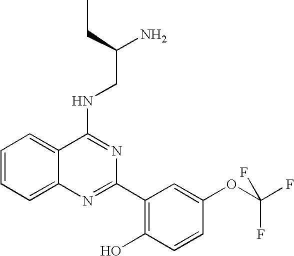 | 2-[4-((R)-2- Amino- butylamino)- quinazolin-2-yl]- 4- trifluoromethoxy- phenol | XX-035 |
| 36 | 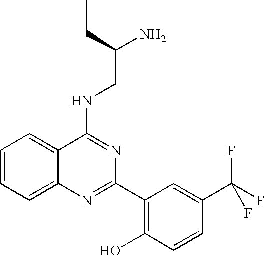 | 2-[4-((R)-2- Amino- butylamino)- quinazolin-2-yl]- 4-trifluoromethyl- phenol | XX-036 |
| 37 | 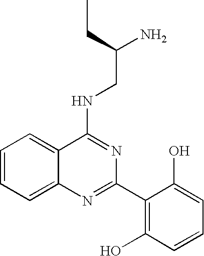 | 2-[4-((R)-2- Amino- butylamino)- quinazolin-2-yl]- benzene-1,3-diol | XX-037 |
| 38 | 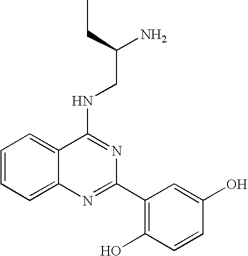 | 2-[4-((R)-2- Amino- butylamino)- quinazolin-2-yl]- benzene-1,4-diol | XX-038 |
| 39 | 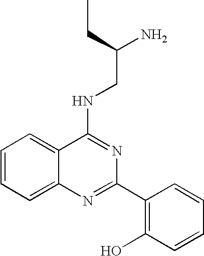 | 2-[4-((R)-2- Amino- butylamino)- quinazolin-2-yl]- phenol | XX-039 |
| 40 | 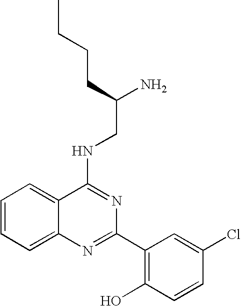 | 2-[4-((R)-2- Amino- hexylamino)- quinazolin-2-yl]- 4-chloro-phenol | XX-040 |
| 41 | 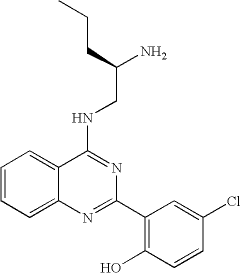 | 2-[4-((R)-2- Amino- pentylamino)- quinazolin-2-yl]- 4-chloro-phenol | XX-041 |
| 42 | 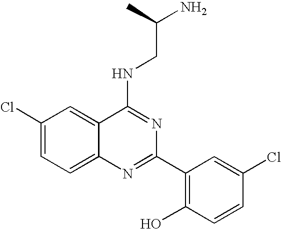 | 2-[4-((R)-2- Amino- propylamino)-6- chloro- quinazolin-2-yl]- 4-chloro-phenol | XX-042 |
| 43 | 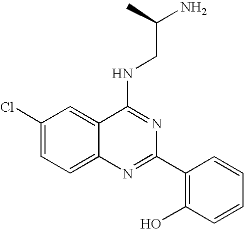 | 2-[4-((R)-2- Amino- propylamino)-6- chloro- quinazolin-2-yl]- phenol | XX-043 |
| 44 | 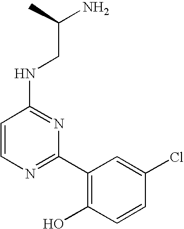 | 2-[4-((R)-2- Amino- propylamino)- pyrimidin-2-yl]-4- chloro-phenol | XX-044 |
| 45 | 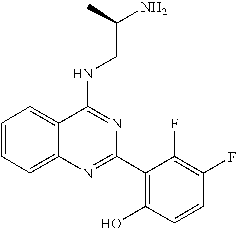 | 2-[4-((R)-2- Amino- propylamino)- quinazolin-2-yl]- 3,4-difluoro- phenol | XX-045 |
| 46 | 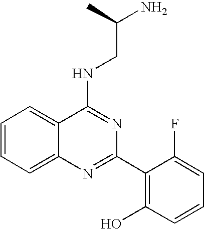 | 2-[4-((R)-2- Amino- propylamino)- quinazolin-2-yl]- 3-fluoro-phenol | XX-046 |
| 47 | 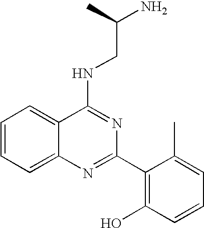 | 2-[4-((R)-2- Amino- propylamino)- quinazolin-2-yl]- 3-methyl-phenol | XX-047 |
| 48 | 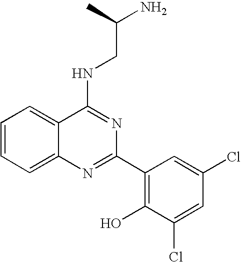 | 2-[4-((R)-2- Amino- propylamino)- quinazolin-2-yl]- 4,6-dichloro- phenol | XX-048 |
| 49 | 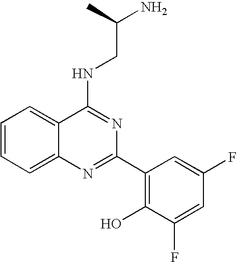 | 2-[4-((R)-2- Amino- propylamino)- quinazolin-2-yl]- 4,6-difluoro- phenol | XX-049 |
| 50 | 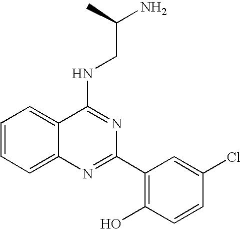 | 2-[4-((R)-2- Amino- propylamino)- quinazolin-2-yl]- 4-chloro-phenol | XX-050 |
| 51 | 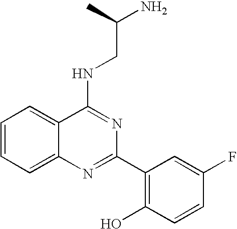 | 2-[4-((R)-2- Amino- propylamino)- quinazolin-2-yl]- 4-fluoro-phenol | XX-051 |
| 52 | 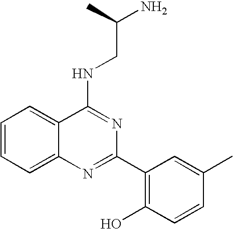 | 2-[4-((R)-2- Amino- propylamino)- quinazolin-2-yl]- 4-methyl-phenol | XX-052 |
| 53 | 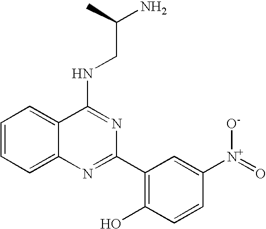 | 2-[4-((R)-2- Amino- propylamino)- quinazolin-2-yl]- 4-nitro-phenol | XX-053 |
| 54 | 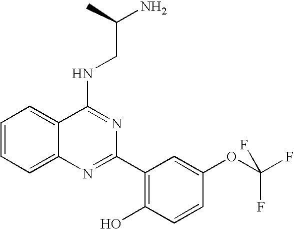 | 2-[4-((R)-2- Amino- propylamino)- quinazolin-2-yl]- 4- trifluoromethoxy- phenol | XX-054 |
| 55 | 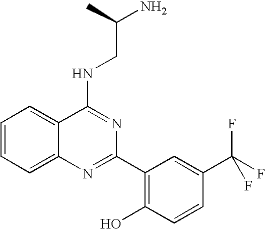 | 2-[4-((R)-2- Amino- propylamino)- quinazolin-2-yl]- 4-trifluoromethyl- phenol | XX-055 |
| 56 | 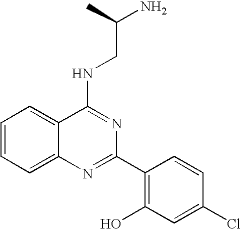 | 2-[4-((R)-2- Amino- propylamino)- quinazolin-2-yl]- 5-chloro-phenol | XX-056 |
| 57 | 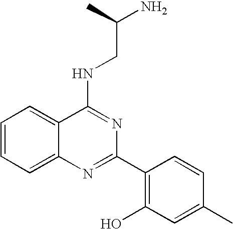 | 2-[4-((R)-2- Amino- propylamino)- quinazolin-2-yl]- 5-methyl-phenol | XX-057 |
| 58 | 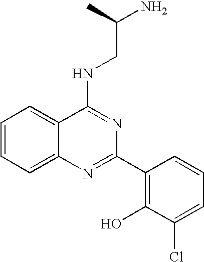 | 2-[4-((R)-2- Amino- propylamino)- quinazolin-2-yl]- 6-chloro-phenol | XX-058 |
| 59 | 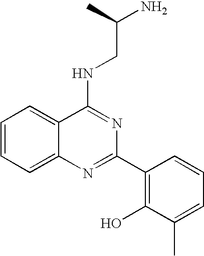 | 2-[4-((R)-2- Amino- propylamino)- quinazolin-2-yl]- 6-methyl-phenol | XX-059 |
| 60 | 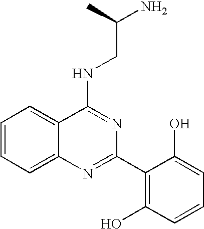 | 2-[4-((R)-2- Amino- propylamino)- quinazolin-2-yl]- benzene-1,3-diol | XX-060 |
| 61 | 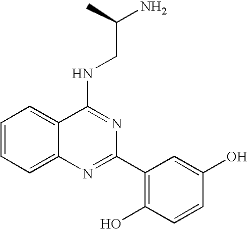 | 2-[4-((R)-2- Amino- propylamino)- quinazolin-2-yl]- benzene-1,4-diol | XX-061 |
| 62 | 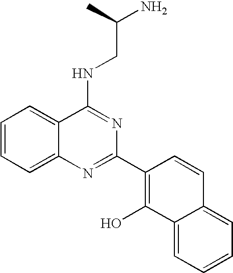 | 2-[4-((R)-2- Amino- propylamino)- quinazolin-2-yl]- naphthalen-1-ol | XX-062 |
| 63 | 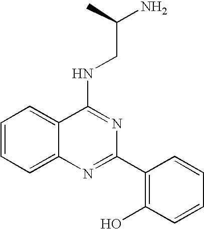 | 2-[4-((R)-2- Amino- propylamino)- quinazolin-2-yl]- phenol | XX-063 |
| 64 | 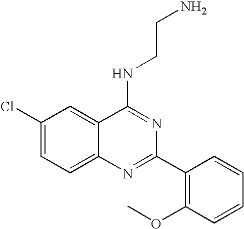 | N1-[6-Chloro-2- (2-methoxy- phenyl)- quinazolin-4-yl]- ethane-1,2- diamine | XX-064 |
| 65 | 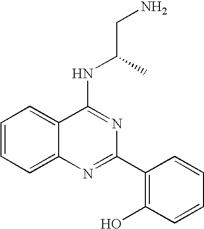 | 2-[4-((S)-2- Amino-1-methyl- ethylamino)- quinazolin-2-yl]- phenol | XX-065 |
| 66 | 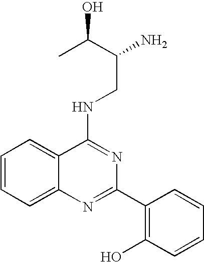 | 2-[4-((2R,3R)-2- Amino-3- hydroxy- butylamino)- quinazolin-2-yl]- phenol | XX-066 |
| 67 | 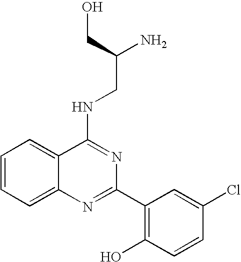 | 2-[4-((S)-2- Amino-3- hydroxy- propylamino)- quinazolin-2-yl]- 4-chloro-phenol | XX-067 |
| 68 | 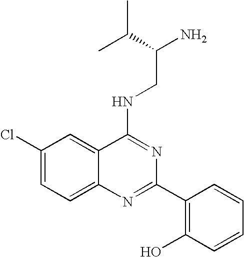 | 2-[4-((S)-2- Amino-3-methyl- butylamino)-6- chloro- quinazolin-2-yl]- phenol | XX-068 |
| 69 | 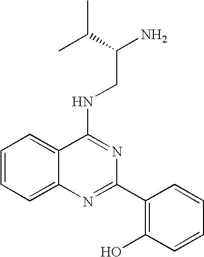 | 2-[4-((S)-2- Amino-3-methyl- butylamino)- quinazolin-2-yl]- phenol | XX-069 |
| 70 | 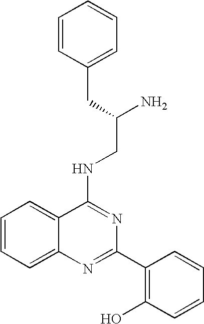 | 2-[4-((S)-2- Amino-3-phenyl- propylamino)- quinzazolin-2-yl]- phenol | XX-070 |
| 71 | 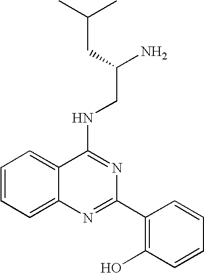 | 2-[4-((S)-2- Amino-4-methyl- pentylamino)- quinazolin-2-yl]- phenol | XX-071 |
| 72 | 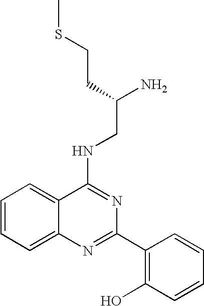 | 2-[4-((S)-2- Amino-4- methylsulfanyl- butylamino)- quinazolin-2-yl]- phenol | XX-072 |
| 73 | 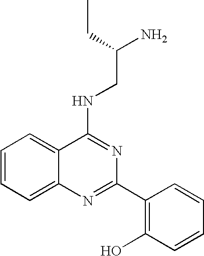 | 2-[4-((S)-2- Amino- butylamino)- quinazolin-2-yl]- phenol | XX-073 |
| 74 | 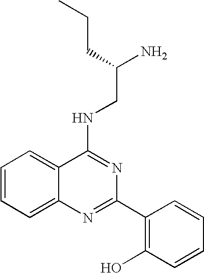 | 2-[4-((S)-2- Amino- pentylamino)- quinazolin-2-yl]- phenol | XX-074 |
| 75 | 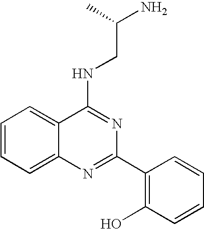 | 2-[4-((S)-2- Amino- propylamino)- quinazolin-2-yl]- phenol | XX-075 |
| 76 | 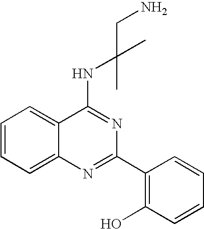 | 2-[4-(2-Amino- 1,1-dimethyl- ethylamino)- quinazolin-2-yl]- phenol | XX-076 |
| 77 | 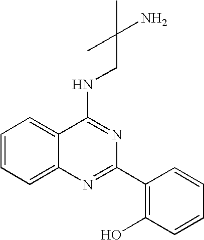 | 2-[4-(2-Amino-2- methyl- propylamino)- quinazolin-2-yl]- phenol | XX-077 |
| 78 | 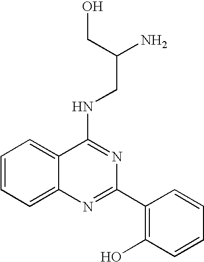 | 2-[4-(2-Amino-3- hydroxy- propylamino)- quinazolin-2-yl]- phenol | XX-078 |
| 79 | 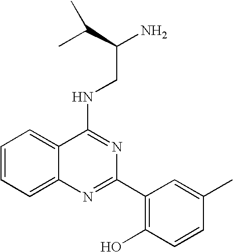 | 2-[4-(2-Amino-3- methyl- butylamino)- quinazolin-2-yl]- 4-methyl-phenol | XX-079 |
| 80 | 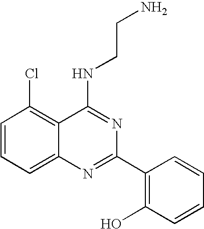 | 2-[4-(2-Amino- ethylamino)-5- chloro- quinazolin-2-yl]- phenol | XX-080 |
| 81 | 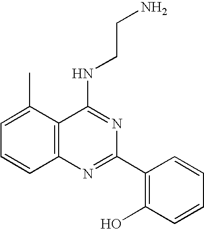 | 2-[4-(2-Amino- ethylamino)-5- methyl- quinazolin-2-yl]- phenol | XX-081 |
| 82 | 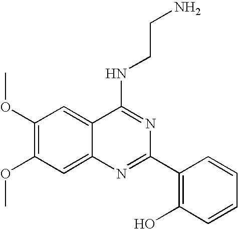 | 2-[4-(2-Amino- ethylamino)-6,7- dimethoxy- quinazolin-2-yl]- phenol | XX-082 |
| 83 | 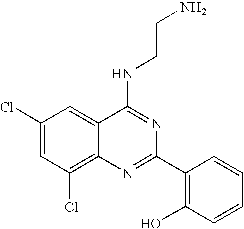 | 2-[4-(2-Amino- ethylamino)-6,8- dichloro- quinazolin-2-yl]- phenol | XX-083 |
| 84 | 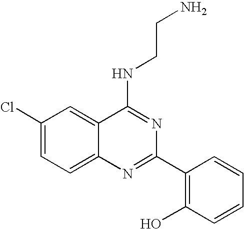 | 2-[4-(2-Amino- ethylamino)-6- chloro- quinazolin-2-yl]- phenol | XX-084 |
| 85 | 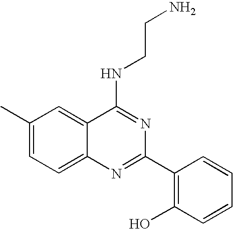 | 2-[4-(2-Amino- ethylamino)-6- methyl- quinazolin-2-yl]- phenol | XX-085 |
| 86 | 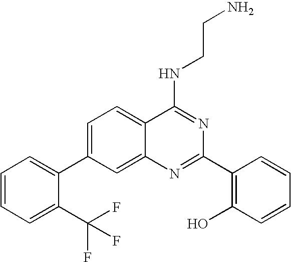 | 2-[4-(2-Amino- ethylamino)-7- (2- trifluoromethyl- phenyl)- quinazolin-2-yl]- phenol | XX-086 |
| 87 | 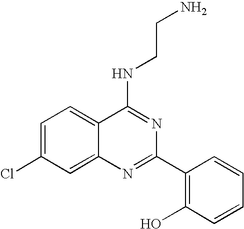 | 2-[4-(2-Amino- ethylamino)-7- chloro- quinazolin-2-yl]- phenol | XX-087 |
| 88 | 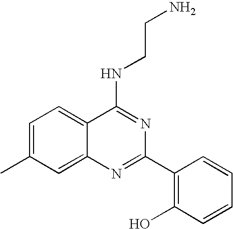 | 2-[4-(2-Amino- ethylamino)-7- methyl- quinazolin-2-yl]- phenol | XX-088 |
| 89 | 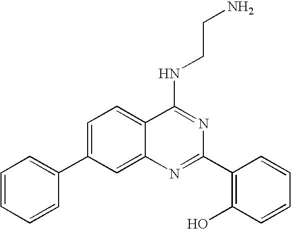 | 2-[4-(2-Amino- ethylamino)-7- phenyl- quinazolin-2-yl]- phenol | XX-089 |
| 90 | 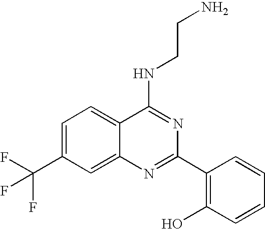 | 2-[4-(2-Amino- ethylamino)-7- trifluoromethyl- quinazolin-2-yl]- phenol | XX-090 |
| 91 | 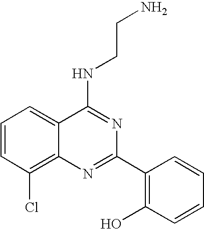 | 2-[4-(2-Amino- ethylamino)-8- chloro- quinazolin-2-yl]- phenol | XX-091 |
| 92 | 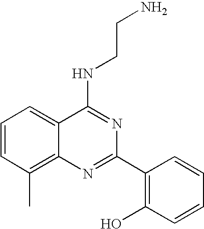 | 2-[4-(2-Amino- ethylamino)-8- methyl- quinazolin-2-yl]- phenol | XX-092 |
| 93 | 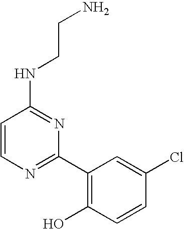 | 2-[4-(2-Amino- ethylamino)- pyrimidin-2-yl]-4- chloro-phenol | XX-093 |
| 94 | 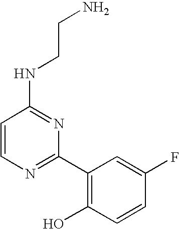 | 2-[4-(2-Amino- ethylamino)- pyrimidin-2-yl]-4- fluoro-phenol | XX-094 |
| 95 | 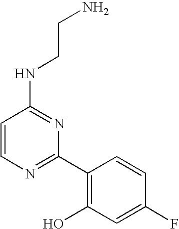 | 2-[4-(2-Amino- ethylamino)- pyrimidin-2-yl]-5- fluoro-phenol | XX-095 |
| 96 | 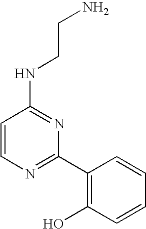 | 2-[4-(2-Amino- ethylamino)- pyrimidin-2-yl]- phenol | XX-096 |
| 97 | 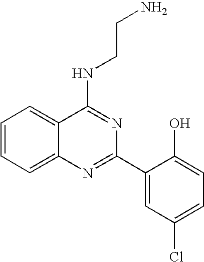 | 2-[4-(2-Amino- ethylamino)- quinazolin-2-yl]- 4-chloro-phenol | XX-097 |
| 98 | 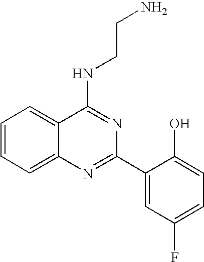 | 2-[4-(2-Amino- ethylamino)- quinazolin-2-yl]- 4-fluoro-phenol | XX-098 |
| 99 | 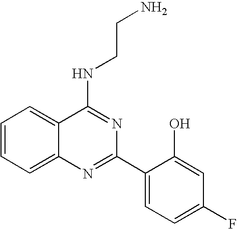 | 2-[4-(2-Amino- ethylamino)- quinazolin-2-yl]- 5-fluoro-phenol | XX-099 |
| 100 | 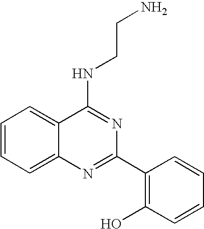 | 2-[4-(2-Amino- ethylamino)- quinazolin-2-yl]- phenol | XX-100 |
| 101 | 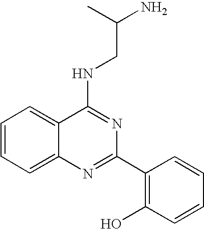 | 2-[4-(2-Amino- propylamino)- quinazolin-2-yl]- phenol | XX-101 |
| 102 | 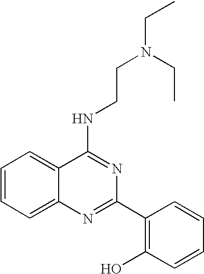 | 2-[4-(2- Diethylamino- ethylamino)- quinazolin-2-yl]- phenol | XX-102 |
| 103 | 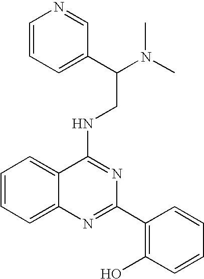 | 2-[4-(2- Dimethylamino- 2-pyridin-3-yl- ethylamino)- quinazolin-2-yl]- phenol | XX-103 |
| 104 | 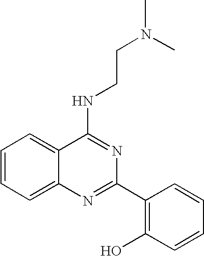 | 2-[4-(2- Dimethylamino- ethylamino)- quinazolin-2-yl]- phenol | XX-104 |
| 105 | 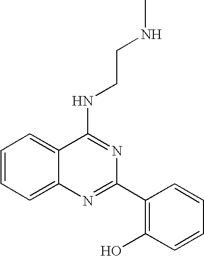 | 2-[4-(2- Methylamino- ethylamino)- quinazolin-2-yl]- phenol | XX-105 |
| 106 | 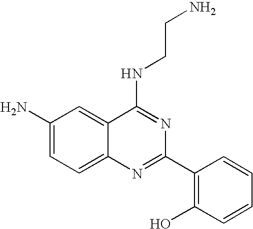 | 2-[6-Amino-4-(2- amino- ethylamino)- quinazolin-2-yl]- phenol | XX-106 |
| 107 | 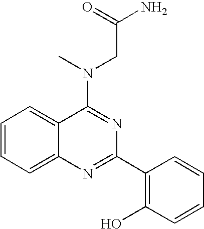 | 2-{[2-(2- Hydroxy- phenyl)- quinazolin-4-yl]- methyl-amino}- acetamide | XX-107 |
| 108 | 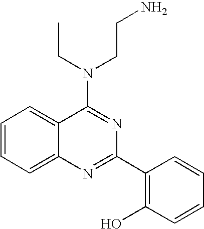 | 2-{4-[(2-Amino- ethyl)-ethyl- amino]- quinazolin-2-yl}- phenol | XX-108 |
| 109 | 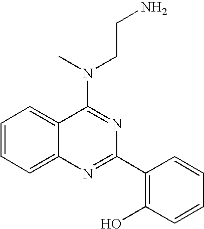 | 2-{4-[(2-Amino- ethyl)-ethyl- amino]- quinazolin-2-yl}- phenol | XX-109 |
| 110 | 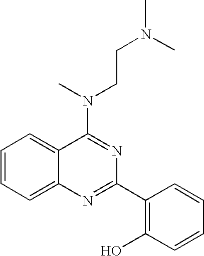 | 2-{4-[(2- Dimethylamino- ethyl)-methyl- amino]- quinazolin-2-yl}- phenol | XX-110 |
| 111 | 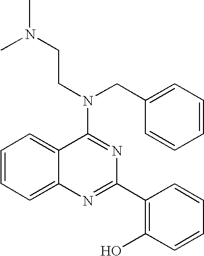 | 2-{4-[Benzyl-(2- dimethylamino- ethyl)-amino]- quinazolin-2-yl}- phenol | XX-111 |
| 112 | 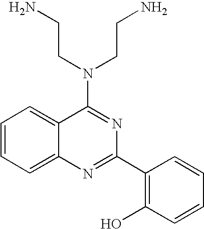 | 2-{4-[Bis-(2- amino-ethyl)- amino]- quinazolin-2-yl}- phenol | XX-112 |
| 113 | 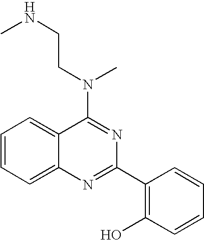 | 2-{4-[Methyl-(2- methylamino- ethyl)-amino]- quinazolin-2-yl}- phenol | XX-113 |
| 114 | 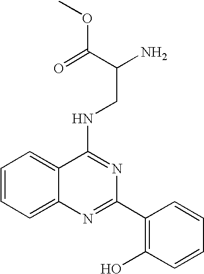 | 2-Amino-3-[2-(2- hydroxy-phenyl)- quinazolin-4- ylamino]- propionic acid methyl ester | XX-114 |
| 115 | 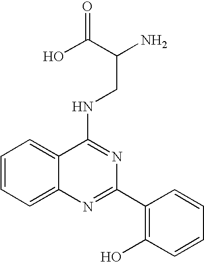 | 2-Amino-3-[2-(2- hydroxy-phenyl)- quinazolin-4- ylamino]- propionic acid | XX-115 |
| 116 | 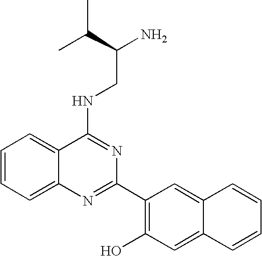 | 3-[4-((R)-2- Amino-3-methyl- butylamino)- quinazolin-2-yl]- naphthalen-2-ol | XX-116 |
| 117 | 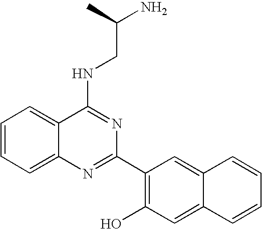 | 3-[4-((R)-2- Amino- propylamino)- quinazolin-2-yl]- naphthalen-2-ol | XX-117 |
| 118 | 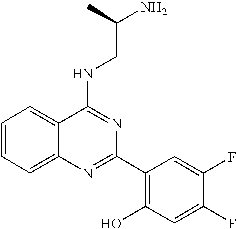 | 2-[4-((R)-2- Amino- propylamino)- quinazolin-2-yl]- 4,5-difluoro- phenol | XX-118 |
| 119 | 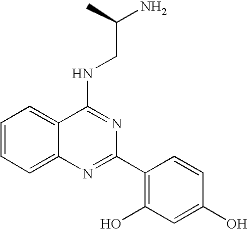 | 4-[4-((R)-2- Amino- propylamino)- quinazolin-2-yl]- benzene-1,3-diol | XX-119 |
| 120 | 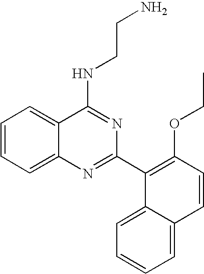 | N*1*-[2-(2- Ethoxy- naphthalen-1-yl)- quinazolin-4-yl]- ethane-1,2- diamine | XX-120 |
| 121 | 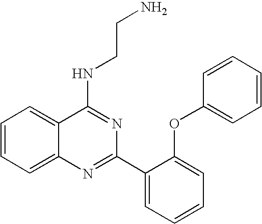 | N*1*-[2-(2- Phenoxy- phenyl)- quinazolin-4-yl]- ethane-1,2- diamine | XX-121 |
| 122 | 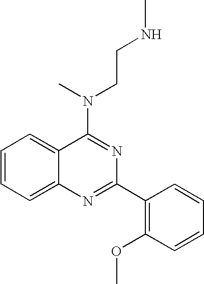 | N-[2-(2-Methoxy- phenyl)- quinazolin-4-yl]- N,N′-dimethyl- ethane-1,2- diamine | XX-122 |
| 123 | 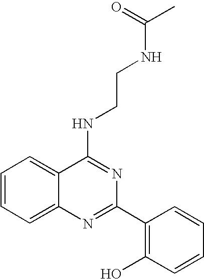 | N-{2-[2-(2- Hydroxy- phenyl)- quinazolin-4- ylamino]-ethyl}- acetamide | XX-123 |
| 124 | 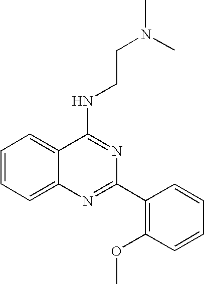 | N′-[2-(2- Methoxy- phenyl)- quinazolin-4-yl]- N,N-dimethyl- ethane-1,2- diamine | XX-124 |
| 125 | 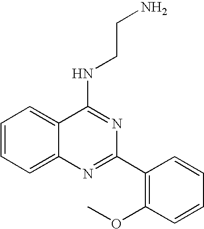 | N1-[2-(2- Methoxy- phenyl)- quinazolin-4-yl]- ethane-1,2- diamine | XX-125 |

Additional examples of some compounds (where J is N) are shown below.

| # | Structure | Name | ID No. |
|---|---|---|---|
| 126 | 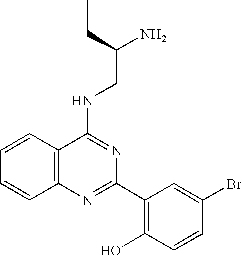 | ((R)-1-{[2-(5-Bromo- 2-hydroxy-phenyl)- quinazolin-4- ylamino]-methyl}- propyl)-carbamic acid tert-butyl ester | XX-126 |
| 127 | 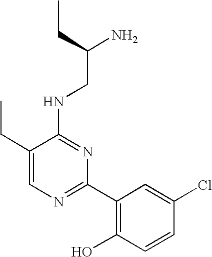 | ((R)-1-{[2-(5-Chloro- 2-hydroxy-phenyl)-5- ethyl-pyrimidin-4- ylamino]-methyl}- propyl)-carbamic acid tert-butyl ester | XX-127 |
| 128 | 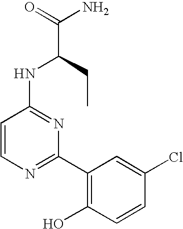 | (R)-2-[2-(5-Chloro-2- hydroxy-phenyl)- pyrimidin-4-ylamino]- butyramide | XX-128 |
| 129 | 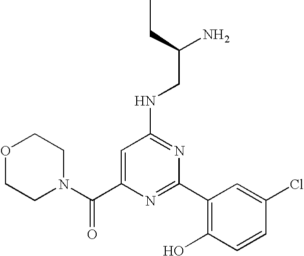 | [6-((R)-2-Amino- butylamino)-2-(5- chloro-2-hydroxy- phenyl)-pyrimidin-4- yl]-morpholin-4-yl- methanone | XX-129 |
| 130 | 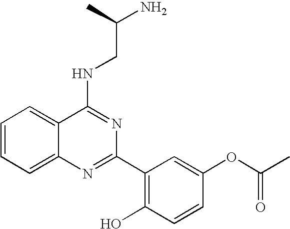 | Acetic acid 3-[4-((R)- 2-amino- propylamino)- quinazolin-2-yl]-4- hydroxy-phenyl ester | XX-130 |
| 131 | 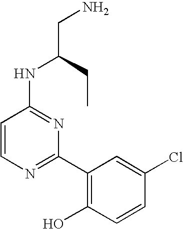 | 2-[4-((R)-1- Aminomethyl- propylamino)- pyrimidin-2-yl]-4- chloro-phenol | XX-131 |
| 132 | 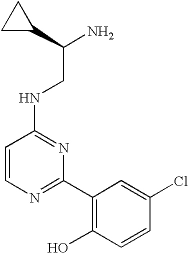 | 2-[4-((R)-2-Amino-2- cyclopropyl- ethylamino)- pyrimidin-2-yl]-4- chloro-phenol | XX-132 |
| 133 | 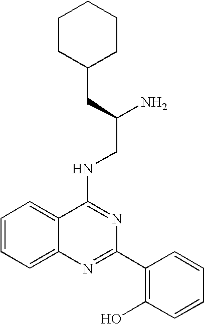 | 2-[4-((R)-2-Amino-3- cyolohexyl- propylamino)- quinazolin-2-yl]- phenol | XX-133 |
| 134 | 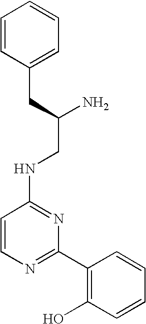 | 2-[4-((R)-2-Amino-3- phenyl- propylamino)- pyrimidin-2-yl]- phenol | XX-134 |
| 135 | 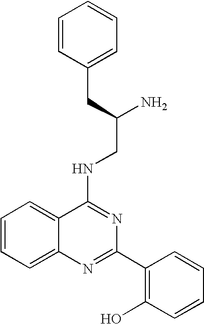 | 2-[4-((R)-2-Amino-3- phenyl- propylamino)- quinazolin-2-yl]- phenol | XX-135 |
| 136 | 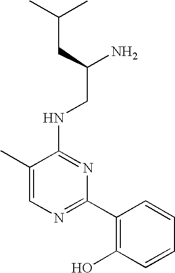 | 2-[4-((R)-2-Amino-4- methyl- pentylamino)-5- methyl-pyrimidin-2- yl]-phenol | XX-136 |
| 137 | 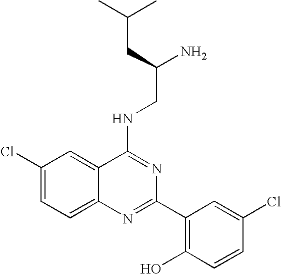 | 2-[4-((R)-2-Amino-4- methyl- pentylamino)-6- chloro-quinazolin-2- yl]-4-chloro-phenol | XX-137 |
| 138 | 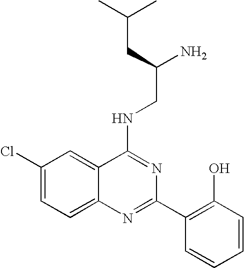 | 2-[4-((R)-2-Amino-4- methyl- pentylamino)-6- chloro-quinazolin-2- yl]-phenol | XX-138 |
| 139 | 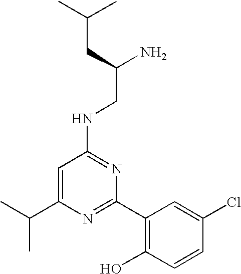 | 2-[4-((R)-2-Amino-4- methyl- pentylamino)-6- isopropyl-pyrimidin- 2-yl]-4-chloro-phenol | XX-139 |
| 140 | 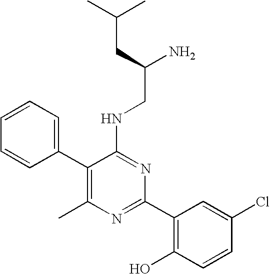 | 2-[4-((R)-2-Amino-4- methyl- pentylamino)-6- methyl-5-phenyl- pyrimidin-2-yl]-4- chloro-phenol | XX-140 |
| 141 | 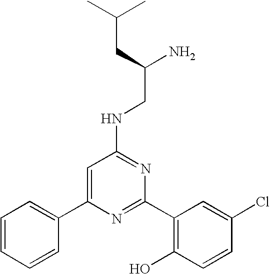 | 2-[4-((R)-2-Amino-4- methyl- pentylamino)-6- phenyl-pyrimidin-2- yl]-4-chloro-phenol | XX-141 |
| 142 | 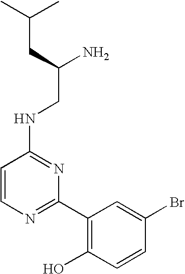 | 2-[4-((R)-2-Amino-4- methyl- pentylamino)- pyrimidin-2-yl]-4- bromo-phenol | XX-142 |
| 143 | 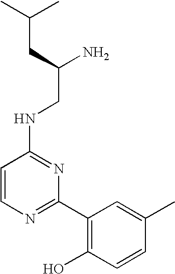 | 2-[4-((R)-2-Amino-4- methyl- pentylamino)- pyrimidin-2-yl]-4- methyl-phenol | XX-143 |
| 144 | 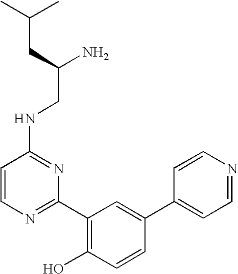 | 2-[4-((R)-2-Amino-4- methyl- pentylamino)- pyrimidin-2-yl]-4- pyridin-4-yl-phenol | XX-144 |
| 145 | 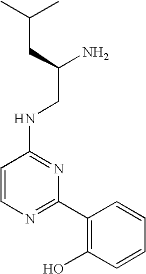 | 2-[4-((R)-2-Amino-4- methyl- pentylamino)- pyrimidin-2-yl]- phenol | XX-145 |
| 146 | 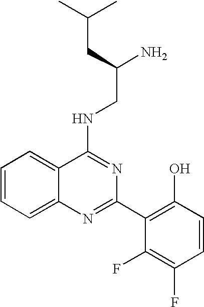 | 2-[4-((R)-2-Amino-4- methyl- pentylamino)- quinazolin-2-yl]-3,4- difluoro-phenol | XX-146 |
| 147 | 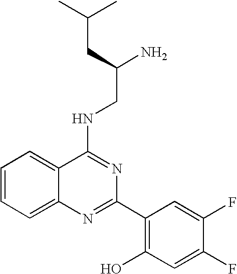 | 2-[4-((R)-2-Amino-4- methyl- pentylamino)- quinazolin-2-yl]-4,5- difluoro-phenol | XX-147 |
| 148 | 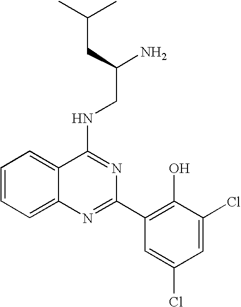 | 2-[4-((R)-2-Amino-4- methyl- pentylamino)- quinazolin-2-yl]-4,6- dichloro-phenol | XX-148 |
| 149 | 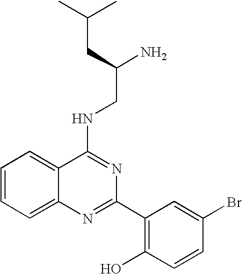 | 2-[4-((R)-2-Amino-4- methyl- pentylamino)- quinazolin-2-yl]-4- bromo-phenol | XX-149 |
| 150 | 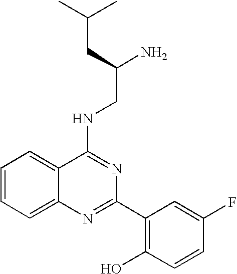 | 2-[4-((R)-2-Amino-4- methyl- pentylamino)- quinazolin-2-yl]-4- fluoro-phenol | XX-150 |
| 151 | 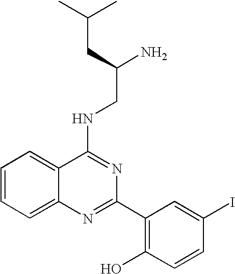 | 2-[4-((R)-2-Amino-4- methyl- pentylamino)- quinazolin-2-yl]-4- iodo-phenol | XX-151 |
| 152 | 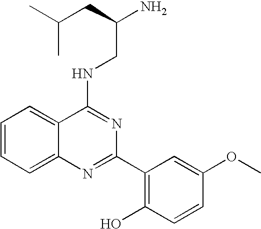 | 2-[4-((R)-2-Amino-4- methyl- pentylamino)- quinazolin-2-yl]-4- methoxy-phenol | XX-152 |
| 153 | 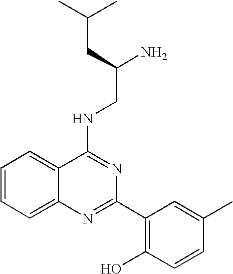 | 2-[4-((R)-2-Amino-4- methyl- pentylamino)- quinazolin-2-yl]-4- methyl-phenol | XX-153 |
| 154 | 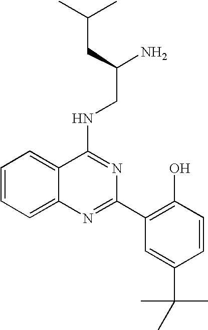 | 2-[4-((R)-2-Amino-4- methyl- pentylamino)- quinazolin-2-yl]-4- tert-butyl-phenol | XX-154 |
| 155 | 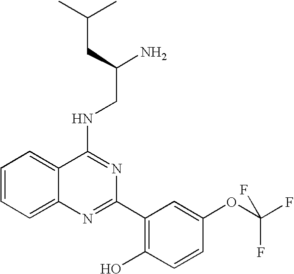 | 2-[4-((R)-2-Amino-4- methyl- pentylamino)- quinazolin-2-yl]-4- trifluoromethoxy- phenol | XX-155 |
| 156 | 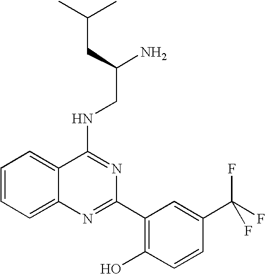 | 2-[4-((R)-2-Amino-4- methyl- pentylamino)- quinazolin-2-yl]-4- trifluoromethyl- phenol | XX-156 |
| 157 | 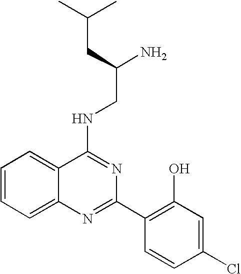 | 2-[4-((R)-2-Amino-4- methyl- pentylamino)- quinazolin-2-yl]-5- chloro-phenol | XX-157 |
| 158 | 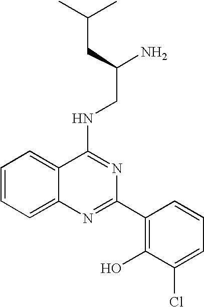 | 2-[4-((R)-2-Amino-4- methyl- pentylamino)- quinazolin-2-yl]-6- chloro-phenol | XX-158 |
| 159 | 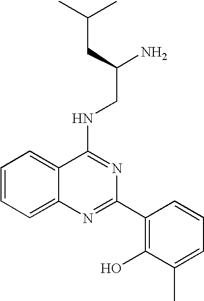 | 2-[4-((R)-2-Amino-4- methyl- pentylamino)- quinazolin-2-yl]-6- methyl-phenol | XX-159 |
| 160 | 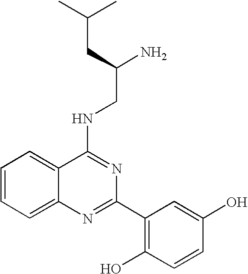 | 2-[4-((R)-2-Amino-4- methyl- pentylamino)- quinazolin-2-yl]- benzene-1,4-diol | XX-160 |
| 161 | 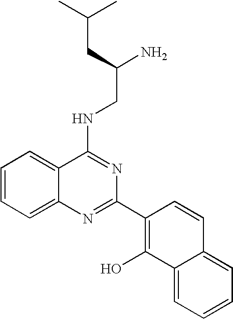 | 2-[4-((R)-2-Amino-4- methyl-pentylamino)- quinazolin-2-yl]- naphthalen-1- ol | XX-161 |
| 162 | 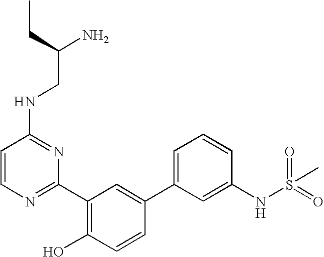 | N-{3′-[4-((R)-2- Amino-butylamino)- pyrimidin-2-yl]-4′- hydroxy-biphenyl-3- yl}- methanesulfonamide | XX-162 |
| 163 | 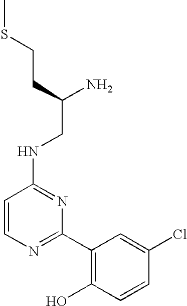 | 2-[4-((R)-2-Amino-4- methylsulfanyl- butylamino)- pyrimidin-2-yl)-4- chloro-phenol | XX-163 |
| 164 | 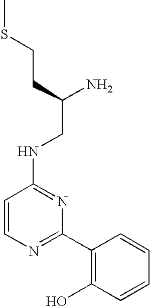 | 2-[4-((R)-2-Amino-4- methylsulfanyl- butylamino)- pyrimidin-2-yl]- phenol | XX-164 |
| 165 | 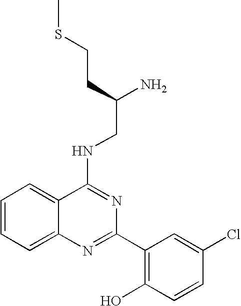 | 2-[4-((R)-2-Amino-4- methylsulfanyl- butylamino)- quinazolin-2-yl]-4- chloro-phenol | XX-165 |
| 166 | 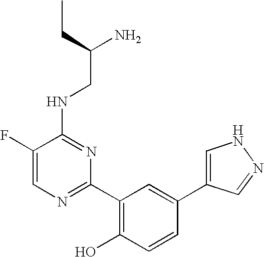 | 2-[4-((R)-2-Amino- butylamino)-5-fluoro- pyrimidin-2-yl]-4- (1H-pyrazol-4-yl)- phenol | XX-166 |
| 167 | 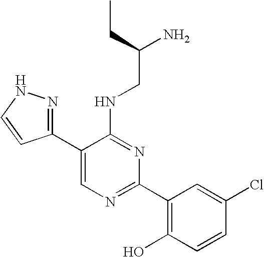 | 2-[4-((R)-2-Amino- butylamino)-5-(1H- pyrazol-3-yl)- pyrimidin-2-yl]-4- chloro-phenol | XX-167 |
| 168 | 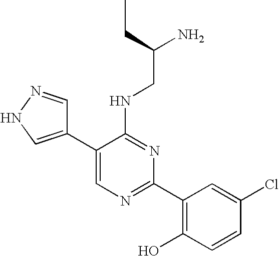 | 2-[4-((R)-2-Amino- butylamlno)-5-(1H- pyrazol-4-yl)- pyrimidin-2-yl]-4- chloro-phenol | XX-168 |
| 169 | 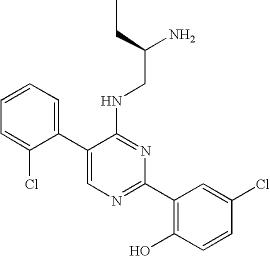 | 2-[4-((R)-2-Amino- butylamino)-5-(2- chloro-phenyl)- pyrimidin-2-yl]-4- chloro-phenol | XX-169 |
| 170 | 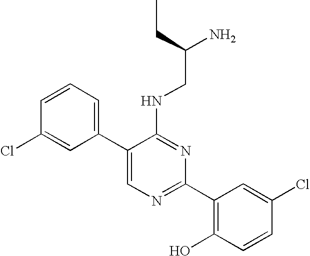 | 2-[4-((R)-2-Amino- butylamino)-5-(3- chloro-phenyl)- pyrimidin-2-yl]-4- chloro-phenol | XX-170 |
| 171 | 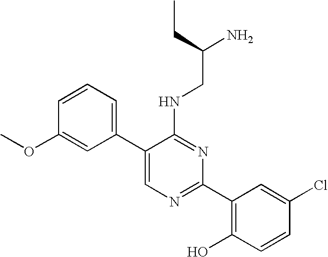 | 2-[4-((R)-2-Amino- butylamino)-5-(3- methoxy-phenyl)- pyrimidin-2-yl]-4- chloro-phenol | XX-171 |
| 172 | 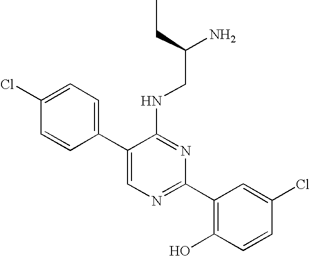 | 2-[4-((R)-2-Amino- butylamino)-5-(4- chloro-phenyl)- pyrimidin-2-yl]-4- chloro-phenol | XX-172 |
| 173 | 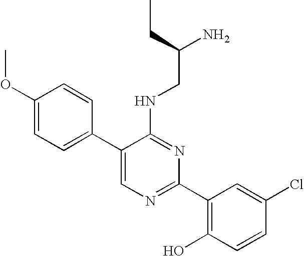 | 2-[4-((R)-2-Amino- butylamino)-5-(4- methoxy-phenyl)- pyrimidin-2-yl]-4- chloro-phenol | XX-173 |
| 174 | 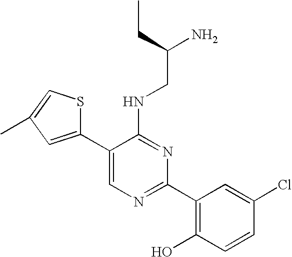 | 2-[4-((R)-2-Amino- butylamino)-5-(4- methyl-thiophen-2- yl)-pyrimidin-2-yl]-4- chloro-phenol | XX-174 |
| 175 | 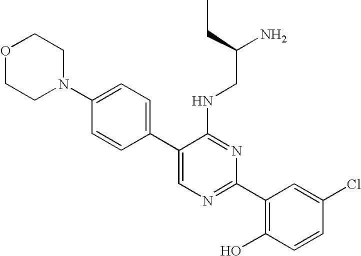 | 2-[4-((R)-2-Amino- butylamino)-5-(4- morpholin-4-yl- phenyl)-pyrimidin-2- yl]-4-chloro-phenol | XX-175 |
| 176 | 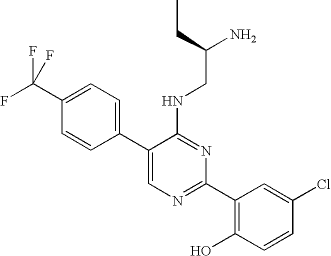 | 2-[4-((R)-2-Amino- butylamino)-5-(4- trifluoromethyl- phenyl)-pyrimidin-2- yl]-4-chloro-phenol | XX-176 |
| 177 | 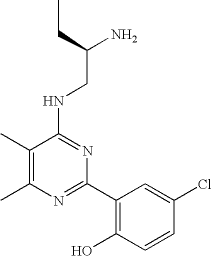 | 2-[4-((R)-2-Amino- butylamino)-5,6- dimethyl-pyrimidin-2- yl]-4-chloro-phenol | XX-177 |
| 178 | 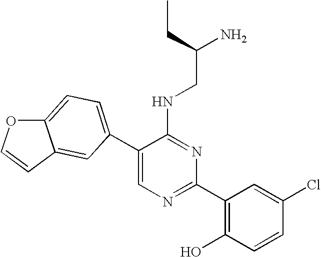 | 2-[4-((R)-2-Amino- butylamino)-5- benzofuran-5-yl- pyrimidin-2-yl]-4- chloro-phenol | XX-178 |
| 179 | 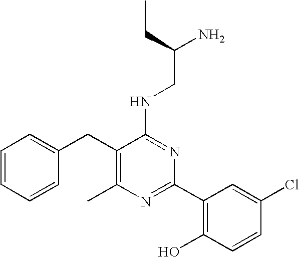 | 2-[4-((R)-2-Amino- butylamino)-5- benzyl-6-methyl- pyrimidin-2-yl]-4- chloro-phenol | XX-179 |
| 180 | 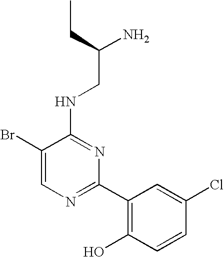 | 2-[4-((R)-2-Amino- butylamino)-5- bromo-pyrimidin-2- yl]-4-chloro-phenol | XX-180 |
| 181 | 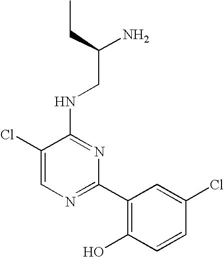 | 2-[4-((R)-2-Amino- butylamino)-5- chloro-pyrimidin-2- yl]-4-chloro-phenol | XX-181 |
| 182 | 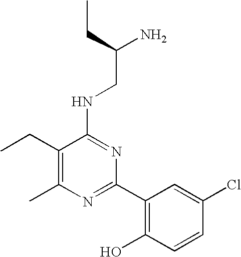 | 2-[4-((R)-2-Amino- butylamino)-5-ethyl- 6-methyl-pyrimidin- 2-yl]-4-chloro-phenol | XX-182 |
| 183 | 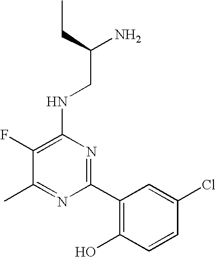 | 2-[4-((R)-2-Amino- butylamino)-5-fluoro- 6-methyl-pyrimidin- 2-yl]-4-chloro-phenol | XX-183 |
| 184 | 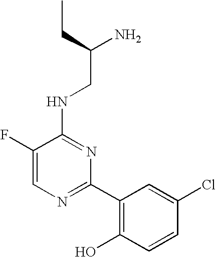 | 2-[4-((R)-2-Amino- butylamino)-5-fluoro- pyrimidin-2-yl]-4- chloro-phenol | XX-184 |
| 185 | 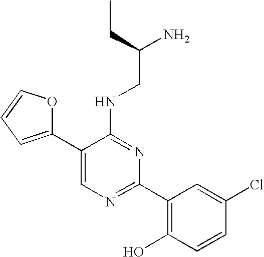 | 2-[4-((R)-2-Amino- butylamino)-5-furan- 2-yl-pyrimidin-2-yl]- 4-chloro-phenol | XX-185 |
| 186 | 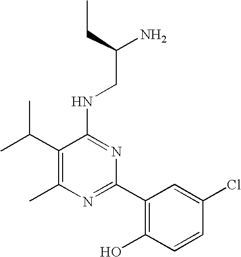 | 2-[4-((R)-2-Amino- butylamino)-5- isopropyl-6-methyl- pyrimidin-2-yl]-4- chloro-phenol | XX-186 |
| 187 | 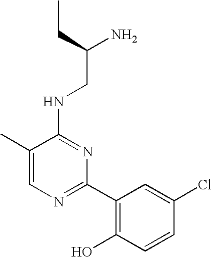 | 2-[4-((R)-2-Amino- butylamino)-5- methyl-pyrimidin-2- yl]-4-chloro-phenol | XX-187 |
| 188 | 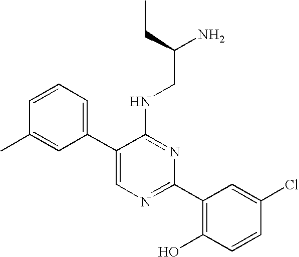 | 2-[4-((R)-2-Amino- butylamino)-5-m- tolyl-pyrimidin-2-yl]- 4-chloro-phenol | XX-188 |
| 189 | 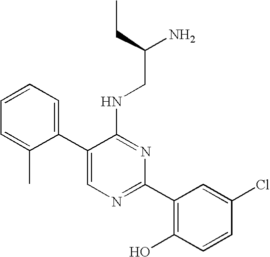 | 2-[4-((R)-2-Amino- butylamino)-5-o- tolyl-pyrimidin-2-yl]- 4-chloro-phenol | XX-189 |
| 190 | 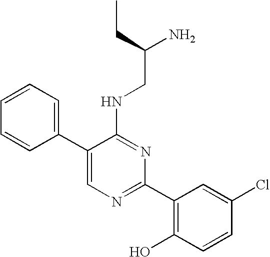 | 2-[4-((R)-2-Amino- butylamino)-5- phenyl-pyrimidin-2- yl]-4-chloro-phenol | XX-190 |
| 191 | 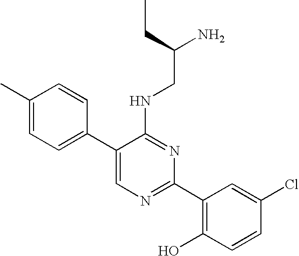 | 2-[4-((R)-2-Amino- butylamino)-5-p- tolyl-pyrimidin-2-yl]- 4-chloro-phenol | XX-191 |
| 192 | 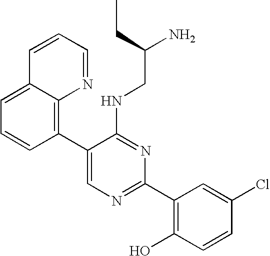 | 2-[4-((R)-2-Amino- butylamino)-5- quinolin-8-yl- pyrimidin-2-yl]-4- chloro-phenol | XX-192 |
| 193 | 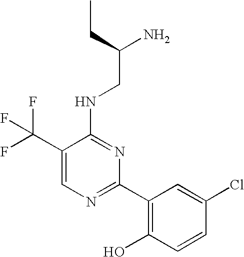 | 2-[4-((R)-2-Amino- butylamino)-5- trifluoromethyl- pyrimidin-2-yl]-4- chloro-phenol | XX-193 |
| 194 | 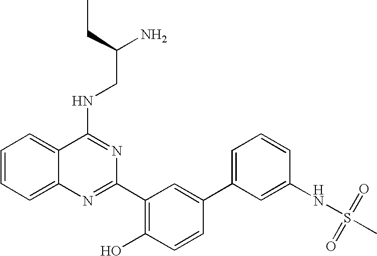 | N-{3′-[4-((R)-2- Amino-butylamino)- quinazolin-2-yl]-4′- hydroxy-biphenyl-3- yl}-methanesulfonide | XX-194 |
| 195 | 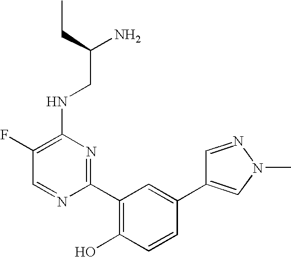 | 2-[4-((R)-2-Amino- butylamino)-5-fluoro- pyrimidin-2-yl]-4-(1- methyl-1H-pyrazol- 4-yl)-phenol | XX-195 |
| 196 | 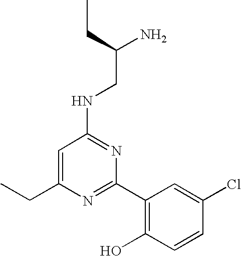 | 2-[4-((R)-2-Amino- butylamino)-6-ethyl- pyrimidin-2-yl]-4- chloro-phenol | XX-196 |
| 197 | 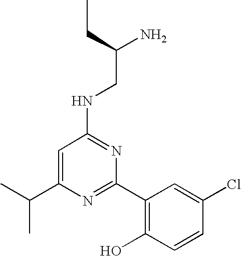 | 2-[4-((R)-2-Amino- butylamino)-6- isopropyl-pyrimidin- 2-yl]-4-chloro-phenol | XX-197 |
| 198 | 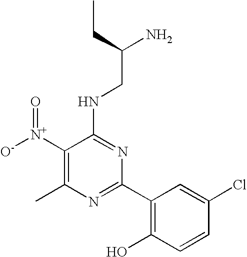 | 2-[4-((R)-2-Amino- butylamino)-6- methyl-5-nitro- pyrimidin-2-yl]-4- chloro-phenol | XX-198 |
| 199 | 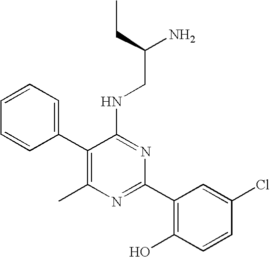 | 2-[4-((R)-2-Amino- butylamino)-6- methyl-5-phenyl- pyrimidin-2-yl]-4- chloro-phenol | XX-199 |
| 200 | 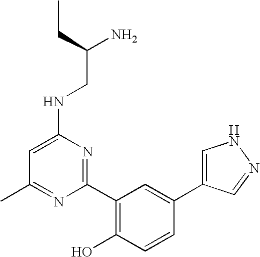 | 2-[4-((R)-2-Amino- butylamino)-6- methyl-pyrimidin-2- yl]-4-(1H-pyrazol-4- yl)-phenol | XX-200 |
| 201 | 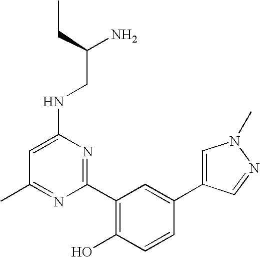 | 2-[4-((R)-2-Amino- butylamino)-6- methyl-pyrimidin-2- yl]-4-(1-methyl-1H- pyrazol-4-yl)-phenol | XX-201 |
| 202 | 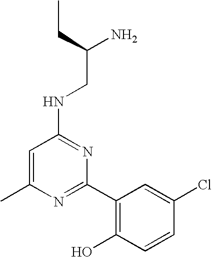 | 2-[4-((R)-2-Amino- butylamino)-6- methyl-pyrimidin-2- yl]-4-chloro-phenol | XX-202 |
| 203 | 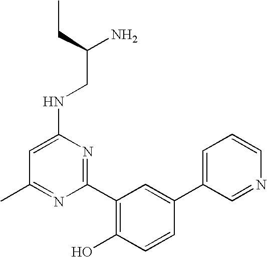 | 2-[4-((R)-2-Amino- butylamino)-6- methyl-pyrimidin-2- yl]-4-pyridin-3-yl- phenol | XX-203 |
| 204 | 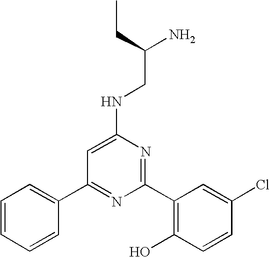 | 2-[4-((R)-2-Amino- butylamino)-6- phenyl-pyrimidin-2- yl]-4-chloro-phenol | XX-204 |
| 205 | 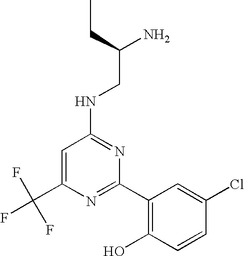 | 2-[4-((R)-2-Amino- butylamino)-6- trifluoromethyl- pyrimidin-yl]-4- chloro-phenol | XX-205 |
| 206 | 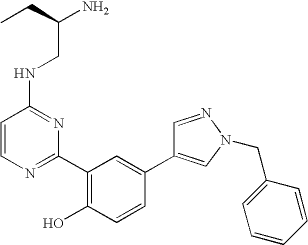 | 2-[4-((R)-2-Amino- butylamino)- pyrimidin-2-yl]-4-(1- benzyl-1H-pyrazol-4- yl)-phenol | XX-206 |
| 207 | 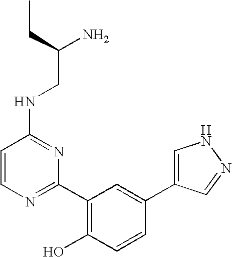 | 2-[4-((R)-2-Amino- butylamino)- pyrimidin-2-yl]-4- (1H-pyrazol-4-yl)- phenol | XX-207 |
| 208 | 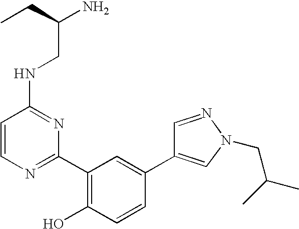 | 2-[4-((R)-2-Amino- butylamino)- pyrimidin-2-yl]-4-(1- isobutyl-1H-pyrazol- 4-yl)-phenol | XX-208 |
| 209 | 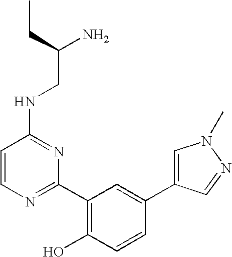 | 2-[4-((R)-2-Amino- butylamino)- pyrimidin-2-yl]-4-(1- methyl-1H-pyrazol- 4-yl)-phenol | XX-209 |
| 210 | 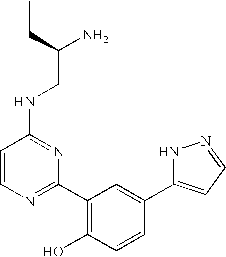 | 2-[4-((R)-2-Amino- butylamino)- pyrimidin-2-yl]-4- (2H-pyrazol-3-yl)- phenol | XX-210 |
| 211 | 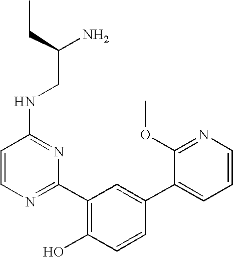 | 2-[4-((R)-2-Amino- butylamino)- pyrimidin-2-yl]-4-(2- methoxy-pyridin-3- yl)-phenol | XX-211 |
| 212 | 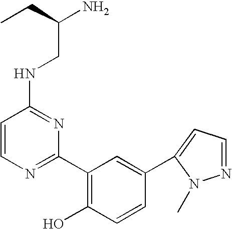 | 2-[4-((R)-2-Amino- butylamino)- pyrimidin-2-yl]-4-(2- methyl-2H-pyrazol- 3-yl)-phenol | XX-212 |
| 213 | 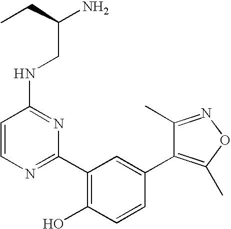 | 2-[4-((R)-2-Amino- butylamino)- pyrimidin-2-yl]-4- (3,5-dimethyl- isoxazol-4-yl)-phenol | XX-213 |
| 214 | 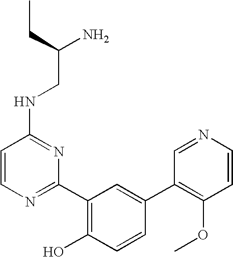 | 2-[4-((R)-2-Amino- butylamino)- pyrimidin-2-yl]-4-(4- methoxy-pyridin-3- yl)-phenol | XX-214 |
| 215 | 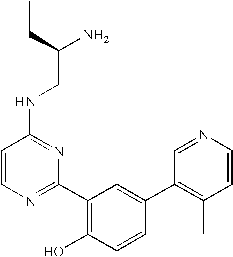 | 2-[4-((R)-2-Amino- butylamino)- pyrimidin-2-yl]-4-(4- methyl-pyridin-3-yl)- phenol | XX-215 |
| 216 | 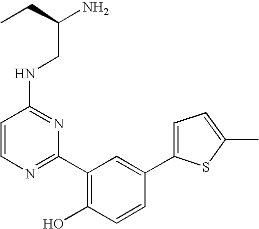 | 2-[4-((R)-2-Amino- butylamino)- pyrimidin-2-yl]-4-(5- methyl-thiophen-2- yl)-phenol | XX-216 |
| 217 | 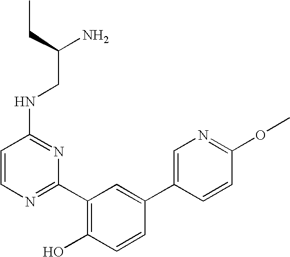 | 2-[4-((R)-2-Amino- butylamino)- pyrimidin-2-yl]-4-(6- methoxy-pyridin-3- yl)-phenol | XX-217 |
| 218 | 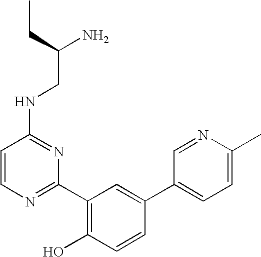 | 2-[4-((R)-2-Amino- butylamino)- pyrimidin-2-yl]-4-(6- methyl-pyridin-3-yl)- phenol | XX-218 |
| 219 | 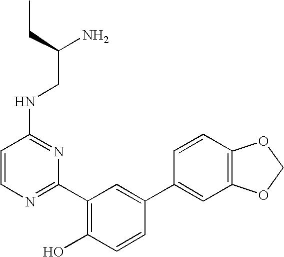 | 2-[4-((R)-2-Amino- butylamino)- pyrimidin-2-yl]-4- benzo[1,3]dioxol-5- yl-phenol | XX-219 |
| 220 | 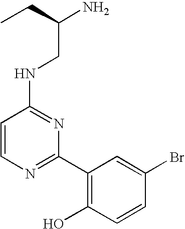 | 2-[4-((R)-2-Amino- butylamino)- pyrimidin-2-yl]-4- bromo-phenol | XX-220 |
| 221 | 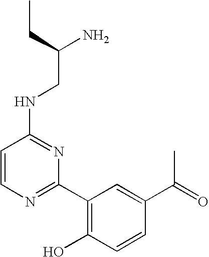 | 2-[4-((R)-2-Amino- butylamino)- pyrimidin-2-yl]-4- ethynyl-phenol | XX-221 |
| 222 | 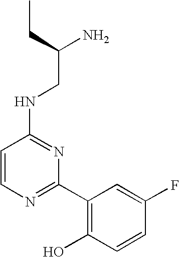 | 2-[4-((R)-2-Amino- butylamino)- pyrimidin-2-yl]-4- fluoro-phenol | XX-222 |
| 223 | 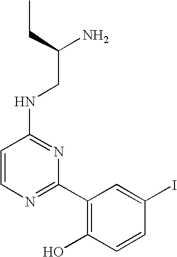 | 2-[4-((R)-2-Amino- butylamino)- pyrimidin-2-yl]-4- iodo-phenol | XX-223 |
| 224 | 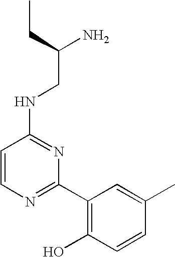 | 2-[4-((R)-2-Amino- butylamino)- pyrimidin-2-yl]-4- methyl-phenol | XX-224 |
| 225 | 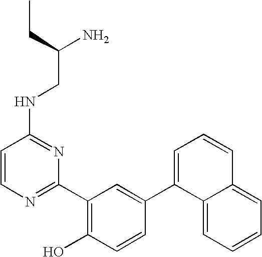 | 2-[4-((R)-2-Amino- butylamino)- pyrimidin-2-yl]-4- naphthalen-1-yl- phenol | XX-225 |
| 226 | 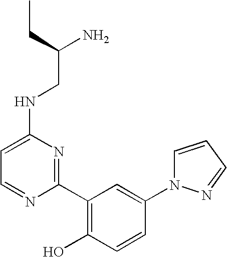 | 2-[4-((R)-2-Amino- butylamino)- pyrimidin-2-yl]-4- pyrazol-1-yl-phenol | XX-226 |
| 227 | 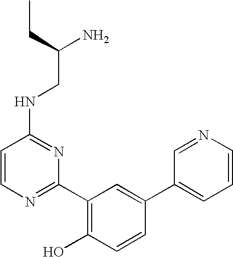 | 2-[4-((R)-2-Amino- butylamino)- pyrimidin-2-yl]-4- pyridin-3-yl-phenol | XX-227 |
| 228 | 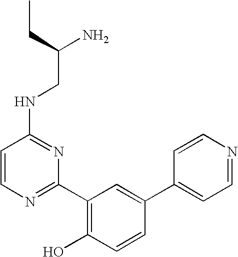 | 2-[4-((R)-2-Amino- butylamino)- pyrimidin-2-yl]-4- pyridin-4-yl-phenol | XX-228 |
| 229 | 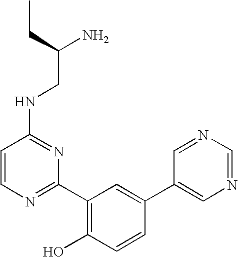 | 2-[4-((R)-2-Amino- butylamino)- pyrimidin-2-yl]-4- pyrimidin-5-yl-phenol | XX-229 |
| 230 | 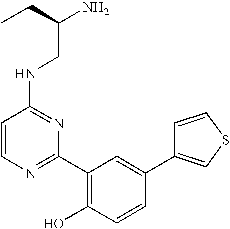 | 2-[4-((R)-2-Amino- butylamino)- pyrimidin-2-yl]-4- thiophen-3-yl-phenol | XX-230 |
| 231 | 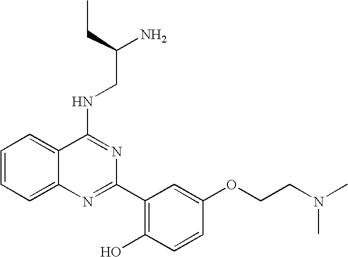 | 2-[4-((R)-2-Amino- butylamino)- quinazolin-2-yl]-4-(2- dimethylamino- ethoxy)-phenol | XX-231 |
| 232 | 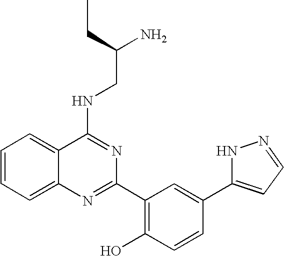 | 2-[4-((R)-2-Amino- butylamino)- quinazolin-2-yl]-4- (2H-pyrazol-3-yl)- phenol | XX-232 |
| 233 | 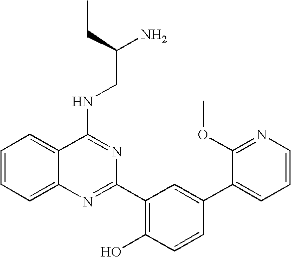 | 2-[4-((R)-2-Amino- butylamino)- quinazolin-2-yl]-4-(2- methoxy-pyridin-3- yl)-phenol | XX-233 |
| 234 | 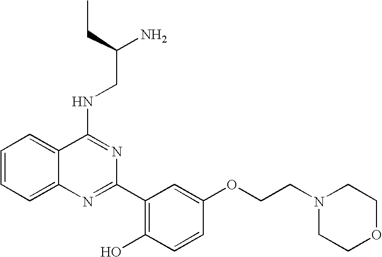 | 2-[4-((R)-2-Amino- butylamino)- quinazolin-2-yl]-4-(2- morpholin-4-yl- ethoxy)-phenol | XX-234 |
| 235 | 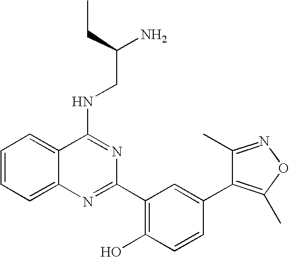 | 2-[4-((R)-2-Amino- butylamino)- quinazolin-2-yl]-4- (3,5-dimethyl- isoxazol-4-yl)-phenol | XX-235 |
| 236 | 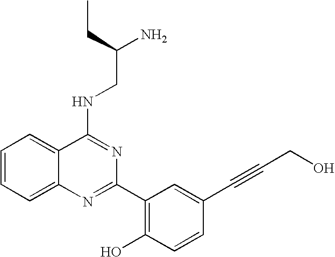 | 2-[4-((R)-2-Amino- butylamino)- quinazolin-2-yl]-4-(3- hydroxy-prop-1- ynyl)-phenol | XX-236 |
| 237 | 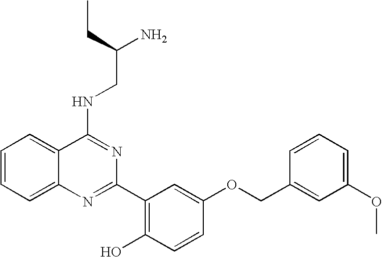 | 2-[4-((R)-2-Amino- butylamino)- quinazolin-2-yl]-4-(3- methoxy-benzyloxy)- phenol | XX-237 |
| 238 | 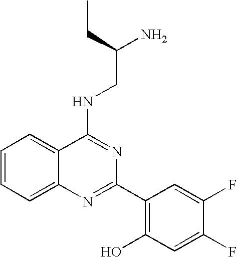 | 2-[4-((R)-2-Amino- butylamino)- quinazolin-2-yl]-4,5- difluoro-phenol | XX-238 |
| 239 | 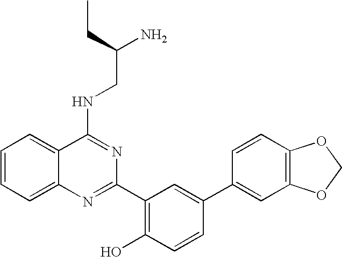 | 2-[4-((R)-2-Amino- butylamino)- quinazolin-2-yl]-4- benzo[1,3]dioxol-5- yl-phenol | XX-239 |
| 240 | 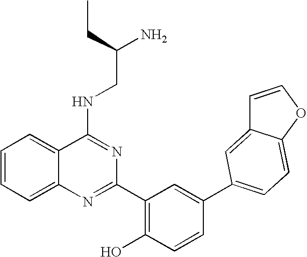 | 2-[4-((R)-2-Amino- butylamino)- quinazolin-2-yl]-4- benzofuran-5-yl- phenol | XX-240 |
| 241 | 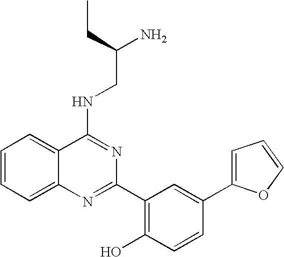 | 2-[4-((R)-2-Amino- butylamino)- quinazolin-2-yl]-4- furan-2-yl-phenol | XX-241 |
| 242 | 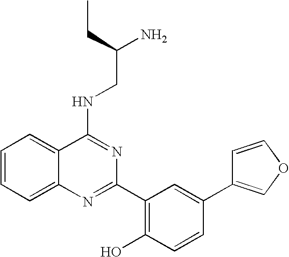 | 2-[4-((R)-2-Amino- butylamino)- quinazolin-2-yl]-4- furan-3-yl-phenol | XX-242 |
| 243 | 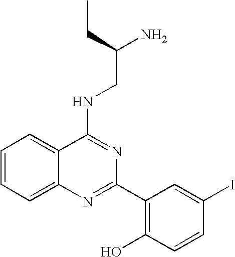 | 2-[4-((R)-2-Amino- butylamino)- quinazolin-2-yl]-4- iodo-phenol | XX-243 |
| 244 | 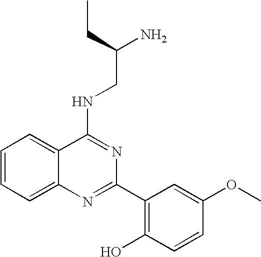 | 2-[4-((R)-2-Amino- butylamino)- quinazolin-2-yl]-4- methoxy-phenol | XX-244 |
| 245 | 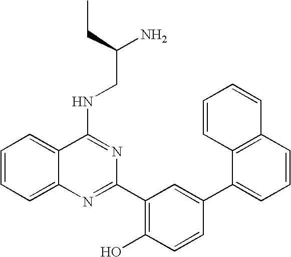 | 2-[4-((R)-2-Amino- butylamino)- quinazolin-2-yl]-4- naphthalen-1-yl- phenol | XX-245 |
| 246 | 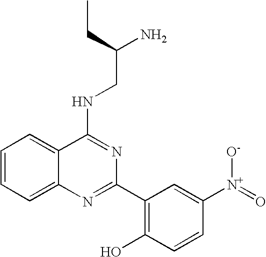 | 2-[4-((R)-2-Amino- butylamino)- quinazolin-2-yl]-4- nitro-phenol | XX-246 |
| 247 | 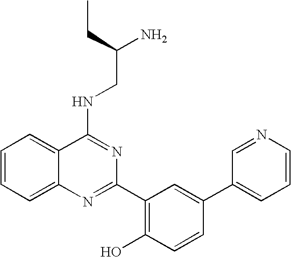 | 2-[4-((R)-2-Amino- butylamino)- quinazolin-2-yl]-4- pyridin-3-yl-phenol | XX-247 |
| 248 | 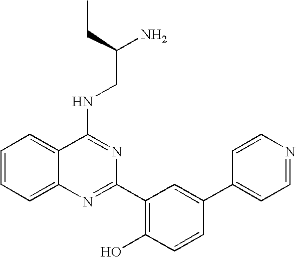 | 2-[4-((R)-2-Amino- butylamino)- quinazolin-2-yl]-4- pyridin-4-yl-phenol | XX-248 |
| 249 | 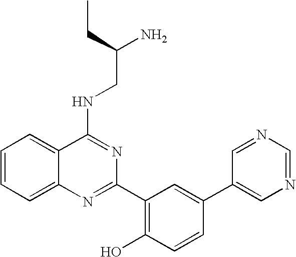 | 2-[4-((R)-2-Amino- butylamino)- quinazolin-2-yl]-4- pyrimidin-5-yl-phenol | XX-249 |
| 250 | 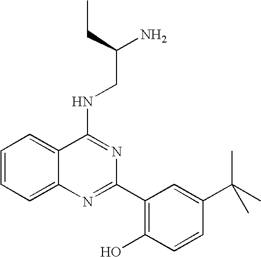 | 2-[4-((R)-2-Amino- butylamino)- quinazolin-2-yl]-4- tert-butyl-phenol | XX-250 |
| 251 | 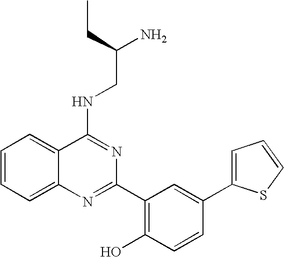 | 2-[4-((R)-2-Amino- butylamino)- quinazolin-2-yl]-4- thiophen-2-yl-phenol | XX-251 |
| 252 | 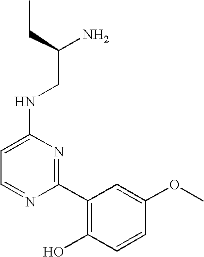 | 2-[4-((R)-2-Amino- butylamino)- pyrimidin-2-yl]-4- methoxy-phenol | XX-252 |
| 253 | 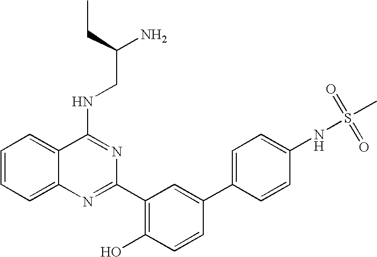 | N-{3′-[4-((R)-2- Amino-butylamino)- quinazolin-2-yl]-4′- hydroxy-biphenyl-4- yl}- methanesulfonamide | XX-253 |
| 254 | 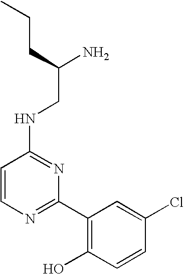 | 2-[4-((R)-2-Amino- pentylamino)- pyrimidin-2-yl]-4- chloro-phenol | XX-254 |
| 255 | 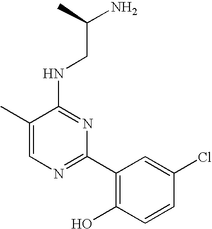 | 2-[4-((R)-2-Amino- propylamino)-5- methyl-pyrimidin-2- yl]-4-chloro-phenol | XX-255 |
| 256 | 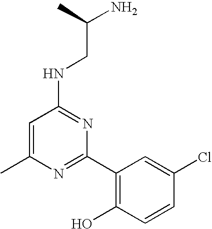 | 2-[4-((R)-2-Amino- propylamino)-6- methyl-pyrimidin-2- yl]-4-chloro-phenol | XX-256 |
| 257 | 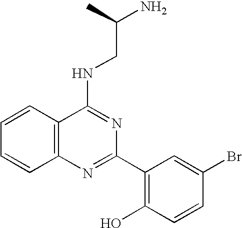 | 2-[4-((R)-2-Amino- propylamino)- quinazolin-2-yl]-4- bromo-phenol | XX-257 |
| 258 | 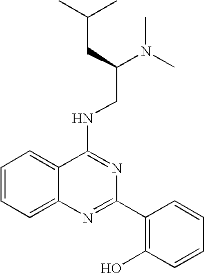 | 2-[4-((R)-2- Dimethylamino-4- methyl- pentylamino)- quinazolin-2-yl]- phenol | XX-258 |
| 259 | 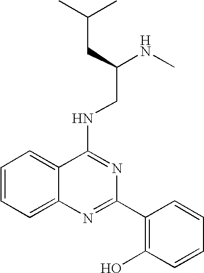 | 2-[4-((R)-4-Methyl-2- methylamino- pentylamino)- quinazolin-2-yl]- phenol | XX-259 |
| 260 | 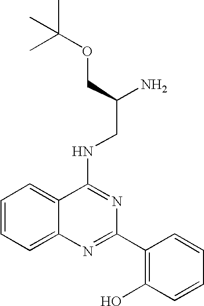 | 2-[4-((S)-2-Amino-3- tert-butoxy- propylamino)- quinazolin-2-yl]- phenol | XX-260 |
| 261 | 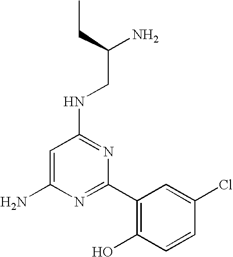 | 2-[4-Amino-6-((R)-2- amino-butylamino)- pyrimidin-2-yl]-4- chloro-phenol | XX-261 |
| 262 | 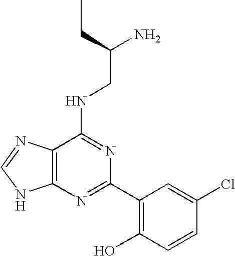 | 2-[6-((R)-2-Amino- butylamino)-9H- purin-2-yl]-4-chloro- phenol | XX-262 |
| 263 | 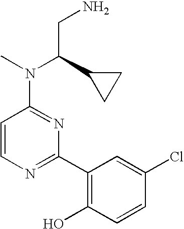 | 2-{4-[((R)-2-Amino- 2-cyclopropyl-ethyl)- methyl-amino]- pyrimidin-2-yl}-4- chloro-phenol | XX-263 |
| 264 | 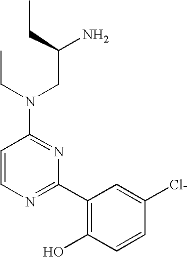 | 2-{4-[((R)-2-Amino- butyl)-ethyl-amino]- pyrimidin-2-yl}-4- chloro-phenol | XX-264 |
| 265 | 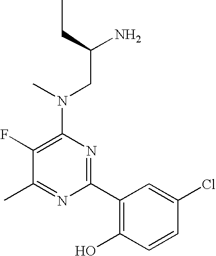 | 2-{4-[((R)-2-Amino- butyl)-methyl- amino]-5-fluoro-6- methyl-pyrimidin-2- yl}-4-chloro-phenol | XX-265 |
| 266 | 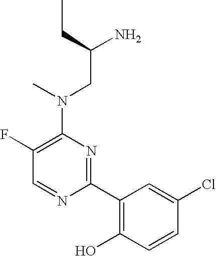 | 2-{4-[((R)-2-Amino- butyl)-methyl- amino]-5-fluoro- pyrimidin-2-yl}-4- chloro-phenol | XX-266 |
| 267 | 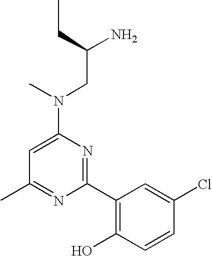 | 2-{4-[((R)-2-Amino- butyl)-methyl- amino)-6-methyl- pyrimidin-2-yl}-4- chloro-phenol | XX-267 |
| 268 | 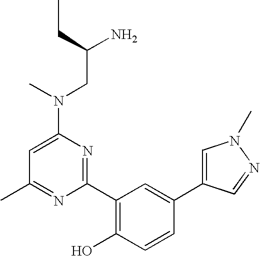 | 2-{4-[((R)-2-Amino- butyl)-methyl- amino]-6-methyl- pyrimidin-2-yl}-4-(1- methyl-1H-pyrazol- 4-yl)-phenol | XX-268 |
| 269 | 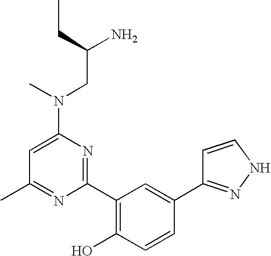 | 2-{4-[((R)-2-Amino- butyl)-methyl- amino]-6-methyl- pyrimidin-2-yl}-4- (1H-pyrazol-3-yl)- phenol | XX-269 |
| 270 | 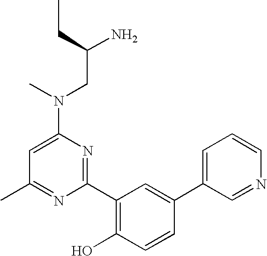 | 2-{4-[((R)-2-Amino- butyl)-methyl- amino]-6-methyl- pyrimidin-2-yl}-4- pyridin-3-yl-phenol | XX-270 |
| 271 | 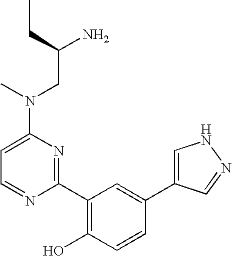 | 2-{4-[((R)-2-Amino- butyl)-methyl- amino]-pyrimidin-2- yl}-4-(1H-pyrazol-4- yl)-phenol | XX-271 |
| 272 | 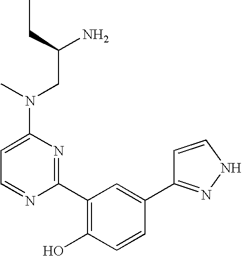 | 2-{4-[((R)-2-Amino- butyl)-methyl- amino]-pyrimidin-2- yl}-4-(1H-pyrazol-3- yl)-phenol | XX-272 |
| 273 | 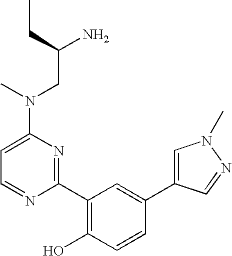 | 2-{4-[((R)-2-Amino- butyl)-methyl- amino]-pyrimidin-2- yl}-4-(1-methyl-1H- pyrazol-4-yl)-phenol | XX-273 |
| 274 | 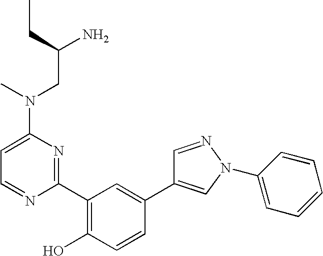 | 2-{4-[((R)-2-Amino- butyl)-methyl- amino]-pyrimidin-2- yl}-4-(1-phenyl-1H- pyrazol-4-yl)-phenol | XX-274 |
| 275 | 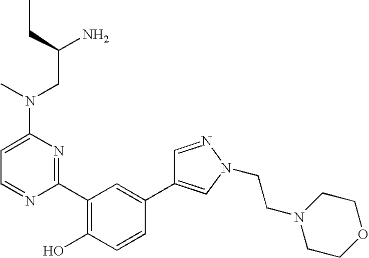 | 2-{4-[((R)-2-Amino- butyl)-methyl- amino]-pyrimidin-2- yl}-4-[1-(2- morpholin-4-yl- ethyl)-1H-pyrazol-4- yl]-phenol | XX-275 |
| 276 | 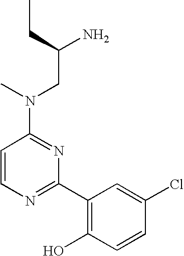 | 2-{4-[((R)-2-Amino- butyl)-methyl- amino]-pyrimidin-2- yl}-4-chloro-phenol | XX-276 |
| 277 | 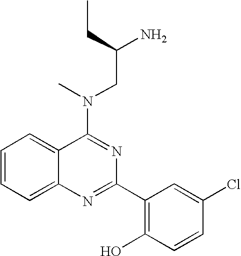 | 2-{4-[((R)-2-Amino- butyl)-methyl- amino]-quinazolin-2- yl}-4-chloro-phenol | XX-277 |
| 278 | 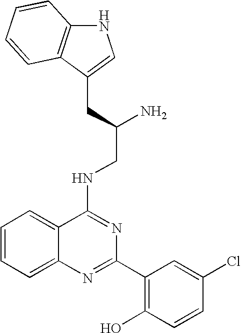 | 2-{4-[(R)-2-Amino-3- (1H-indol-3-yl)- propylamino]- quinazolin-2-yl}-4- chloro-phenol | XX-278 |
| 279 | 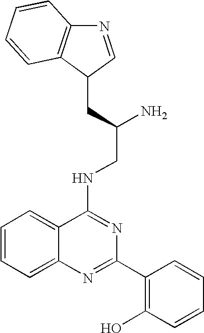 | 2-{4-[(R)-2-Amino-3- (3H-indol-3-yl)- propylamino]- quinazolin-2-yl}- phenol | XX-279 |
| 280 | 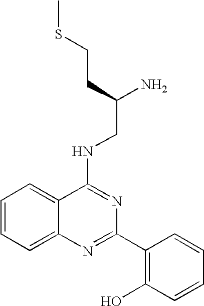 | 2-{4-[(R)-2-Amino-3- (4H-imidazol-4-yl)- propylamino]- quizolin-2-yl}-phenol | XX-280 |
| 281 | 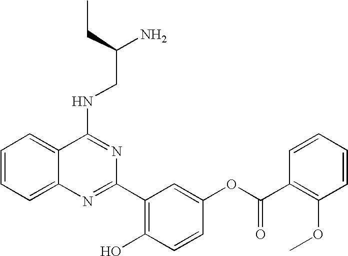 | 2-Methoxy-benzoic acid 3-[4-((R)-2- amino-butylamino)- quinazolin-2-yl]-4- hydroxy-phenyl ester | XX-281 |
| 282 | 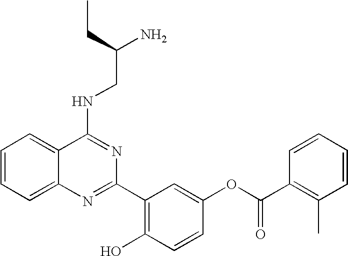 | 2-Methyl-benzoic acid 3-[4-((R)-2- amino-butylamino)- quinazolin-2-yl]-4- hydroxy-phenyl ester | XX-282 |
| 283 | 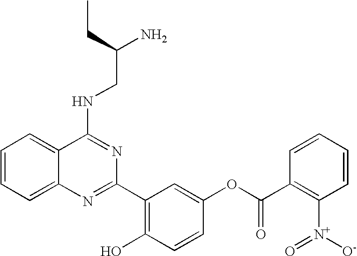 | 2-Nitro-benzoic acid 3-[4-((R)-2-amino- butylamino)- quinazolin-2-yl]-4- hydroxy-phenyl ester | XX-283 |
| 284 | 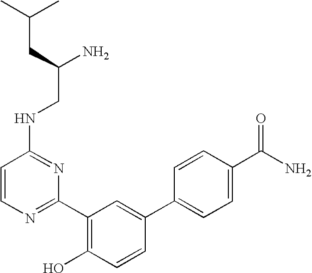 | 3′-[4-((R)-2-Amino-4- methyl- pentylamino)- pyrimidin-2-yl]-4′- hydroxy-biphenyl-4- carboxylic acid amide | XX-284 |
| 285 | 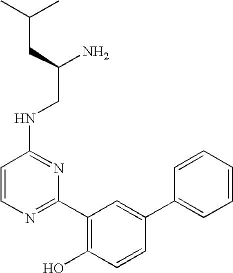 | 3-[4-((R)-2-Amino-4- methyl- pentylamino)- pyrimidin-2-yl]- biphenyl-4-ol | XX-285 |
| 286 | 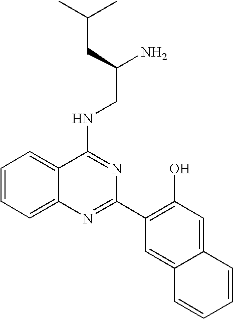 | 3-[4-((R)-2-Amino-4- methyl- pentylamino)- quinazolin-2-yl]- naphthalen-2-ol | XX-286 |
| 287 | 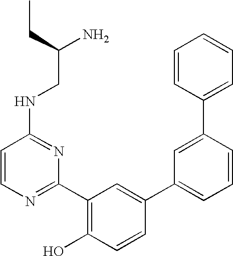 | 3-[4-((R)-2-Amino- butylamino)- pyrimidin-2-yl]- [1,1′;3′,1″]terphey-4- ol | XX-287 |
| 288 | 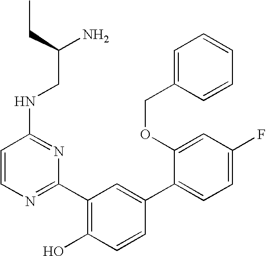 | 3-[4-((R)-2-Amino- butylamino)- pyrimidin-2-yl]-2′- benzyloxy-4′-fluoro- biphenyl-4-ol | XX-288 |
| 289 | 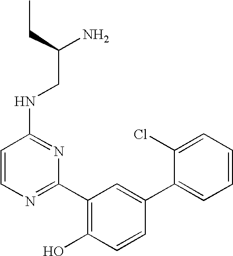 | 3-[4-((R)-2-Amino- butylamino)- pyrimidin-2-yl]-2′- chloro-biphenyl-4-ol | XX-289 |
| 290 | 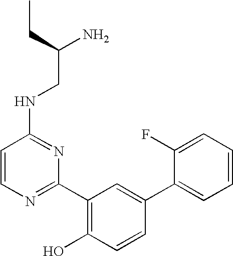 | 3-[4-((R)-2-Amino- butylamino)- pyrimidin-2-yl]-2′- fluoro-biphenyl-4-ol | XX-290 |
| 291 | 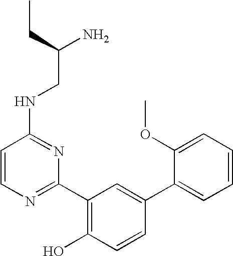 | 3-[4-((R)-2-Amino- butylamino)- pyrimidin-2-yl]-2′- methoxy-biphenyl-4- ol | XX-291 |
| 292 | 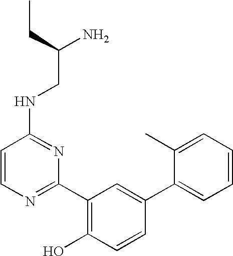 | 3-[4-((R)-2-Amino- butylamino)- pyrimidin-2-yl]-2′- methyl-biphenyl-4-ol | XX-292 |
| 293 | 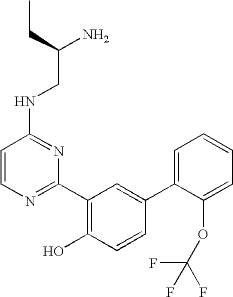 | 3-[4-((R)-2-Amino- butylamino)- pyrimidin-2-yl]-2′- trifluoromethoxy- biphenyl-4-ol | XX-293 |
| 294 | 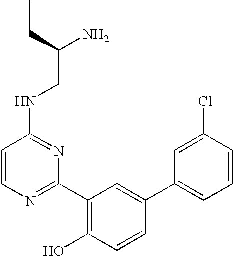 | 3-[4-((R)-2-Amino- butylamino)- pyrimidin-2-yl]-3′- chloro-biphenyl-4-ol | XX-294 |
| 295 | 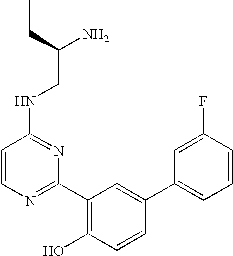 | 3-[4-((R)-2-Amino- butylamino)- pyrimidin-2-yl]-3′- fluoro-biphenyl-4-ol | XX-295 |
| 296 | 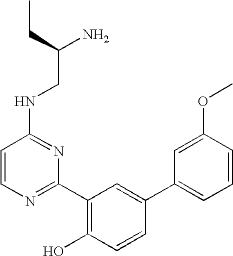 | 3-[4-((R)-2-Amino- butylamino)- pyrimidin-2-yl]-3′- methoxy-biphenyl-4- ol | XX-296 |
| 297 | 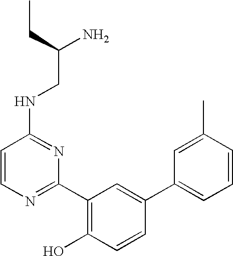 | 3-[4-((R)-2-Amino- butylamino)- pyrimidin-2-yl]-3′- methyl-biphenyl-4-ol | XX-297 |
| 298 | 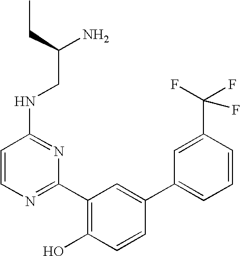 | 3-[4-((R)-2-Amino- butylamino)- pyrimidin-2-yl]-3′- trifluoromethyl- biphenyl-4-ol | XX-298 |
| 299 | 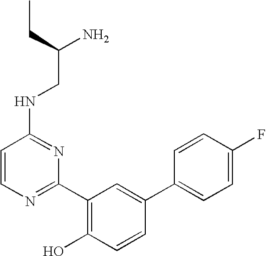 | 3-[4-((R)-2-Amino- butylamino)- pyrimidin-2-yl]-4′- fluoro-biphenyl-4-ol | XX-299 |
| 300 | 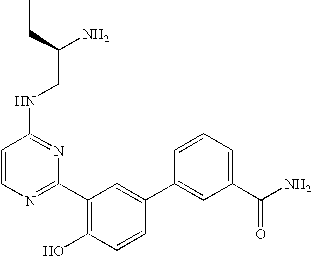 | 3′-[4-((R)-2-Amino- butylamino)- pyrimidin-2-yl]-4′- hydroxy-biphen | XX-300 |
| 301 | 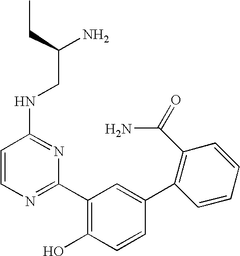 | 3′-[4-((R)-2-Amino- butylamino)- pyrimidin-2-yl]-4′- hydroxy-biphenyl-2- carboxylic acid amide | XX-301 |
| 302 | 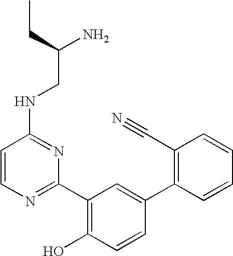 | 3′-[4-((R)-2-Amino- butylamino)- pyrimidin-2-yl]-4′- hydroxy-biphenyl-2- carbonitrile | XX-302 |
| 303 | 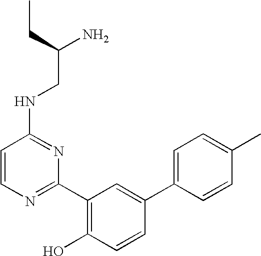 | 3-[4-((R)-2-Amino- butylamino)- pyrimidin-2-yl]-4′- methyl-biphenyl-4-ol | XX-303 |
| 304 | 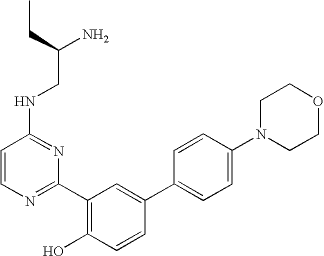 | 3-[4-((R)-2-Amino- butylamino)- pyrimidin-2-yl]-4′- morpholin-4-yl- biphenyl-4-ol | XX-304 |
| 305 | 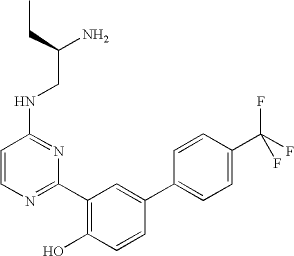 | 3-[4-((R)-2-Amino- butylamino)- pyrimidin-2-yl]-4′- trifluoromethyl- biphenyl-4-ol | XX-305 |
| 306 | 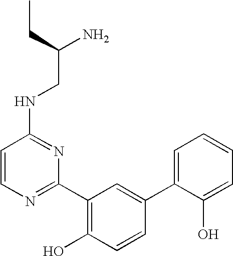 | 3′-[4-((R)-2-Amino- butylamino)- pyrimidin-2-yl]- biphenyl-2,4′-diol | XX-306 |
| 307 | 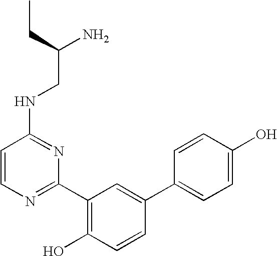 | 3-[4-((R)-2-Amino- butylamino)- pyrimidin-2-yl]- biphenyl-4,4′-diol | XX-307 |
| 308 | 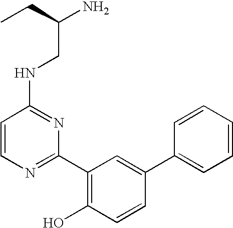 | 3-[4-((R)-2-Amino- butylamino)- pyrimidin-2-yl]- biphenyl-4-ol | XX-308 |
| 309 | 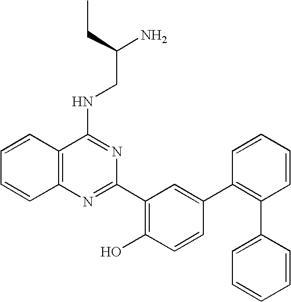 | 3-[4-((R)-2-Amino- butylamino)- quinazolin-2-yl]- [1,1′;2′,1″]terphey-4- ol | XX-309 |
| 310 | 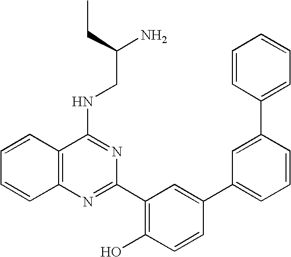 | 3-[4-((R)-2-Amino- butylamino)- quinazolin-2-yl]- [1,1′;3′,1″]terpheyl-4- ol | XX-310 |
| 311 | 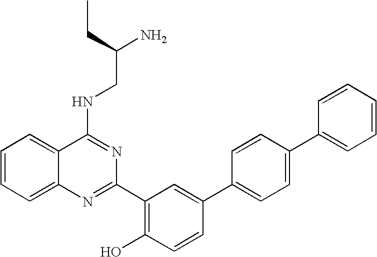 | 3-[4-((R)-2-Amino- butylamino)- quinazolin-2-yl]- [1,1′;4′,1″]terphey-4- ol | XX-311 |
| 312 | 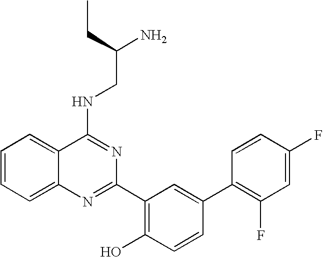 | 3-[4-((R)-2-Amino- butylamino)- quinazolin-2-yl]-2′,4′- difluoro-biphenyl-4- ol | XX-312 |
| 313 | 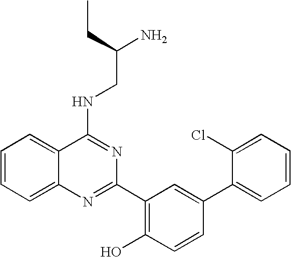 | 3-[4-((R)-2-Amino- butylamino)- quinazolin-2-yl]-2′- chloro-biphenyl-4-ol | XX-313 |
| 314 | 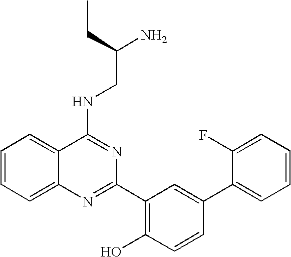 | 3-[4-((R)-2-Amino- butylamino)- quinazolin-2-yl]-2′- fluoro-biphenyl-4-ol | XX-314 |
| 315 | 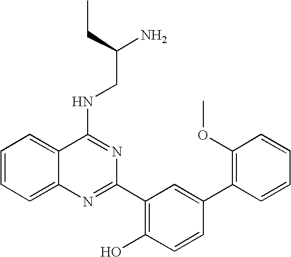 | 3-[4-((R)-2-Amino- butylamino)- quinazolin-2-yl]-2′- methoxy-biphenyl-4- ol | XX-315 |
| 316 | 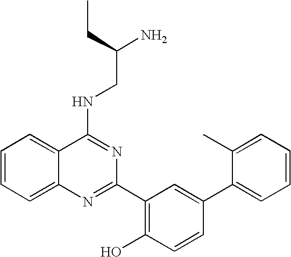 | 3-[4-((R)-2-Amino- butylamino)- quinazolin-2-yl]-2′- methyl-biphenyl-4-ol | XX-316 |
| 317 | 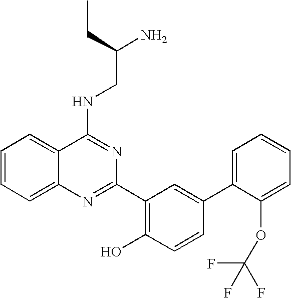 | 3-[4-((R)-2-Amino- butylamino)- quinazolin-2-yl]-2′- trifluoromethoxy- biphenyl-4-ol | XX-317 |
| 318 | 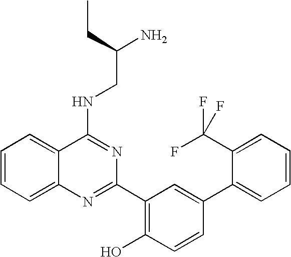 | 3-[4-((R)-2-Amino- butylamino)- quinazolin-2-yl]-2′- trifluoromethyl- biphenyl-4-ol | XX-318 |
| 319 | 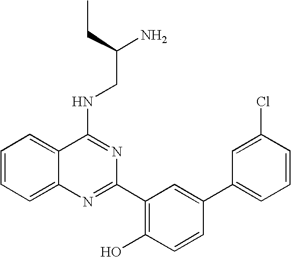 | 3-[4-((R)-2-Amino- butylamino)- quinazolin-2-yl]-3′- chloro-biphenyl-4-ol | XX-319 |
| 320 | 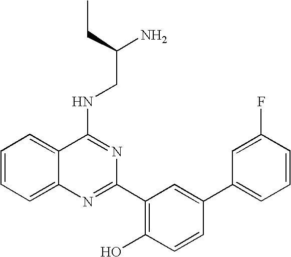 | 3-[4-((R)-2-Amino- butylamino)- quinazolin-2-yl]-3′- fluoro-biphenyl-4-ol | XX-320 |
| 321 | 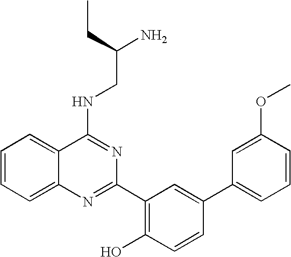 | 3-[4-((R)-2-Amino- butylamino)- quinazoiin-2-yl]-3′- methoxy-biphenyl-4- ol | XX-321 |
| 322 | 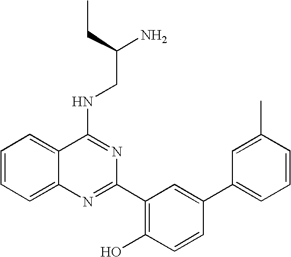 | 3-[4-((R)-2-Amino- butylamino)- quinazolin-2-yl]-3′- methyl-biphenyl-4-ol | XX-322 |
| 323 | 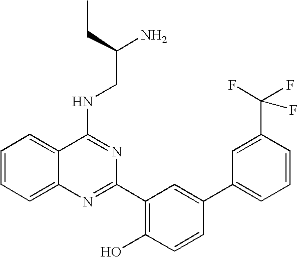 | 3-[4-((R)-2-Amino- butylamino)- quinazolin-2-yl]-3′- trifluoromethyl- biphenyl-4-ol | XX-323 |
| 324 | 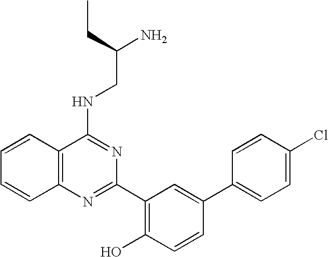 | 3-[4-((R)-2-Amino- butylamino)- quinazolin-2-yl]-4′- chloro-biphenyl-4-ol | XX-324 |
| 325 | 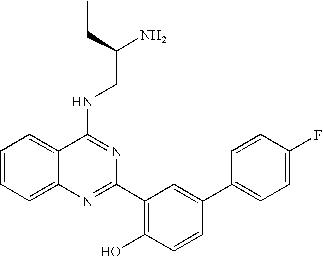 | 3-[4-((R)-2-Amino- butylamino)- quinazolin-2-yl]-4′- fluoro-biphenyl-4-ol | XX-325 |
| 326 | 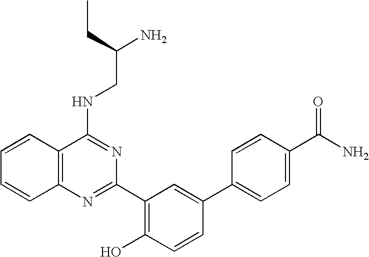 | 3′-[4-((R)-2-Amino- butylamino)- quinazolin-2-yl]-4′- hydroxy-biphenyl-4- carboxylic acid amide | XX-326 |
| 327 | 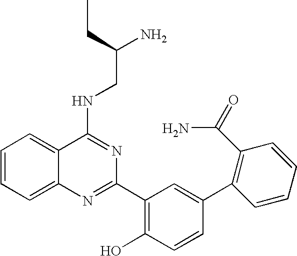 | 3′-[4-((R)-2-Amino- butylamino)- quinazolin-2-yl]-4′- hydroxy-biphenyl-2- carboxylic acid amide | XX-327 |
| 328 | 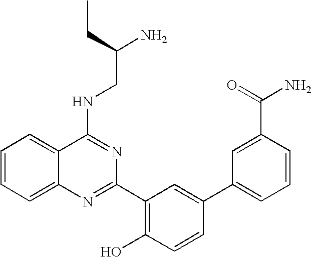 | 3′-[4-((R)-2-Amino- butylamino)- quinazoiin-2-yl]-4′- hydroxy-biphenyl-3- carboxylic acid amide | XX-328 |
| 329 | 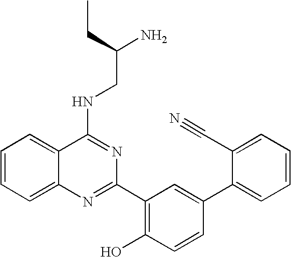 | 3′-[4-((R)-2-Amino- butylamino)- quinazolin-2-yl]-4′- hydroxy-biphenyl-2- carbonitrile | XX-329 |
| 330 | 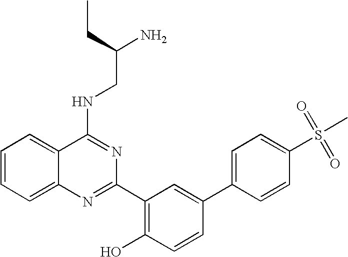 | 3-[4-((R)-2-Amino- butylamino)- quinazolin-2-yl]-4′- methanesulfonyl- biphenyl-4-ol | XX-330 |
| 331 | 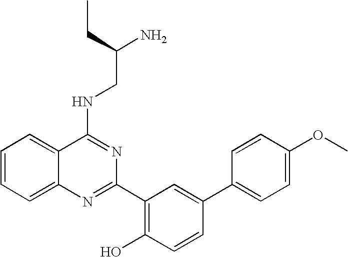 | 3-[4-((R)-2-Amino- butylamino)- quinazolin-2-yl]-4′- methoxy-biphenyl-4- ol | XX-331 |
| 332 | 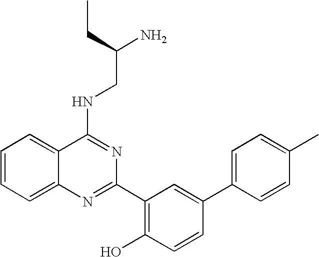 | 3-[4-((R)-2-Amino- butylamino)- quinazolin-2-yl]-4′- methyl-biphenyl-4-ol | XX-332 |
| 333 | 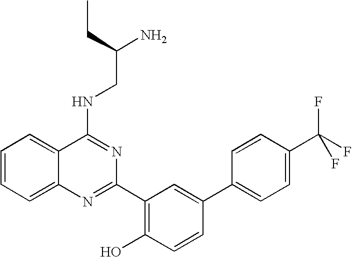 | 3-[4-((R)-2-Amino- butylamino)- quinazolin-2-yl]-4′- trifluoromethyl- biphenyl-4-ol | XX-333 |
| 334 | 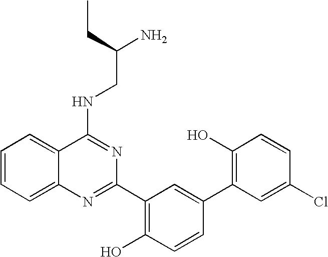 | 3′-[4-((R)-2-Amino- butylamino)- quinazolin-2-yl]-5- chloro-biphenyl-2,4′- diol | XX-334 |
| 335 | 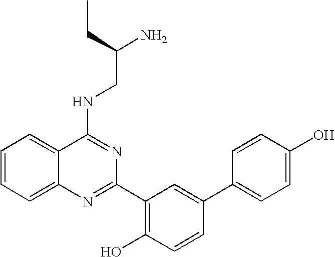 | 3-[4-((R)-2-Amino- butylamino)- quinazolin-2-yl]- biphenyl-4,4′-diol | XX-335 |
| 336 | 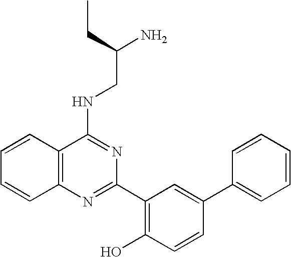 | 3-[4-((R)-2-Amino- butylamino)- quinazolin-2-yl]- biphenyl-4-ol | XX-336 |
| 337 | 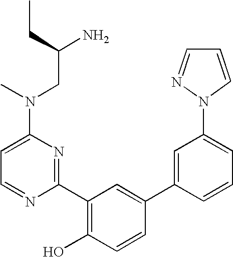 | 3-{4-[((R)-2-Amino- butyl)-methyl- amino]-pyrimidin-2- yl}-3′-pyrazol-1-yl- biphenyl-4-ol | XX-337 |
| 338 | 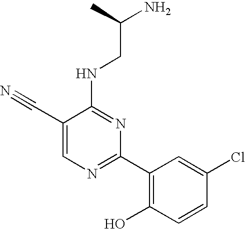 | 4-((R)-2-Amino- propylamino)-2-(5- chloro-2-hydroxy- phenyl)-pyrimidine- 5-carbonitrile | XX-338 |
| 339 | 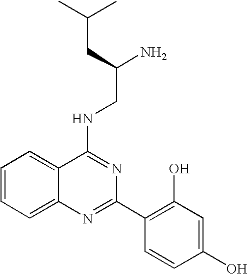 | 4-[4-((R)-2-Amino-4- methyl- pentylamino)- quinazolin-2-yl]- benzene-1,3-diol | XX-339 |
| 340 | 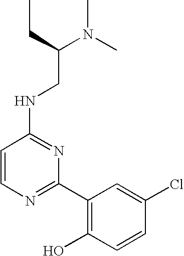 | 4-Chloro-2-[4-((R)-2- methylamino- butylamino)- pyrimidin-2-yl]- phenol | XX-340 |
| 341 | 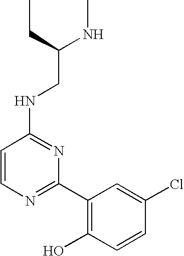 | 4-Chloro-2-[4-((R)-2- methylamino- butylamino)- pyrimidin-2-yl]- phenol | XX-341 |
| 342 | 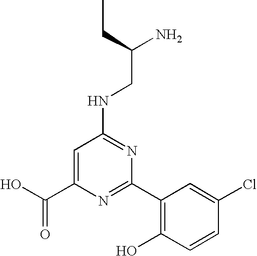 | 6-((R)-2-Amino- butylamino)-2-(5- chloro-2-hydroxy- phenyl)-pyrimidine- 4-carboxylic acid | XX-342 |
| 343 | 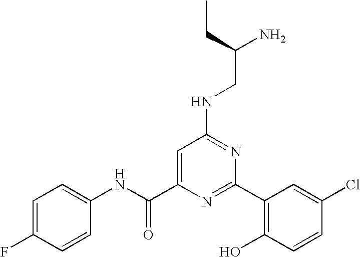 | 6-((R)-2-Amino- butylamino)-2-(5- chloro-2-hydroxy- phenyl)-pyrimidine- 4-carboxylic acid (3- methoxy-phenyl)- amide | XX-343 |
| 344 | 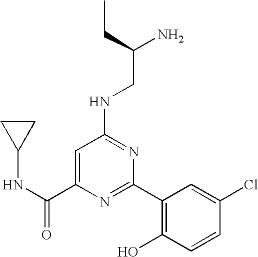 | 6-((R)-2-Amino- butylamino)-2-(5- chloro-2-hydroxy- phenyl)-pyrimidine- 4-carboxylic acid cyclopropylamide | XX-344 |

Examples of some compounds (where J is CH) are shown below.

| # | Structure | Name | |
|---|---|---|---|
| 1 | 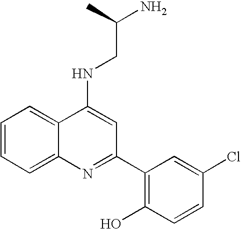 | 2-[4-((R)- 2-Amino- propylamino)- quinolin-2- yl]-4- chloro-phenol | YY-001 |
| 2 | 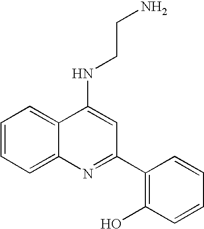 | 2-[4-(2-Amino- ethylamino)- quinolin- 2-yl]-phenol | YY-002 |
| 3 | 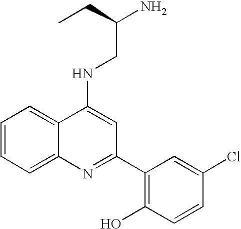 | 2-[4-((R)- 2-Amino- butylamino)- quinolin-2-yl]- 4-chloro-phenol | YY-003 |

### Includes Other Forms

Unless otherwise specified, a reference to a particular group also includes the well known ionic, salt, hydrate, solvate, and protected forms thereof. For example, a reference to carboxylic acid (—COOH) also includes the anionic (carboxylate) form (—COO⁻), a salt or hydrate or solvate thereof, as well as conventional protected forms. Similarly, a reference to an amino group includes the protonated form (—N⁺HR¹R²), a salt or hydrate or solvate of the amino group, for example, a hydrochloride salt, as well as conventional protected forms of an amino group. Similarly, a reference to a hydroxyl group also includes the anionic form (—O⁻), a salt or hydrate or solvate thereof, as well as conventional protected forms.

### Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

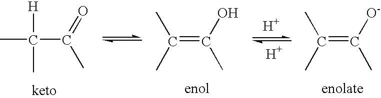

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Solvates and Hydrates

It may be convenient or desirable to prepare, purify, and/or handle a corresponding solvate of the compound. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate and hydrate forms thereof.

### Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulfonyl)ethyloxy amide (—NH-Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

### Prodrugs

It may be convenient or desirable to prepare, purify, and/or handle an active compound in the form of a prodrug. The term “prodrug,” as used herein, pertains to a compound which, when metabolised (e.g., in vivo), yields the desired active compound. Typically, the prodrug is inactive, or less active than the active compound, but may provide advantageous handling, administration, or metabolic properties.

Unless otherwise specified, a reference to a particular compound also includes prodrugs thereof.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Chemical Synthesis

Several methods for the chemical synthesis of AEAA compounds of the present invention are described herein. These and/or other well known methods may be modified and/or adapted in known ways in order to facilitate the synthesis of additional compounds within the scope of the present invention.

### Uses

The AEAA compounds described herein are useful, for example, in the treatment of diseases and conditions that are ameliorated by the inhibition of PKD (e.g., PKD1, PKD2, PKD3), such as, for example, proliferative conditions, cancer, etc.

### Use in Methods of Inhibiting PKD

One aspect of the present invention pertains to a method of inhibiting PKD (e.g., PKD1, PKD2, PKD3) in a cell, in vitro or in vivo, comprising contacting the cell with an effective amount of an AEAA compound, as described herein.

Suitable assays for determining PKD (e.g., PKD1, PKD2, PKD3) inhibition are known in the art and/or are described herein.

### Use in Methods of Inhibiting Cell Proliferation, Etc.

The AEAA compounds described herein, e.g., (a) regulate (e.g., inhibit) cell proliferation; (b) inhibit cell cycle progression; (c) promote apoptosis; or (d) a combination of one or more of these.

One aspect of the present invention pertains to a method of regulating (e.g., inhibiting) cell proliferation (e.g., proliferation of a cell), inhibiting cell cycle progression, promoting apoptosis, or a combination of one or more these, in vitro or in vivo, comprising contacting cells (or the cell) with an effective amount of an AEAA compound, as described herein.

In one embodiment, the method is a method of regulating (e.g., inhibiting) cell proliferation (e.g., proliferation of a cell), in vitro or in vivo, comprising contacting cells (or the cell) with an effective amount of an AEAA compound, as described herein.

In one embodiment, the method is performed in vitro.

In one embodiment, the method is performed in vivo.

In one embodiment, the AEAA compound is provided in the form of a pharmaceutically acceptable composition.

Any type of cell may be treated, including but not limited to, lung, gastrointestinal (including, e.g., bowel, colon), breast (mammary), ovarian, prostate, liver (hepatic), kidney (renal), bladder, pancreas, brain, and skin.

One of ordinary skill in the art is readily able to determine whether or not a candidate compound regulates (e.g., inhibits) cell proliferation, etc. For example, assays which may conveniently be used to assess the activity offered by a particular compound are described herein.

For example, a sample of cells (e.g., from a tumour) may be grown in vitro and a compound brought into contact with said cells, and the effect of the compound on those cells observed. As an example of “effect,” the morphological status of the cells (e.g., alive or dead, etc.) may be determined. Where the compound is found to exert an influence on the cells, this may be used as a prognostic or diagnostic marker of the efficacy of the compound in methods of treating a patient carrying cells of the same cellular type.

### Use in Methods of Therapy

Another aspect of the present invention pertains to an AEAA compound as described herein for use in a method of treatment of the human or animal body by therapy.

### Use in the Manufacture of Medicaments

Another aspect of the present invention pertains to use of an AEAA compound, as described herein, in the manufacture of a medicament for use in treatment.

In one embodiment, the medicament comprises the AEAA compound.

### Methods of Treatment

Another aspect of the present invention pertains to a method of treatment comprising administering to a patient in need of treatment a therapeutically effective amount of an AEAA compound as described herein, preferably in the form of a pharmaceutical composition.

### Conditions Treated—Conditions Mediated by PKD

In one embodiment (e.g., of use in methods of therapy, of use in the manufacture of medicaments, of methods of treatment), the treatment is treatment of a disease or condition that is mediated by PKD (e.g., PKD1, PKD2, PKD3).

### Conditions Treated—Conditions Ameliorated by the Inhibition of PDK

In one embodiment (e.g., of use in methods of therapy, of use in the manufacture of medicaments, of methods of treatment), the treatment is treatment of: a disease or condition that is ameliorated by the inhibition of PKD (e.g., PKD1, PKD2, PKD3).

### Conditions Treated—Proliferative Conditions and Cancer

In one embodiment (e.g., of use in methods of therapy, of use in the manufacture of medicaments, of methods of treatment), the treatment is treatment of: a proliferative condition.

The term “proliferative condition,” as used herein, pertains to an unwanted or uncontrolled cellular proliferation of excessive or abnormal cells which is undesired, such as, neoplastic or hyperplastic growth.

In one embodiment, the treatment is treatment of: a proliferative condition characterised by benign, pre-malignant, or malignant cellular proliferation, including but not limited to, neoplasms, hyperplasias, and tumours (e.g., histocytoma, glioma, astrocyoma, osteoma), cancers (see below), psoriasis, bone diseases, fibroproliferative disorders (e.g., of connective tissues), pulmonary fibrosis, atherosclerosis, smooth muscle cell proliferation in the blood vessels, such as stenosis or restenosis following angioplasty.

In one embodiment, the treatment is treatment of: cancer.

In one embodiment, the treatment is treatment of: lung cancer, small cell lung cancer, non-small cell lung cancer, gastrointestinal cancer, stomach cancer, bowel cancer, colon cancer, rectal cancer, colorectal cancer, thyroid cancer, breast cancer, ovarian cancer, endometrial cancer, prostate cancer, testicular cancer, liver cancer, kidney cancer, renal cell carcinoma, bladder cancer, pancreatic cancer, brain cancer, glioma, sarcoma, osteosarcoma, bone cancer, skin cancer, squamous cancer, Kaposi's sarcoma, melanoma, malignant melanoma, lymphoma, or leukemia.

In one embodiment, the treatment is treatment of:
a carcinoma, for example a carcinoma of the bladder, breast, colon (e.g., colorectal carcinomas such as colon adenocarcinoma and colon adenoma), kidney, epidermal, liver, lung (e.g., adenocarcinoma, small cell lung cancer and non-small cell lung carcinomas), oesophagus, gall bladder, ovary, pancreas (e.g., exocrine pancreatic carcinoma), stomach, cervix, thyroid, prostate, skin (e.g., squamous cell carcinoma);
a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma;
a hematopoietic tumor of myeloid lineage, for example acute and chronic myelogenous leukemias, myelodysplastic syndrome, or promyelocytic leukemia;
a tumour of mesenchymal origin, for example fibrosarcoma or habdomyosarcoma;
a tumor of the central or peripheral nervous system, for example astrocytoma, neuroblastoma, glioma or schwannoma;
melanoma; seminoma; teratocarcinoma; osteosarcoma; xenoderoma pigmentoum; keratoctanthoma; thyroid follicular cancer; or Kaposi's sarcoma.

In one embodiment, the treatment is treatment of solid tumour cancer.

The anti-cancer effect may arise through one or more mechanisms, including but not limited to, the regulation of cell proliferation, the inhibition of cell cycle progression, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death). The compounds of the present invention may be used in the treatment of the cancers described herein, independent of the mechanisms discussed herein.

In one embodiment (e.g., of use in methods of therapy, of use in the manufacture of medicaments, of methods of treatment), the treatment is treatment of: a hyperproliferative skin disorder.

In one embodiment, the treatment is treatment of: psoriasis, actinic keratosis, and/or non-melanoma skin cancer.

### Conditions Treated—Conditions Characterised by Pathological Angiogenesis

In one embodiment (e.g., of use in methods of therapy, of use in the manufacture of medicaments, of methods of treatment), the treatment is treatment of: a disease or condition that is characterised by inappropriate, excessive, and/or undesirable angiogenesis (as “anti-angiogenesis agents”).

Examples of such conditions include macular degeneration, cancer (solid tumours), psoriasis, and obesity.

### Conditions Treated—Inflammation etc.

In one embodiment (e.g., of use in methods of therapy, of use in the manufacture of medicaments, of methods of treatment), the treatment is treatment of: an inflammatory disease.

In one embodiment, the treatment is treatment of: an inflammatory disease involving pathological activation of T- and B-cell lymphocytes, neutrophils, and/or Mast cells.

In one embodiment, the treatment is treatment of: an inflammatory disease, such as rheumatoid arthritis, osteoarthritis, rheumatoid spondylitis, gouty arthritis, traumatic arthritis, rubella arthritis, psoriatic arthritis, and other arthritic conditions; Alzheimer's disease; toxic shock syndrome, the inflammatory reaction induced by endotoxin or inflammatory bowel disease; tuberculosis; atherosclerosis; muscle degeneration; Reiter's syndrome; gout; acute synovitis; sepsis; septic shock; endotoxic shock; gram negative sepsis; adult respiratory distress syndrome; cerebral malaria; chronic pulmonary inflammatory disease; silicosis; pulmonary sarcoisosis; bone resorption diseases; reperfusion injury; graft versus host reaction; allograft rejections; fever and myalgias due to infection, such as influenza, cachexia, in particular cachexia secondary to infection or malignancy, cachexia secondary to acquired immune deficiency syndrome (AIDS); AIDS; ARC (AIDS related complex); keloid formation; scar tissue formation; Crohn's disease; ulcerative colitis; pyresis; chronic obstructive pulmonary disease (COPD); acute respiratory distress syndrome (ARDS); asthma; pulmonary fibrosis; bacterial pneumonia.

In one preferred embodiment, the treatment is treatment of: an arthritic condition, including rheumatoid arthritis and rheumatoid spondylitis; inflammatory bowel disease, including Crohn's disease and ulcerative colitis; and chronic obstructive pulmonary disease (COPD).

In one preferred embodiment, the treatment is treatment of: an inflammatory disorder characterized by T-cell proliferation (T-cell activation and growth), for example, tissue graft rejection, endotoxin shock, and glomerular nephritis.

### Conditions Treated—Heart Failure

The AEAA compounds of the present invention are useful in the treatment of conditions associated with heart remodelling.

In one embodiment, the treatment is treatment of: myocyte hypertrophy of the heart, impaired contractility of the heart, and/or pump failure of the heart.

In one embodiment, the treatment is treatment of: pathologic cardiac hypertrophy.

In one embodiment, the treatment is treatment of: heart failure.

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviatiation of symptoms of the condition, amelioration of the condition, and cure of the condition. Treatment as a prophylactic measure (i.e., prophylaxis) is also included. For example, use with patients who have not yet developed the condition, but who are at risk of developing the condition, is encompassed by the term “treatment.”

For example, treatment of cancer includes the prophylaxis of cancer, reducing the incidence of cancer, alleviating the symptoms of cancer, etc.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

### Combination Therapies

The term “treatment” includes combination treatments and therapies, in which two or more treatments or therapies are combined, for example, sequentially or simultaneously. For example, the AEAA compounds described herein may also be used in combination therapies, e.g., in conjunction with other agents, for example, cytotoxic agents, anticancer agents, etc. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets.

For example, it may be beneficial to combine treatment with an AEAA compound as described herein with one or more other (e.g., 1, 2, 3, 4) agents or therapies that regulates cell growth or survival or differentiation via a different mechanism, thus treating several characteristic features of cancer development.

One aspect of the present invention pertains to an AEAA compound as described herein, in combination with one or more additional therapeutic agents, as described below.

The particular combination would be at the discretion of the physician who would select dosages using his common general knowledge and dosing regimens known to a skilled practitioner.

The agents (i.e., the AEAA compound described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., the AEAA compound described here, plus one or more other agents) may be formulated together in a single dosage form, or alternatively, the individual agents may be formulated separately and presented together in the form of a kit, optionally with instructions for their use.

### Other Uses

The AEAA compounds described herein may also be used as cell culture additives to inhibit PKD (e.g., PKD1, PKD2, PKD3), to inhibit cell proliferation, etc.

The AEAA compounds described herein may also be used as part of an in vitro assay, for example, in order to determine whether a candidate host is likely to benefit from treatment with the compound in question.

The AEAA compounds described herein may also be used as a standard, for example, in an assay, in order to identify other compounds, other PKD (e.g., PKD1, PKD2, PKD3) inhibitors, other anti-proliferative agents, other anti-cancer agents, etc.

### Kits

One aspect of the invention pertains to a kit comprising (a) an AEAA compound as described herein, or a composition comprising a compound as described herein, e.g., preferably provided in a suitable container and/or with suitable packaging; and (b) instructions for use, e.g., written instructions on how to administer the compound or composition.

The written instructions may also include a list of indications for which the active ingredient is a suitable treatment.

### Routes of Administration

The AEAA compound or pharmaceutical composition comprising the AEAA compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

### Formulations

While it is possible for the AEAA compound to be administered alone, it is preferable to present it as a pharmaceutical formulation (e.g., composition, preparation, medicament) comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one AEAA compound, as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or admixed with one or more other pharmaceutically acceptable ingredients. The compound may be presented in a liposome or other microparticulate which is designed to target the compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the compound in a flavored basis, usually sucrose and acacia or tragacanth. Pastilles typically comprise the compound in an inert matrix, such as gelatin and glycerin, or sucrose and acacia. Mouthwashes typically comprise the compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated or scored and may be formulated so as to provide slow or controlled release of the compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the compound and a paraffinic or a water-miscible ointment base.

Creams are typically prepared from the compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the compound through the skin or other affected areas. Examples of such dermal penetration enhancers include dimethylsulfoxide and related analogues.

Emulsions are typically prepared from the compound and an oily phase, which may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichloro-tetrafluoroethane, carbon dioxide, or other suitable gases.

Formulations suitable for ocular administration include eye drops wherein the compound is dissolved or suspended in a suitable carrier, especially an aqueous solvent for the compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additional contain other pharmaceutically acceptable ingredients, such as anti-oxidants, buffers, preservatives; stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the AEAA compounds, and compositions comprising the AEAA compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the AEAA compound is in the range of about 100 μg to 10 about 250 mg (more typically about 100 μg to about 25 mg) per kilogram body weight of the subject per day. Where the compound is a salt, an ester, an amide, a prodrug, or the like, the amount administered is calculated on the basis of the parent compound and so the actual weight to be used is increased proportionately.

### EXAMPLES

The following examples are provided solely to illustrate the present invention and are not intended to limit the scope of the invention, as described herein.

### SYNTHESIS EXAMPLES

### General Methods

### Flash Chromatography

Flash chromatography was performed using BDH silica gel 60.

### General Methods

### NMR

Proton NMR spectra were recorded using a Bruker AMX-300 NMR machine at 300 MHz. Shifts were reported in ppm values relative to an internal standard of tetramethylsilane (TMS) or residual protic solvent. The following abbreviations were used to describe the splitting patterns: s (singlet), d (doublet), t (triplet), q (quartet), m (multiplet), dd (double-doublet), dt (double-triplet), br (broad).

### General Methods

### LCMS Methods

Samples analysed by High Performance Liquid Chromatography-Mass Spectrometry employed the following conditions.

### Method 1

Method 1 employed Gilson 306 pumps, Gilson 811C mixer, Gilson 806 manometric module and Gilson UV/VIS 152 detector at 254 nm wavelength. The mass spectrometer was a Finnigan AQA and a Phenomenex Luna, 5 μm pore size, C18 column of dimensions 50×4.60 mm was used. The injection volume was 10 μL.

The mobile phase consisted of a mixture of water and acetonitrile containing 0.1% formic acid. The eluent flow rate was 1 mL/min, using 95% water: 5% acetonitrile, changed linearly to 2% water: 98% acetonitrile over 3 minutes and then maintained at this mixture for 5 minutes.

### Method 2

Method 2 employed Gilson 306 pumps, Gilson 811C mixer, Gilson 806 manometric module, and Gilson UV/VIS 152 detector at 254 nm wavelength. The mass spectrometer was a Finnigan AQA and a Waters SunFire, 5 μm pore size, C18 column of dimensions 50×4.60 mm was used. The injection volume was 10 μL.

The mobile phase consisted of a mixture of water and acetonitrile containing 0.1% formic acid. The eluent flow rate was 1.5 mL/min, using 95% water: 5% acetonitrile, changed linearly to 5% water: 95% acetonitrile over 5.5 minutes and then maintained at this mixture for 2 minutes.

### Method 3

Method 3 employed Waters 515 pumps, a Waters 2525 mixer and a Waters 2996 diode array detector. The detection was performed between 210 nm and 650 nm. The mass spectrometer was a Waters micromass ZQ and a Waters SunFire, 5 μm pore size, C18 column of dimensions 50×4.60 mm was used. The injection volume was 10 μL.

The mobile phase consisted of a mixture of water and acetonitrile containing 0.1% formic acid. The eluent flow rate was 1.5 mL/min, using 95% water: 5% acetonitrile, changed linearly to 5% water: 95% acetonitrile over 5.5 minutes and then maintained at this mixture for 2 minutes.

### General Methods

### Preparatory HPLC

Samples purified by Mass Spectrometry directed High Performance Liquid Chromatography employed the following conditions.

Waters 515 pumps, a Waters 2525 mixer and a Waters 2996 diode array detector. The detection was performed between 210 nm and 650 nm. The mass spectrometer was a Waters micromass ZQ and a SunFire, 5 μm pore size, C18 column of dimensions 50×19 mm was used. The injection volume was up to 500 μL of solution at a maximum concentration of 50 mg/mL. The mobile phase consisted of a mixture of water and acetonitrile containing 0.1% formic acid. The eluent flow rate was 25 mL/min using 95% water, 5% acetonitrile, changing linearly over 5.3 minutes to 95% MeCN, 5% water, and maintaining for 0.5 minutes.

### General Synthesis Procedure A

Compounds were synthesised starting from either the commercially available anthranilic acids or bis-chloroquinazoline, following the scheme illustrated below.

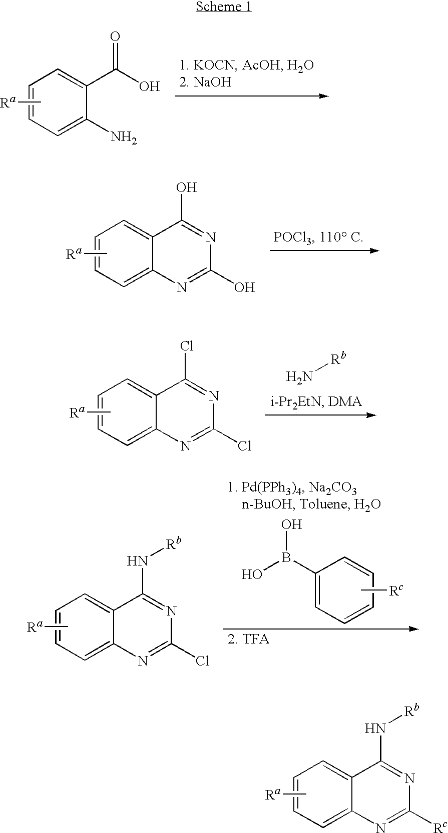

### Synthesis 1

### 7-Trifluoromethyl-quinazoline-2,4-diol

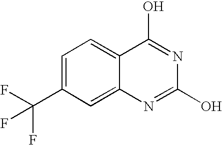

To a stirred suspension of 4-trifluoromethyl anthranilic acid (6.15 g, 30 mmol) in water (200 mL) was added acetic acid (2 mL) and potassium cyanate (3.11 g, 38 mmol) with stirring. The suspension was allowed to stir at room temperature for 16 hours. Solid sodium hydroxide (40 g) was added portionwise with ice cooling. The reaction mixture was allowed to stir for a further 15 hours at room temperature. A precipitate had formed and this was filtered off and dissolved in hot water (100 mL). The solution was treated with acetic acid to pH 5 causing the product to precipitate. The suspension was cooled in ice and the solid was filtered off and washed well with water on the filter. The solid was transferred to a round bottomed flask, suspended in toluene and methanol and evaporated to a white solid. Yield: 1.5 g, 22%. Analytical LCMS method 1, retention time: 5.09 min. ¹H NMR (d-6 DMSO) δ: 11.48 (br, s, 2H), 8.08 (d, 1H), 7.48 (d, 1H), 7.45 (s, 1H). The product was used in the subsequent step without further purification.

### Synthesis 2

### 2,4-Dichloro-7-trifluoromethyl-quinazoline

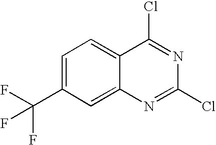

In a round bottomed flask was placed 7-trifluoromethyl-quinazoline-2,4-diol (1.61 g, 7.0 mmol) which was treated with phosphorus oxychloride (20 mL) and heated at 105° C. under nitrogen for 15 hours. The reaction mixture was allowed to cool to room temperature and the phosphorus oxychloride was evaporated under reduced pressure to a pale brown solid. This was treated with ice water (70 mL) and extracted with dichloromethane (2×100 mL). The organics were dried over magnesium sulfate filtered and evaporated to a white solid. Yield: 0.45 g, 24%. Analytical LCMS method 1, retention time 5.61 min, M+H=248. The product was used in the subsequent step without further purification.

### Synthesis 3

### [2-(2-Chloro-7-trifluoromethyl-quinazolin-4-yl-amino)-ethyl]-carbamic acid tert-butyl ester

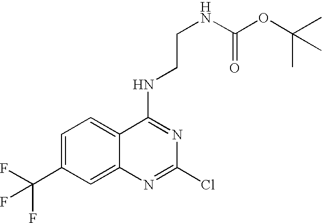

In a round bottomed flask 2,4-dichloro-7-trifluoromethyl-quinazoline (0.45 g, 1.70 mmol) was dissolved in N,N-dimethylacetamide (5 mL) and treated with (2-amino-ethyl)-carbamic acid tert-butyl ester (0.32 mL, 2.0 mmol) and di-iso-propylethylamine (0.91 mL, 5.1 mmol) and allowed to stir at room temperature for 2 days. The reaction mixture was poured into a separating funnel containing water (100 mL) and extracted with ethyl acetate (2×50 mL). The organics were washed with water (100 mL) and then brine (100 mL), dried with magnesium sulfate, filtered and evaporated under reduced pressure to a yellow gum. Yield: 0.45 g, 68%. Analytical LCMS method 1, retention time 6.23 min, M+H=391. The product was used in the subsequent step without further purification.

### Synthesis 4

### 2-[4-(2-Amino-ethylamino)-7-trifluoromethyl-quinazolin-2-yl]-phenol (XX-090)

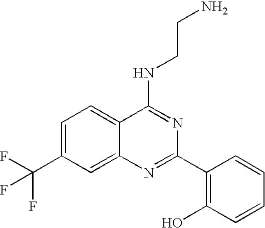

In a round bottomed flask was added toluene (1 mL), n-butanol (1.5 mL) and 2 M sodium carbonate solution (1.5 mL) the mixture was degassed by bubbling nitrogen gas through the mixture for 20 minutes. In another flask was added [2-(2-chloro-7-trifluoromethyl-quinazolin-4-ylamino)-ethyl]-carbamic acid tert-butyl ester (0.20 g, 0.50 mmol), 2-hydroxybenzeneboronic acid (0.20 g, 1.50 mmol) and palladium tetrakistriphenyl phoshine (0.040 g, 0.035 mmol). The flask was evacuated and back filled with nitrogen twice and the solvent was added to the solid reagents under nitrogen. The reaction flask was fitted with a reflux condenser and the reaction mixture was heated at 110° C. under nitrogen for 15 hours. The reaction mixture was cooled to room temperature. The reaction mixture was diluted with water (20 mL) and extracted with ethyl acetate (2×20 mL). The organics were combined, washed with brine (20 mL), dried with magnesium sulfate, filtered and evaporated to a brown solid. This was dissolved in ethyl acetate and passed through a short column of silica gel eluting with 1:1 ethyl acetate:cyclohexane. The solvent was evaporated and the residue was treated with trifluoroacetic acid (1 mL) and allowed to stir at room temperature for 15 hours. The reaction mixture was added directly to a saturated solution of saturated sodium bicarbonate and mixed well to precipitate a yellow solid. Ethyl acetate (20 mL) was added to dissolve the solid and the layers were separated. The organics were washed with brine (20 mL), dried over magnesium sulfate, filtered and evaporated to a yellow solid. The crude product was purified by preparatory HPLC. Analytical LCMS method 1, retention time 4.91 min, M+H=349.

The following compounds were synthesised using the same general method.

TABLE S-1

| | Analytical LCMS | | |
|---|---|---|---|
| ID No. | retention time (min) | M + H | Method |
| XX-050 | 4.88 | 329 | 2 |
| XX-051 | 4.76 | 313 | 1 |
| XX-080 | 4.7 | 315 | 1 |
| XX-082 | 4.35 | 341 | 1 |
| XX-085 | 4.52 | 295 | 1 |
| XX-088 | 3.01 | 295 | 1 |
| XX-090 | 4.91 | 349 | 1 |
| XX-091 | 4.81 | 315 | 1 |
| XX-092 | 4.64 | 295 | 1 |
| XX-097 | 4.78 | 315 | 1 |
| XX-098 | 4.65 | 299 | 1 |
| XX-099 | 4.28 | 299 | 1 |
| XX-120 | 4.16 | 359 | 1 |
| XX-121 | 4.28 | 357 | 1 |

### General Synthesis Procedure B

Compounds were synthesised, starting from the commercially available benzonitriles, following the scheme illustrated below.

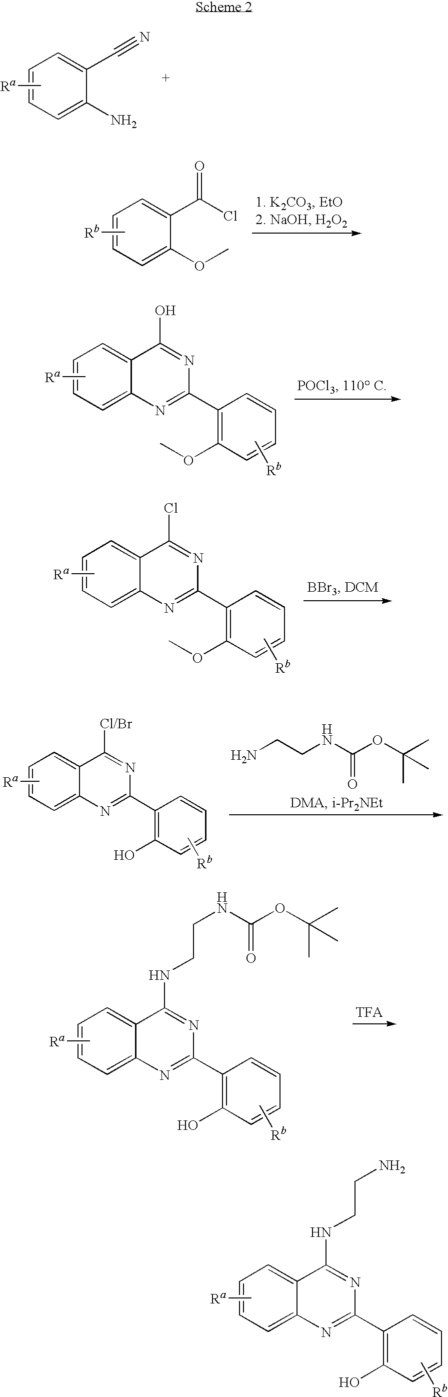

### Synthesis 5

### 7-Chloro-2-(2-methoxy-phenyl)-quinazolin-4-ol

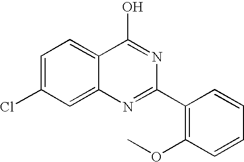

In a 3 neck round bottomed flask 2-amino-4-chlorobenzonitrile (7.63 g, 50.0 mmol) and potassium carbonate were added in diethyl ether (400 mL) and heated to reflux. The suspension was treated dropwise over 5 minutes with 2-methoxybenzoyl chloride (8.05 mL, 60 mmol). The resultant mixture was heated at reflux under nitrogen for 24 hours and allowed to cool to room temperature and the solid precipitate was filtered off and washed well with water on the sinter followed by diethyl ether. A pale yellow solid was obtained. Analytical LCMS method 1, retention time 6.68 min, M+H=287.

The solid was suspended in 16% sodium hydroxide (200 mL) and hydrogen peroxide (50 mL) and heated at reflux for 15 hours. The reaction mixture was allowed to cool to room temperature and filtered. The filtrates were treated with acetic acid to pH 6 and the resultant precipitate was filtered and washed well with water. The solid was washed with diethyl ether (50 mL) and then dissolved in methanol and evaporated to give a yellow solid. Yield: 3.7 g, 26%. Analytical LCMS method 1, retention time 6.19 min, M+H=287. ¹H NMR (d-6 DMSO) δ: 8.12, (d, 1H), 7.74 (s, 1H), 7.66 (dd, 1H), 7.56-7.50 (m, 2H), 7.18 (d, 1H), 7.09 (t, 1H). The product was used in the next step without further purification.

### Synthesis 6

### 4,7-Dichloro-2-(2-methoxy-phenyl)-quinazoline

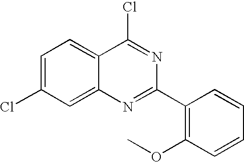

7-Chloro-2-(2-methoxy-phenyl)-quinazolin-4-ol (3.73 g, 13 mmol) was weighed into a round bottomed flask and treated with phosphorus oxychloride (60 mL). The resultant suspension was heated at 110° C. for 4.5 hours. The reaction mixture was allowed to cool and then evaporated to an orange oil that was treated with ice water (150 mL) and saturated sodium hydrogen carbonate (150 mL). The mixture was extracted with ethyl acetate (2×150 mL), washed with brine (200 mL), dried over magnesium sulfate, filtered and evaporated to a yellow solid. Yield: 3.20 g, 81%. Analytical LCMS method 1, retention time 6.64 min, M+H=305. The product was used without further purification.

### Synthesis 7

### 2-(4,7-Dichloro-quinazolin-2-yl)-phenol and 2-(4-Bromo-7-chloro-quinazolin-2-yl)-phenol

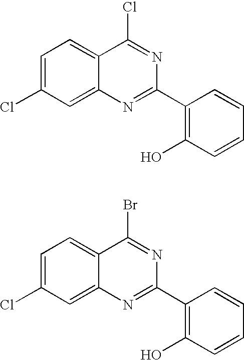

A 3 necked round bottomed flask was charged with 4,7-dichloro-2-(2-methoxy-phenyl)-quinazoline (0.92 g, 3 mmol) and dissolved in dichloromethane (10 mL). The flask was flushed with nitrogen and treated with boron tribromide (30 mL, 30 mmol, 1 M in dichloromethane) dropwise from a pressure equalising dropping funnel at −5° C. under nitrogen. The reaction mixture was allowed to warm to room temperature and stirred for 35 hours. The reaction mixture was added to ice (300 mL) and extracted with dichloromethane (3×150 mL). The organics were washed with brine (50 mL), dried with magnesium sulfate, filtered and evaporated to a yellow solid. Yield: 0.82 g, 94%. Analytical LCMS method 1, retention time 7.58 min, M+H=291. (chloro analog), retention time 7.84 min, M+H=335 (bromo analog). The crude material was used without further purification in the next step.

### Synthesis 8

### {2-[7-Chloro-2-(2-hydroxy-phenyl)-quinazolin-4-ylamino]-ethyl}-carbamic acid tert-butyl ester

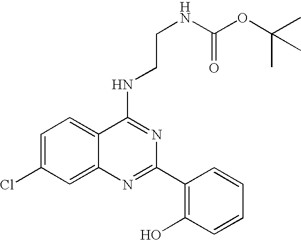

A mixture of 2-(4,7-dichloro-quinazolin-2-yl)-phenol and 2-(4-bromo-7-chloro-quinazolin-2-yl)-phenol (0.80 g, 2.75 mmol) was weighed into a round bottomed flask, dissolved in N,N-dimethylacetamide (5 mL) and treated with (2-amino-ethyl)-carbamic acid tert-butyl ester (0.53 g, 3.3 mmol), di-iso-propylethylamine (1.42 mL, 8.25 mmol) and allowed to stir at room temperature for 2 hours. The reaction mixture was poured into a separating funnel containing water (150 mL) and extracted with ethyl acetate (2×150 mL). The organics were washed with water (150 mL) and brine (150 mL), dried with magnesium sulfate, filtered and evaporated to a yellow solid. Yield: 0.9 g, 79%. Analytical LCMS method 1, retention time 6.85 min, M+H=415. ¹H NMR (d-6 DMSO) δ: 14.36 (s, 1H), 8.78 (t, 1H), 8.50 (d, 1H), 8.27 (d, 1H), 7.88 (s, 1H), 7.58 (d, 1H), 7.37 (t, 1H), 7.04 (t, 1H), 6.94-6.88 (m, 2H), 3.70-3.66 (m, 2H), 3.35-3.31 (m, 2H), 1.34 (s, 9H).

### Synthesis 9

### 2-[4-(2-Amino-ethylamino)-7-chloro-quinazolin-2-yl]-phenol (XX-087)

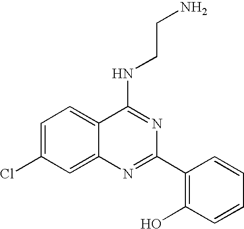

{2-[7-chloro-2-(2-hydroxy-phenyl)-quinazolin-4-ylamino]-ethyl}-carbamic acid tert-butyl ester (0.12 g, 0.05 mmol) was treated with trifluoroacetic acid (1 mL) and allowed to stir for 2 hours at room temperature. The reaction mixture was poured into a saturated solution of sodium bicarbonate and extracted with ethyl acetate (2×50 mL). The organics were filtered, as there was a small quantity of a yellow solid. The filtrates were dried with magnesium sulfate, filtered and evaporated to a pale yellow solid. The crude product was purified by preparatory HPLC. Analytical LCMS method 1, retention time 4.80 min, M+H=315.

The following compounds were synthesised using the same general method.

TABLE S-2

| | Analytical LCMS | | |
|---|---|---|---|
| ID No. | retention time (min) | M + H | Method |
| XX-081 | 4.45 | 295 | 1 |
| XX-083 | 5 | 349 | 1 |
| XX-087 | 4.8 | 315 | 1 |

XX-106 was synthesised according to method B omitting the boron tribromide mediated demethylation step to afford intermediate {2-[2-(2-Methoxy-phenyl)-6-nitro-quinazolin-4-ylamino]-ethyl}-carbamic acid tert-butyl ester. This was then further manipulated as follows:

### Synthesis 10

### {2-[6-Amino-2-(2-methoxy-phenyl)-quinazolin-4-ylamino]-ethyl}-carbamic acid tert-butyl ester

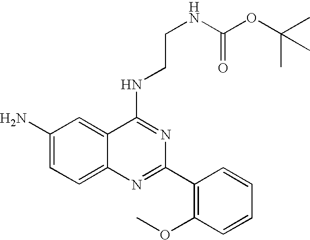

To a round bottomed flask was added {2-[2-(2-methoxy-phenyl)-6-nitro-quinazolin-4-ylamino]-ethyl}-carbamic acid tert-butyl ester (0.13 g, 0.3 mmol) a magnetic stirrer bar and palladium on carbon 5% (5 mg). The flask was evacuated and backfilled with nitrogen twice. The flask was flushed with hydrogen from a balloon and then left to stir for 15 hours at room temperature. The solution was filtered through a pad of celite and evaporated to yield a yellow foam. Yield=0.105 g, 83%. Analytical LCMS method 1, retention time 4.87 min, M+H=410. The product was used directly in the next step.

### Synthesis 11

### 2-[6-Amino-4-(2-amino-ethylamino)-quinazolin-2-yl]-phenol

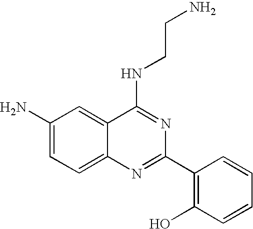

In a 25 mL round bottomed flask was added {2-[6-amino-2-(2-methoxy-phenyl)-quinazolin-4-ylamino]-ethyl}-carbamic acid tert-butyl ester (0.25 g, 0.10 mmol) which was dissolved in dichloromethane (5 mL) and cooled to −78° C. and treated with boron tribromide (1.25 mL, 1.25 mmol, 1 M in dichloromethane). The reaction mixture was allowed to stir at −78° C. for 1 hour then allowed to warm slowly to room temperature. Stirring was continued for a further 3 days. The solution was treated with saturated sodium bicarbonate (20 mL) and the aqueous layer was extracted twice with dichloromethane (2×20 mL), dried over magnesium sulfate, filtered and evaporated. The residue was purified by preparatory HPLC. The yellow solid was triturated with acetonitrile. Analytical LCMS method 1, retention time 0.84 min, M+H=296.

### General Synthesis Procedure C

Additional compounds were synthesised, first using Procedure B to obtain {2-[7-chloro-2-(2-hydroxy-phenyl)-quinazolin-4-ylamino]-ethyl}-carbamic acid tert-butyl ester, followed by the additional steps of Procedure C, as illustrated in following scheme.

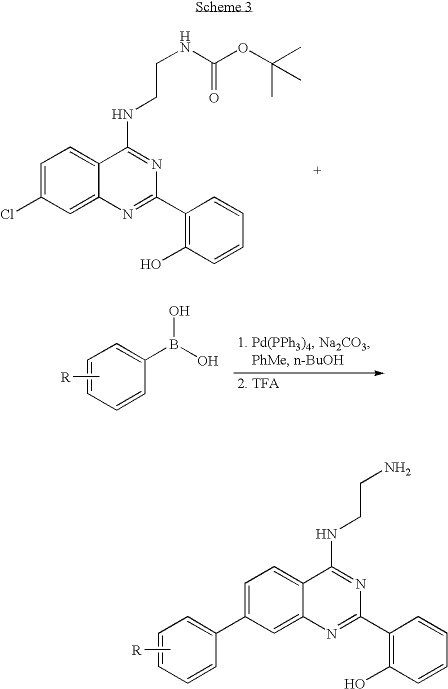

### Synthesis 12

### 2-[4-(2-Amino-ethylamino)-7-phenyl-quinazolin-2-yl]-phenol (XX-089)

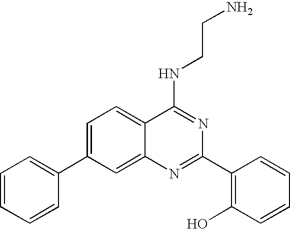

In a round bottomed flask was added toluene (1 mL), n-butanol (1.5 mL) and 2 M sodium carbonate solution (1.5 mL) the mixture was degassed by bubbling nitrogen gas through the mixture for 20 minutes. In a Radleys greenhouse tube was added {2-[7-chloro-2-(2-hydroxy-phenyl)-quinazolin-4-ylamino]-ethyl}-carbamic acid tert-butyl-ester (0.08 g, 0.20 mmol), benzeneboronic acid (0.073 g, 0.60 mmol) and palladium tetrakistriphenyl phoshine (0.010 g, 0.012 mmol). The tube was placed inside the greenhouse under an atmosphere of nitrogen. The degassed solvent was added to the solid reagents through a septum under nitrogen. The reaction mixture was heated at 100° C. for 24 hours. The layers were allowed to separate and the organic layer was decanted using a pipette and passed through an SPE cartridge containing silica eluting with 1:1 ethyl acetate:hexanes. The solvent was evaporated under reduced pressure. The residue was treated with trifluoroacetic acid (1 mL) and allowed to stir for 3 hours at room temperature. The reaction mixture was added to a saturated solution of sodium bicarbonate (50 mL) with stirring. The aqueous layer was extracted with ethyl acetate (2×15 mL). The organics were washed with brine (20 mL), separated and evaporated under reduced pressure. The residue was dissolved in DMSO (2 mL) and purified by preparatory HPLC. Analytical LCMS method 1, retention time 4.93 min, M+H=357.

The following compounds were synthesised using the same general method.

TABLE S-3

| | Analytical LCMS | | |
|---|---|---|---|
| ID No. | retention time (min) | M + H | Method |
| XX-086 | 5.04 | 425 | 1 |
| XX-089 | 4.93 | 357 | 1 |

### General Synthesis Procedure D

Compounds were synthesised, starting from commercially available anthranilamides, acid chlorides (commercially available or synthesised from their corresponding salicylic acids following literature procedures), and commercially available amines, alcohols, or thiols or known amines (synthesised from their corresponding amino amides or amino acids following literature procedures), following the scheme illustrated below.

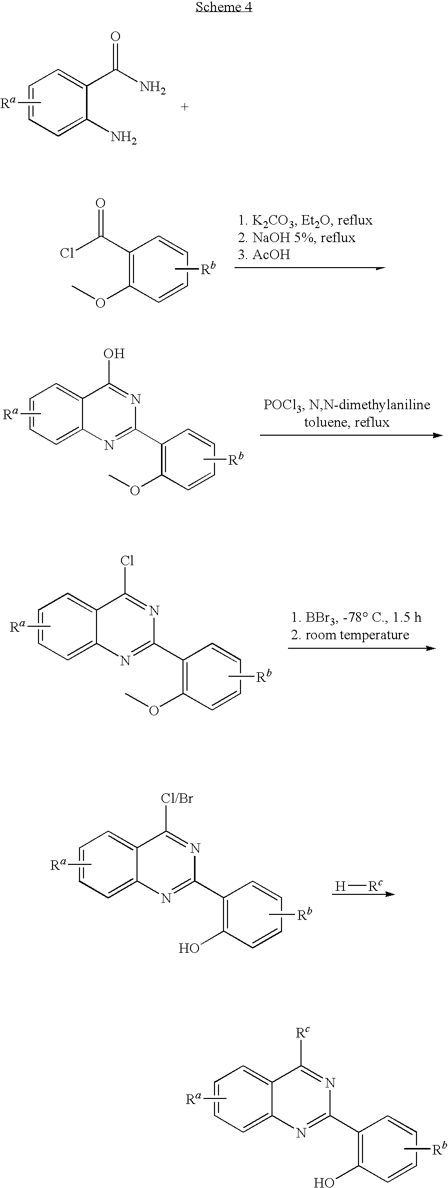

### Synthesis 13

### 2-(2-Methoxy-phenyl)-quinazolin-4-ol

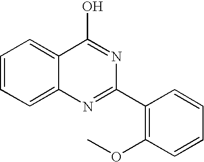

Anthranilamide (10.21 g, 75 mmol) was dissolved in diethyl ether (500 mL) with potassium carbonate (14.51 g, 105 mmol) and treated over 5 minutes with o-anisoyl chloride (95 mmol, 12.7 mL). The reaction mixture was refluxed for 5 hours and allowed to cool to room temperature. The diethyl ether was removed under reduced pressure and the residue was suspended in 5% sodium hydroxide solution (300 mL) and heated at reflux for 1.5 hours. The reaction mixture was allowed cool to room temperature and then further ice cooled and neutralised to pH 6 with acetic acid. The resulting suspension was filtered and washed with water (5×100 mL). The wet solid was transferred to a round bottom flask using methanol to dissolve and evaporated. The solid was then suspended in toluene and evaporated to dryness twice. The product was obtained as an off white powder. Yield: 14 g, 75%. Analytical LCMS method 1, retention time 5.78 min, M+H=253.2. ¹H NMR (d-6 DMSO) δ: 12.12 (s, br, 1H) 8.14 (dd, 1H), 7.85-7.80 (m, 1H), 7.71-7.68 (m, 2H), 7.56-7.50 (m, 2H), 7.19 (d, 1H), 7.09 (dt, 1H), 3.86 (s, 3H). The product was used without further purification in the next step.

### Synthesis 14

### 4-Chloro-2-(2-methoxy-phenyl)-quinazoline

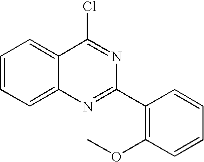

N,N-dimethylaniline (10.5 mL, 83.2 mmol) was added to a solution of 2-(2-methoxy-phenyl)-quinazolin-4-ol (14 g, 55.5 mmol) in toluene (250 mL), and the resultant solution was heated at 90° C. for 1 hour. The reaction mixture was allowed to cool to room temperature and treated with phosphorus oxychloride (5.1 mL, 55.5 mmol). The reaction mixture was heated at 90° C. for 3 hours. After cooling to room temperature the reaction mixture was poured onto ice and neutralised with sodium hydrogen carbonate. The layers were separated and the aqueous was extracted with toluene (3×150 mL). The organics were washed with brine (300 mL), dried over magnesium sulfate, filtered and evaporated to give an orange oil. This was cooled in the refrigerator overnight and a solid crystallised. This was filtered off and triturated with cyclohexane 5 times and once with diethyl ether to yield a pink solid. The filtrates were evaporated and purified by flash column chromatography (1:9 ethyl acetate cyclohexane increasing gradually to 2:3 ethyl acetate: cyclohexane). Yield 6.5 g, 60%. Analytical LCMS method 1, retention time 6.24 min, M+H=271. ¹H NMR (CDCl₃) δ: 8.31-8.28 (m, 1H), 8.15-8.12 (m, 1H), 7.98-7.93 (m, 1H), 7.81-7.68 (m, 2H), 7.48-7.42 (m, 1H), 7.12-7.04 (m, 2H), 3.89 (s, 3H).

### Synthesis 15

### 2-(4-Chloroquinazolin-2-yl)-phenol & 2-(4-Bromoquinazolin-2-yl)-phenol

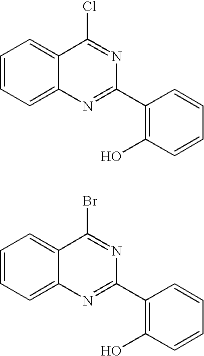

A 3 neck round bottom flask was charged with 4-chloro-2-(2-methoxy-phenyl)-quinazoline (5 g, 18.47 mmol). The flask was fitted with a low temperature thermometer, pressure equalising dropping funnel and nitrogen inlet. The flask was nitrogen flushed and dichloromethane (100 mL) was added. The resultant solution was cooled to −78° C. and treated dropwise over 10 minutes with boron tribromide (1 M in dichloromethane, 92.3 mL, 92.34 mmol). The solution was allowed to stir for 1.5 hours at this temperature and then the cooling bath was removed. The solution was left to stir under nitrogen at room temperature for 3.5 hours and then poured slowly into a beaker containing ice and sodium hydrogen carbonate solution. The resultant suspension was poured into a separating funnel and extracted with dichloromethane (3×150 mL). The organics were washed with brine (200 mL), separated, dried over magnesium sulfate, filtered and evaporated to a yellow solid. Yield 2.9 g. Analytical LCMS method 1, retention time 7.02 min, M+H=257 (chloro analog); retention time 7.18 min, M+H=301 (bromo analog). The mixture was used without further purification.

### Synthesis 16

### 2-[4-((R)-2-Aminopropylamino)-quinazolin-2-yl]-phenol (XX-063)

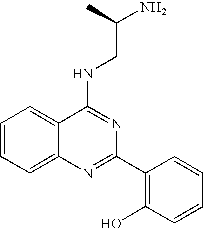

In a test tube was added the mixture of 2-(4-chloroquinazolin-2-yl)phenol and 2-(4-bromoquinazolin-2-yl)phenol (0.09 g, 0.35 mmol) and N,N-dimethylacetamide (2 mL). The solution was treated with (R)-propane-1,2-diamine dihydrochloride (0.37 mL, 2.10 mmol) and di-iso-propylethylamine (0.16 mL, 1 mmol) and allowed to stir at room temperature for 18 hours. The solution was treated with water (50 mL) and extracted twice with ethyl acetate. The organics were washed twice with brine, dried over magnesium sulfate, and concentrated under reduced pressure. The residue was purified by preparatory HPLC. Analytical LCMS method 1, retention time 4.50 min, M+H=309. ¹H NMR (d-6DMSO) δ: 14.90 (brs, 1H) 9.25 (brs, 1H), 8.50-8.34 (m, 3H), 7.85-7.75 (m, 2H), 7.56 (t, 1H), 7.36 (td, 1H), 6.92 (t, 2H), 3.83-3.65 (m, 2H), 3.57-3.50 (m, 1H), 1.23 (d, 3H).

The following compounds were synthesised using the same general method.

TABLE S-4

| | Analytical LCMS | | |
|---|---|---|---|
| ID No. | retention time (min) | M + H | Method |
| XX-001 | 5 | 309 | 1 |
| XX-002 | 4.88 | 309 | 1 |
| XX-003 | 3.58 | 373 | 2 |
| XX-004 | 0.42 | 373 | 2 |
| XX-005 | 0.46 | 359 | 2 |
| XX-006 | 0.43 | 345 | 2 |
| XX-007 | 4.83 | 295 | 1 |
| XX-008 | 6.60 | 372 | 2 |
| XX-009 | 3.43 | 329 | 2 |
| XX-010 | 4.51 | 295 | 1 |
| XX-011 | 3.66 | 355 | 2 |
| XX-012 | 3.45 | 339 | 1 |
| XX-013 | 4.01 | 391 | 2 |
| XX-015 | 3.77 | 359 | 1 |
| XX-016 | 3.7 | 357 | 2 |
| XX-017 | 3.52 | 341 | 2 |
| XX-018 | 3.96 | 391 | 2 |
| XX-019 | 3.97 | 407 | 2 |
| XX-020 | 3.79 | 357 | 2 |
| XX-021 | 3.81 | 357 | 2 |
| XX-022 | 4.46 | 339 | 2 |
| XX-023 | 3.16 | 323 | 2 |
| XX-024 | 2.97 | 355 | 2 |
| XX-025 | 3.84 | 373 | 2 |
| XX-026 | 5.07 | 371 | 2 |
| XX-027 | 3.56 | 353 | 2 |
| XX-028 | 4.91 | 337 | 1 |
| XX-029 | 3.83 | 378 | 2 |
| XX-031 | 3.99 | 378 | 2 |
| XX-032 | 3.64 | 343 | 2 |
| XX-033 | 3.37 | 227 | 2 |
| XX-034 | 3.15 | 323 | 2 |
| XX-035 | 3.93 | 393 | 2 |
| XX-036 | 3.86 | 377 | 2 |
| XX-037 | 3.3 | 325 | 2 |
| XX-038 | 0.47 | 325 | 2 |
| XX-039 | 4.65 | 309 | 1 |
| XX-040 | 6.59 | 372 | 2 |
| XX-041 | 3.81 | 357 | 2 |
| XX-042 | 3.76 | 363 | 2 |
| XX-043 | 4.82 | 329 | 1 |
| XX-045 | 3 | 331 | 2 |
| XX-046 | 0.44 | 313 | 2 |
| XX-047 | 0.35 | 309 | 3 |
| XX-048 | 3.94 | 363 | 2 |
| XX-049 | 3.39 | 331 | 2 |
| XX-052 | 2.86 | 309 | 2 |
| XX-053 | 3.35 | 340 | 1 |
| XX-054 | 3.77 | 379 | 2 |
| XX-055 | 3.76 | 363 | 2 |
| XX-056 | 3.49 | 329 | 2 |
| XX-057 | 2.36 | 308 | 2 |
| XX-058 | 3.52 | 239 | 2 |
| XX-059 | 3.38 | 309 | 2 |
| XX-060 | 3.17 | 311 | 2 |
| XX-061 | 0.67 | 311 | 2 |
| XX-062 | 3.75 | 345 | 2 |
| XX-063 | 4.48 | 295 | 1 |
| XX-064 | 4.1 | 329 | 1 |
| XX-065 | 4.52 | 295 | 1 |
| XX-066 | 4.6 | 325 | 1 |
| XX-068 | 5.07 | 357 | 1 |
| XX-069 | 4.69 | 323 | 1 |
| XX-070 | 4.82 | 371 | 1 |
| XX-071 | 4.74 | 371 | 1 |
| XX-072 | 4.76 | 355 | 1 |
| XX-073 | 4.58 | 309 | 1 |
| XX-074 | 4.77 | 323 | 1 |
| XX-075 | 4.51 | 295 | 1 |
| XX-076 | 4.64 | 309 | 1 |
| XX-077 | 4.45 | 309 | 1 |
| XX-079 | 3.31 | 337 | 2 |
| XX-084 | 4.82 | 315 | 1 |
| XX-100 | 4.39 | 281 | 1 |
| XX-101 | 4.51 | 295 | 1 |
| XX-102 | 4.61 | 337 | 1 |
| XX-103 | 4.86 | 386 | 1 |
| XX-104 | 4.5 | 309 | 1 |
| XX-105 | 4.48 | 295 | 1 |
| XX-107 | 4.76 | 309 | 1 |
| XX-108 | 4.55 | 309 | 1 |
| XX-109 | 4.4 | 295 | 1 |
| XX-110 | 4.47 | 323 | 1 |
| XX-111 | 5 | 399 | 1 |
| XX-112 | 4.42 | 324 | 1 |
| XX-113 | 4.44 | 309 | 1 |
| XX-114 | 4.6 | 339 | 1 |
| XX-116 | 3.69 | 373 | 2 |
| XX-117 | 2.73 | 344 | 2 |
| XX-118 | 3.72 | 331 | 2 |
| XX-119 | 0.45 | 311 | 1 |
| XX-122 | 0.77 | 323 | 1 |
| XX-123 | 4.9 | 323 | 1 |
| XX-124 | 0.84 | 323 | 1 |

The following compounds were also synthesised using the same general method.

TABLE S-5

| | Analytical LCMS | | |
|---|---|---|---|
| ID No. | retention time (min) | M + H | Method |
| XX-130 | 3.02 | 353 | 2 |
| XX-257 | 3.46 | 373 | 2 |
| XX-126 | 3.66 | 389 | 2 |
| XX-250 | 3.58 | 365 | 2 |
| XX-283 | 3.80 | 474 | 2 |
| XX-282 | 3.97 | 443 | 2 |
| XX-281 | 3.72 | 459 | 2 |
| XX-231 | 0.37 | 396 | 2 |
| XX-236 | 3.30 | 363 | 2 |
| XX-237 | 3.72 | 445 | 2 |
| XX-234 | 3.55 | 438 | 2 |
| XX-277 | 3.67 | 357 | 2 |
| XX-238 | 3.65 | 345 | 2 |
| XX-324 | 4.03 | 419 | 1 |
| XX-326 | 3.30 | 428 | 1 |
| XX-243 | 3.78 | 435 | 2 |
| XX-246 | 3.59 | 354 | 2 |
| XX-248 | 0.53 | 386 | 2 |
| XX-241 | 3.65 | 375 | 2 |
| XX-347 | 3.50 | 478 | 1 |
| XX-240 | 3.88 | 425 | 1 |
| XX-245 | 4.06 | 435 | 1 |
| XX-330 | 3.56 | 463 | 1 |
| XX-327 | 3.19 | 428 | 1 |
| XX-321 | 3.79 | 415 | 1 |
| XX-331 | 3.75 | 415 | 1 |
| XX-314 | 3.82 | 403 | 1 |
| XX-316 | 3.87 | 399 | 1 |
| XX-319 | 4.03 | 409 | 1 |
| XX-328 | 3.30 | 428 | 1 |
| XX-317 | 4.05 | 469 | 1 |
| XX-313 | 3.95 | 419 | 2 |
| XX-332 | 4.01 | 399 | 2 |
| XX-322 | 3.96 | 399 | 2 |
| XX-325 | 3.92 | 403 | 2 |
| XX-333 | 4.26 | 453 | 2 |
| XX-323 | 4.25 | 453 | 2 |
| XX-318 | 4.02 | 453 | 2 |
| XX-315 | 3.73 | 415 | 2 |
| XX-329 | 3.73 | 387 | 2 |
| XX-348 | 3.56 | 478 | 2 |
| XX-334 | 3.72 | 435 | 2 |
| XX-251 | 3.81 | 391 | 2 |
| XX-249 | 3.19 | 387 | 2 |
| XX-232 | 3.17 | 375 | 2 |
| XX-233 | 3.55 | 416 | 2 |
| XX-247 | 2.84 | 386 | 2 |
| XX-235 | 3.54 | 404 | 2 |
| XX-242 | 3.62 | 375 | 2 |
| XX-312 | 3.96 | 421 | 2 |
| XX-239 | 3.81 | 429 | 2 |
| XX-335 | 3.40 | 401 | 1 |
| XX-310 | 4.07 | 461 | 1 |
| XX-311 | 4.28 | 461 | 2 |
| XX-309 | 4.13 | 461 | 2 |
| XX-278 | 3.94 | 444 | 2 |
| XX-320 | 3.93 | 403 | 2 |
| XX-336 | 3.75 | 385 | 2 |
| XX-166 | 3.73 | 389 | 2 |
| XX-280 | 2.45 | 355 | 3 |
| XX-164 | 1.96 | 305 | 3 |
| XX-137 | 3.21 | 405 | 3 |
| XX-158 | 3.01 | 371 | 3 |
| XX-150 | 2.80 | 355 | 3 |
| XX-160 | 2.20 | 353 | 3 |
| XX-154 | 3.00 | 393 | 3 |
| XX-147 | 3.05 | 373 | 3 |
| XX-146 | 2.70 | 373 | 3 |
| XX-157 | 3.08 | 371 | 3 |
| XX-155 | 3.20 | 421 | 3 |
| XX-339 | 2.11 | 353 | 3 |
| XX-153 | 2.68 | 349 | 3 |
| XX-159 | 2.98 | 351 | 3 |
| XX-286 | 2.95 | 387 | 3 |
| XX-148 | 3.40 | 405 | 3 |
| XX-161 | 3.23 | 387 | 3 |
| XX-138 | 3.81 | 371 | 3 |
| XX-151 | 3.15 | 463 | 3 |
| XX-135 | 2.65 | 371 | 3 |
| XX-156 | 3.29 | 405 | 3 |
| XX-133 | 2.81 | 377 | 3 |
| XX-279 | 2.56 | 410 | 3 |
| XX-130 | 2.57 | 383 | 3 |
| XX-260 | 2.55 | 367 | 3 |
| XX-152 | 3.31 | 367 | 3 |
| XX-244 | 2.94 | 399 | 2 |

Compounds bearing an aryl or heteroaryl group as R^{b} were synthesised starting from the commercially available 5-bromo-2-hydroxy-benzoic acid and transformed into 5-bromo-2-methoxy-benzoyl chloride by methods known in the literature. The final step to convert the aryl bromide into the final products where R^{b} is aryl or heteroaryl was performed as for the example shown in Procedure K, Synthesis 54.

XX-345, XX-283, XX-282, and XX-281 were synthesised using Procedure D to give 2-(4-chloro-quinazolin-2-yl)-benzene-1,4-diol and then further manipulated as described in Procedure T.

XX-231, XX-234, and XX-237 were synthesised by Procedure D and then further manipulated as described in Procedure S.

XX-236 was synthesised by Procedure D to obtain ((R)-1-{[2-(2-Hydroxy-5-iodo-phenyl)-quinazolin-4-ylamino]-methyl}-propyl)-carbamic acid tert-butyl ester and coupled to propargyl alcohol as described in general synthesis procedure U omitting the TBAF deprotection step.

XX-152 and XX-244 were synthesised by way of Procedure D and then further manipulated into the final compounds using Procedure P.

XX-125 was synthesised using Procedure D to afford intermediate 4-chloro-2-(2-methoxy-phenyl)-quinazoline. This was then further manipulated as follows:

### Synthesis 17

### 1-[2-(2-Methoxy-phenyl)-quinazolin-4-yl]-ethane-1,2-diamine (XX-125)

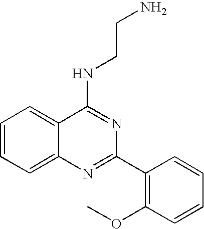

To a solution of 4-chloro-2-(2-methoxy-phenyl)-quinazoline (0.05 g, 0.2 mmol) in N,N-dimethylacetamide (1 mL), (2-amino-ethyl)-carbamic acid tert-butyl ester (0.16 g, 1 mmol) was added and the mixture was stirred at room temperature for 18 hours. The solution was treated with water (50 mL) and extracted with ethyl acetate (2×50 mL). The organics were washed twice with brine (50 mL), dried over magnesium sulfate, and concentrated under reduced pressure. The crude was then dissolved in trifluoroacetic acid (1 mL) and left to stir at room temperature for 4 hours. The solvent was removed under reduced pressure and the residue was purified by preparatory HPLC. Analytical LCMS method 1, retention time 0.73 min, M+H=295.

### Synthesis 18

### (±)-2-Amino-3-[2-(2-hydroxy-phenyl)-quinazolin-4-ylamino]-propionic acid (XX-115)

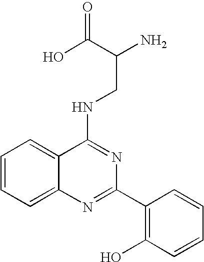

XX-115 was synthesised from XX-114 as follows. To a solution of 2-amino-3-[2-(2-hydroxy-phenyl)-quinazolin-4-ylamino]-propionic acid methyl ester (0.05 g, 0.147 mmol) in a mixture tetrahydrofuran/water 10:1 (3 mL), lithium hydroxide monohydrate (0.007 g, 0.15 mmol) was added. The solution was stirred for 18 hours and then acidified with 1 M HCl. Dichloromethane was added and the two layers were separated. The organics were dried over magnesium sulfate and concentrated under reduced pressure. The crude product was purified by preparatory HPLC. Analytical LCMS method 1, retention time 4.60 min, M+H=325. ¹H NMR (DMSO) δ: 3.88 (dd, 1H), 3.99 (dd, 1H), 4.12-4.25 (m, 1H), 6.88-6.92 (m, 2H), 7.34 (td, 1H), 7.51 (td, 1H), 7.73-7.82 (m, 2H), 8.28 (d, 1H), 8.55 (dd, 1H), 9.22 (brs, 1H), 14.78 (s, 1H).

### Synthesis 19

### (±)-2-[4-(2-Amino-3-hydroxy-propylamino)-quinazolin-2-yl]-phenol (XX-078)

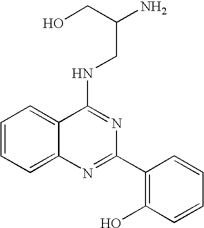

XX-078 was synthesised from XX-115 as follows. To a suspension of lithium aluminium hydride (0.01 1 g, 0.3 mmol) in tetrahydrofuran (2 mL), 2-amino-3-[2-(2-hydroxy-phenyl)-quinazolin-4-ylamino]-propionic acid methyl ester (0.05 g, 0.15 mmol) was added. The mixture was stirred for 18 hours at room temperature and hydrolysed by 0.05 mL of NaOH 2M and 0.1 mL of water. The aluminates were filtered through a celite pad and the filtrate concentrated under reduced pressure. The crude product was purified by preparatory HPLC. Analytical LCMS method 1, retention time 4.43 min, M+H=311. ¹H NMR (DMSO) δ: 3.50-3.88 (m, 5H), 6.84-6.92 (m, 2H), 7.36 (t, 1H), 7.54 (t, 1H), 7.74-7.84 (m, 2H), 8.35 (d, 1H), 8.41 (s, 1H), 8.50 (dd, 1H), 9.30 (brs, 1H).

### Synthesis 20

### 2-[4-((S)-2-Amino-3-hydroxy-propylamino)-quinazolin-2-yl]-4-chloro-phenol (XX-067)

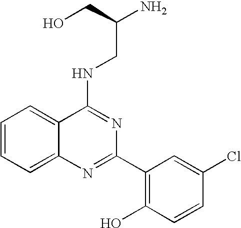

XX-067 was synthesised using the methods described above for XX-115 and XX-078, starting from (S)-2-amino-3-[2-(5-chloro-2-hydroxy-phenyl)-quinazolin-4-ylamino]-propionic acid methyl ester (XX-003). Analytical LCMS method 2, retention time 3.50 min, M+H=339.

### General Synthesis Procedure E

Compounds were synthesised, starting from the commercially available dichloro-pyrimidines, following the scheme illustrated below.

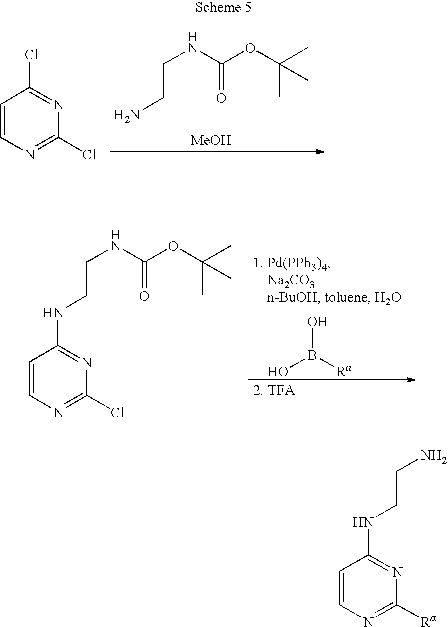

### Synthesis 21

### [2-(2-Chloro-pyrimidin-4-ylamino)-ethyl]-carbamic acid tert-butyl ester

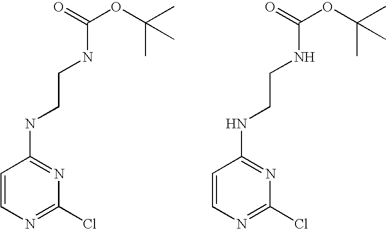

A round-bottomed flask was charged with 2,4-dichloropyrimidine (1.49 g, 10.0 mmol) and methanol (10 mL). The solution was cooled to 0° C. and (2-amino-ethyl)-carbamic acid tert-butyl ester (3.52 g, 22 mmol) was added drop-wise over 2 minutes and the solution was allowed to stir at 0° C. for 15 minutes. The cooling bath was removed and the solution was stirred at room temperature until TLC analysis showed 100% conversion of the starting material. The solvent was evaporated and taken up in ethyl acetate (100 mL). The organics were washed with water (100 mL). The aqueous layer was extracted with ethyl acetate (2×100 mL) and then the organics were washed with brine (200 mL). The organics were dried with magnesium sulfate, filtered and evaporated to yield an oil. This was purified by flash column chromatography using 98% ethyl acetate 2% triethylamine as eluent. The product was obtained as a white solid. Yield: 1.51 g, 55%. Analytical LCMS method 1, retention time 5.79 min, M+H=273. ¹H NMR (CDCl₃) δ: 7.94 (s, br, 1H), 6.27-6.26 (m, br, 2H), 5.10 (s, br, 1H), 3.47 (s, br, 2H), 3.37-3.31 (m, 2H), 1.40 (s, 9H). The product was used without further purification in the subsequent step.

### Synthesis 22

### 2-[4-(2-Amino-ethylamino)-pyrimidin-2-yl]-phenol (XX-096)

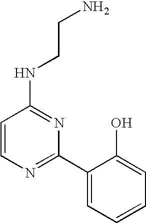

In a round bottomed flask was added toluene (1 mL), n-butanol (1 mL) and 2 M sodium carbonate solution (1 mL) and the mixture was degassed by bubbling nitrogen gas through the mixture for 20 minutes. Another flask was charged with [2-(2-chloro-pyrimidin-4-ylamino)-ethyl]-carbamic acid tert-butyl ester (0.08 g, 0.3 mmol), 2-hydroxybenzene boronic acid (0.124 g, 0.9 mmol) and palladium tetrakistriphenylphosphine (0.017 g, 0.015 mmol). The flask was evacuated and back filled with nitrogen twice and the solvent was added to the solid reagents under nitrogen. The reaction flask was fitted with a reflux condenser and the reaction mixture was heated at 110° C. under nitrogen for 15 hours. The reaction mixture was cooled to room temperature; the organic layer was separated off and evaporated. The residue was passed through a short column of silica gel and evaporated again. The residue was treated with trifluoroacetic acid (1 mL) and allowed to stir at room temperature for 2 hours; the solution was quenched into a saturated solution of sodium bicarbonate (40 mL) and extracted with ethyl acetate (2×20 mL). The organics were evaporated and purified by preparatory HPLC to yield a white solid. Analytical LCMS method 1, retention time 0.74 min, M+H=231.

The following compounds were synthesised using the same general method.

TABLE S-6

| | Analytical LCMS | | |
|---|---|---|---|
| ID No. | retention time (min) | M + H | Method |
| XX-093 | 1.95 | 265 | 1 |
| XX-094 | 0.84 | 249 | 1 |
| XX-095 | 1.76 | 249 | 1 |
| XX-096 | 0.74 | 231 | 1 |

The following compounds were also synthesised using the same general method.

TABLE S-7

| | Analytical LCMS | | |
|---|---|---|---|
| ID No. | retention time (min) | M + H | Method |
| XX-255 | 2.48 | 293 | 2 |
| XX-256 | 2.90 | 293 | 2 |
| XX-202 | 3.00 | 307 | 2 |
| XX-187 | 3.48 | 307 | 2 |
| XX-184 | 3.35 | 311 | 2 |
| XX-181 | 3.51 | 327 | 2 |
| XX-222 | 3.13 | 277 | 2 |
| XX-262 | 3.25 | 333 | 2 |
| XX-177 | 3.21 | 321 | 2 |
| XX-127 | 3.17 | 321 | 2 |
| XX-342 | 2.79 | 337 | 2 |
| XX-129 | 3.35 | 406 | 2 |
| XX-343 | 4.05 | 430 | 2 |
| XX-344 | 3.56 | 376 | 2 |
| XX-193 | 3.86 | 402 + MeCN | 2 |
| XX-198 | 3.72 | 352 | 2 |
| XX-205 | 3.96 | 361 | 2 |
| XX-136 | 2.15 | 301 | 3 |
| XX-266 | 3.43 | 325 | 2 |
| XX-267 | 3.21 | 321 | 2 |
| XX-261 | 0.43 | 408 | 2 |

### General Synthesis Procedure F

Compounds were synthesised, starting from the commercially available dichloro-pyrimidines and protected diamines, following the schemes illustrated below.

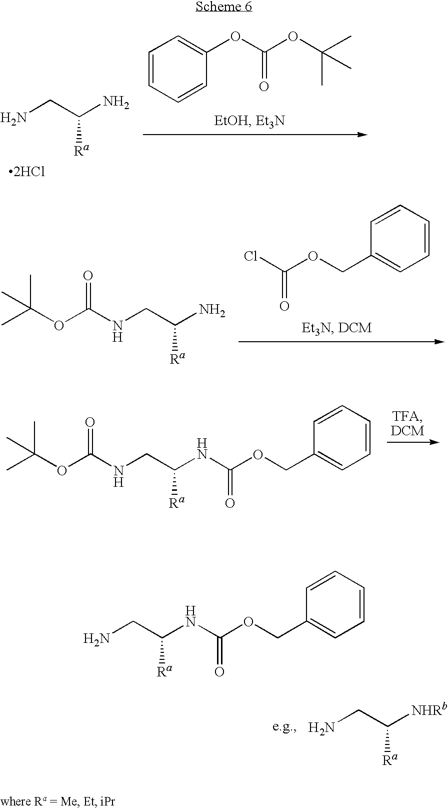

### Synthesis 23

### (R)-(2-Amino-propyl)-carbamic acid tert-butyl ester

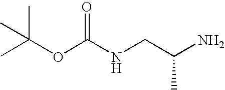

(R)-Propane-1,2-diamine dihydrochloride (40 mmol, 5.88 g) was dissolved in ethanol (150 mL) and treated with triethylamine (21.04 mL, 150 mmol) and tert-butylphenylcarbonate (80 mmol, 50.54 g). The reaction mixture was heated to reflux under nitrogen for 48 hours, allowed to cool, and diluted with water (150 mL) and carefully acidified to pH 3 with 1 M hydrochloric acid. The aqueous phase was extracted with dichloromethane (2×100 mL), basified with 2 M sodium hydroxide to pH 11 and extracted again with dichloromethane (3×150 mL). The organic fractions were combined, dried over magnesium sulfate, filtered, and evaporated. Analytical LCMS method 1, M+H=175. The compound was used without further purification

### Synthesis 24

### (R)-2-(Benzyloxycarbonylamino-propyl)-carbamic acid tert-butyl ester

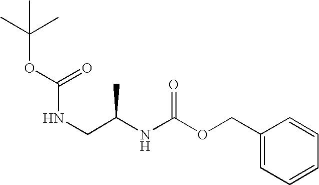

(R)-2-Amino-propyl)-carbamic acid tert-butyl ester (14.7 mmol, 2.56 g) was dissolved in dichloromethane (70 mL), and benzylchloroformate (17.64 mmol, 2.48 g) and triethylamine (28.4 mmol, 4.08 g) were added. The reaction was stirred at room temperature for 3 hours, and poured onto water, treated with dilute ammonia, and extracted several times with ethyl acetate. The organic fractions were combined, dried over magnesium sulfate, and concentrated to give a colorless gum. Analytical LCMS method 1, M+H=309. The compound was used without further purification.

### Synthesis 25

### (R)-2-(Amino-1-methyl-ethyl)-carbamic acid benzyl ester

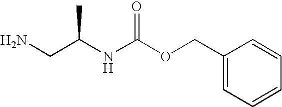

(R)-2-(Benzyloxycarbonylamino-propyl)-carbamic acid tert-butyl ester (14.7 mmol, 4.53 g) was dissolved in trifluoroacetic acid (50 mL) and stirred at room temperature for 16 hours. The trifluoroacetic acid was removed under vacuum, the remaining residue was added to a saturated solution of sodium bicarbonate, solid sodium bicarbonate was added until the pH was basic, and the mixture was then extracted with dichloromethane several times. The organic fractions were combined, dried with magnesium sulfate, and evaporated. Analytical LCMS method 2, M+H=209. The product was used without further purification.

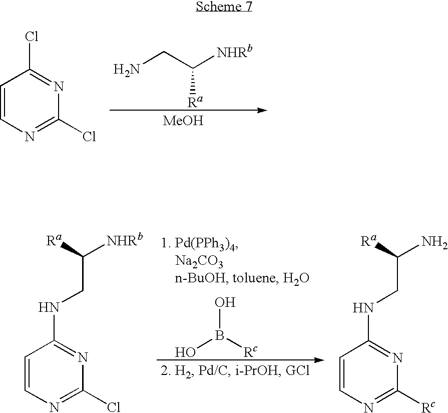

### Synthesis 26

### (R)-2-[(2-Chloro-pyrimidin-4-ylamino)-1-methyl-ethyl]-carbamic acid benzyl ester

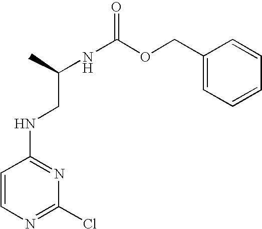

2,4-Dichloropyrimidine (3.35 mmol, 0.5 g) was dissolved in N,N-dimethylacetamide (20 mL) and ((R)-2-amino-1-methyl-ethyl)-carbamic acid benzyl ester (5.02 mmol, 1.05 g) was added and the reaction mixture stirred overnight. Di-iso-propylethylamine (1.2 mL, 6.7 mmol) was added and the reaction stirred at room temperature for a further 48 hours. The reaction mixture was poured onto water and extracted several times with ethyl acetate, dried over magnesium sulfate, and concentrated under vacuum. The crude compound was purified by flash column chromatography using an eluent system of 7:3 ethyl acetate: cyclohexane. Yield: 282 mg. Analytical LCMS method 1, retention time 4.33 min, M+H=321. The product was used without further purification.

### Synthesis 27

### {(R)-2-[2-(5-Chloro-2-hydroxy-phenyl)-pyrimidin-4-ylamino]-1-methyl-ethyl}-carbamic acid benzyl ester

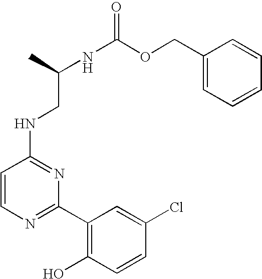

(5-Chloro-2-hydroxyphenyl)boronic acid (0.6 mmol, 0.1 g) and palladium tetrakistriphenylphosphine (0.01 mmol, 0.011 g) were weighed into a tube and placed under a nitrogen atmosphere. A previously de-gassed sodium carbonate solution (2 M, 1 mL) was added. (R)-2-(2-Chloro-pyrimidin-4-ylamino)-1-methyl-ethyl]-carbamic acid benzyl ester (0.2 mmol, 0.06 g) was dissolved in a de-gassed mixture of toluene (1 mL) and butanol (1 mL) and added to the other reagents. The reaction mixture was heated to 10° C. for 48 hours. The reaction mixture was allowed to cool to room temperature, water was added in a test tube, a small amount of ethyl acetate was added, and the organic layer was decanted off and filtered through a plug of silica and eluted with ethyl acetate. The solvent was removed under reduced pressure to yield the crude material. Analytical LCMS method 1, retention time 5.01 min, M+H=413. The product was used in the next step without further purification.

### Synthesis 28

### 2-[4-((R)-2-Amino-propylamino)-pyrimidin-2-yl]-4-chloro-phenol (XX-044)

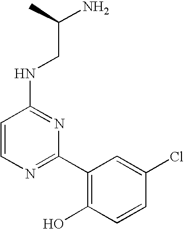

A round bottomed flask was evacuated and backfilled with nitrogen twice before {(R)-2-[2-(5-chloro-2-hydroxy-phenyl)-pyrimidin-4-ylamino]-1-methyl-ethyl}-carbamic acid benzyl ester (0.339 mmol, 0.14 g) dissolved in ethyl acetate (2 mL) was added via a syringe through the septum, a small amount of palladium on carbon 5% was added from the tip of a spatula, and a flow of hydrogen was passed through the flask for 5 minutes. The flask was then fitted with a hydrogen balloon and allowed to react at room temperature overnight. The reaction had not gone to completion, so 2 drops of hydrochloric acid and another spatula tip of palladium on carbon 5% were added and the mixture was left to stir for 24 hours. The reaction mixture was filtered through celite to remove the palladium, washed through with ethyl acetate and concentrated under reduced pressure. The crude product was purified by preparatory HPLC. Analytical LCMS method 2, retention time 0.50 min, M+H=279.

The following compounds were synthesised using the same general method.

TABLE S-8

| | Analytical LCMS | | |
|---|---|---|---|
| ID No. | retention time (min) | M + H | Method |
| XX-014 | 3.38 | 307 | 2 |
| XX-030 | 3.25 | 293 | 2 |
| XX-044 | 0.50 | 279 | 2 |

### General Synthesis Procedure G

Compounds were synthesised, starting from the commercially available anilines and alkoxyacetophenones, following, for example, the scheme illustrated below.

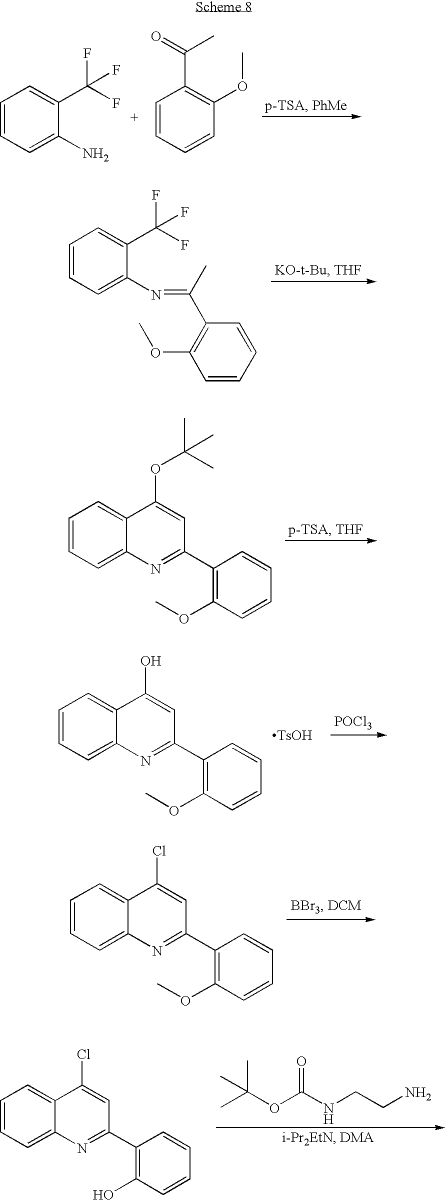

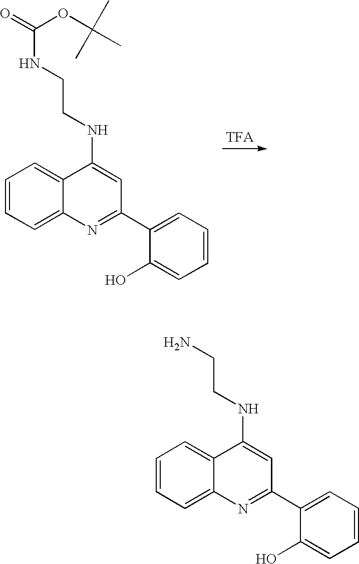

### Synthesis 29

### [1-(2-Methoxy-phenyl)-eth-(E)-ylidene]-(2-trifluoromethyl-phenyl)-amine

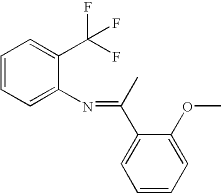

A 500 mL round bottom flask was charged with 2-(trifluoromethyl)-aniline (9.67 g, 60 mmol), 2-methoxyacetophenone (9.99 mL, 72 mmol), para-toluenesulfonic acid (0.11 g, 0.57 mmol) and toluene (250 mL). The solution was heated under Dean-Stark conditions under nitrogen for 18 hours. The reaction mixture was allowed to cool and the solvent was evaporated to provide a thick brown oil. This was purified by kugelrohr distillation to provide a pale green oil. Toluene (2 mL) and hexane (10 mL) were added and the solution was left to stand in the fridge overnight whereby a white solid crystallised. The solid was filtered off and washed with hexanes to produce a white crystalline solid. Yield: 4.2 g, 24%. This compound was used in the subsequent step without further purification.

### Synthesis 30

### 4-tert-Butoxy-2-(2-methoxy-phenyl)-quinoline

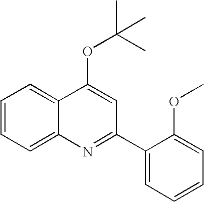

A 500 mL round bottomed flask was charged with [1-(2-methoxy-phenyl)-eth-(E)-ylidene]-(2-trifluoromethyl-phenyl)-amine (4.99 g, 17 mmol) tetrahydrofuran (250 mL) and potassium-tert-butoxide (9.53 g, 85 mmol). The solution was refluxed for 3 hours under nitrogen and then allowed to cool to room temperature. The solvent was evaporated under reduced pressure and the residue was dissolved in ethyl acetate (100 mL). Water (100 mL) was added and the layers were separated. The aqueous was further extracted with ethyl acetate (2×100 mL). The organic fractions were combined and washed with 2 M hydrochloric acid (200 mL), the layers were separated, and the organics were neutralised with solid sodium bicarbonate. The organics were separated and washed with ethyl acetate (2×100 mL). The combined organics were washed with brine (300 mL), dried with magnesium sulfate, filtered, and evaporated to give a light brown oil. The crude product was purified by flash column chromatography using 1:4 ethyl acetate hexanes with 2% triethylamine as eluent. Yield: 2.6 g, 48% of a yellow oil. ¹H NMR: (CDCl₃) δ: 8.20 (1H, dd), 8.08 (1H, d), 7.91 (1H, dd), 7.69-7.63 (1H, m), 7.50-7.39 (2H, m), 7.14 (1H, t), 7.03 (1H, d), 3.87 (3H, s), 1.66 (9H, s), 1.43 (9H, s). The compound was used without further purification in the subsequent step.

### Synthesis 31

### 2-(2-Methoxy-phenyl)-quinolin-4-ol toluene-4-sulfonic acid salt

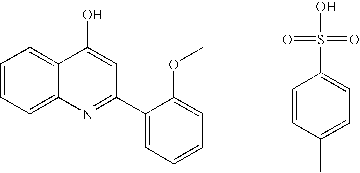

A round bottomed flask was charged with 4-tert-butoxy-2-(2-methoxy-phenyl)-quinoline (2.49 g, 8.1 mmol) and para-toluenesulfonic acid (2.31 g, 12.15 mmol). Tetrahydrofuran (50 mL) was added and the mixture was refluxed for 4 hours and allowed to cool overnight. After cooling further in the fridge the solid was filtered and washed with cyclohexane. Analytical LCMS method 1, retention time 5.02 min, M+H=252. The product was used in subsequent reactions without further purification.

### Synthesis 32

### 4-Chloro-2-(2-methoxy-phenyl)-quinoline

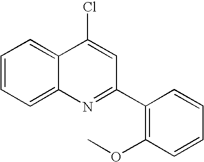

A round bottomed flask was charged with 2-(2-methoxy-phenyl)-quinolin-4-ol toluene-4-sulfonic acid salt (1.49 g, 3.5 mmol). Phosphorus oxychloride (10 mL) was added and the mixture was heated at 100° C. for 2 hours and allowed to cool to room temperature. The phosphorus oxychloride was evaporated under reduced pressure and the residue was added to a saturated solution of sodium bicarbonate (75 mL) and was extracted with ethyl acetate (3×75 mL). The organics were washed with brine (100 mL), dried over magnesium sulfate, filtered, and evaporated to a white solid. The material was purified by flash column chromatography (1:9 ethyl acetate: cyclohexane) to yield the product as a white solid. Yield: 0.40 g, 73% of pure material was obtained and used in the subsequent reaction.

### Synthesis 33

### 2-(4-Chloro-quinolin-2-yl)-phenol

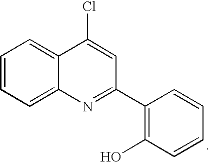

A round bottomed flask was charged with 4-chloro-2-(2-methoxy-phenyl)-quinoline (0.26 g, 0.96 mmol). The flask was nitrogen flushed and dichloromethane (2.5 mL) was added. The resultant solution was cooled to −78° C. and treated drop-wise over 10 minutes with boron tribromide (1 M in dichloromethane, 2.9 mL, 3 mmol). The solution was allowed to stir for 1 hour at this temperature and then the cooling bath was removed. The solution was left to stir under nitrogen at room temperature for 3 hours and then poured slowly into a beaker containing ice. The mixture was neutralised with solid sodium hydrogen carbonate. The resultant suspension was poured into a separating funnel and extracted with dichloromethane (3×50 mL). The organics were washed with brine (100 mL), dried with magnesium sulfate, filtered, and evaporated to give a yellow solid. Yield 0.15 g, 62%. Analytical LCMS method 1, retention time 7.16 min, M+H=256. ¹H NMR (CDCl₃) δ: 14.77 (1H, s), 8.22 (1H, dd), 8.11 (1H, s), 8.04 (1H, s), 7.88 (1H, dd), 7.82-7.77 (1H, m), 7.66-7.61 (1H, m), 7.41-7.35 (1H, m), 7.08 (1H, dd), 7.00-6.94 (1H, m).

### Synthesis 34

### {2-[2-(2-Hydroxy-phenyl)-quinolin-4-ylamino]-ethyl}-carbamic acid tert-butyl ester

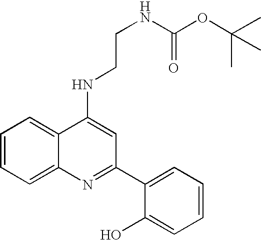

2-(4-Chloro-quinolin-2-yl)-phenol (0.10 g, 0.38 mmol), (2-amino-ethyl)-carbamic acid tert-butyl ester (0.31 g, 1.94 mmol,) and N,N-dimethylacetamide (1 mL) were added to a microwave tube and the reaction was heated to 180° C. for 15 minutes. The crude product was purified by preparatory HPLC. Analytical LCMS method 1, retention time 4.84 min, M+H=380.

### Synthesis 35

### 2-[4-(2-Amino-ethylamino)-quinolin-2-yl]-phenol (YY-002)

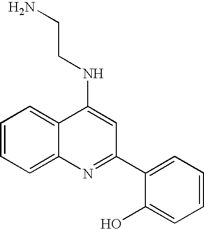

{2-[2-(2-Hydroxy-phenyl)-quinolin-4-ylamino]-ethyl}-carbamic acid tert-butyl ester (0.04 g, 0.1 mmol) was dissolved in trifluoroacetic acid (1 mL) and stirred at room temperature for 4 hours. The trifluoroacetic acid was removed in a Genevac evaporator. Analytical LCMS method 1, retention time 5.88 min, M+H=280.

### General Synthesis Procedure H

Compounds were synthesised, starting from the commercially available 2,4-quinolinediols, following, for example, the scheme illustrated below.

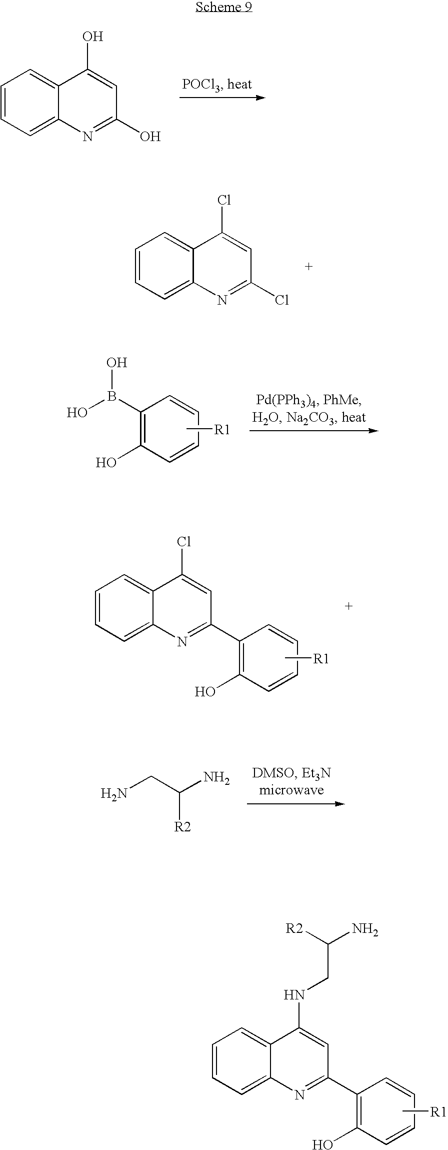

### Synthesis 36

### 2,4-Dichloroquinoline

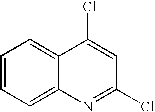

2,4-Quinolinediol (3.22 g, 20 mmol) was suspended in phosphorus oxychloride (50 mL) and heated at 110° C. for 5 hours. The reaction mixture was allowed to cool to room temperature and the phosphorus oxychloride was removed under reduced pressure. The residual oil was added to ice and then extracted with dichloromethane (3×100 mL). The organics were combined, washed with water (100 mL), then brine (200 mL), dried with magnesium sulfate, filtered, and evaporated to give a pale red solid. Analytical LCMS method 2, retention time 6.15 min, M+H=198. Yield 2.50 g, 63%. The product was used without further purification in the next step.

### Synthesis 37

### 4-Chloro-2-(4-chloro-quinolin-2-yl)-phenol

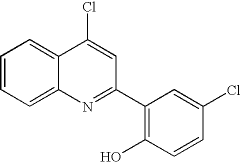

2,4-Dichloroquinoline (0.29 g, 1.5 mmol), 2-hydroxy-5-chloro boronic acid (0.24 g, 1.44 mmol), sodium carbonate (0.31 g, 3 mmol) and palladium tetrakistriphenylphosphine (0.086 g, 0.075 mmol) were suspended in a previously degassed mixture of toluene (3 mL) and water (1 mL). The reaction mixture was refluxed under nitrogen for 15 hours and allowed to cool to room temperature. The contents of the flask were dissolved in ethyl acetate (100 mL) and water (100 mL) and the layers were separated. The aqueous layer was extracted with another portion of ethyl acetate (100 mL), the organics were combined, washed with water (100 mL) and brine (100 mL), dried with magnesium sulfate, filtered, and evaporated to give a pale red solid. The solid was triturated with ethyl acetate to give a yellow solid. Yield: 100 mg, 22%. Analytical LCMS method 2 retention time 7.91 min, M+H=290.

### Synthesis 38

### 2-(4-(R)-2-amino-propylamino)-quinolin-2-yl)-4-chloro-phenol (YY-001)

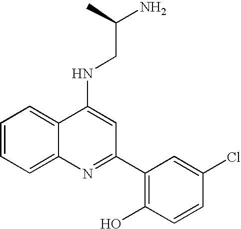

4-Chloro-2-(4-chloro-quinolin-2-yl)-phenol (0.06 g, 0.2 mmol) was dissolved in dimethylsulfoxide (1 mL). R-(+)-Propylenediamine dihydrochloride (0.15 g, 1 mmol) and triethylamine (0.7 mL, 5 mmol) in DMSO (2 mL) were stirred for 5 minutes and then 4-chloro-2-(4-chloro-quinolin-2-yl)-phenol in dimethylsulfoxide (1 mL) was added. The suspension was heated in a microwave reactor at 200° C. for 5 minutes. The ethyl acetate was evaporated and the residue was purified by preparatory HPLC. The compound was obtained as a brown oil that was dissolved in methanol and stirred with activated carbon for 2 hours. The solution was filtered through celite and evaporated to a brown solid. Analytical LCMS method 2 retention time 0.38 min, M+1=328.

The following compound was synthesised by the same general procedure.

TABLE S-9

| | Analytical LCMS | | |
|---|---|---|---|
| ID No. | retention time (min) | M + H | Method |
| YY-003 | 0.40 | 342 | 2 |

### General Synthesis Procedure I

Additional compounds were prepared by the following methods. Primary amines were synthesised by the procedure shown below. It was also possible to synthesise secondary amines by the additional final two steps shown below.

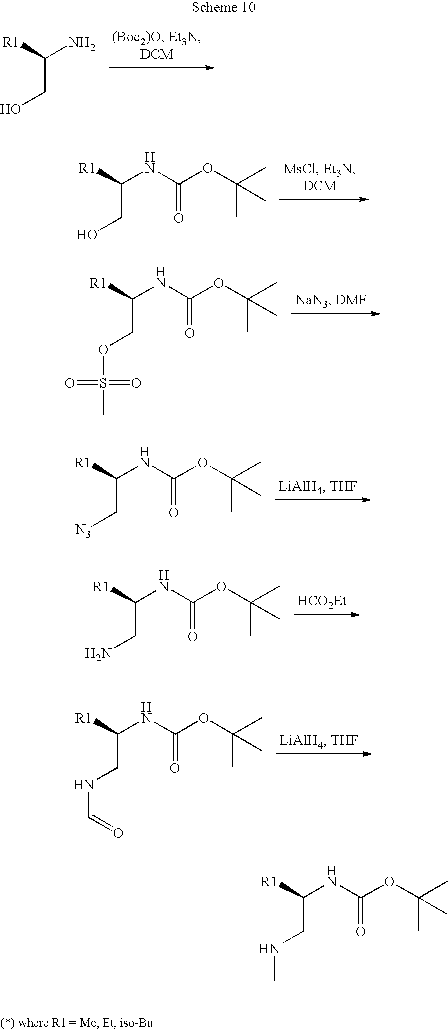

### Synthesis 39

### ((R)-1-Hydroxymethyl-propyl)-carbamic acid tert-butyl ester

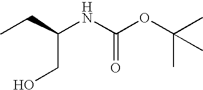

Di-tert-butyl dicarbonate (144 mmol, 31.42 g) was dissolved in dichloromethane (200 mL) and triethylamine (144 mmol, 20.13 ml) and cooled to 0° C. The solution was treated with (R)-(−)-2-Amino-1-butanol (120 mmol, 10.70 g) dropwise over 5 minutes. The reaction mixture was allowed to stir at room temperature for 1 hour and then allowed to stir under nitrogen for 15 hours. The reaction mixture was treated with water (200 mL) and the layers were separated. The aqueous layer was extracted with 3×200 mL of dichloromethane, combined, washed with brine 400 mL, dried with MgSO₄, filtered, and evaporated to a colourless oil. This was purified by flash column chromatography eluting with 1:4 ethyl acetate:cyclohexane to yield the title compound as a colourless oil. Yield 17.2 g, 76%. Analytical LCMS method 2, retention time 5.30 min, M+H=190. ¹H NMR: (CDCl₃) δ: 4.62 (s, br, 1H), 3.69-3.65 (m, 1H), 3.58-3.54 (m, 2H) 2.49 (s, br, 1H), 1.69-1.39 (m, 11H), 0.95 (t, 3H).

### Synthesis 40

### Methanesulfonic acid (R)-2-tert-butoxycarbonylamino-butyl ester

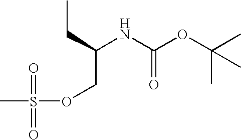

((R)-1-Hydroxymethyl-propyl)-carbamic acid tert-butyl ester (135 mmol, 25.55 g) was dissolved in dichloromethane (750 mL) and treated with triethylamine (148.5 mmol, 20.83 mL). The reaction mixture was cooled to 0° C. and treated with methanesulfonyl chloride (270 mmol, 21.03 mL). The reaction mixture was left to stir at 0° C. for 1 hour and then a further 2 hours at room temperature. The solution was treated with water (500 mL) and the layers were separated. The aqueous was extracted with dichloromethane (2×250 mL), then the organics were combined, washed with brine 500 mL, dried with MgSO₄, filtered, and evaporated to give a gummy white solid. Trituration with cyclohexane afforded the title compound as a white solid. Yield 26.9 g, 75%. Analytical LCMS method 2, retention time 4.72 min, M+H=285.

### Synthesis 41

### ((R)-1-Azidomethyl-propyl)-carbamic acid tert-butyl ester

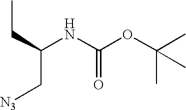

Methanesulfonic acid (R)-2-tert-butoxycarbonylamino-butyl ester (100 mmol, 26.74 g) was dissolved in dimethyl formamide (200 mL) and treated with sodium azide (500 mmol, 32.50 g). The reaction mixture was heated at 80° C. for 4 hours. The reaction mixture was allowed to cool and then poured into a separating funnel that contained water (1 L). The aqueous layer was extracted with ethyl acetate (6×400 mL), the organics were combined, washed with brine 500 mL, dried with MgSO₄, filtered, and evaporated to a yellow oil. Purification by flash column chromatography eluting with 1:1 ethyl acetate:cyclohexane provided the title compound as a yellow oil. Yield 15.0 g, 70%. Analytical LCMS method 2, retention time 5.35 min, M+H=215. ¹H NMR: (CDCl₃) δ: 4.54 (s, br, 1H), 3.62 (s, br, 1H), 3.41-3.36 (m, 2H), 1.59-1.44 (m, 11H), 0.94 (t, 3H).

### Synthesis 42

### ((R)-1-Aminomethyl-propyl)-carbamic acid tert-butyl ester

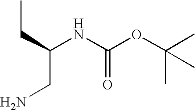

Lithium aluminium hydride (179 mmol, 6.8 g) was suspended in tetrahydrofuran (300 mL) and cooled to 0° C. and treated with ((R)-1-azidomethyl-propyl)-carbamic acid tert-butyl ester (70 mmol, 15 g) in THF 20 mL dropwise over 20 minutes. The resultant solution was allowed to stir at 0° C. for 3 hours. The reaction mixture was cooled to −40° C. and treated dropwise with 6.8 mL of 2 M sodium hydroxide followed by 6.90 mL of water; the thick suspension was left to stir at room temperature over the weekend. The reaction mixture was filtered through a plug of celite and evaporated to give a pale yellow oil. Yield 13.05 g, 99%. Analytical LCMS method 2, retention time 0.60 min, M+H=189. ¹H NMR: (CDCl₃) δ: 4.54 (s, br, 1H), 3.44 (s, br, 1H), 2.75 (dd, 1H), 2.61 (dd, 1H), 1.52-1.31 (m, 13H), 0.92 (t, 3H).

### Synthesis 43

### ((R)-1-Formylaminomethyl-propyl)-carbamic acid tert-butyl ester

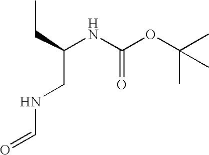

((R)-1-Aminomethyl-propyl)-carbamic acid tert-butyl ester (5.0 mmol, 0.94 g) was dissolved in ethyl formate (25 mL) and heated at 60° C. for 16 hours. The reaction mixture was allowed to cool. Analytical LCMS method 1, retention time 3.50 min, M=217. The solvent was removed under reduced pressure to yield the title compound as a colourless gum. Yield 1.08 g.

### Synthesis 44

### ((R)-1-Methylaminomethyl-propyl)-carbamic acid tert-butyl ester

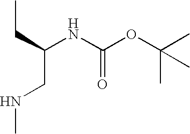

Lithium aluminium hydride (15 mmol, 0.56 g) was suspended in tetrahydrofuran (15 mL) and cooled under nitrogen to 0° C. The suspension was treated with ((R)-1-formylaminomethyl-propyl)-carbamic acid tert-butyl ester (5.0 mmol, 1.08 g) in tetrahydrofuran (10 mL) dropwise over 5 minutes. The resultant suspension was allowed to stir for 1 hour at 0° C. and then 4 hours at room temperature. The reaction mixture was cooled to −20° C. and treated dropwise with sodium hydroxide 2 M (0.56 mL) and then water (0.56 mL). The reaction mixture was allowed to stir at room temperature for 2 days and then filtered through a plug of celite, washing with diethyl ether (200 mL). The solvent was evaporated under reduced pressure to yield the title compound as a pale yellow oil. Yield 1.0 g, 99%. Analytical LCMS method 2, retention time 0.60 min, M+H=203.

### General Synthesis Procedure J

Additional compounds were prepared by the following methods. The amines shown below were synthesised using the procedure illustrated below and were used without purification.

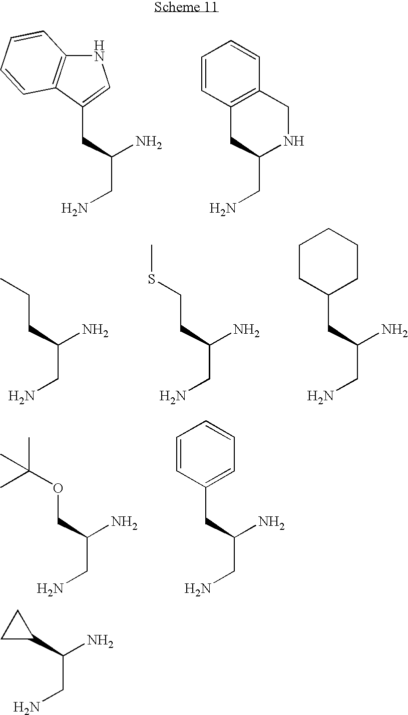

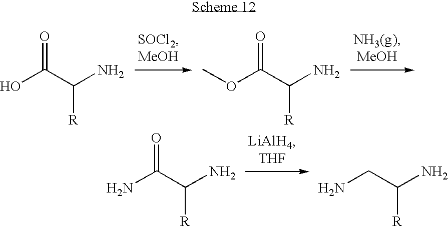

### Synthesis 45

### (R)-2-Amino-3-cyclohexyl-propionic acid methyl ester hydrochloride

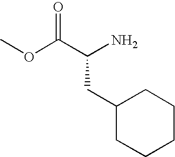

Cyclohexyl-D-alanine-D-2-Amino-3-cyclohexyl-propionic acid (5.85 mmol, 1 g) was suspended in methanol (20 mL) and cooled to 0° C. The reaction mixture was treated dropwise with thionyl chloride (121.0 mmol, 0.9 mL) and stirred at room temperature under nitrogen for 15 hours. The solvent was evaporated to give the title compound as an off white solid. Yield 1 g. Analytical LCMS method 2, retention time 1.03 min, M+H=186.3.

### Synthesis 46

### (R)-2-Amino-3-cyclohexyl-propionamide

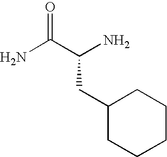

(R)-2-Amino-3-cyclohexyl-propionic acid methyl ester hydrochloride (5.85 mmol, 1.00 g) was dissolved in methanol (10 mL) in a sealable vial and cooled to 0° C. Nitrogen gas was bubbled through the solution for 30 minutes before the vial was sealed. The reaction mixture was left to stir at room temperature for 15 hours. The solvent was evaporated under reduced pressure to give the title compound as a white solid. Analytical LCMS method 2, retention time 0.78 min, M+H=171.3.

### Synthesis 47

### (R)-3-Cyclohexyl-propane-1,2-diamine

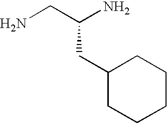

Lithium aluminium hydride (14.6 mmol, 0.55 g) was suspended in tetrahydrofuran (40 mL) and cooled to 0° C. (R)-2-Amino-3-cyclohexyl-propionamide was added portionwise to the reaction mixture. After the addition was complete, the reaction mixture was heated to reflux overnight and then cooled to 0° C. The reaction was quenched with 2 M NaOH (0.5 mL) and water (2×0.5 mL). The reaction mixture was filtered through a plug of celite, washing with ethyl acetate, dichloromethane, and methanol to yield the title compound as a colourless gum. Yield 0.91 g. Analytical LCMS method 2, retention time 1.34 min, M+H=157.3.

### General Synthesis Procedure K

Additional compounds were prepared according to the following methods, using commercially available benzonitriles or benzaldehydes. The amines were synthesised using the methods described herein.

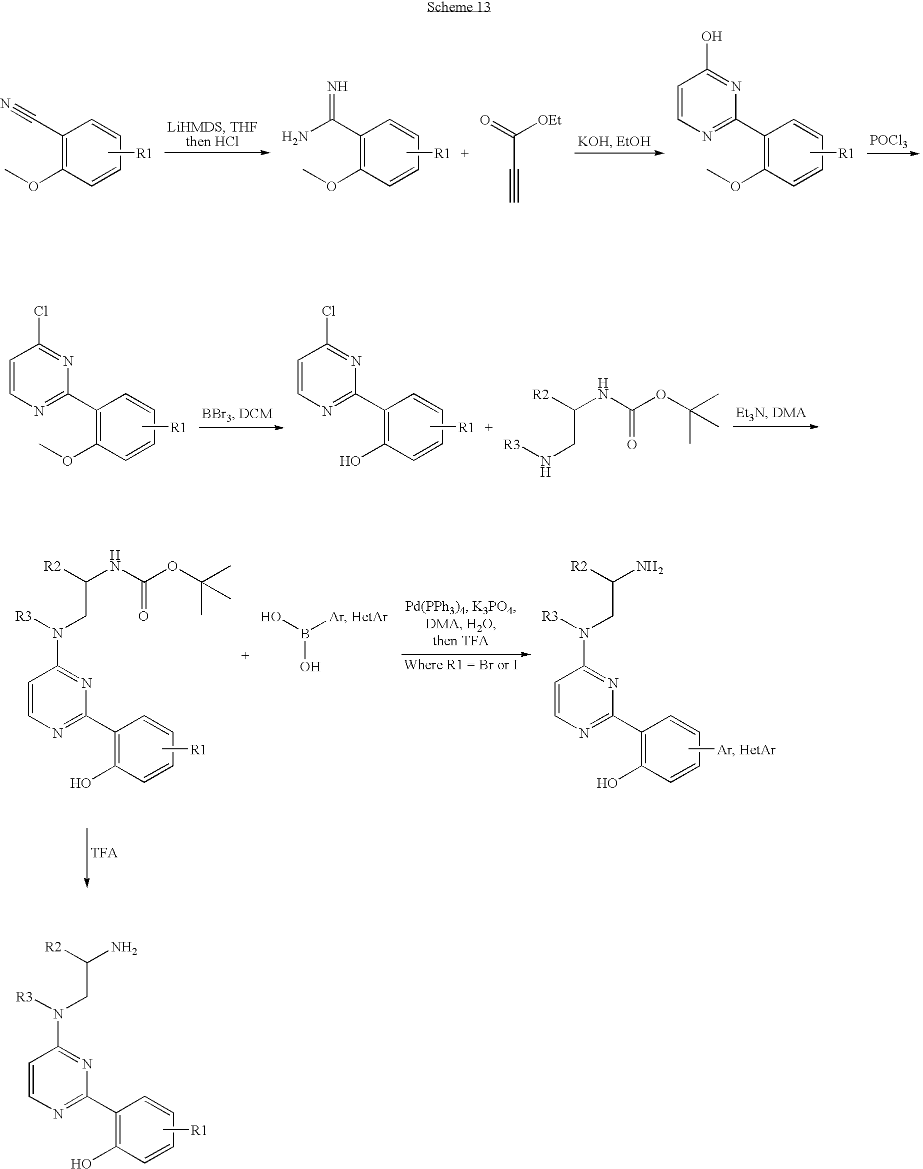

### Synthesis 48

### 5-Bromo-2-methoxy-benzonitrile

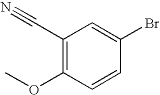

5-Bromo-2-methoxybenzaldehyde (20.00 mmol, 4.30 g) was dissolved in formic acid (20 mL) and treated with hydroxylamine hydrochloride (21.00 mmol, 1.45 g) and sodium acetate (26.00 mmol, 2.13 g). The reaction mixture was heated at reflux for 15 hours. The solvent was removed under reduced pressure and the residue was taken up in ethyl acetate (250 mL). The organics were washed with saturated sodium bicarbonate solution (2×200 mL) and then dried with MgSO₄. The organics were filtered and evaporated under reduced pressure to yield the title compound as a white solid. Yield=4.10 g, 97%. Analytical LCMS method 2, retention time 6.07 min, M+H=214, 216. ¹H NMR: (CDCl₃) δ: 7.66-7.61 (m, 2H), 6.87 (d, 1H), 3.92 (s, 3H).

### Synthesis 49

### 5-Bromo-2-methoxy-benzamidine

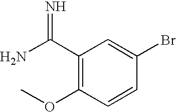

A 1 M solution of lithium bis-hexamethylsilazide (220 mmol, 220 mL) in tetrahydrofuran was transferred into a 3 neck round bottom flask. The solution was cooled to 0° C. and treated dropwise with 5-bromo-2-methoxy-benzonitrile (100 mmol 21.21 g) in tetrahydrofuran over 20 minutes. The reaction mixture was allowed to stir at 0° C. for 30 minutes, then allowed to stir at room temperature for 4 hours. The reaction mixture was cooled to 0° C. and treated with 2 M hydrochloric acid (350 mL) dropwise. The reaction mixture was allowed to stir for 15 hours at room temperature and poured into a separating funnel. The layers were separated and the organics were washed with 2 M hydrochloric acid (100 mL). The aqueous layer was treated with 2 M NaOH solution (400 mL). The aqueous layer was extracted with 3×200 mL of chloroform. The organics were dried with MgSO₄, filtered, and evaporated to give a dark yellow solid. This was triturated with diethyl ether to provide the title compound as a yellow solid. Yield 9.8 g, 43%. Analytical LCMS method 2, retention time 0.40 min, M+H=229. ¹H NMR: (CDCl₃) δ: 7.71 (d, 1H), 7.46 (dd, 1H), 6.83 (d, 1H), 5.48 (br, s, 3H), 3.86 (s, 3H).

### Synthesis 50

### 2-(5-Bromo-2-methoxy-phenyl)-pyrimidin-4-ol

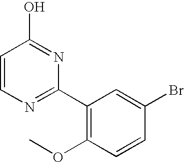

5-Bromo-2-methoxy-benzonitrile (25.00 mmol, 5.73 g) was dissolved in ethanol (35 mL) and treated with ethyl propiolate (28.75 mmol, 2.93 g). The reaction mixture was heated at 60° C. for 30 minutes and then treated with a solution of potassium hydroxide (28.75 mmol, 1.63 g) in ethanol (35 mL). The reaction mixture was then heated at reflux for 3 hours and then allowed to cool to room temperature. The reaction mixture was concentrated under reduced pressure and taken up in water 300 mL. The mixture was adjusted to pH 4 with conc hydrochloric acid and the resultant solid was filtered off and washed with water. The solid was transferred to a round bottom flask, suspended in toluene and evaporated to dryness twice to provide the title compound as an off white solid. Yield=4.4 g, 63%. Analytical LCMS method 2, retention time 4.28 min, M+H=281. ¹H NMR: (CDCl₃) δ: 11.0 (br, s, 1H), 8.57 (d, 1H), 8.04 (d, 1H), 7.61 (dd, 1H), 6.95 (d, 1H), 6.37 (d, 1H), 4.04 (s, 3H).

### Synthesis 51

### 2-(5-Bromo-2-methoxy-phenyl)-4-chloro-pyrimidine

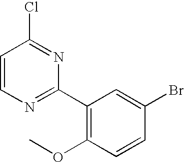

2-(5-Bromo-2-methoxy-phenyl)-pyrimidin-4-ol (15.65 mmol, 4.40 g) and N,N-dimethylaniline (21.90 mmol, 2.76 mL) were dissolved in toluene (120 mL) and heated at reflux for 1 hour. The reaction mixture was then treated with phosphorus oxychloride (18.70 mmol, 2.14 mL) and heated at 110° C. for 4 hours and then allowed to cool to room temperature. The reaction mixture was evaporated under reduced pressure and the residue was added to ice water (250 mL) and extracted with ethyl acetate (3×200 mL). The organics were washed with brine (200 mL), dried with MgSO₄, filtered, and evaporated to give a brown solid. This was triturated with di-iso-propylether to yield the title compound as a light brown solid. Yield 3.8 g, 81%. Analytical LCMS method 2, retention time 5.49 min, MI=299. ¹H NMR: (CDCl₃) δ: 8.72 (d, 1H), 7.87 (d, 1H), 7.53 (dd, 1H), 7.30 (d, 1H), 6.91 (d, 1H), 3.86 (s, 3H).

### Synthesis 52

### ((R)-1-{[2-(5-Bromo-2-hydroxy-phenyl)-pyrimidin-4-ylamino]-methyl}-propyl)-carbamic acid tert-butyl ester

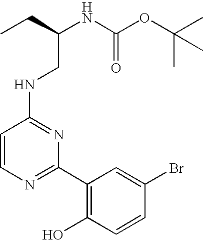

4-Bromo-2-(4-chloro-pyrimidin-2-yl)-phenol (12.50 mmol, 3.57 g) was dissolved in N,N-dimethylacetamide (10 mL) and treated with ((R)-1-aminomethyl-propyl)-carbamic acid tert-butyl ester (15.00 mmol, 2.82 g) and triethylamine (15.00 mmol, 2.10 g). The reaction mixture was allowed to stir at room temperature for 3 hours. The reaction mixture was poured into a separating funnel that contained water 700 mL. The aqueous was extracted with ethyl acetate (3×250 mL), washed with brine, dried with MgSO₄, filtered, and evaporated under reduced pressure to afford the title compound as an orange solid. Yield 5.04 g, 92%. Analytical LCMS method 2, retention time 6.15 min, MI=437. ¹H NMR: (CDCl₃) δ: 8.45 (s, 1H), 8.07 (br, s, 1H), 7.39 (dd, 1H), 7.26 (s, 1H), 6.86 (d, 1H), 6.27 (br, s, 1H), 5.95 (br, s, 1H), 4.59 (br, s, 1H), 3.75 (br, s, 1H), 3.55 (br, s, 1H), 1.73-1.45, (m, 2H), 1.45 (s, 9H), 1.04 (t, 3H).

### Synthesis 53

### 2-[4-((R)-2-Amino-butylamino)-pyrimidin-2-yl]-4-bromo-phenol (XX-220)

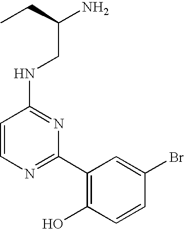

((R)-1-{[2-(5-Bromo-2-hydroxy-phenyl)-pyrimidin-4-ylamino]-methyl}-propyl)-carbamic acid tert-butyl ester (0.2 mmol, 0.09 g) was dissolved in trifluoroacetic acid (0.5 mL) and allowed to stir at room temperature for 3 hours. The solution was added to an aqueous solution of sodium carbonate (50 mL) and extracted with ethyl acetate (2×30 mL). The organics were evaporated to give a yellow solid that was dissolved in dimethylsulfoxide and purified by preparatory HPLC to give the title compound. Analytical LCMS method 2, retention time 2.88 min, MI=337. ¹H NMR: (d-6 DMSO) δ: 8.49 (br, s, 1H), 8.39 (d, 1H), 8.33 (s, 1H), 7.47 (dd, 1H), 6.86 (d, 1H), 6.54 (d, 1H), 3.72-3.67 (m, 1H), 3.40-3.37 (m, 1H), 3.15-3.11 (m, 1H), 1.58-1.53 (m, 2H), 1.00, (t, 3H).

### Synthesis 54

### 3-[4-((R)-2-Amino-butylamino)-pyrimidin-2-yl]-4′-fluoro-biphenyl-4-ol (XX-299)

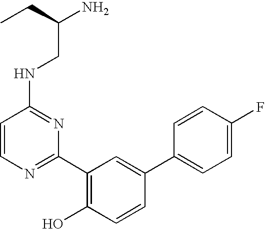

4-Fluorobenzeneboronic acid (0.120 mmol, 0.016 g), potassium phosphate (0.240 mmol, 0.051 g) and palladium tetrakistriphenylphosphine (0.024 mmol, 0.027 g) were weighed into a microwave reactor tube and treated with a solution of ((R)-1-{[2-(5-Bromo-2-hydroxy-phenyl)-pyrimidin-4-ylamino]-methyl}-propyl)-carbamic acid tert-butyl ester (0.120 mmol, 0.05 g) in N,N-dimethylacetamide (0.7 ml) and water (0.3 ml). The tube was capped and the reaction mixture was heated at 150° C. for 10 minutes in a microwave reactor. The reaction mixture was filtered through a short pre-packed column of silica eluting with ethyl acetate. The organics were evaporated and treated with trifluoroacetic acid (0.5 mL) and allowed to stir at room temperature for two hours. The reaction mixture was quenched into a solution of sodium carbonate (5 mL) and extracted with ethyl acetate (2×5 mL). The organics were dried with MgSO₄, filtered, and evaporated. The residue was dissolved in dimethylsulfoxide and purified by preparatory HPLC to yield the title compound as a yellow solid. Analytical LCMS method 2, retention time 3.48 min, M+H=353. ¹H NMR: (d-6 DMSO) δ: 8.55 (dd, 1H), 8.33 (s, 1H), 8.16 (d, 1H), 7.68-7.62 (m, 2H), 7.27 (m, 1H), 6.98 (t, 1H), 6.54 (d, 1H), 3.77 (m, 1H), 3.37-3.16 (m, 2H), 1.58-1.52 (m, 1H), 0.99 (t, 3H).

The following compounds were synthesised using the same general method.

TABLE S-10

| | Analytical LCMS | | |
|---|---|---|---|
| ID No. | retention time (min) | M + H | Method |
| XX-220 | 2.88 | 337 | 2 |
| XX-308 | 3.15 | 335 | 2 |
| XX-294 | 3.71 | 369.5 | 2 |
| XX-228 | 0.36 | 336 | 2 |
| XX-292 | 3.45 | 349 | 2 |
| XX-297 | 3.46 | 349 | 2 |
| XX-299 | 3.48 | 353 | 2 |
| XX-290 | 3.35 | 353 | 2 |
| XX-295 | 3.36 | 353 | 2 |
| XX-305 | 3.76 | 403 | 2 |
| XX-298 | 3.68 | 403 | 2 |
| XX-291 | 3.16 | 365 | 2 |
| XX-227 | 0.39 | 336 | 2 |
| XX-296 | 3.28 | 365 | 2 |
| XX-301 | 0.5 | 378 | 2 |
| XX-300 | 0.53 | 378 | 2 |
| XX-225 | 3.6 | 385 | 2 |
| XX-302 | 3.17 | 360 | 2 |
| XX-293 | 3.61 | 419 | 2 |
| XX-346 | 3.03 | 428 | 2 |
| XX-223 | 3.41 | 385 | 2 |
| XX-210 | 0.44 | 325 | 2 |
| XX-304 | 3.08 | 420 | 2 |
| XX-229 | 0.44 | 337 | 2 |
| XX-214 | 0.36 | 366 | 2 |
| XX-217 | 2.95 | 366 | 2 |
| XX-215 | 0.37 | 350 | 2 |
| XX-218 | 0.4 | 350 | 2 |
| XX-219 | 3.24 | 380 | 2 |
| XX-307 | 2.71 | 351 | 2 |
| XX-306 | 3.12 | 351 | 2 |
| XX-211 | 2.82 | 363 | 2 |
| XX-289 | 3.42 | 369 | 2 |
| XX-303 | 0.45 | 349 | 2 |
| XX-288 | 3.74 | 459 | 2 |
| XX-224 | 0.43 | 273 | 2 |
| XX-287 | 3.83 | 411 | 2 |
| XX-221 | 0.73 | 301 | 2 |
| XX-132 | 2.98 | 305 | 2 |
| XX-254 | 3.19 | 307 | 2 |
| XX-276 | 3.13 | 307 | 2 |
| XX-230 | 3.12 | 341 | 2 |
| XX-213 | 2.80 | 354 | 2 |
| XX-216 | 3.44 | 355 | 2 |
| XX-163 | 3.15 | 339 | 2 |
| XX-199 | 5.03 | 383 | 2 |
| XX-263 | 3.11 | 319 | 2 |
| XX-264 | 4.85 | 320 | 2 |
| XX-212 | 0.58 | 339 | 2 |
| XX-128 | 3.85 | 307 | 2 |
| XX-131 | 2.82 | 293 | 2 |
| XX-207 | 0.36 | 325 | 2 |
| XX-209 | 0.40 | 339 | 2 |
| XX-341 | 3.03 | 307 | 2 |
| XX-340 | 3.19 | 320 | 2 |
| XX-164 | 1.96 | 305 | 3 |
| XX-145 | 2.11 | 287 | 3 |
| XX-134 | 2.21 | 321 | 3 |
| XX-143 | 2.14 | 301 | 3 |
| XX-142 | 3.58 | 365 | 3 |
| XX-285 | 3.43 | 363 | 3 |
| XX-144 | 3.47 | 363 | 3 |
| XX-284 | 2.57 | 406 | 3 |
| XX-206 | 3.14 | 415 | 2 |
| XX-208 | 3.03 | 381 | 2 |
| XX-271 | 2.15 | 339 | 2 |
| XX-272 | 2.44 | 339 | 2 |
| XX-273 | 2.43 | 353 | 2 |
| XX-259 | 3.41 | 351 | 3 |
| XX-258 | 3.50 | 365 | 3 |
| XX-226 | 2.60 | 325 | 2 |
| XX-275 | 0.4 | 452 | 2 |
| XX-337 | 3.29 | 415 | 2 |
| XX-274 | 3.23 | 415 | 2 |
| XX-252 | 1.14 | 289 | 2 |

XX-221 was transformed into the final product following Procedure K then Procedure U.

XX-223 was derived from commercially available 5-iodosalicylaldehyde and transformed into the 5-iodo-2-methoxy-benzaldehyde by known literature methods.

XX-341 and XX-259 were synthesised following Procedure K and then converted into the final products by Procedure L.

XX-340 and XX-258 were synthesised following Procedure K and then converted into the final products by Procedure M.

XX-128 and XX-131 were synthesised using Procedure K to obtain 4-chloro-2-(4-chloro-pyrimidin-2-yl)-phenol, and then transformed into the final products using Procedure V.

XX-263 was synthesised using Procedure K to obtain 4-chloro-2-(4-chloro-pyrimidin-2-yl)-phenol, and then transformed into the final product using (R)-2-amino-2-cyclopropyl-acetamide synthesised by Procedure Q.

XX-226 was synthesised using Procedure K. The starting material, 2-methoxy-5-pyrazol-1-yl-benzonitrile, was synthesised used Procedure W.

XX-252 was synthesised using Procedure K and then further manipulated using Procedure P.

### General Synthesis Procedure L

Additional compounds were prepared by the following methods.

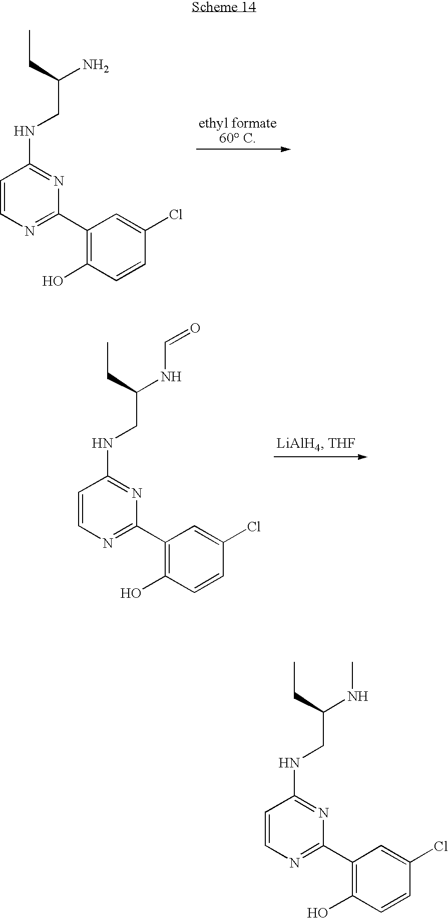

### Synthesis 55

### N—((R)-1-{[2-(5-Chloro-2-hydroxy-phenyl)-pyrimidin-4-ylamino]-methyl}propyl)-formamide

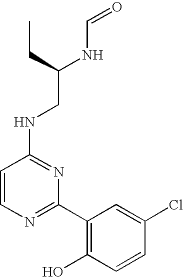

2-[4-((R)-2-Amino-butylamino)-pyrimidin-2-yl]-4-chloro-phenol (1.02 mmol, 300 mg) was dissolved in ethyl formate (50 mL) and heated at 60° C. for 8 hours. The solvent was removed under reduced pressure to yield the title compound as a pale yellow solid. The crude product was used without further purification. Analytical LCMS method 2 retention time 4.09 min, MI=421.

### Synthesis 56

### 4-Chloro-2-[4-((R)-2-methylamino-butylamino)-pyrimidin-2-yl]-phenol (XX-341)

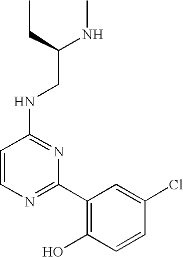

N—((R)-1-{[2-(5-Chloro-2-hydroxy-phenyl)-pyrimidin-4-ylamino]-methyl}-propyl)-formamide (0.51 mmol, 160 mg) was dissolved in tetrahydrofuran (10 mL) and cooled to 0° C. under nitrogen, lithium aluminium hydride (2.57 mmol, 100 mg) was added and stirred at 0° C. for 1 hour and then at room temperature for 15 hours. The reaction mixture was cooled to —78° C. and quenched with 2 M sodium hydroxide (0.1 mL) then water (0.1 mL) and allowed to stir at room temperature for 1 hour. The solution was diluted with ethyl acetate and filtered through celite. The solvent was removed under reduced pressure to yield a yellow oil. The crude product was dissolved in DMSO and purified by mass directed HPLC. Analytical LCMS method 2, retention time 3.03 min, MI=307. ¹H NMR (d-6 DMSO) δ: 8.53 (1H, s), 8.12-8.10 (1H, d), 7.35-7.31 (1H, m), 6.90-6.87 (1H, d), 6.56-6.51 (1H, d), 3.76-3.55 (2H, m), 3.07 (1H, s), 2.46 (3H, s), 1.66-1.52 (2H, m), 1.01-0.96 (3H, t).

### General Synthesis Procedure M

Additional compounds were prepared by the following methods.

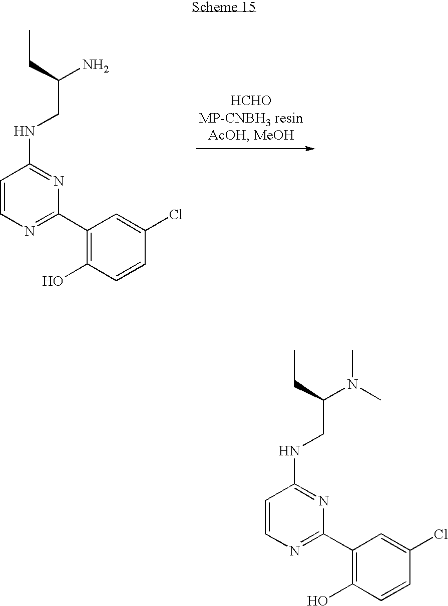

### Synthesis 57

### 4-Chloro-2-[4-((R)-2-dimethylamino-butylamino)-pyrimidin-2-yl]-phenol (XX-340)

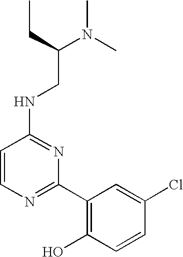

2-[4-((R)-2-Amino-butylamino)-pyrimidin-2-yl]-4-chloro-phenol (0.165 mmol, 50 mg) was dissolved in methanol (1 mL) and treated with formaldehyde (1 M in methanol, 0.18 mL) and macroporous polymer supported cyanoborohydride resin (0.33 mmol, 165 mg) followed by acetic acid (1 mL). The reaction mixture was left to stir overnight. The reaction mixture was filtered and evaporated under reduced pressure. The residue was purified by mass directed HPLC to yield the title compound as a yellow solid. Analytical LCMS method 2, retention time 3.19 min, M+H=321. ¹H NMR d-6 (d-6 DMSO) δ: 8.27-8.02 (4H, m), 7.36-7.33 (1H, m), 6.92-6.89 (1H, d), 6.55-6.53 (1H, d), 3.51-3.47 (1H, m), 2.74-2.70 (1H, m), 2.36 (6H, s), 1.60-1.54 (1H, m), 1.41-1.31 (1H, m), 0.99-0.95 (3H, t).

### General Synthesis Procedure N

Additional compounds were prepared by the following methods. The pyrimidinol was synthesised using Procedure K and then elaborated as shown.

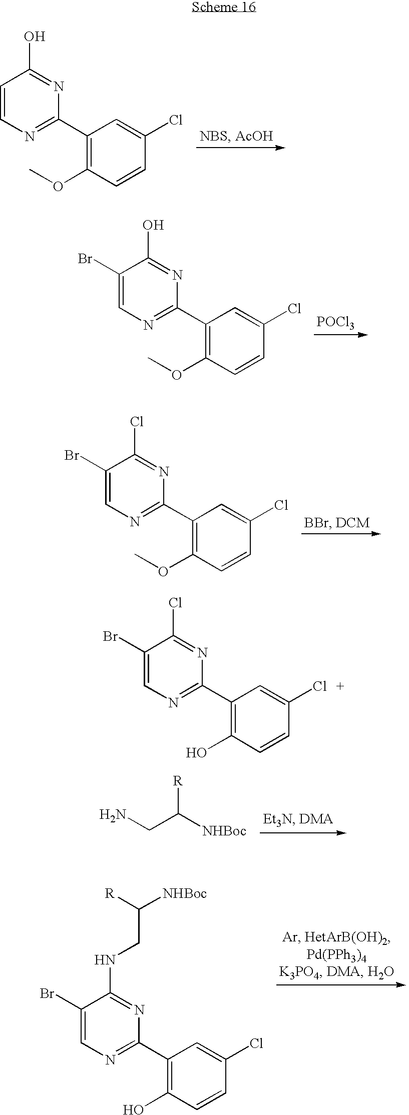

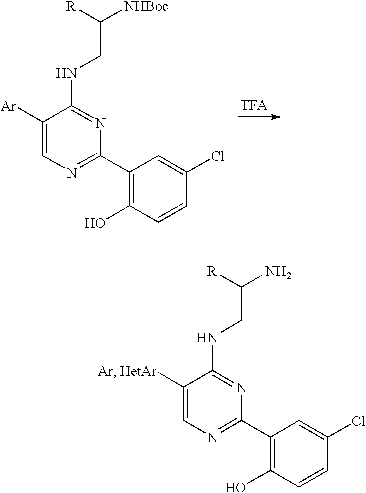

### Synthesis 58

### 5-Bromo-2-(5-chloro-2-methoxy-phenyl)-pyrimidin-4-ol

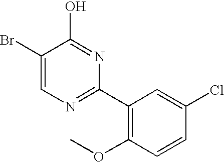

2-(5-Chloro-2-methoxy-phenyl)-pyrimidin-4-ol (29.79 mmol, 7.05 g) was dissolved in the minimum amount of acetic acid, cooled to 0° C. and treated with bromine (208.53 mmol, 16.7 mL) dropwise over 15 minutes. The reaction was allowed to warm to room temperature and stirred for 48 hours. The reaction mixture was quenched with a saturated solution of sodium thiosulfate at 0° C. The yellow precipitate was collected by filtration and the filtrate was extracted several times with dichloromethane. The organic fractions were combined, dried over MgSO₄, filtered, and concentrated under reduced pressure to give an orange solid. This was combined with the solid precipitate and azeotroped with toluene (3×100 mL) to give the title compound as an orange solid. Yield 8.08 g, 86%. Analytical LCMS method 1, retention time 5.14 min, M+H=317. ¹H NMR (d-6 DMSO) δ: 8.37 (1H, s), 7.64-7.63 (1H, m) 7.57-7.53 (1H, m), 7.21-7.18 (2H, d), 1.36 (3H, m).

### Synthesis 59

### 5-Bromo-4-chloro-2-(5-chloro-2-methoxy-phenyl)pyrimidine

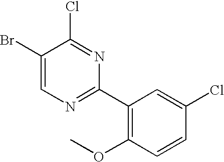

5-Bromo-2-(5-chloro-2-methoxy-phenyl)-pyrimidin-4-ol (23.99 mmol, 7.57 g) was dissolved in phosphorus oxychloride (100 mL) and heated to 110° C. for 5 hours. The reaction mixture was allowed to cool to room temperature and the phosphorus oxychloride was removed under reduced pressure. The residual oil was added to ice and then extracted with dichloromethane (3×400 mL). The organics were combined, washed with water (400 mL), then brine (500 mL), dried with magnesium sulfate, filtered, and evaporated to give a pale red solid. Analytical LCMS method 2, retention time 5.74 min, M+H=335.

### Synthesis 60

### 2-(5-Bromo-4-chloro-pyrimidin-2-yl)-4-chloro-phenol

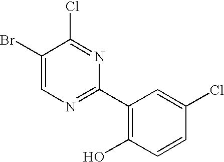

A round bottom flask was charged with 5-bromo-4-chloro-2-(5-chloro-2-methoxy-phenyl)-pyrimidine (17.18 mmol, 5.74 g). The flask was nitrogen flushed and dichloromethane (100 mL) was added. The resultant solution was cooled to −78° C. and treated drop-wise over 10 minutes with boron tribromide (1 M in dichloromethane, 60 mmol, 60 mL). The solution was allowed to stir for 1 hour at this temperature and then the cooling bath was removed. The solution was left to stir under nitrogen at room temperature for 2 hours and then poured slowly into a beaker containing ice. The resultant suspension was poured into a separating funnel and extracted with dichloromethane (3×500 mL). The organics were washed with brine (500 mL), dried with magnesium sulfate, filtered, and evaporated to give the title compound as a brown solid. Yield 5.30 g, 98%. Analytical LCMS method 2, retention time 7.09 min, no ionisation.

### Synthesis 61

### ((R)-1-{[5-Bromo-2-(5-chloro-2-hydroxy-phenyl)-pyrimidin-4-ylamino]-methyl}-propyl)-carbamic acid tert-butyl ester

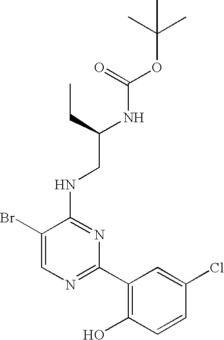

A round bottom flask was charged with 2-(5-bromo-4-chloro-pyrimidin-2-yl)-4-chloro-phenol (9.38 mmol, 3.0 g), (R)-1-aminomethyl-propyl)-carbamic acid tert-butyl ester, triethylamine and N,N-dimethylacetamide, and the reaction mixture was allowed to stir at room temperature overnight. The reaction mixture was added to water and extracted with ethyl acetate (3×500 mL). The organics were combined, dried over MgSO₄, filtered, and concentrated under reduced pressure to give the title compound. Yield 4.20 g, 94%. Analytical LCMS method 2, retention time 7.38 min, M+H=473. ¹H NMR (d-6 DMSO) δ: 8.48 (1H, s), 7.66-7.63 (1H, m), 7.42-7.38 (1H, m) 6.96-6.91 (1H, d), 3.75-3.71 (1H, m), 3.60-3.37 (2H, m), 3.44-3.37 (1H, m), 1.35 (9H, s), 1.15 (2H, t), 0.92-0.88 (3H, m).

### Synthesis 62

### 2-[4-((R)-2-Amino-butylamino)-5-phenyl-pyrimidin-2-yl]-4-chloro-phenol (XX-173)

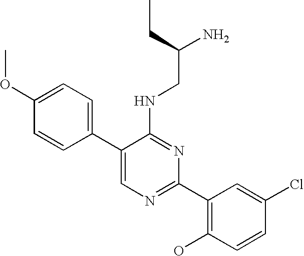

4-Methoxybenzeneboronic acid (0.095 mmol, 14.44 mg) was weighed into a microwave vial, ((R)-1-{[5-Bromo-2-(5-chloro-2-hydroxy-phenyl)-pyrimidin-4-ylamino]-methyl}-propyl)-carbamic acid tert-butyl ester (0.095 mmol, 40 mg) dissolved in N,N-dimethylacetamide (0.7 mL) and potassium phosphate (0.19 mmol, 20 mg) dissolved in water (0.3 mL) were added. Palladium tetrakistriphenylphosphine (0.004 mmol, 4.6 mg) was added and the tube was sealed and heated in the microwave at 150° C. for 10 minutes. Water (2 mL) was added to the tube and the aqueous was extracted with ethyl acetate (3×2 mL). The organics were combined, filtered through a plug of silica, and concentrated under reduced pressure to yield a yellow oil. Trifluoroacetic acid (1 mL) was added to the yellow oil and the reaction mixture was allowed to stir at room temperature overnight. The trifluoroacetic acid was removed under reduced pressure and a saturated solution of sodium carbonate was added. The aqueous was extracted with ethyl acetate (3×5 mL), the organics were combined, and concentrated under reduced pressure. The crude product was dissolved in DMSO and purified by mass directed HPLC to give the title compound. Analytical LCMS method 2 retention time 3.85 min, MI=399. ¹H NMR (d-6 DMSO) δ: 8.31-8.30 (2H, m), 8.10 (1H, s), 7.49-7.46 (2H, m), 7.41-7.37 (1H, m), 7.32-7.18 (1H, bs), 7.10-7.07 (2H, d), 6.97-6.94 (1H, d), 6.82 (3H, s), 3.75-3.69 (1H, m), 3.40-3.15 (2H, m), 1.57-1.50 (2H, m), 1.04-0.99 (3H, t).

The following compounds were synthesised using the same general method.

TABLE S-11

| | Analytical LCMS | | |
|---|---|---|---|
| ID No. | retention time (min) | M + H | Method |
| XX-180 | 3.78 | 373 | 2 |
| XX-190 | 3.80 | 369 | 2 |
| XX-173 | 3.85 | 399 | 2 |
| XX-171 | 3.87 | 399 | 2 |
| XX-191 | 3.95 | 384 | 2 |
| XX-189 | 3.87 | 383 | 2 |
| XX-188 | 4.12 | 383 | 2 |
| XX-167 | 3.37 | 359 | 2 |
| XX-169 | 3.91 | 403 | 2 |
| XX-170 | 4.08 | 403 | 2 |
| XX-172 | 4.01 | 403 | 2 |
| XX-178 | 3.94 | 409 | 2 |
| XX-176 | 4.06 | 437 | 2 |
| XX-175 | 3.79 | 454 | 2 |
| XX-174 | 3.96 | 389 | 2 |
| XX-168 | 3.33 | 359 | 2 |
| XX-185 | 3.71 | 359 | 2 |
| XX-192 | 3.66 | 420 | 2 |

### General Synthesis Procedure O

Additional compounds were prepared by the following methods. The amidines were synthesised as previously described in Procedure K and then transformed as follows from the commercially available aceto acetates. The pyrimidinols were then transformed into the final products as described in Procedure K.

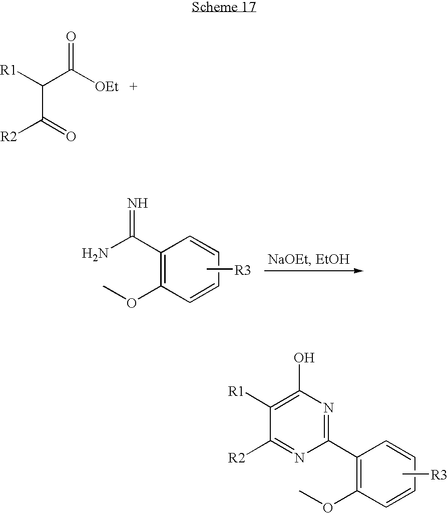

### Synthesis 63

### 2-(5-Chloro-2-methoxy-phenyl)-6-phenyl-pyrimidin-4-ol

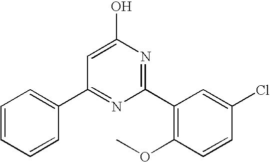

5-Chloro-2-methoxy-benzamidine (5.50 mmol, 1.02 g) was dissolved in ethanol (5 mL) and treated with ethyl benzoylacetate (5.80 mmol, 1 mL) and sodium ethoxide 6.60 mmol, 0.45 g). The reaction mixture was heated at 80° C. for 15 hours. The reaction mixture was evaporated, adjusted to pH 5 by the addition of 2 M hydrochloric acid and diluted with water (100 mL). The aqueous was extracted with ethyl acetate (3×75 mL). The organics were combined, washed with brine (100 mL), dried with MgSO₄, filtered, and evaporated to give a gummy brown solid that was triturated with di-iso-propylether to provide the title compound as a pale brown solid. Analytical LCMS method 2, retention time 5.77 min, M+H=313. ¹H NMR d-6 (d-6 DMSO) δ: 8.62 (d, 1H), 8.07-8.04 (m, 2H), 7.51-7.41 (m, 4H), 7.02 (d, 1H), 6.80 (s, 1H), 4.06 (s, 3H).

The following compounds were synthesised using the same general method.

TABLE S-12

| | Analytical LCMS | | |
|---|---|---|---|
| ID No. | retention time (min) | M + H | Method |
| XX-197 | 3.70 | 335 | 2 |
| XX-196 | 3.50 | 321 | 2 |
| XX-338 | 3.25 | 304 | 2 |
| XX-204 | 4.04 | 369 | 2 |
| XX-182 | 3.39 | 335 | 2 |
| XX-186 | 3.55 | 349 | 2 |
| XX-195 | 3.67 | 333 | 2 |
| XX-179 | 3.90 | 397 | 2 |
| XX-139 | 3.02 | 363 | 3 |
| XX-140 | 3.26 | 411 | 3 |
| XX-183 | 3.74 | 325 | 2 |
| XX-265 | 3.65 | 339 | 2 |
| XX-141 | 4.11 | 397 | 3 |
| XX-200 | 2.25 | 339 | 2 |
| XX-201 | 2.55 | 353 | 2 |
| XX-203 | 1.57 | 350 | 2 |
| XX-268 | 2.67 | 368 | 2 |
| XX-269 | 2.70 | 353 | 2 |
| XX-270 | 2.29 | 364 | 2 |

### General Synthesis Procedure P

Additional compounds were prepared by the following methods. The quinazoline starting materials were synthesised following Procedure D and the pyrimidine starting materials were synthesized following Procedure K and then elaborated into the final products in the following manner.

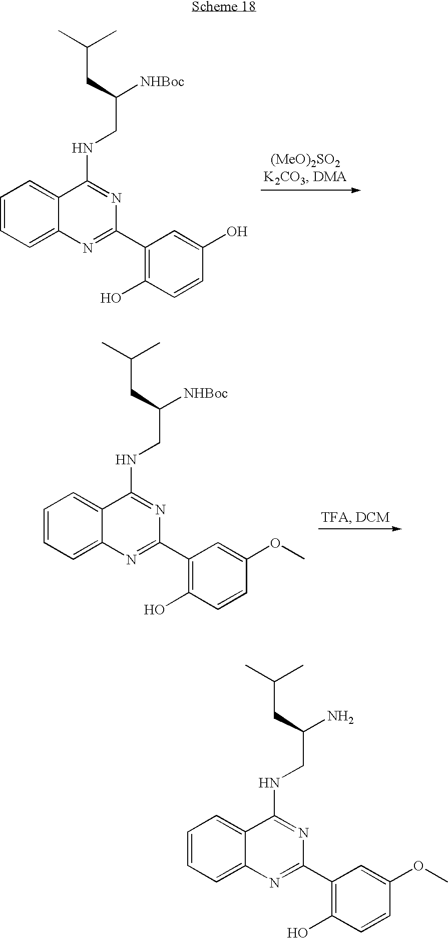

### Synthesis 64

### 2-[4-((R)-2-Amino-4-methyl-pentylamino)-quinazolin-2-yl]-4-methoxy-phenol (XX-152)

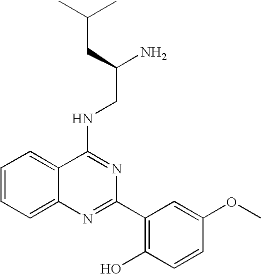

((R)-1-{[2-(2,5-Dihydroxy-phenyl)-quinazolin-4-ylamino]-methyl}-3-methyl-butyl)-carbamic acid tert-butyl ester (0.073 mmol, 30 mg) was dissolved in acetonitrile (0.37 mL). Potassium carbonate (1.09 mmol, 15.1 mg) was added and reaction was heated to reflux for 20 minutes. Dimethyl sulfate (0.080 mmol, 7.6 μL) was added dropwise and the reaction was left to stir at reflux overnight. The solvent was evaporated and the residue was partitioned between ethyl acetate and water. The organic layer was separated, dried with MgSO₄, filtered, and evaporated under reduced pressure. The residue was dissolved in dichloromethane (1 mL) and trifluoroacetic acid (1 mL) and left to stir for 4 hours at room temperature. The solvent was evaporated and the residue was taken up in water, treated with potassium carbonate, and extracted with ethyl acetate. The organic layer was separated, dried with MgSO₄, filtered, and evaporated under reduced pressure. The residue was purified by mass directed HPLC to yield the title compound. Analytical LCMS method 2, retention time 3.31 min, M+H=367. ¹H NMR (d-6 DMSO) δ: 8.35-8.32 (d, 1H), 7.98 (d, 1H), 7.82-7.44 (m, 2H), 7.54 (t, 1H), 6.02-7.98 (m, 1H), 6.86 (d, 1H), 3.74-3.85 (m, 1H), 3.32, (s, 2H), 1.90-1.80 (m, 1H), 1.39-1.32 (t, 2H), 0.92-0.84 (dd, 6H).

### General Synthesis Procedure Q

Additional compounds were prepared by the following methods. (R)-2-amino-2-cyclopropyl-acetamide was synthesised as shown below.

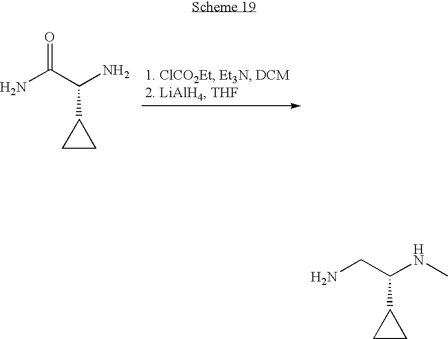

### Synthesis 65

### (R)-1-Cyclopropyl-N*1*-methyl-ethane-1,2-diamine

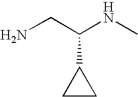

Triethylamine (2.63 mmol, 0.37 mL) and ethyl chloroformate (2.1 mmol, 0.2 mL) were added to a solution of (R)-2-amino-2-cyclopropyl-acetamide (1.75 mmol, 0.20 g) in dichloromethane (5 mL) at 0° C., and the resultant solution was allowed to warm to room temperature and was stirred overnight. The solvent was removed under reduced pressure and the crude was dissolved in tetrahydrofuran (5 mL). At 0° C., lithium aluminium hydride (8.76 mmol, 0.33 g) was added portionwise and the mixture was stirred overnight. The mixture was hydrolysed with 0.4 mL of 2 M NaOH and 0.8 mL of H₂O and stirred overnight. The white precipitate was filtered through a celite pad and the filtrate was concentrated under reduced pressure to give the title compound as a colourless oil (0.2 g, 100%) that was used without further purification. Analytical LCMS method 2, retention time 0.45 min, M+H=115, no UV trace.

### General Synthesis Procedure R

Additional compounds were prepared by the following methods. The dichloro pyrimidine was obtained from commercial sources and was transformed into the final products in the following manner.

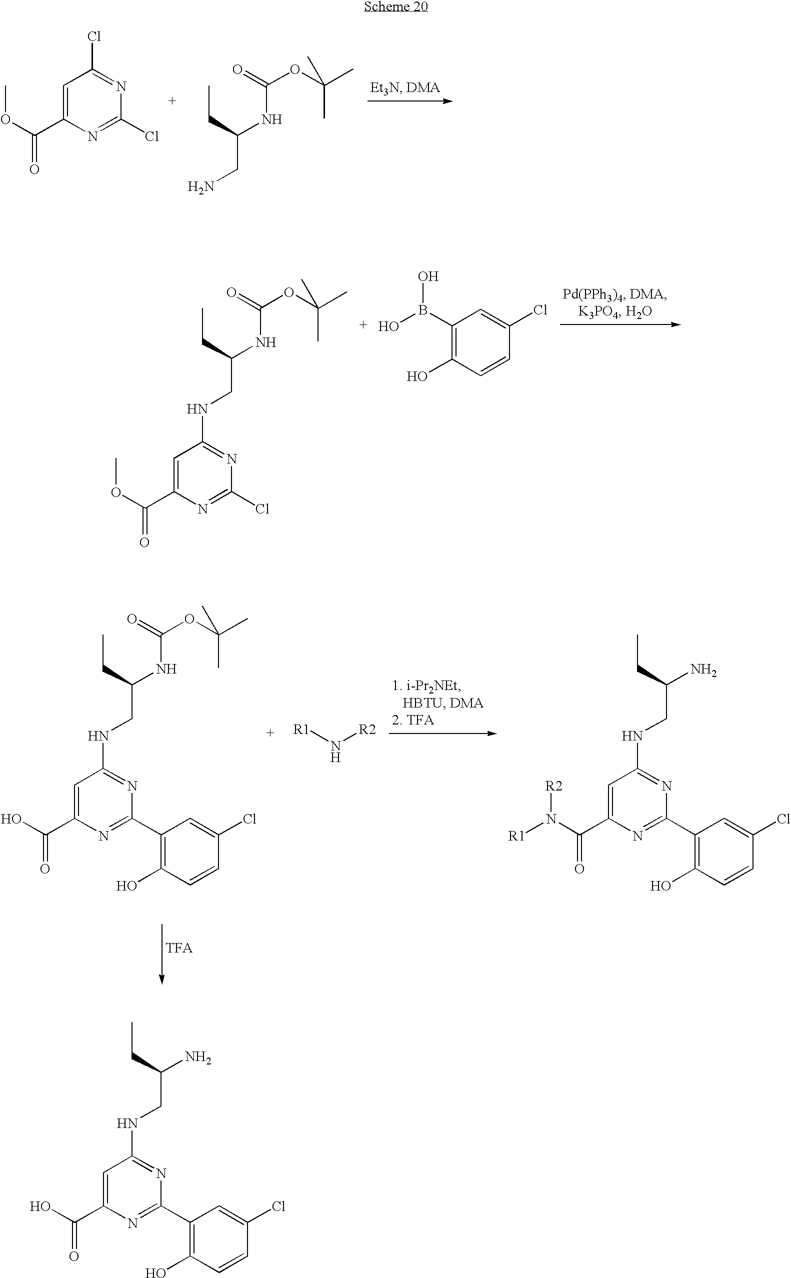

### Synthesis 66

### 6-((R)-2-Amino-butylamino)-2-(5-chloro-2-hydroxy-phenyl)-pyrimidine-4-carboxylic acid (4-fluoro-phenyl)-amide (XX-343)

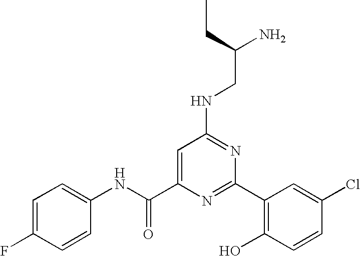

To a solution of 6-((R)-2-tert-Butoxycarbonylamino-butylamino)-2-(5-chloro-2-hydroxy-phenyl)-pyrimidine-4-carboxylic acid (0.137 mmol, 0.06 g) in dimethylacetamide (2 mL), 4-fluoroaniline, di-iso-propylethylamine, and HBTU were added successively and the mixture was stirred overnight at room temperature. Water was added and the compound was extracted with ethyl acetate and dried over MgSO₄ and concentrated under reduced pressure. Trifluoroacetic acid was added to the residue and the solution was stirred for 1 hour at room temperature. The solution was treated with saturated sodium hydrogen carbonate solution followed by extraction with ethyl acetate, dried over MgSO₄, and concentrated under reduced pressure. The residue was purified by preparatory HPLC to give the title compound. Analytical LCMS method 2, retention time 4.05 min, M+H=430. ¹H NMR (d-6 DMSO) δ: 0.97 (t, 3H), 1.49-1.58 (m, 2H), (side chain protons are under the water peak), 6.97 (d, 1H), 7.16-7.27 (m, 4H), 7.36 (dd, 1H), 7.83-7.96 m, 2H).

The following compounds were synthesised using the same general method.

TABLE S-13

| | Analytical LCMS | | |
|---|---|---|---|
| ID No. | retention time (min) | M + H | Method |
| XX-342 | 2.79 | 337 | 2 |
| XX-129 | 3.35 | 406 | 2 |
| XX-343 | 4.05 | 430 | 2 |
| XX-344 | 3.56 | 376 | 2 |

### General Synthesis Procedure S

Additional compounds were prepared by the following methods. The quinazolines were synthesised using Procedure D and elaborated into the final products in the following manner.

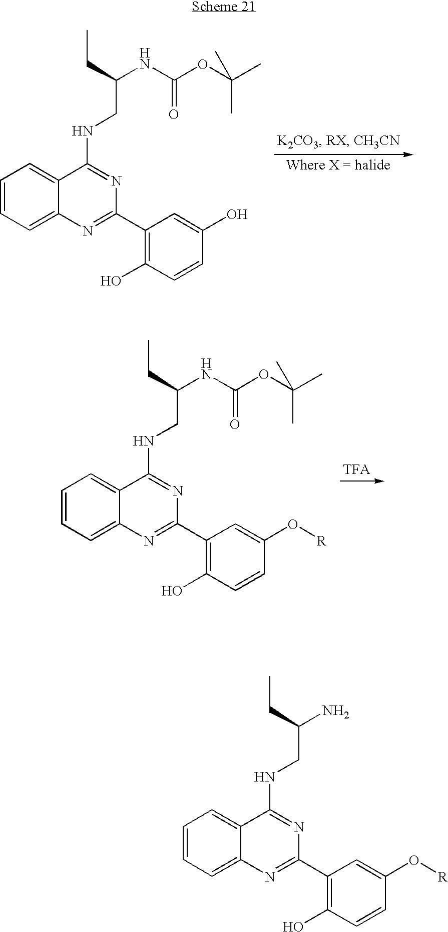

((R)-1-{[2-(2,5-Dihydroxy-phenyl)-quinazolin-4-ylamino]-methyl}-propyl)-carbamic acid tert-butyl ester was synthesised using Procedure D and elaborated as follows.

### Synthesis 67

### 2-[4-((R)-2-Amino-butylamino)-quinazolin-2-yl]-4-(2-morpholin-4-yl-ethoxy)-phenol

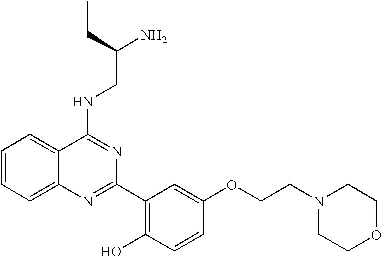

To a solution of ((R)-1-{[2-(2,5-Dihydroxy-phenyl)-quinazolin-4-ylamino]-methyl}-propyl)-carbamic acid tert-butyl ester (0.141 mmol, 0.06 g) in acetonitrile (2 mL), potassium carbonate (2.83 mmol, 0.04 g) and 4-(2-chloroethyl)morpholine hydrochloride (2.12 mmol, 0.04 g) were added successively. The reaction mixture was stirred overnight and the mixture was filtered and concentrated under reduced pressure. The residue was treated with trifluoroacetic acid (1 mL) and the solution was stirred for 1 hour and then treated with a saturated solution of NaHCO₃. After extraction with ethyl acetate, the organic layer was dried over MgSO₄ and concentrated under reduced pressure. The residue was purified by preparatory HPLC. Analytical LCMS method 2, retention time 3.55 min, M+H=438. ¹H NMR (d-6 DMSO) δ: 1.08 (t, 3H), 1.66-1.73 (m, 2H), 2.98-4.07 (m, 16H), 4.32 (brs, 1H), 6.90 (d, 1H), 7.10 (dd, 1H), 7.59 (t, 1H), 7.78-7.88 (m, 2H), 8.00 (d, 1H), 8.28 (d, 1H), 8.81 (brs, 1H).

### General Synthesis Procedure T

Additional compounds were prepared by the following methods. The chloro quinazoline was synthesised following Procedure D and elaborated into the final products in the following manner.

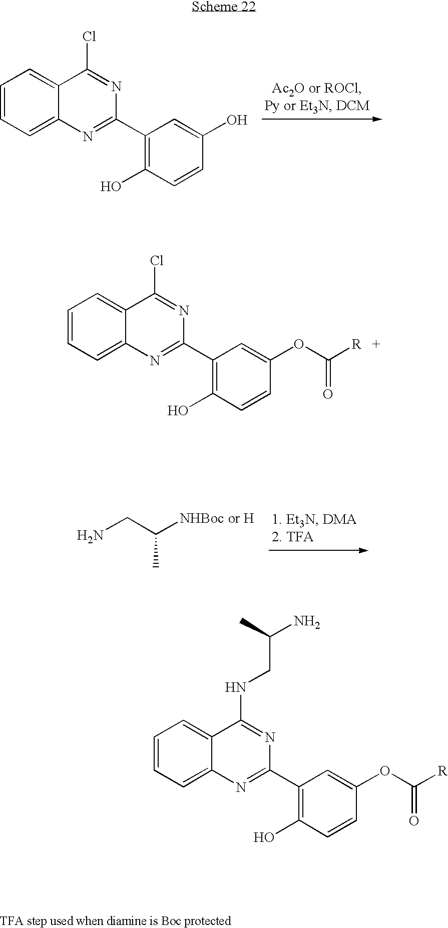

2-(4-chloro-quinazolin-2-yl)-benzene-1,4-diol was synthesised using Procedure D and elaborated as follows.

### Synthesis 68

### Acetic acid 3-(4-chloro-quinazolin-2-yl)-4-hydroxy-phenyl ester

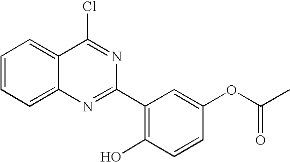

To a solution of 2-(4-chloro-quinazolin-2-yl)-benzene-1,4-diol (0.183 mmol, 0.05 g,) in dichloromethane (5 mL), acetic anhydride (0.2 mmol, 0.02 mL,) and pyridine (0.275 mmol, 0.022 mL,) were added successively. The mixture was stirred overnight, hydrolysed and extracted with dichloromethane. The organics were dried over MgSO₄ and used without further purification. Analytical LCMS method 2, retention time 6.51 min, M+H=315.

### Synthesis 69

### Acetic acid 3-[4-((R)-2-amino-propylamino)-quinazolin-2-yl]-4-hydroxy-phenyl ester (XX-130)

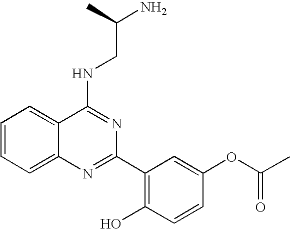

To a solution of acetic acid 3-(4-chloro-quinazolin-2-yl)-4-hydroxy-phenyl ester (0.19 mmol, 0.06 g) in dimethylacetamide (2 mL), R-(+)-1,2-propylenediamine dihydrochloride (0.34 mmol, 0.05 g) and triethylamine (0.5 mL) were added and the mixture was stirred overnight. The solution was hydrolysed and extracted with ethyl acetate and dried over MgSO₄. The residue was purified by preparatory HPLC to give the title compound. Analytical LCMS method 2, retention time 3.02 min, M+H=353. ¹H NMR (d-6 DMSO) δ: 1.15 (d, 3H), 2.28 (s, 3H), 3.54-3.60 (m, 1H), 3.71-3.86 (m, 2H), 6.92 (d, 1H), 7.12 (dd, 1H), 7.55 (t, 1H), 7.76-7.85 (m, 2H), 8.14 (d, 1H), 8.37 (d, 1H), 8.47 (brs, 1H).

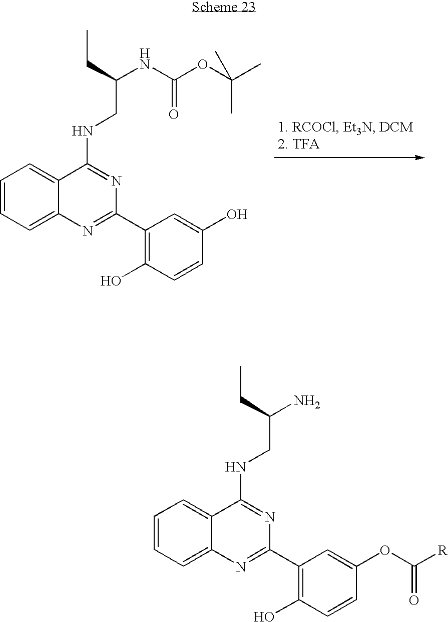

((R)-1-{[2-(2,5-dihydroxy-phenyl)-quinazolin-4-ylamino]-methyl}-propyl)-carbamic acid tert-butyl ester was synthesised using general Procedure D and elaborated as follows.

### Synthesis 70

### 2-Methoxy-benzoic acid 3-[4-((R)-2-amino-butylamino)-quinazolin-2-yl]-4-hydroxy-phenyl ester (XX-281)

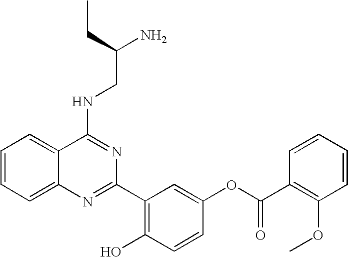

To a solution of ((R)-1-{[2-(2,5-dihydroxy-phenyl)-quinazolin-4-ylamino]-methyl}-propyl)-carbamic acid tert-butyl ester (0.1 mmol, 0.04 g) in dichloromethane (2 mL), triethylamine (0.15 mmol, 0.03 mL) and 2-methoxy-benzoyl chloride (0.12 mmol, 0.02 g) were added successively and the solution was stirred overnight. To the solution, trifluoroacetic acid (1 mL) was added and the mixture was stirred for 2 hours. The solution was treated with a saturated sodium hydrogen carbonate solution followed by extraction with ethyl acetate, dried over MgSO₄ and concentrated under reduced pressure. The residue was purified by preparatory HPLC. Analytical LCMS method 2, retention time 3.72 min, M+H=459. ¹H NMR (d-6 DMSO) δ: 0.96 (t, 1H), 1.57-1.65 (m, 1H), 3.38-3.40 (m, 1H), 3.54 (dd, 1H), 3.88 (s, 3H), 4.03 (d, 1H), 6.99 (d, 1H), 7.01 (t, 1H), 7.22-7.27 (m, 2H), 7.53-7.63 (m, 2H), 7.77-7.83 (m, 2H), 7.88 (d, 1H), 8.22 (d, 1H), 8.36 (d, 1H), 8.45 (s, 1H).

### General Synthesis Procedure U

Additional compounds were prepared by the following methods. The ((R)-1-{[2-(2-Hydroxy-5-iodo-phenyl)-pyrimidin-4-ylamino]-methyl}-propyl)-carbamic acid tert-butyl ester was synthesised using Procedure K and elaborated into the final products in the following manner.

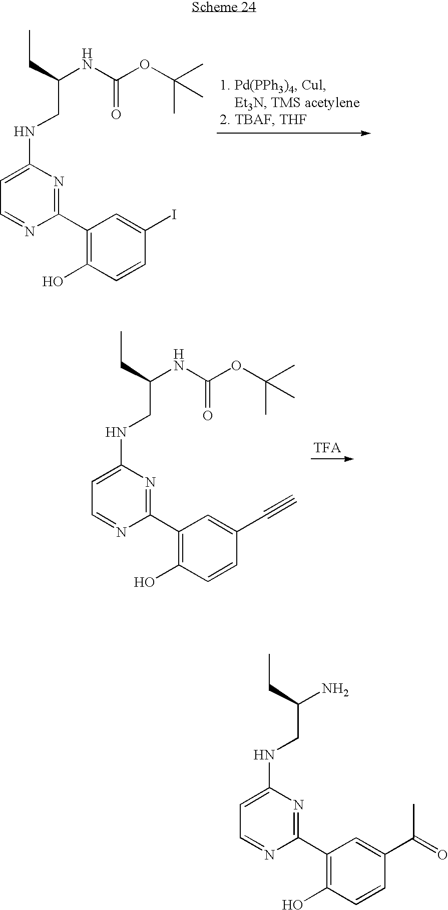

((R)-1-{[2-(2-Hydroxy-5-iodo-phenyl)-pyrimidin-4-ylamino]-methyl}-propyl)-carbamic acid tert-butyl ester was synthesised using Procedure K and elaborated as follows.

### Synthesis 71

### ((R)-1-{[2-(5-Ethynyl-2-hydroxy-phenyl)-pyrimidin-4-ylamino]-methyl}-propyl)-carbamic acid tert-butyl ester

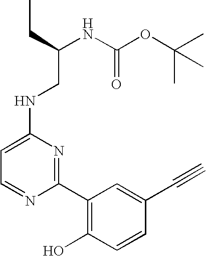

((R)-1-{[2-(2-Hydroxy-5-iodo-phenyl)-pyrimidin-4-ylamino]-methyl}-propyl)-carbamic acid tert-butyl ester (0.15 mmol, 0.070 g), copper iodide (0.010 mmol, 0.0019 g), and palladium tetrakistriphenylphosphine (0.007 mmol, 0.0086 g) were weighed into a microwave vial and treated with (trimethylsilyl)-acetylene (0.45 mmol, 0.064 mL), triethylamine (0.75 mmol, 0.105 mL) and acetonitrile (0.5 mL). The vial was capped and heated at 150° C. for 10 minutes. The reaction mixture was allowed to cool. The reaction mixture was diluted with water (20 mL) and extracted with ethyl acetate (3×20 mL), washed with brine (40 ml), dried with MgSO₄, filtered through celite, and evaporated to give a brown gum. The residue was dissolved in THF (1 mL) and treated with tetrabutylammonium fluoride (1.5 mmol, 1.5 mL, 1 M in THF) and allowed to stir at room temperature for 4 hours. The reaction mixture was diluted with water (20 mL) and extracted with ethyl acetate (3×20 mL), washed with brine (40 mL), dried with MgSO₄, filtered, and evaporated to give the title compound as a brown gum. Analytical LCMS method 2, retention time 7.34 min, M+H=455.

### Synthesis 72

### 1-{3-[4-((R)-2-Amino-butylamino)-pyrimidin-2-yl]-4-hydroxy-phenyl}-ethanone (XX-221)

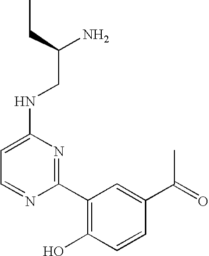

((R)-1-{[2-(5-Ethynyl-2-hydroxy-phenyl)-pyrimidin-4-ylamino]-methyl}-propyl)-carbamic acid tert-butyl ester (0.15 mmol, 0.060 g) was treated with trifluoroacetic acid (1 mL) and allowed to stir at room temperature for 2 hours the reaction mixture was quenched into 2 M sodium carbonate solution (20 mL) and extracted with ethyl acetate (2×25 mL). The organics were washed with brine (50 mL), dried with MgSO₄, filtered, and evaporated to give the title compound as a brown gum. Analytical LCMS method 2, retention time 0.73 min, M+H=301.

### General Synthesis Procedure V

Additional compounds were prepared by the following methods.

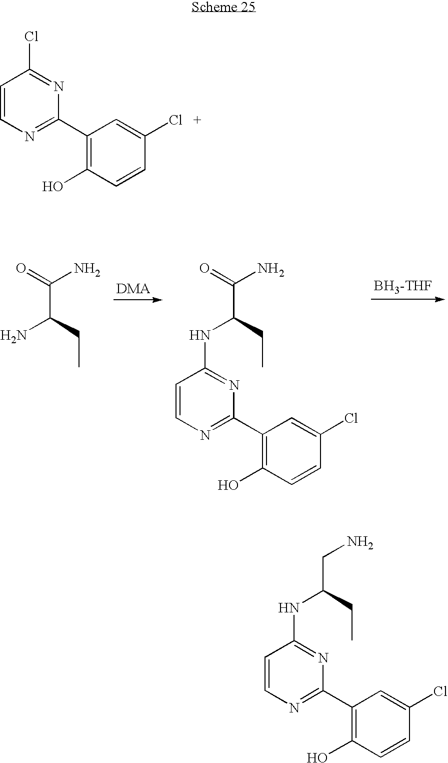

### Synthesis 73

### (R)-2-[2-(5-Chloro-2-hydroxy-phenyl)-pyrimidin-4-ylamino]-butyramide (XX-128)

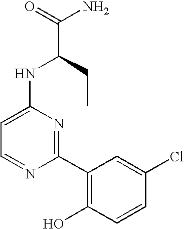

To a solution of 4-chloro-2-(4-chloro-pyrimidin-2-yl)-phenol (0.622 mmol, 0.15 g) in dimethylacetamide (2 mL), (R)-2-amino-butyramide (0.746 mmol, 0.077 g) was added and the mixture was stirred overnight at room temperature. The solution was diluted with water (20 mL) and then extracted with ethyl acetate (3×20 mL). The organics were evaporated and the residue purified by preparatory LCMS to give the title compound. Analytical LCMS method 2, retention time 3.85 min, M+H=307.

### Synthesis 74

### 2-[4-((R)-1-Aminomethyl-propylamino)-pyrimidin-2-yl]-4-chloro-phenol (XX-131)

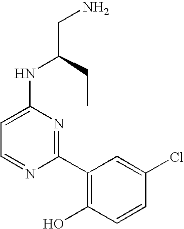

To (R)-2-[2-(5-Chloro-2-hydroxy-phenyl)-pyrimidin-4-ylamino]-butyramide (0.326 mmol, 0.1 g), a 1 M solution of BH₃-THF (1.63 mmol, 1.63 ml,) was added dropwise and the solution was stirred for 4 hours and then quenched with methanol. The solvent was removed under reduced pressure and the residue was purified by preparatory HPLC to give the title compound. Analytical LCMS method 2, retention time 2.82 min, M+H=293.

### General Synthesis Procedure W

XX-226 was synthesised using Procedure K with the inclusion of the additional procedure shown below. The iodo benzonitrile was synthesised from commercially available 5-iodosalicylaldehyde using literature procedures.

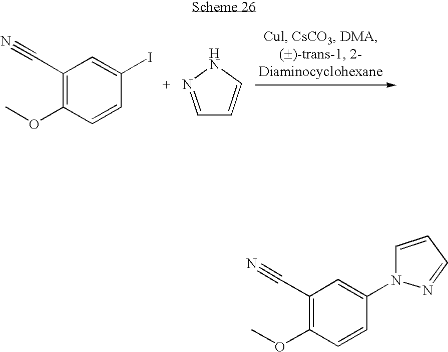

### Synthesis 75

### 2-Methoxy-5-pyrazol-1-yl-benzonitrile

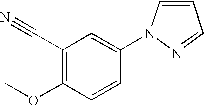

5-Iodo-2-methoxy-benzonitrile (7 mmol, 1.81 g), pyrazole (10.5 mmol, 0.72 g), caesium carbonate (14 mmol, 4.56 g), and copper iodide were weighed into a microwave vial. The mixture was treated with (±)-trans-1,2-diaminocyclohexane (1.40 mmol, 0.16 g) and dimethylacetamide (1 mL). The vial was sealed and heated at 180° C. for 15 minutes. The reaction mixture was allowed to cool. The reaction mixture was poured into a separating funnel containing water (500 mL) and then extracted with ethyl acetate (4×100 mL). The organics were washed with brine (100 mL), dried with MgSO₄, filtered, and evaporated to a dark oil. This was purified by flash column chromatography eluting with 1:4 ethyl acetate: cyclohexane to give the title compound as a pale yellow solid. Analytical LCMS method 2, retention time 4.34 min, M+H=200. ¹H NMR CDCl₃ δ: 7.90-7.84 (m, 3H), 7.72 (d, 1H), 7.06 (d, 1H), 6.48 (dd, 1H), 3.98 (s, 3H).

### General Synthesis Procedure X

Additional compounds were prepared by the following methods.

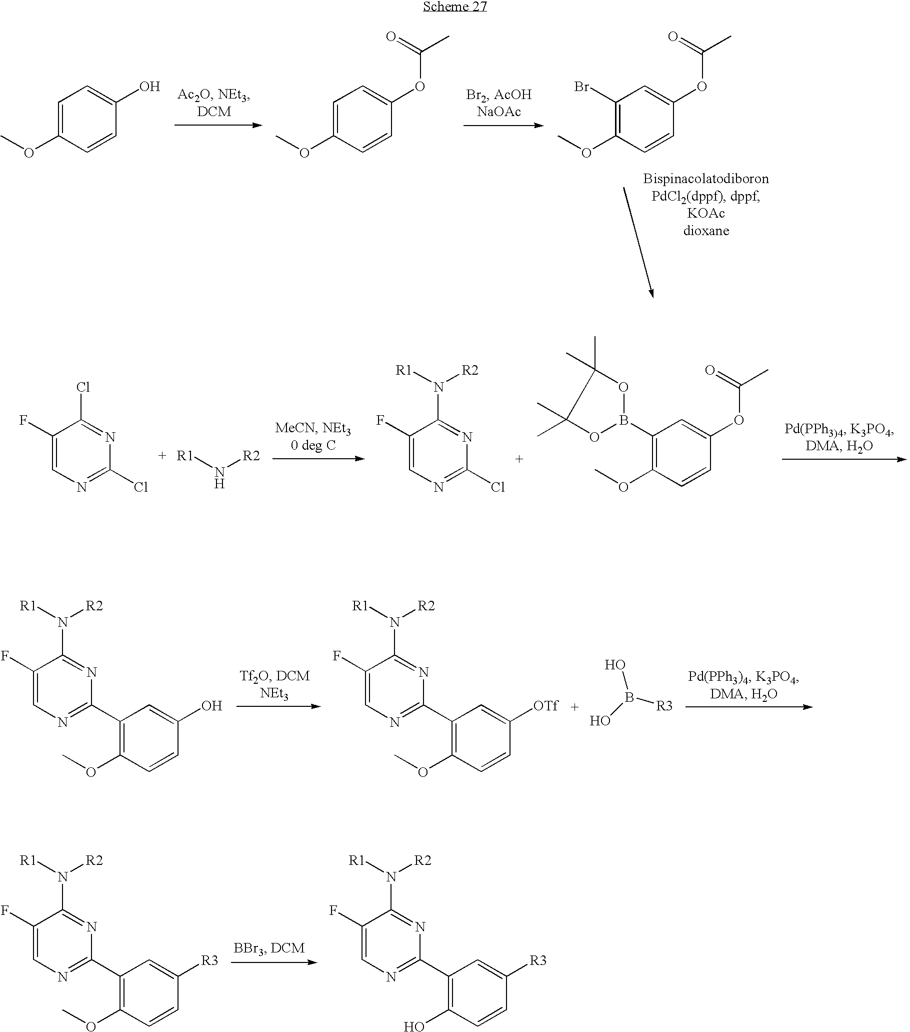

### Synthesis 76

### Acetic acid 4-methoxy-phenyl ester

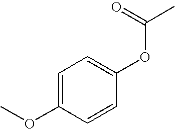

4-Methoxyphenol (100 mmol, 12.41 g) was dissolved in dichloromethane (250 mL), cooled to 0° C. and treated with triethylamine (120 mmol, 16.83 mL) and then portionwise with acetyl chloride (110 mmol, 7.85 mL). The reaction mixture was allowed to warm to room temperature and stirred for 3 days at room temperature. The reaction mixture was poured into a separating funnel containing water (250 mL) and the layers were separated. The aqueous was extracted with (1×100 mL) of dichloromethane. The organics were washed with saturated sodium bicarbonate solution (100 mL), then brine (100 mL), dried with MgSO₄, filtered, and evaporated under reduced pressure to yield the title compound as a brown oil that solidified upon standing. Analytical LCMS method 2, retention time 4.51 min, M+H=208.

### Synthesis 77

### Acetic acid 3-bromo-4-methoxy-phenyl ester

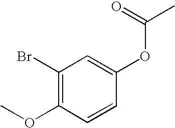

Acetic acid 4-methoxy-phenyl ester (100 mmol, 16.6 g) was dissolved in acetic acid (70 mL) and treated with sodium acetate (200 mmol, 16.4 g). The reaction mixture was cooled to 0° C. in an ice bath and treated dropwise with bromine (120 mmol, 6.1 mL) in acetic acid (70 mL) over 30 minutes. The reaction mixture was allowed to stir at room temperature for 16 hours. The reaction mixture was diluted with water (500 mL) and extracted with ethyl acetate (2×250 mL). The organics were washed with a saturated solution of sodium bicarbonate (300 mL) and then with saturated sodium thiosulfate solution (200 mL). The organics were dried with MgSO₄, filtered, and evaporated to yield the title compound as an orange oil which solidified on standing. Analytical LCMS method 2, retention time 4.94 min, M+H=no ionisation. ¹H NMR CDCl₃ δ: 7.33-7.31 (m, 1H), 7.04-6.99 (m, 1H), 6.90-6.86 (m, 1H), 3.88 (s, 3H), 2.27 (s, 3H).

### Synthesis 78

### Acetic acid 4-methoxy-3-(4,4,5,5-tetramethyl-[1,3,2]dioxaborolan-2-yl)-phenyl ester

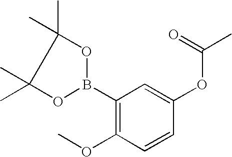

Acetic acid 3-bromo-4-methoxy-phenyl ester (2 mmol, 0.49 g), bis(pinacolato)diboron (3 mmol, 0.76 g), PdCl₂(dppf) (0.20 mmol, 0.14 g), dppf (0.2 mmol, 0.11 g) and potassium acetate (3 mmol, 0.29 g) were weighed into a round bottomed flask and treated with dioxane (5 mL). The reaction mixture was heated at reflux for 24 hours. The reaction mixture was filtered through a short plug of silica eluting with ethyl acetate and evaporated to give a dark brown oil. The residue was purified by flash column chromatography (1:1 ethyl acetate:cyclohexane, visualising with KMnO₄ dip) to yield the title compound as a brown solid. Yield 0.42, 72%. Analytical LCMS method 2, retention time 5.26 min, M+H=293.

### Synthesis 79

### {(R)-1-[(2-Chloro-5-fluoro-pyrimidin-4-ylamino)-methyl]-propyl}-carbamic acid #tert!-butyl ester

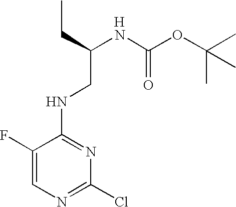

2,4-Dichloro-5-fluoropyrimidine (3 mmol, 0.50 g) was dissolved in acetonitrile (4 mL) and cooled to 0° C. The solution was then treated with triethylamine (4.2 mmol, 0.58 mL) and ((R)-1-aminomethyl-propyl)-carbamic acid tert-butyl ester (4.2 mmol, 0.58 g) dissolved in acetonitrile (1 mL) that had been cooled prior to the addition in an ice bath. The reaction mixture was allowed to stir at 0° C. for 3 hours and then allowed to warm to room temperature. The solvent was removed under reduced pressure and water (100 mL) was added to the residue. The aqeuous was extracted with ethyl acetate (3×50 mL) and the organics were washed with brine (100 mL). The organics were dried with MgSO₄, filtered and evaporated to yield the title compound as a clear oil. Yield 0.96, 100%. Analytical LCMS method 2, retention time 4.98 min, M+H=319.

### Synthesis 80

### ((R)-1-{[5-Fluoro-2-(5-hydroxy-2-methoxy-phenyl)-pyrimidin-4-ylamino]-methyl}-propyl)-carbamic acid tert-butyl ester

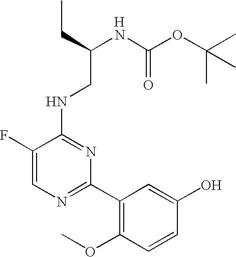

{(R)-1-[(2-Chloro-5-fluoro-pyrimidin-4-ylamino)-methyl]-propyl}-carbamic acid tert-butyl ester (1.0 mmol, 0.32 g), acetic acid 4-methoxy-3-(4,4,5,5-tetramethyl-[1,3,2]dioxaborolan-2-yl)-phenyl ester (1.1 mmol, 0.32 g), potassium phosphate (2 mmol, 0.42 g) and palladium tetrakistriphenylphosphine (0.2 mmol, 0.23 g) were weighed into a microwave vial and treated with dimethylacetamide (2 mL) and water (0.5 mL). The reaction mixture was heated at 150° C. for 15 minutes in a microwave reactor, allowed to cool and diluted with water (20 mL) and extracted with ethyl acetate (3×20 mL). The organics were combined, washed with brine (50 mL), dried with MgSO₄, filtered and evaporated to a brown oil. This was purified by flash column chromatography using ethyl acetate to elute and yielded the title compound as a white solid. Yield 0.36, 88%. Analytical LCMS method 2, retention time 3.17 min, M+H=407.

### Synthesis 81

### Trifluoro-methanesulfonic acid 3-[4-((R)-2-tert-butoxycarbonylamino-butylamino)-5-fluoro-pyrimidin-2-yl]-4-methoxy-phenyl ester

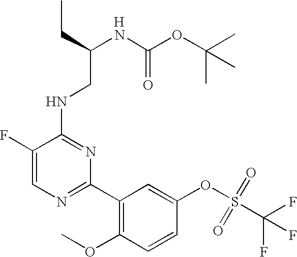

((R)-1-{[5-Fluoro-2-(5-hydroxy-2-methoxy-phenyl)-pyrimidin-4-ylamino]-methyl}-propyl)-carbamic acid tert-butyl ester (0.2 mmol, 0.08 g) was dissolved in DCM (2 mL) and treated with triethylamine (2 mmol, 0.28 mL) and trifluoromethanesulphonic anhydride (0.4 mmol, 0.068 mL). The reaction mixture was allowed to stir at room temperature for 15 hours. Another equivalent (0.4 mmol, 0.068 mL) of trifluoromethanesulphonic anhydride was added and allowed to stir for 3 hours at room temperature. Water (5 mL) was added and the reaction mixture was added to a phase separating cartridge and the aqueous was washed with (2×5 mL) of DCM. The solvent was evaporated to yield the title compound as a brown oil. Yield 0.11 g, 100%. Analytical LCMS method 2, retention time 4.83 min, M+H=539.

### Synthesis 82

### [(R)-1-({5-Fluoro-2-[2-methoxy-5-(1-methyl-1H-pyrazol-4-yl)-phenyl]-pyrimidin-4-ylamino}-methyl)-propyl]-carbamic acid tert-butyl ester

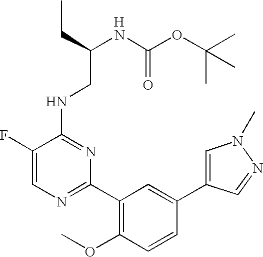

Trifluoro-methanesulfonic acid 3-[4-((R)-2-tert-butoxycarbonylamino-butylamino)-5-fluoro-pyrimidin-2-yl]-4-methoxy-phenyl ester (0.20 mmol, 0.11 g), methyl-4-(4,4,5,5-tetramethyl-1,3,2-dioxaborolan-2-yl)-1H-pyrazole (0.20 mmol, 0.041 g), potassium phosphate (0.40 mmol, 0.084 g) and palladium tetrakis triphenyl phosphine (0.040 mmol, 0.046 g) was weighed into a microwave vial and treated with dimethylacetamide (1 mL). The reaction mixture was heated in a microwave reactor at 150° C. for 15 minutes. The reaction mixture was diluted with water (30 mL) and extracted with (2×30 mL) ethyl acetate. The organics were washed with brine (30 mL), dried with MgSO₄, filtered, and evaporated to give a brown oil. The oil was purified by flash column chromatography (eluting with 1:1 ethyl acetate: cyclohexane then 100% ethyl acetate) to provide the title compound as a yellow gum. Yield 28 mg, 33%. Analytical LCMS method 2, retention time 3.37 min, M+H=471.

### Synthesis 83

### 2-[4-((R)-2-Amino-butylamino)-5-fluoro-pyrimidin-2-yl]-4-(1-methyl-1H-pyrazol-4-yl)-phenol

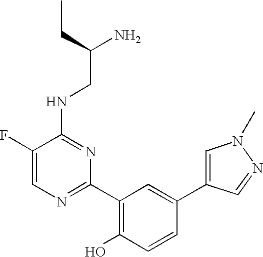

[(R)-1-({5-Fluoro-2-[2-methoxy-5-(1-methyl-1H-pyrazol-4-yl)-phenyl]-pyrimidin-4-ylamino}-methyl)-propyl]-carbamic acid tert-butyl ester (0.060 mmol, 0.03 g) was dissolved in DCM (2 mL) and cooled to −78° C. The solution was treated with boron tribromide (0.6 mmol, 0.6 mL, 1 M in DCM) dropwise and left to stir at −78° C. for 2 hours and then at room temperature for 4 hours. The reaction mixture was carefully poured into a solution of saturated sodium bicarbonate solution (20 mL) and extracted with ethyl acetate (2×20 mL). The organics were dried with MgSO₄, filtered, and evaporated to give a brown oil which was purified by mass directed HPLC. Evaporation of the fractions provided the title compound as a yellow solid. Analytical LCMS method 2, retention time 3.00 min, M+H=357.

The following compounds were synthesised using the same general method.

TABLE S-14

| | Analytical LCMS | | |
|---|---|---|---|
| ID No. | retention time (min) | M + H | Method |
| XX-166 | 2.85 | 343 | 2 |
| XX-195 | 3.00 | 357 | 2 |

### Biological Methods

### Expression and Purification of PKD1 Protein

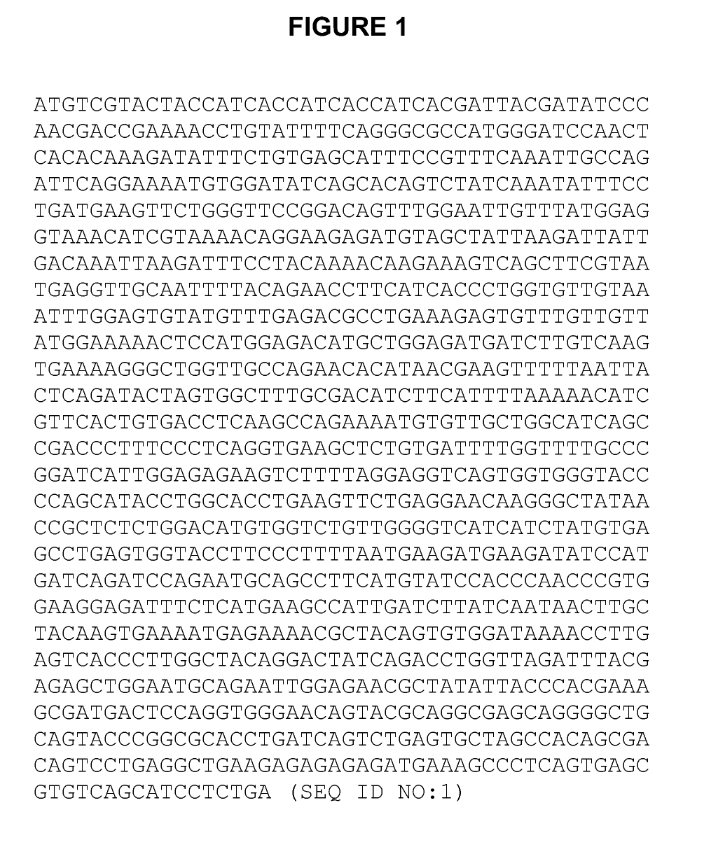

The DNA sequence corresponding to murine PKD1 (see FIG. 1) was inserted into pFastBAc Htb (Invitrogen, USA) at BamH1 and EcoR1 sites using standard molecular biology techniques.

The PKD1 described above was expressed as a hexahistidine tagged protein construct using a commercially available baculoviral expression system that induces protein production in insect cell culture (Bac-to-Bac® HT Baculovirus Expression System, Invitrogen). Protein was typically expressed by inoculating 1 L of sf9 cells with a genetically modified baculovirus containing the gene for the kinase domain of PKD1. Sf9 cells were obtained from ICR Ltd.

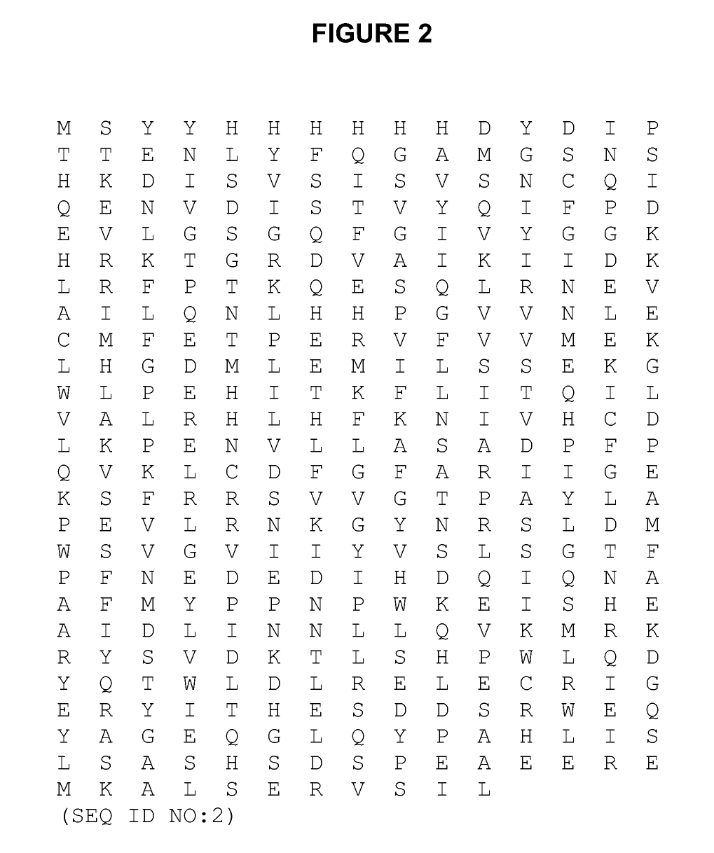

Purification of PKD1 was achieved by standard chromatographic procedures. Capture from crude centrifuged lysed cell supernatant was achieved using metal affinity chromatography (GE Healthcare Life Sciences, HiTrap Chelating chromatography column), and fractions showing PKD1 (as assessed by gel electrophoresis and western blot) were further purified by a single purification step. This was a performed using a mono Q anion exchange chromatography system (GE Healthcare Life Sciences, HiTrap HP Q column). Purified PKD1 (the amino acid sequence is shown in FIG. 2) was tested for activity in a commercially available kinase assay (Molecular Devices IMAP kinase assay kit; see, e.g., Singh et al., 2005). This protocol describes the method for screening compounds as inhibitors of Protein Kinase D activity in a 384 well microplate format fluorescence polarisation IMAP assay performed using the Biomek FX.

### PKD1 (Murine Kinase Domain) Enzyme Activity Assay

### Reagents

Kinase Assay Reaction Buffer: This consisted of 0.22 μM filtered 25 mM HEPES and 2 mM MgCl₂ pH 7.5.

Kinase Enzyme Murine PKD1 kinase domain at ˜100 μg/mL, purified from baculovirus (as described above), obtained from aliquots stored at −70° C. PKD1 was prepared with a final concentration of 0.1 μg/mL by diluting 1:300 in Reaction Buffer (30 μL per 9 mL-5 mL per plate with an additional 4 mL dead volume) and vortexing prior to use. It was necessary to check this concentration regularly in case of enzyme degradation.

Substrate: Fluorescein labelled glycogen synthase-derived peptide (FI)-KKLNRTLSVA (also known as MAPKAP K2 substrate), obtained from Molecular Devices (Product code R7127). Used at 300 nM by diluting 20 μM stock 1:66 in Kinase/Reaction Buffer (135 μL per 9 mL; 75 μL per 5 mL Reaction Buffer for blank wells requiring <1 mL per place with an additional 4 mL dead volume).

ATP: Supplied by Sigma (product code A-7699). A 1 mM ATP stock in Reaction Buffer was prepared from a 10 mM stock in 20 mM NaOH and stored as aliquots at −70° C. It was used at 40 μM by diluting 1 mM stock 1:25 in Reaction Buffer (240 μL per 6 mL-2 mL per plate with an additional 4 mL dead volume) and vortexed prior to use.

IMAP Reagents: IMAP Binding Reagent (product code R7207) and Binding Buffer (product code R7208) were obtained from Molecular Devices. Both were stored at +4° C. The beads were gently re-suspended before diluting by 1:400 in buffer (Binding Buffer is supplied as a 5× stock and so was diluted with water prior to use) and then vortexed before addition to wells. 16 mL water with 4 mL Binding Buffer and 50 μL Binding Reagent were used per plate (17 mL per plate with an additional 3 mL dead volume).

### Method

13 μL Kinase/Substrate in Reaction Buffer were added to ‘test’ and all ‘control’ wells of a Corning black low binding 384 well (90 μL volume) microplate to give 0.2 μg/mL and 200 nM reaction concentration respectively. 13 μL Substrate in Reaction Buffer was added to ‘blank’ wells to give 200 nM reaction concentration. 2 μL test compounds in 10% DMSO/water were added to ‘test’ wells to give final concentrations ranging from 100 to 0.001 μM. 2 μL 10% DMSO/water was added to ‘blank’ and ‘control’ wells. 5 μL ATP in Reaction Buffer was added to all wells to give 10 μM reaction concentration. The reaction mixture was then incubated at room temperature for 25 minutes. The incubation period was followed by the addition of 40 μL IMAP Binding Reagent in Binding Buffer to all wells. The reaction was further incubated at room temperature for 30 minutes. The fluorescence polarisation of the substrate in each well was recorded using an analyst microplate reader (Molecular Devices) with a single read at Ex485 Em535 (Analyst settings: Z Height 5 mm, G Factor 0.95, Reads/well 1, Integration 100000 μs, Gain Sensitivity 2).

Percentage inhibition was calculated based on activity of the test sample minus the average values in the blank wells relative to the average values measured in control wells minus the average values in the blank wells.

IC₅₀ values were calculated from 10 point dose sigmoid ‘dose-response’ curves using XIfit software (IDBS inc, USA). Data were fitted to a 4 parameter logistic model/sigmoidal dose response:

$$Fit = A + ( \frac{( B - A )}{( 1 + {( \frac{C}{X} )}^{D} )} )$$

where:
A=fit minimum (locked to 0);
B=fit maximum (locked to 100);
C=fit midpoint (pre-fit to 1);
D=slope at linear portion of curve, hillslope (pre-fit to 0.1)

The value for C represents the IC₅₀ of the test compound

### PKD1 (Human Full Length) Enzyme Activity Assay

Kinase Assay Reaction Buffer: This consisted of 0.22 μM filtered 25 mM HEPES and 2 mM MgCl₂ pH 7.5.

Kinase Enzyme Human full length PKD1 at ˜100 μg/mL purchased from Upstate Ltd (Product code 14-508) was obtained from aliquots stored at −70° C. It was prepared with a final concentration of 0.3 μg/mL by diluting 1:300 in Reaction Buffer (30 μL per 9 mL-5 mL per plate with an additional 4 mL dead volume) and vortexing prior to use.

Substrate: Fluorescein labelled glycogen synthase-derived peptide (FI)-KKLNRTLSVA (also known as MAPKAP K2 substrate) was obtained from Molecular Devices (Product code R7127). It was used at 200 nM by diluting 20 μM stock 1:66 in Kinase/Reaction Buffer (135 μL per 9 mL; 75 μL per 5 mL Reaction Buffer for blank wells requiring <1 mL per place with an additional 4 mL dead volume).

ATP: (Obtained from Sigma, product code A-7699). A 1 mM ATP stock in Reaction Buffer was prepared from a 10 mM stock in 20 mM NaOH and stored as aliquots at −70° C. It was used at 40 μM by diluting 1 mM stock 1:25 in Reaction Buffer (240 μL per 6 mL-2 mL per plate with an additional 4 mL dead volume) and vortexing prior to use.

IMAP Reagents: IMAP Binding Reagent (product code R7207) and Binding Buffer (product code R7208) were obtained from Molecular Devices, and stored at +4° C. The beads were gently re-suspended before diluting by 1:400 in buffer (Binding Buffer was supplied as a 5× stock and so was diluted with water prior to use) and then vortexing before addition to wells. 16 mL water with 4 mL Binding Buffer and 50 μL Binding Reagent were used per plate (17 mL per plate with an additional 3 mL dead volume).

### Method

13 μL Kinase/Substrate in Reaction Buffer was added to ‘test’ and all ‘control’ wells of a Corning black low binding 384 well (90 μL volume) microplate to give 0.2 μg/mL and 200 nM reaction concentration respectively. 13 μL Substrate in Reaction Buffer was added to ‘blank’ wells to give 200 nM reaction concentration. 2 μL test compound in 10% DMSO/water was added to ‘test’ wells to give final concentrations ranging from 100 to 0.001 μM. 2 μL 10% DMSO/water was added to ‘blank’ and ‘control’ wells. 5 μL ATP in Reaction Buffer was added to all wells to give 10 μM reaction concentration. The reaction mixture was then incubated at room temperature for 25 minutes. The incubation period was followed by the addition of 40 μL IMAP Binding Reagent in Binding Buffer to all wells. The reaction was then further incubated at room temperature for ≧30 minutes.

The fluorescence polarisation of the substrate in each well was recorded using an analyst microplate reader (Molecular Devices) with a single read at Ex485 Em535 (Analyst settings: Z Height 5 mm, G Factor 0.95, Reads/well 1, Integration 100000 μs, Gain Sensitivity 2).

Percentage inhibition was calculated based on activity of the test sample minus the average values in the blank wells relative to the average values measured in control wells minus the average values in the blank wells.

IC₅₀ values were calculated from 10 point dose sigmoid ‘dose-response’ curves using XIfit software (IDBS inc, USA). Data were fitted to a 4 parameter logistic model/sigmoidal dose response:

$$Fit = A + ( \frac{( B - A )}{( 1 + {( \frac{C}{X} )}^{D} )} )$$

where:
A=fit minimum (locked to 0);
B=fit maximum (locked to 100);
C=fit midpoint (pre-fit to 1);
D=slope at linear portion of curve, hillslope (pre-fit to 0.1)

The value for C represents the IC₅₀ of the test compound

### PKD2 (Human Full Length) Enzyme Activity Assay

### Reagents

Kinase Assay Reaction Buffer: This consisted of 0.22 μM filtered 25 mM HEPES and 10 mM MgCl₂ pH 7.5.

Kinase: Human full length PKD2 at ˜100 μg/mL was purchased from Upstate Ltd (Product code 14-506), and obtained from aliquots stored at −70° C. It was prepared with a final concentration of 0.1 μg/mL by diluting 1:300 in Reaction Buffer (30 μL per 9 mL-5 mL per plate with an additional 4 mL dead volume) and vortexing prior to use. It is necessary to check this concentration regularly in case of enzyme degradation.

Substrate: Fluorescein labelled glycogen synthase-derived peptide (FI)-KKLNRTLSVA (also known as MAPKAP K2 substrate) was obtained from Molecular Devices (Product code R7127). It was used at 2 μM by diluting 20 μM stock 1:10 in Kinase/Reaction Buffer (900 μL per 9 mL; 500 μL per 5 mL Reaction Buffer for blank wells requiring <1 mL per place with an additional 4 mL dead volume).

ATP: (Obtained from Sigma, product code A-7699). A 1 mM ATP stock in Reaction Buffer was prepared from a 10 mM stock in 20 mM NaOH and stored as aliquots at −70° C. It was used at 600 μM by diluting 100 mM stock 1:166.6 in Reaction Buffer (36 μL per 6 mL-2 mL per plate with an additional 4 mL dead volume) and vortexing prior to use.

IMAP Reagents: IMAP Binding Reagent (product code R7207) and Binding Buffer (product code R7208) were obtained from Molecular Devices. Both were stored at +4° C. The beads were gently re-suspended before diluting by 1:400 in buffer (Binding Buffer is supplied as a 5× stock and so is diluted with water prior to use) and then vortexing before addition to wells. 16 mL water with 4 mL Binding Buffer and 50 μL Binding Reagent were used per plate (17 mL per plate with an additional 3 mL dead volume).

### Method

5 μL Kinase/Substrate in Reaction Buffer was added to ‘test’ and all ‘control’ wells of a Corning black low binding 384 well (90 μL volume) microplate to give 0.1 μg/mL and 2 μM reaction concentration respectively. 5 μL Substrate in Reaction Buffer was added to ‘blank’ wells to give 2 μM reaction concentration. 1 μL test compounds in 40% DMSO/water was added to ‘test’ wells to give final concentrations ranging from 100 to 0.001 μM. 1 μL 10% DMSO/water was added to ‘blank’ and ‘control’ wells. 4 μL ATP in Reaction Buffer was added to all wells to give 10 μM reaction concentration. The reaction mixture was then incubated at room temperature for 90 minutes. The incubation period was followed by the addition of 90 μL of cold 1× Reaction Buffer. 20 μL of the resulting solution was subsequently transferred to a fresh identical microplate. 40 μL IMAP Binding Reagent in Binding Buffer was added to all wells of this new microplate. The reaction was further incubated at room temperature for ≧30 minutes. The fluorescence polarisation of the peptide substrate was measured using an analyst (Molecular devices) microplate reader with a single read at Ex485 Em535 (Analyst settings: Z Height 5 mm, G Factor 0.95, Reads/well 1, Integration 100000 μs, Gain Sensitivity 2).

Percentage inhibition was calculated based on activity of the test sample minus the average values in the blank wells relative to the average values measured in control wells minus the average values in the blank wells.

IC₅₀ values were calculated from 10 point dose sigmoid ‘dose-response’ curves using XIfit software (IDBS inc, USA). Data were fitted to a 4 parameter logistic model/sigmoidal dose response:

$$Fit = A + ( \frac{( B - A )}{( 1 + {( \frac{C}{X} )}^{D} )} )$$

where:
A=fit minimum (locked to 0);
B=fit maximum (locked to 100);
C=fit midpoint (pre-fit to 1);
D=slope at linear portion of curve, hillslope (pre-fit to 0.1)

The value for C represents the IC₅₀ of the test compound.

### Western Blot 916 (Phospho-Ser916 PKD1) Assay

PANC-1 (ATCC CRL-1469) cells were seeded in 6 well plates. After overnight serum starvation of cells, cells were washed twice in 1 mL serum-free media per well, then treatments were added in serum-free media.

Cells were treated with 2 μM, 5 μM, 10 μM, or 30 μM of an amino-ethyl-amino-aryl (AEAA) compound or with 3 μM GF1 (2-[1-(3-Dimethylaminopropyl)-1H-indol-3-yl]-3-(1H-indol-3-yl)-maleimide, a PKC inhibitor), for comparison purposes, for 1 hour. Then, 200 nM PDBu (phorbol, 12,13-dibutyrate) was added to the wells for 10 minutes. Two wells were used per treatment.

Cells were then scraped into lysis buffer (40 μL per well), samples were homogenised, and protein concentration determined. Equal amounts of protein lysate (26 μg) were loaded onto on pre-cast gels (10%) for western analysis using an anti-PKD1 (human) Antibody (Cell Signaling Technology, No. 2052, Lot 3) and anti-phospho-PKD1 (human) (Ser916) Antibody (Cell Signaling Technology, No. 2051, Lot 3).

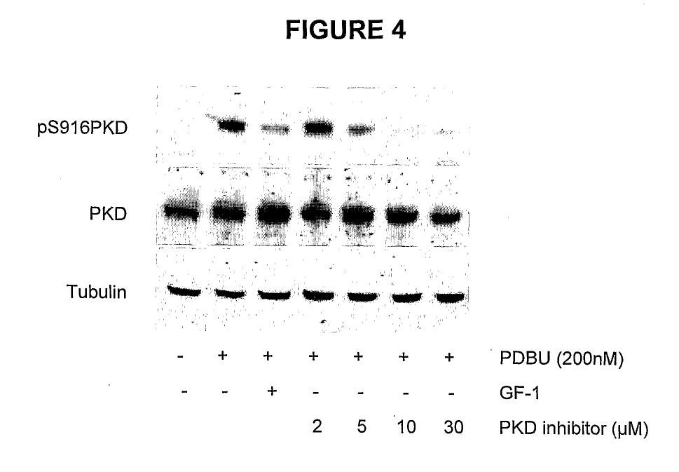

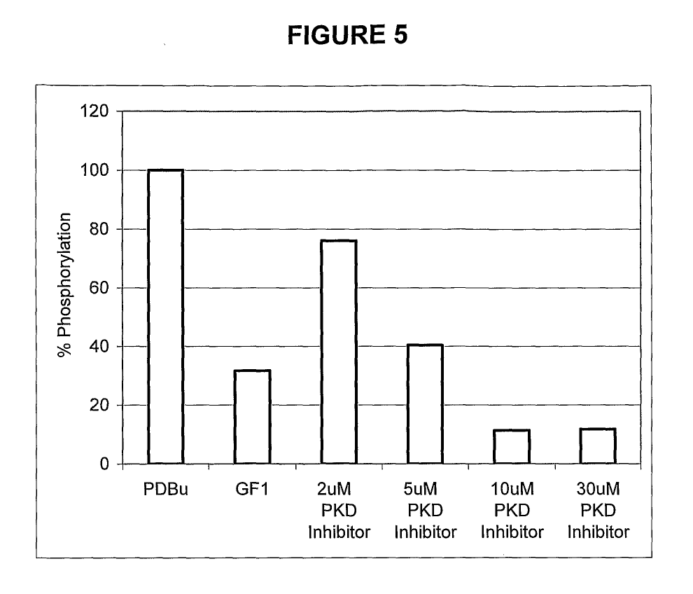

The results are shown in FIG. 4 and FIG. 5.

FIG. 4 is a photographic depiction of the western blot analysis of cell lysates of PANC-1 cells which were treated with increasing amounts (2, 5, 10, 30 μM) of an amino-ethyl-amino-aryl (AEAA) compound (XX-032). Cell lysates were analysed using an anti-PKD1 Antibody (middle panel), anti-phospho-PKD1 (Ser916) Antibody (top panel) and anti-tubulin antibody (lower panel).

FIG. 5 is a depiction of the quantification of the western blot as shown in FIG. 4. The shown columns represent the percentage phosphorylation as measured by densitometry of phospho-PKD1 (Ser916) levels. The results were normalised to the measured PKD1 levels and expressed as a percentage of the level of phosphorylation in the PDBu-stimulated control.

Both figures show that the amino-ethyl-amino-aryl (AEAA) compound inhibited PDBu-stimulated PKD1 Ser916 phosphorylation in PANC-1 cells in a dose-responsive fashion with an IC₅₀ of approximately 4 μM.

### Neurotensin Proliferation Assay

Neurotensin-stimulated proliferation of PANC-1 cells has previously been shown to be mediated by a PKD-dependent pathway (see, e.g., Guha et al., 2002).

BrdU incorporation into PANC-1 cells was used to measure DNA synthesis and thus the level of cell proliferation. Cells were seeded in 10 cm² cell culture dishes in (1×10⁶ cells/well in E4+10% FCS). After serum starvation (24 hours), cells were treated with an amino-ethyl-amino-aryl (AEAA) compound for 1 hour before addition of neurotensin (50 nM final concentration). Cells were then incubated for a further 23 hours before addition of BrdU (10 μM) for the final 1 hour. Samples were fixed using 70% ethanol and labelled with an anti-BrdU antibody (Becton Dickinson Cat No. 347580, Lot No. 13467) followed by a rabbit polyclonal anti-mouse-FITC antibody (DakoCytomation Cat No. F0313, Lot No. 00015066) and propidium iodide. Samples were then analysed by Fluorescent Activated Cell Sorting (FACS). The measured fluorescence was directly proportional to the incorporated BrdU.

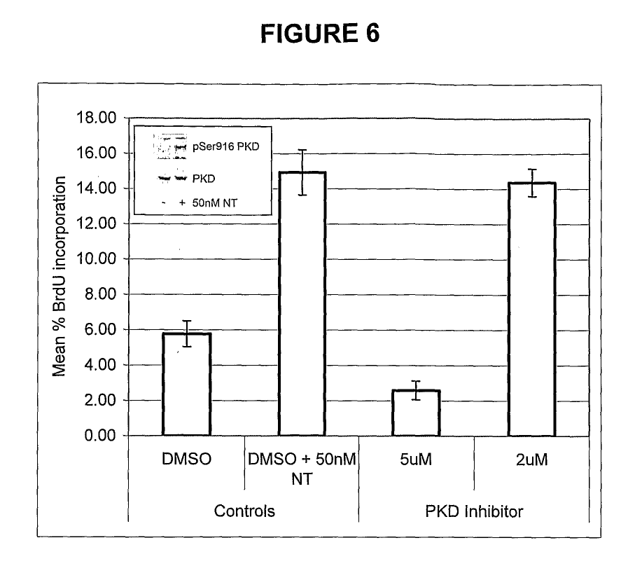

Stimulation of serum-starved cells with 50 nM neurotensin (NT) resulted in ˜2.7 fold increase in cell proliferation. Neurotensin-stimulated PANC-1 proliferation was inhibited to basal levels by treatment of cells with 5 μM amino-ethyl-amino-aryl (AEAA) compound (XX-032). Western blotting demonstrated that 50 nM neurotensin was sufficient to stimulate measurable levels of pSer916 PKD1 phosphorylation and thus PKD activity (see FIG. 6).

FIG. 6 is a graphic representation of the results of the neurotensin proliferation assay. The columns in the graph represent the mean percentage of BrdU incorporation into PANC-1 cells as a measure for cell proliferation. The two left-hand columns represent the controls of DMSO (basal level of non-stimulated cell proliferation) and DMSO plus 50 nM neurotensin (stimulated cell proliferation). The two right-hand columns represent the effect of two different concentrations (5 μM and 2 μM) of an amino-ethyl-amino-aryl (AEAA) compound on the neurotensin-stimulated cell proliferation. The graph illustrates that an increasing the amount of the amino-ethyl-amino-aryl (AEAA) compound inhibited stimulated cell proliferation.

### MTT Assay

The MTT (3-[4,5-dimethylthiazol-2-yl]-2,5-diphenyltetrazolium bromide) assay can be used to assess the effect of compounds on cell viability and proliferation and to determine whether a compound is cytotoxic. In living cells, the tetrazolium salt (MTT) is reduced to a coloured formazan product (1-[4,5-dimethylthiazol-2-yl]3,5-diphenylformazan), which can be quantified. The reduction of MTT is attributed to the mitochondrial function of cells.

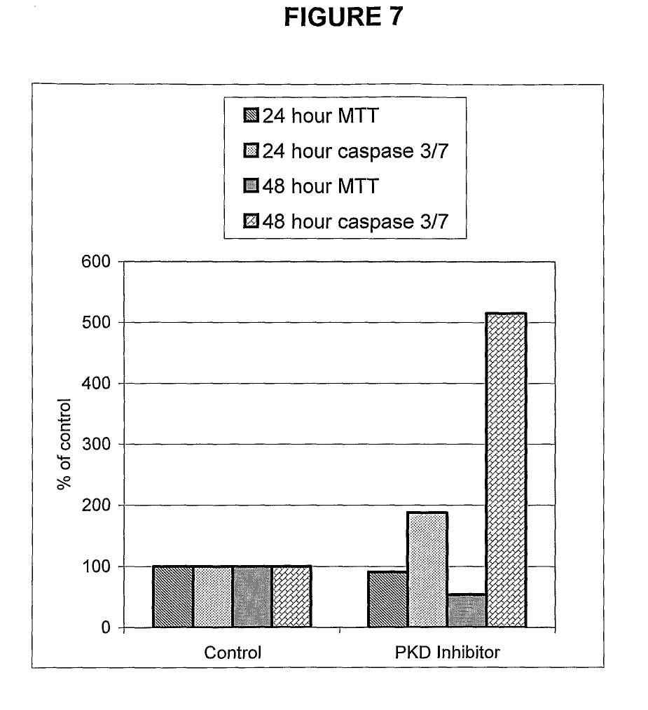

PANC-1 cells were seeded into 96 well plates (1×10⁴ cells/well in E4+10% FCS) and placed in an incubator at 37° C., 5% CO₂ overnight. Cells were serum starved (E4+0.5% FCS) for 16 hours and then treated with an amino-ethyl-amino-aryl (AEAA) compound (XX-032) in full media for 1 hour prior to treatment with Neurotensin (50 nM) for a further 23 hours or 47 hours (in E4+0.5% FCS; total exposure to test compound was 24 hours or 48 hours; 37° C. 5% CO₂). At the appropriate timepoint plates were removed from the incubator and the media was aspirated off. 50 μL per well of 2 mg/mL MTT solution was added and the plates were placed back into the incubator for 2.5 to 4 hours. After incubation, the plates were removed from the incubator and the MTT solution was completely aspirated off the cells. 50 μL DMSO was added to each well and the plates agitated vigorously for 1 minute without introducing bubbles to the wells. The plates were read in a 96 well plate reader at 562 nm (Lab Systems, Ascent Multiscan). The results are shown in FIG. 7

### Apoptosis Assay

PKD2 has been shown to play a role in cell survival through increasing cellular resistance to apoptosis (see, e.g., Trauzold et al., 2003; Storz et al., 2005). In addition, results from an siRNA screen of human kinases has identified PKD2 as a survival kinase (Mackeigan et al., 2005).

PANC-1 cells were seeded into 96 well plates (1×10⁴ cells/well in E4+10% FCS) and placed in an incubator at 37° C., 5% CO₂ overnight. Cells were serum starved (E4+0.5% FCS) for 16 hours and then treated with an amino-ethyl-amino-aryl (AEAA) compound in full media for 1 hour prior to treatment with Neurotensin (NT; 50 nM) for a further 23 hours or 47 hours (in E4+0.5% FCS; total exposure to test compound was 24 hours or 48 hours). Cells were then assayed for Caspase3/7 activity (Caspase-Glo; Promega) according to the manufacturer's instructions.

The Caspase-Glo assay is a homogenous luminescent assay that measures caspase 3 and 7 activities. The assay kit provides luminegenic caspase 3 and 7 substrate, which contains the tetrapeptide DEVD in a reagent optimised by the manufacturer for caspase activity, luciferase activity, and cell lysis. When added to the cell samples, these reagents result in cell lysis, followed by caspase cleavage of the substrate and generation of a luminescent signal produced by luciferase, whereby the luminescence is proportional to the amount of caspase activity present. An increase of caspase activity is proportional to increased apoptosis.

Treatment of PANC-1 cells with 5 μM amino-ethyl-amino-aryl (AEAA) compound (XX-032) for 48 hours resulted in a 5-fold increase in caspase 3/7 activity and a corresponding 2-fold decrease in cell viability. These data suggest that tested compounds induced cell death by apoptosis (see FIG. 7).

FIG. 7 shows a graphic representation of the results obtained in this assay. The depicted columns show the change in viability or induction of apotosis in the presence of an amino-ethyl-amino-aryl (AEAA) compound (XX-032). Cell viability was measured by the MTT assay at two different time points (24 and 48 hours) and induction of apoptosis was measured by the caspase assay at two different time points (24 and 48 hours). The data are expressed as a percentage of the level in the corresponding control.

### Permeability

The lipid-PAMPA (Parallel Artificial Membrane Permeation Assay) method is a non-cell based assay designed to predict passive, transcellular permeability of drugs. Methanol was used as “biological membrane” control.

Test compound-containing donor solution (500 μM) was prepared in 1.5 mL Eppendorf tube by dilution of 10 mM stock of the test compound in PBS (150 μL/well) as shown in the Table below. The aqueous acceptor buffer was a 5% DMSO solution in PBS (pH 7.4; 300 μL/well), prepared in 30 mL tube.

TABLE 1
Preparation of drug-containing donor solution

| | Triplicate | 2 × triplicate |
|---|---|---|
| 10 mM stock in DMSO | 25 μL | 50 μL |
| PBS | 475 μL | 950 μL |

Lecithin was kept at −20° C. under inert gas (nitrogen). In a 1.5 mL Eppendorf tube, a 1% solution (w/v; 5 mg/500 μL) of lecithin in dodecane (±500 μL/plate) was prepared. The mixture was sonicated to ensure complete dissolution (until the lecithin solution approaches the clarity of water). The underdrain of the multiscreen filter plate (donor plate) was removed. The donor plate was set in the vacuum manifold so that the underside of the membrane did not make contact with any surfaces. 5 μL of the lecithin/dodecane mixture was added into each “donor” plate well and 5 μL of methanol as control. Immediately after the application of the artificial membrane (within 10 minutes maximum in the case of lecithin/dodecane and 3 minutes in the case of methanol), 150 μL of test compound-containing donor solution was added to each well of the donor plate. 300 μL of the aqueous acceptor buffer was added to each well of the acceptor plate. The test compound-filled donor plate was placed into the acceptor plate, making sure the underside of the membrane was in contact with the buffer in all wells. The plate lid was replaced and the plate incubated at room temperature for 16 hours. After incubation, 50 μL/well of donor and acceptor solution was transferred to a 96-well plate for UV-star and 50 μL of DMSO was added. The plate was scanned after this. The permeability rates (Pₑ) was determined as follows. If the standard calibration generated a linear curve with a correlation coefficient (r²) greater than 0.85, then the permeability could be determined using UV/Vis spectroscopy; otherwise (r²<0.85), an alternate method (HPLC) was more appropriate.

Final drug concentration in the donor and acceptor compartments were determined by subtracting the Y intercept of the calibration curve from the measured absorbance, and by dividing this result by the slope of the calibration curve, and multiplying by the dilution factor:

$$Concentration ( µM ) = ( \frac{( Abs - Y intercept )}{Slope} ) \times dilution$$

From the concentration values in the donor and acceptor compartment, the permeability Pₑ (cm/s) can be calculated using the equation:

$$log {P}_{e} = log \{ C \times - ln ( 1 - \frac{{[ drug ]}_{acceptor}}{{[ drug ]}_{equilibrium}} ) \}$$

$$C = ( \frac{{V}_{D} \times {V}_{A}}{( {V}_{D} + {V}_{A} ) \times Area \times time} )$$

and wherein:

| | | |
|---|---|---|
| V_{D} | Volume of donor | Expressed in cm³; 150 μL = |
| | compartment | 0.15 cm³ |
| V_{A} | Volume of acceptor | Expressed in cm³; 300 μL = |
| | compartment | 0.3 cm³ |
| Area | Active surface area of | Defined as membrane |
| | membrane | area × porosity: |
| | | 0.24 cm² × 100% |
| Time | Incubation time for the | Expressed in seconds |
| | assay | |
| [drug]_{acceptor} | Concentration of | Concentration = (Abs./ |
| | compound in the acceptor | Molar absorbance |
| | compartment at the | coefficient) × 10⁶ |
| | completion of the assay | |
| [drug]_{equilibrium} | Concentration of | Concentration = |
| | compound at theoretical | {dose [μmol]/ |
| | equilibrium | (V_{D} + V_{A}) [μL]} × 10⁶ |

Permeability (Pe) was measured for the following 17 compounds:

| No. | ID No. |
|---|---|
| 1 | XX-030 |
| 2 | XX-039 |
| 3 | XX-043 |
| 4 | XX-044 |
| 5 | XX-050 |
| 6 | XX-051 |
| 7 | XX-063 |
| 8 | XX-069 |
| 9 | XX-073 |
| 10 | XX-077 |
| 11 | XX-084 |
| 12 | XX-093 |
| 13 | XX-094 |
| 14 | XX-096 |
| 15 | XX-100 |
| 16 | XX-101 |
| 17 | XX-106 |

The permeability (Pe) values were as follows:

at least 3 compounds tested have a permeability of at least 10⁻⁵ cm/s.
at least 6 compounds tested have a permeability of at least 5×10⁻⁶ cm/s.
at least 12 compounds tested have a permeability of at least 10⁻⁶ cm/s.

### Additional Biological Data

Biological data were obtained using the PKD1 (Murine Kinase Domain) Enzyme Activity Assay described above for the following 127 compounds: XX-001 through XX-125 and YY-001 through YY-002.

For the PKD1 (Murine Kinase Domain) Enzyme Activity Assay, the IC50 (μM) values are as follows:

at least 11 compounds tested had an IC50 of 0.001 μM or less;
at least 28 compounds tested had an IC50 of 0.01 μM or less;
at least 57 of the compounds tested had an IC50 of 0.1 μM or less;
at least 97 of the compounds tested had an IC50 of 1 μM or less.
at least 114 of the compounds tested had an IC50 of 10 μM or less.

For the PKD1 (Murine Kinase Domain) Enzyme Activity Assay, the IC50 (μM) value for XX-097 was 0.016 μM.

Biological data were obtained using the PKD1 (Murine Kinase Domain) Enzyme Activity Assay described above for the following 346 compounds: XX-001 through XX-143 and XX-145 through XX-344 and YY-001 through YY-003.

For the PKD1 (Murine Kinase Domain) Enzyme Activity Assay, the IC50 (μM) values are as follows:

at least 47 compounds tested had an IC50 of 0.001 μM or less;
at least 153 compounds tested had an IC50 of 0.01 μM or less;
at least 228 of the compounds tested had an IC50 of 0.1 μM or less;
at least 305 of the compounds tested had an IC50 of 1 μM or less.
at least 333 of the compounds tested had an IC50 of 10 μM or less.

Biological data were obtained using the PKD1 (Human Full Length) Enzyme Activity Assay described above for the following 16 compounds: XX-026, XX-168, XX-183, XX-184, XX-190, XX-201, XX-202, XX-207, XX-209, XX-210, XX-227, XX-230, XX-265, XX-266, XX-267, XX-276.

For the PKD1 (Human Full Length) Enzyme Activity Assay, the IC50 (μM) values are as follows:

at least 9 of the compounds tested had an IC50 of 0.001 μM or less;
all of the compounds tested had an IC50 of 0.01 μM or less.

For the PKD1 (Human Full Length) Enzyme Activity Assay, compound XX-276 had an IC50 (μM) value of 0.0009 μM.

Biological data were obtained using the PKD2 (Human Full Length) Enzyme Activity Assay described above for the following 16 compounds: XX-207, XX-210, XX-202, XX-230, XX-209, XX-168, XX-276, XX-227, XX-267, XX-190, XX-184, XX-183, XX-201, XX-026, XX-265, XX-266.

For the PKD2 (Human Full Length) Enzyme Activity Assay, the IC₅₀ (μM) values are as follows:

at least 9 of the compounds tested had an IC50 of 0.01 μM or less;
all of the compounds tested had an IC50 of 0.1 μM or less.

For the PKD2 (Human Full Length) Enzyme Activity Assay, compound XX-276 had an IC50 (μM) value of 0.0041 μM.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
Bollag W B, Dodd M E, Shapiro B A. (2004). Protein kinase D and keratinocyte proliferation. Drug News Perspect. March; 17(2):117-26.
Bowden, E. T., Barth, M., Thomas, D., Glazer, R. I. & Mueller, S. C. (1999). An invasion-related complex of cortactin, paxillin and PKCμ associates with invadopodia at sites of extracellular matrix degradation. Oncogene 18(31), 4440-9.
Doppler H, Storz P, Li J, Comb M J, Toker A., (2005), A phosphorylation state-specific antibody recognizes Hsp27, a novel substrate of protein kinase D. J Biol Chem. 280(15):15013-15019.
Gamido C, Schmitt E, Cande C, Vahsen N, Parcellier A, Kroemer G. N (2003). HSP27 and HSP70: potentially oncogenic apoptosis inhibitors. Cell Cycle. 6, 579-584.
Gonzales, J. E., et al., “Quinazolines Useful as Modulators of Ion Channels,” international patent publication number WO 2004/078733, published 16 Sep. 2004.
Guha S, Lunn J A, Santiskulvong C and Rozengurt, E (2003). Neurotensin stimulates PKC-dependent mitogenic signaling in human pancreatic carcinoma cell line PANC-1. Cancer Research 63, 2379-2387.
Guha, S, Rey, O and Rozengurt, E (2002). Neurotensin induces Protein Kinase C-dependent Protein Kinase D activation and DNA synthesis in human pancreatic carcinoma cell line, PANC-1. Cancer Research 62, 1632-1640.
Hurd C, Rozengurt E. (2003) Uncoupling of protein kinase D from suppression of EGF-dependent c-Jun phosphorylation in cancer cells. Biochem Biophys Res Commun. 302, 800-804.
Hurd C, Waldron R T, Rozengurt E. (2002). Protein kinase D complexes with C-Jun N-terminal kinase via activation loop phosphorylation and phosphorylates the C-Jun N-terminus. Oncogene. 21, 2154-2160.
Huynh Q K, McKinsey T A. (2006) Protein kinase D directly phosphorylates histone deacetylase 5 via a random sequential kinetic mechanism. Arch Biochem Biophys. 450, 141-148.
Johannes, F. J., Horn, J., Link, G., Haas, E., Siemienski, K., Wajant, H. & Pfizenmaier, K. (1998). Protein kinase Cmu downregulation of tumor-necrosis-factor-induced apoptosis correlates with enhanced expression of nuclear-factor-kappaB-dependent protective genes. Eur J Biochem. 257(1), 47-54.
Kennett, S. B., Roberts, J. D. & Olden, K. (2004). Requirement of PKCmu activation and calpain-mediated proteolysis for arachidonic acid-stimulated adhesion of MDA-MB-435 human mammary carcinoma cells to collagen type IV. J Biol Chem. November 7 [Epub ahead of print].
MacFarlane, D. E., et al., “Antagonism of Immunostimulatory CPG-Oligonucleotides by 4-Aminoquinolines and Other Weak Bases,” international patent publication number WO 2000/076982, published 21 Dec. 2000.
MacKeigan J P, Murphy L O, Blenis J. (2005), Sensitized RNAi screen of human kinases and phosphatases identifies new regulators of apoptosis and chemoresistance. Nat Cell Biol. 2005 June; 7(6): 591-600.
Matthews, S. Rozengurt, E. & Cantrell, D. (2000b). Protein Kinase D: a selective target for antigen receptors and a downstream target for Protein Kinase C in lymphocytes. J Exp. Med. 191, 2075-82.
Matthews, S., Iglesias, T., Rozengurt, E. & Cantrell, D. (2000a). Spatial and temporal regulation of Protein Kinase D (PKD). EMBO J. 19, 2935-45.
McKinsey, T A and Olson, E N. (2005). Toward transcriptional therapies for the failing heart: chemical screens to modulate genes. J. Clin. Invest. 115:538-546.
Mihailovic T, Marx M, Auer A, Van Lint J, Schmid M, Weber, C and Seufferlein T (2004). Protein kinase D2 mediates activation of NfkappaB by Bcr-Abl in Bcr-Abl+ human myeloid leukemia cells. Cancer Research 64, 8939-8944.
Paolucci, L and Rozengurt, E (1999). Protein Kinase D in small cell lung cancer cells: Rapid activation through Protein Kinase C. Cancer Research 59, 572-577.
Qin L, Zeng H, Zhao D. (2006). Requirement of protein kinase D tyrosine phosphorylation for VEGF-A165-induced angiogenesis through its interaction and regulation of phospholipase Cgamma phosphorylation. J. Biol. Chem., 281, 32550-32558.
Rennecke, J., Rehberger, P. A., Furstenberger, G., Johannes, F. J., Stohr, M., Marks, F. & Richter, K. H. (1999). Protein kinase Cμ expression correlates with enhanced keratinocyte proliferation in normal and neoplastic mouse epidermis and in cell culture. Int J. Cancer 80, 98-103.
Ristich V L, Bowman P H, Dodd M E, Bollag W B. (2006) Protein kinase D distribution in normal human epidermis, basal cell carcinoma and psoriasis. Br J Dermatol. 154, 586-593.
Singh P, Lillywhite B, Bannaghan C, Broad P., 2005, “Using IMAP technology to identify kinase inhibitors: comparison with a substrate depletion approach and analysis of the nature of false positives,” Comb Chem High Throughput Screen. Vol. 8, No. 4, pp. 319-25.
Stewart J R and O'Brian C A (2004). Resveratrol antagonizes EGFR-dependent Erk1/2 activation in androgen-independent prostate cancer cells with associated isozyme-selective PKCα inhibition. Invest. New Drugs 22, 107-117.
Storz P, Doppler H, Toker, A (2005), Protein kinase D mediates mitochondrion-to-nucleus signaling and detoxification from mitochondrial reactive oxygen species. Mol Cell Biol 25(19):8520-8530.
Storz P, Doppler H and Toker A (2004a). Protein kinase Cδ selectively regulates protein kinase D-dependent activation of NfkappaB in oxidative stress signaling. Mol. Cell. Biol. 24, 2614-2626.
Storz P, Doppler H, Toker A. (2004b). Activation loop phosphorylation controls protein kinase D-dependent activation of nuclear factor kappaB. Mol Pharmacol October; 66(4):870-879.
Storz, P and Toker, A (2003). Protein kinase D mediates a stress-induced NF-κB activation and survival pathway. EMBO J. 22, 109-120.
Trauzold A, Schmiedel S, Sipos B, Wermann H, Westphal S, Roder C, Klapper W, Arlt A, Lehnert L, Ungefroren H, Johannes F J, Kalthoff H. (2003) PKCmu prevents CD95-mediated apoptosis and enhances proliferation in pancreatic tumour cells. Oncogene 22(55):8939-8947.
Van Lint, J., Rykx, A., Maeda, Y., Vantus, T., Sturany, S., Malhotra, V., Vandenheede, J. R. & Seufferlein, T. (2002). Protein Kinase D: an intracellular traffic regulator on the move. Trends Cell Biol. 12(4), 193-200.
Van Lint, J. V., Sinnett-Smith, J., & Rozengurt, E. (1995). Expression and characterization of PKD, a phorbol ester and diacylglycerol-stimulated serine protein kinase. J Biol Chem. 270(3), 1455-61.
Vega R B, Harrison B C, Meadows E, Roberts C R, Papst P J, Olson E N and McKinsey T A (2004). Protein kinases C and D mediate agonist-dependent cardiac hypertrophy through nuclear export of histone deacetylase 5. Mol. Cell. Biol 24, 8374-8385.
Wang Y, Waldron R T, Dhaka A, Patel A, Riley M M, Rozengurt E, Colicelli J. (2002). The RAS effector RIN1 directly competes with RAF and is regulated by 14-3-3 proteins. Mol Cell Biol. 22, 916-226.
Wong C, Jin Z G (2005). Protein kinase C-dependent protein kinase D activation modulates ERK signal pathway and endothelial cell proliferation by vascular endothelial growth factor. J Biol Chem. September 30; 280(39):33262-9.
Wood C D, Marklund U, Cantrell D A (2005). Dual phospholipase C/diacylglycerol requirement for protein kinase D1 activation in lymphocytes. J Biol Chem. February 18; 280(7):6245-51.
Zugaza, J. L., Sinnett-Smith, J., Van Lint, J. & Rozengurt, E. (1996). Protein Kinase D (PKD) activation in intact cells through a protein kinase C-dependent signal transduction pathway. EMBO J. 15, 6220-30.
Zugaza, J. L., Waldron, R. T., Sinnett-Smith, J. & Rozengurt, E. (1997). Bombesin, vasopressin, endothelin, bradykinin, and platelet-derived growth factor rapidly activate protein kinase D through a protein kinase C-dependent signal transduction pathway. J Biol Chem. 272(38), 23952-60.

1. A compound selected from compounds of the following formula and pharmaceutically acceptable salts thereof: 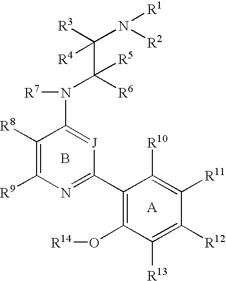
wherein:
J is independently N or CH;
and wherein:
(1) each of R⁸ and R⁹ is independently —H or a Ring B substituent;
or:
(2) R⁸ and R⁹, taken together with the atoms to which they are attached, form an aromatic Ring C having exactly 5 ring atoms or exactly 6 ring atoms, wherein each ring atom is a carbon ring atom or a nitrogen ring atom, wherein Ring C has exactly 0, exactly 1, or exactly 2 ring nitrogen atoms, and wherein Ring C is fused to Ring B;
and wherein:
(1) each of R¹⁰, R¹¹, R¹², and R¹³ is independently —H or a Ring A substituent;
or:
(2) each of R¹² and R¹³ is independently —H or a Ring A substituent; and R¹⁰ and R¹¹, taken together with the atoms to which they are attached, form an aromatic Ring D having exactly 6 ring atoms, wherein each ring atom is a carbon ring atom, and wherein Ring D is fused to Ring A;
or:
(3) each of R¹⁰ and R¹³ is independently —H or a Ring A substituent; and R¹¹ and R¹², taken together with the atoms to which they are attached, form an aromatic Ring E having exactly 6 ring atoms, wherein each ring atom is a carbon ring atom, and wherein Ring E is fused to Ring A;
or:
(4) each of R¹⁰ and R¹¹ is independently —H or a Ring A substituent; and R¹² and R¹³, taken together with the atoms to which they are attached, form an aromatic Ring F having exactly 6 ring atoms, wherein each ring atom is a carbon ring atom, and wherein Ring F is fused to Ring A;
and wherein:
each of R¹, R², R³, R⁴, R⁵, R⁶, and R⁷ is independently —H or a group G;
and additionally wherein:
each of R³, R⁴, R⁵, and R⁶ may be a group Y;
each of R¹, R², and R⁷ may be a group Z;
R³ and R⁴, taken together, may form a group ═O;
R⁵ and R⁶, taken together, may form a group ═O;
and wherein:
R¹⁴ is independently —H or a group W;
wherein:
each Ring A substituent, if present, is independently a 1° carbo-substituent or a 1° hetero-substituent;
each Ring B substituent, if present, is independently a 1° carbo-substituent or a 1° hetero-substituent;
the group W, if present, is independently a 1° carbo-substituent;
each group G, if present, is independently a 1° carbo-substituent;
each group Y, if present, is independently a 1° hetero-substituent;
each group Z, if present, is independently a 1° hetero-substituent selected from: (H-10), (H-12), (H-13), and (H-18);
wherein:
each 1° carbo-substituent is independently selected from:
(C-1) C₁₋₇alkyl,
(C-2) C₂₋₇alkenyl,
(C-3) C₂₋₇alkynyl,
(C-4) C₃₋₇cycloalkyl,
(C-5) C₃₋₇cycloalkenyl,
(C-6) C₃₋₁₄heterocyclyl,
(C-7) C₆₋₁₄carboaryl,
(C-8) C₅₋₁₄heteroaryl,
(C-9) C₆₋₁₄carboaryl-C₁₋₇alkyl, and
(C-10) C₅₋₁₄heteroaryl-C₁₋₇alkyl;
wherein each C₁₋₇alkyl, C₂₋₇alkenyl, C₂₋₇alkynyl, C₃₋₇cycloalkyl, and C₃₋₇cycloalkenyl, is independently unsubstituted or substituted with one or more substituents selected from 1° hetero-substituents; and
wherein each C₃₋₁₄heterocyclyl, C₆₋₁₄carboaryl, and C₅₋₁₄heteroaryl is independently unsubstituted or substituted with one or more substituents selected from 1° hetero-substituents and 2° carbo-substituents;
each 2° carbo-substituent is independently as defined for 1° carbo-substituent, except that:
each C₁₋₇alkyl, C₂₋₇alkenyl, C₂₋₇alkynyl, C₃₋₇cycloalkyl, and C₃₋₇cycloalkenyl, is independently unsubstituted or substituted with one or more substituents selected from 2° hetero-substituents; and
each C₃₋₁₄heterocyclyl, C₆₋₁₄carboaryl, and C₅₋₁₄heteroaryl is independently unsubstituted or substituted with one or more substituents selected from 2° hetero-substituents and 3° carbo-substituents;
each 3° carbo-substituent is independently as defined for 1° carbo-substituent, except that:
each C₁₋₇alkyl, C₂₋₇alkenyl, C₂₋₇alkynyl, C₃₋₇cycloalkyl, and C₃₋₇cycloalkenyl, is unsubstituted; and
each C₃₋₁₄heterocyclyl, C₆₋₁₄carboaryl, and C₅₋₁₄heteroaryl is unsubstituted;
each 1° hetero-substituent is independently selected from:
(H-1) —F, —Cl, —Br, —I;
(H-2) —OH;
(H-3) —OR^{A1}, wherein R^{A1} is independently a 2° carbo-substituent;
(H-4) —SH;
(H-5) —SR^{A2}, wherein R^{A2} is independently a 2° carbo-substituent;
(H-6) —NH₂, —NHR^{A3}, —NR^{A4}R^{A5}, wherein each of R^{A3}, R^{A4}, and R^{A5} is independently a 2° carbo-substituent; or R^{A4} and R^{A5} taken together with the nitrogen atom to which they are attached form a ring having from 3 to 7 ring atoms;
(H-7) —NHC(═O)R^{A6}, —NR^{A7}C(═O)R^{A6}, wherein each of R^{A6} and R^{A7} is independently a 2° carbo-substituent;
(H-8) —NHC(═O)OR^{A9}, —NR^{A10}C(═O)OR^{A9}, wherein each of R^{A9} and R^{A10} is independently a 2° carbo-substituent;
(H-9) —NHC(═O)NH₂, —NR^{A10}C(═O)NH₂, —NHC(═O)NHR^{A11}, —NR^{A10}C(═O)NHR^{A11}, —NHC(═O)NR^{A11}R^{A12}, —NR^{A10}C(═O)NHR^{A11}R^{A12}, wherein each of R^{A10}, R^{A11}, and R^{A12} is independently a 2° carbo-substituent; or R^{A11} and R^{A12} taken together with the nitrogen atom to which they are attached form a ring having from 3 to 7 ring atoms;
(H-10) —C(═O)R^{A13}, wherein R^{A13} is independently a 2° carbo-substituent;
(H-11) —C(═O)OH;
(H-12) —C(═O)OR^{A14}, wherein R^{A14} is independently a 2° carbo-substituent;
(H-13) —C(═O)NH₂, —C(═O)NHR^{A15}, —C(═O)NR^{A15}R^{A16}, wherein each of R^{A15} and R^{A16} is independently a 2° carbo-substituent; or R^{A15} and R^{A16} taken together with the nitrogen atom to which they are attached form a ring having from 3 to 7 ring atoms;
(H-14) —OC(═O)R^{A17}, wherein R^{A17} is independently a 2° carbo-substituent
(H-15) —OC(═O)NH₂, —OC(═O)NH^{A18}, —OC(═O)NR^{A18}R^{A19}, wherein each of R^{A18} and R^{A19} is independently a 2° carbo-substituent; or R^{A18} and R^{A19} taken together with the nitrogen atom to which they are attached form a ring having from 3 to 7 ring atoms;
(H-16) —S(═O)₂NH₂, —S(═O)₂NHR^{A20}, —S(═O)₂NR^{A20}R^{A21}, wherein each of R^{A20} and R^{A21} is independently a 2° carbo-substituent; or R^{A20} and R^{A21} taken together with the nitrogen atom to which they are attached form a ring having from 3 to 7 ring atoms;
(H-17) —NHS(═O)₂R^{A22}, —NR^{A23}S(═O)₂R^{A22}, wherein each of R^{A22} and R^{A23} is independently a 2° carbo-substituent;
(H-18) —S(═O)₂R^{A24}, wherein R^{A24} is independently a 2° carbo-substituent;
(H-19) —S(═O)₂OH;
(H-20) —S(═O)₂OR^{A25}, —OS(═O)₂R^{A26}, wherein each of R^{A25} and R^{A26} is independently a 2° carbo-substituent;
(H-21) —NO₂;
each 2° hetero-substituent is independently as defined for 1° hetero-substituent, except that: each 2° carbo-substituent is a 3° carbo-substituent;
with the proviso that the compound is not:
(A1) 2-{7-chloro-4-[isopropyl-(2-isopropylamino-ethyl)-amino]-quinazolin-2-yl}-phenol;
(A2) 2-{7-methyl-4-[isopropyl-(2-isopropylamino-ethyl)-amino]-quinazolin-2-yl}-phenol;
(A3) 2-{6-Fluoro-4-[isopropyl-(2-isopropylamino-ethyl)-amino]-quinazolin-2-yl}-phenol;
(A4) 2-{4-[(2-Dimethylamino-ethyl)-methyl-amino]-quinazolin-2-yl}-phenol (XX-110);
(A5) 2-{4-[Benzyl-(2-dimethylamino-ethyl)-amino]-quinazolin-2-yl}-phenol (XX-111);
(A6) 2-{4-[Methyl-(2-methylamino-ethyl)-amino]-quinazolin-2-yl}-phenol (XX-113);
(B1) 2-[4-(2-diethylamino-ethylamino)-quinazolin-2-yl]-6-methoxy-phenol;
(B2) 2-[4-(2-diethylamino-ethylamino)-quinazolin-2-yl]-phenol;
(B3) 2-{4-[2-(2-amino-ethylamino)-ethylamino]-quinazolin-2-yl}-phenol;
(B4) 2-[4-(2-amino-ethylamino)-quinazolin-2-yl]-phenol (XX-100);
(C1) 2-{4-[2-(2-amino-ethylamino)-ethylamino]-6-methyl-pyrimidin-2-yl}-phenol;
(C2) 2-[4-(2-Amino-ethylamino)-6-methyl-pyrimidin-2-yl]-phenol;
(D1) N′-[2-(2,6-dimethoxy-phenyl)-quinolin-4-yl]-N,N-dimethyl-ethane-1,2-diamine; or
(E1) N′-[2-(2,6-dimethoxy-phenyl)-quinolin-4-yl]-N,N-dimethyl-ethane-1,2-diamine bis(hydrobromide).

2. A compound according to claim 1, wherein J is independently N.

3. A compound according to claim 1, wherein J is independently CH.

4. A compound according to claim 1, wherein each of R⁸ and R⁹ is independently —H or a Ring B substituent.

5. (canceled)

6. A compound according to claim 4, where each Ring B substituent, if present, is independently selected from: —F, —Cl, —Br, —I, —OH, —O—C₁₋₇alkyl, —O—C₁₋₇haloalkyl, —S—C₁₋₇alkyl, —NH₂, —NH—C₁₋₁₇alkyl, —N(C₁₋₇alkyl)₂, —C(═O)OH, —C(═O)O—C₁₋₇alkyl, —C(═O)NH₂, —OC(═O)—C₁₋₇alkyl, —NO₂, C₁₋₇alkyl, —C₁₋₇haloalkyl, —CH₂-Ph, -Ph, -Ph-C₁₋₇haloalkyl.

7. (canceled)

8. A compound according to claim 1, wherein:
R⁸ is independently selected from: —H, —F, —Cl, —Br, —I, C₁₋₇alkyl, pyrazole, or phenyl; wherein each pyrazole and phenyl, if present, is optionally substituted, for example, with one or more substituents selected from: —F, —Cl, —Br, —I, —OH, C₁₋₇alkyl, and —O—C₁₋₄alkyl; and
R⁹ is independently selected from: —H and C₁₋₄alkyl.

9. A compound according to claim 1, wherein each of R⁸ and R⁹ is independently H.

10. (canceled)

11. A compound according to claim 1, wherein R⁸ and R⁹, taken together with the atoms to which they are attached, form an aromatic Ring C having exactly 6 ring atoms, wherein each ring atom is a carbon ring atom, and wherein Ring C is fused to Ring B.

12. (canceled)

13. A compound according to claim 11, wherein Ring C independently is unsubstituted, or is substituted with one or more Ring C substituents, wherein each Ring C substituent, if present, is independently selected from: —F, —Cl, —Br, —I, —OH, —O—C₁₋₇alkyl, —O—C₁₋₇haloalkyl, —S—C₁₋₇alkyl, —NH₂, —NH—C₁₋₇alkyl, —N(C₁₋₇alkyl)₂, —C(═O)OH, —C(═O)O—C₁₋₇alkyl, —C(═O)NH₂, —OC(═O)—C₁₋₇alkyl, —NO₂, C₁₋₇alkyl, —C₁₋₇haloalkyl, —CH₂-Ph, -Ph, -Ph-C₁₋₇haloalkyl.

14. A compound according to claim 11, wherein Ring C is unsubstituted.

15. A compound according to claim 1, wherein each of R¹⁰, R¹¹, R¹², and R¹³ is independently —H or a Ring A substituent.

16. (canceled)

17. A compound according to claim 1, wherein each of R¹⁰, R¹², and R¹³ is independently —H, and R¹¹ is independently a Ring A substituent.

18. A compound according to claim 1, wherein:
each of R¹⁰, R¹², and R¹³ is independently —H, and
R¹¹ is independently —F, —Cl, —Br, —I, phenyl, pyrazolyl, or pyridyl; wherein said phenyl, pyrazolyl, or pyridyl is optionally substituted with one or more substituents independently selected from: —F, —Cl, —Br, —I, C₁₋₆alkyl, —CF₃, —OH, —O—C₁₋₆alkyl, and —OCF₃.

19. A compound according to claim 1, wherein:
each of R¹⁰, R¹², and R¹³ is independently —H, and
R¹¹ is independently pyrazolyl, wherein said pyrazolyl is optionally substituted with one or more C₁₋₆alkyl groups.

20. (canceled)

21. A compound according to claim 1, wherein each of R¹² and R¹³ is independently —H or a Ring A substituent; and R¹⁰ and R¹¹, taken together with the atoms to which they are attached, form an aromatic Ring D having exactly 6 ring atoms, wherein each ring atom is a carbon ring atom, and wherein Ring D is fused to Ring A.

22-25. (canceled)

26. A compound according to claim 1, wherein each of R¹⁰ and R¹³ is independently —H or a Ring A substituent; and R¹¹ and R¹², taken together with the atoms to which they are attached, form an aromatic Ring E having exactly 6 ring atoms, wherein each ring atom is a carbon ring atom, and wherein Ring E is fused to Ring A.

27-30. (canceled)

31. A compound according to claim 1, wherein each of R¹⁰ and R¹¹ is independently —H or a Ring A substituent; and R¹² and R¹³, taken together with the atoms to which they are attached, form an aromatic Ring F having exactly 6 ring atoms, wherein each ring atom is a carbon ring atom, and wherein Ring F is fused to Ring A.

32-35. (canceled)

36. A compound according to claim 1, wherein each Ring A substituent, if present, is independently:
—F, —Cl, —Br, —I,
—R^{D1},
—CF₃,
—OH,
-L¹-OH,
—OR^{D1},
-L¹-OR^{D1},
—OCF₃,
—SH,
—SR^{D1},
—SCF₃,
—CN,
—NO₂,
—NH₂, —NHR^{D1}, —NR^{D1}₂, —NR^{N1}R^{N2},
-L¹-NH₂, -L¹-NHR^{D1}, -L¹-NR^{D1}₂, -L¹-NR^{N1}R^{N2},
—C(═O)OH,
—C(═O)OR^{D1},
—C(═O)NH₂, —C(═O)NHR^{D1}, —C(═O)NR^{D1}₂, —C(═O)NR^{N1}R^{N2},
—NHC(═O)R^{D1}, —NR^{D1}C(═O)R^{D1},
—OC(═O)R^{D1},
—C(═O)R^{D},
—NHS(═O)₂R^{D1}, —NR^{D1}S(═O)₂R^{D1},
—S(═O)₂NH₂, —S(═O)₂NHR^{D1}, —S(═O)₂NR^{D1}₂, —S(═O)₂NR^{N1}R^{N2},
—S(═O)₂R^{D1},
—OS(═O)₂R^{D1}, or
—S(═O)₂OR^{D1},
and additionally, two adjacent Ring A substituents, if present, may together form a group —O-L²-O—;
wherein:
each -L¹- is independently saturated aliphatic C₂₋₅alkylene;
each -L²- is independently saturated aliphatic C₁₋₃alkylene;
in each group —NR^{N1}R^{N2}, R^{N1} and R^{N2}, taken together with the nitrogen atom to which they are attached, form a 5-, 6-, or 7-membered non-aromatic ring having exactly 1 ring heteroatom or exactly 2 ring heteroatoms, wherein one of said exactly 2 ring heteroatoms is N, and the other of said exactly 2 ring heteroatoms is independently N or O;
each —R^{D1} is independently:
—R^{E1}, —R^{E2}, —R^{E3}, —R^{E4}, —R^{E5}, —R^{E6}, —R^{E7}, —R^{E8},
-L³-R^{E4}, -L³-R^{E5}, -L³-R^{E6}, -L³-R^{E7}, or L³-R^{E8};
wherein:
each —R^{E1} is independently saturated aliphatic C₁₋₆alkyl;
each —R^{E2} is independently aliphatic C₂₋₆alkenyl;
each —R^{E3} is independently aliphatic C₂₋₆alkynyl;
each —R^{E4} is independently saturated C₃₋₆cycloalkyl;
each —R^{E5} is independently C₃₋₆cycloalkenyl;
each —R^{E6} is independently non-aromatic C₃₋₇heterocyclyl;
each —R^{E7} is independently C₆₋₁₄carboaryl;
each —R^{E8} is independently C₅₋₁₄heteroaryl;
each -L³- is independently saturated aliphatic C₁₋₃alkylene;
and wherein:
each C₁₋₆alkyl, C₂₋₆alkenyl, C₂₋₆alkynyl, C₃₋₆cycloalkyl, C₃₋₆cycloalkenyl, non-aromatic C₃₋₇heterocyclyl, C₆₋₁₄carboaryl, C₅₋₁₄heteroaryl, and C₁₋₃alkylene is optionally substituted with one or more substituents selected from:
—F, —Cl, —Br, —I,
R^{F1},
—CF₃,
CF₃,
—OH,
OR^{F1},
—OCF₃,
—SH,
—SR^{F1},
—SCF₃,
—CN,
—NO₂,
—NH₂, —NHR^{F1}, —NR^{F1}₂, —NR^{N3}R^{N4},
—C(═O)OH,
—C(═O)OR^{F1},
—C(═O)NH₂, —C(═O)NHR^{F1}, —C(═O)NR^{F1}₂, —C(═O)NR^{N3}R^{N4},
-L⁴-OH, -L⁴-OR^{F1},
-L⁴-NH₂, -L⁴-NHR^{F1}, -L⁴-NR^{F1}₂, or -L⁴-NR^{N3}R^{N4};
wherein:
each —R^{F1} is independently saturated aliphatic C₁₋₄alkyl;
each -L⁴- is independently saturated aliphatic C₂₋₅alkylene; and
in each group —NR^{N3}R^{N4}, R^{N3} and R^{N4}, taken together with the nitrogen atom to which they are attached, form a 5-, 6-, or 7-membered non-aromatic ring having exactly 1 ring heteroatom or exactly 2 ring heteroatoms, wherein one of said exactly 2 ring heteroatoms is N, and the other of said exactly 2 ring heteroatoms is independently N or O.

37. A compound according to claim 1, wherein each Ring A substituent, if present, is independently selected from: —F, —Cl, —Br, —I, —OH, —O—C₁₋₇alkyl, —O—C₁₋₇haloalkyl, —S—C₁₋₇alkyl, —NH₂, —NH—C₁₋₇alkyl, —N(C₁₋₇alkyl)₂, —C(═O)OH, —C(═O)O—C₁₋₇alkyl, —C(═O)NH₂, —OC(═O)—C₁₋₇alkyl, —NO₂, C₁₋₇alkyl, —C₁₋₇haloalkyl, —CH₂-Ph, -Ph, -Ph-C₁₋₇haloalkyl.

38-45. (canceled)

46. A compound according to claim 17, wherein R¹¹ is independently —R^{H7} or —R^{H8}, and wherein —R^{H7}, if present, is independently phenyl and —R^{H8}, if present, is independently pyrazolyl or pyridyl; and
wherein said phenyl, pyrazolyl, or pyridyl is optionally substituted with one or more substituents selected from:
—F, —Cl, —Br, —I,
—R^{J1},
—CF₃,
—OH,
—OR^{J1},
—OCF₃,
—SH,
—SR^{J1},
—SCF₃,
—CN,
—NO₂,
—NH₂, —NHR^{J1}, —NR^{J1}₂, —NR^{N5}R^{N6},
—C(═O)OH,
—C(═O)OR^{J1},
—C(═O)NH₂, —C(═O)NHR^{J1}, —C(═O)NR^{J1}₂, —C(═O)NR^{N5}R^{N6},
-L⁵-OH, -L⁵-OR^{J1},
-L⁵-NH₂, -L⁵-NHR^{J1}, -L⁵-NR^{J1}₂, or -L⁵-NR^{N5}R^{N6};
wherein:
each —R^{J1} is independently saturated aliphatic C₁₋₄alkyl;
each -L⁵- is independently saturated aliphatic C₂₋₅alkylene; and
in each group —NR^{N5}R^{N6}, R^{N5} and R^{N6}, taken together with the nitrogen atom to which they are attached, form a 5-, 6-, or 7-membered non-aromatic ring having exactly 1 ring heteroatom or exactly 2 ring heteroatoms, wherein one of said exactly 2 ring heteroatoms is N, and the other of said exactly 2 ring heteroatoms is independently N or O.

47-51. (canceled)

52. A compound according to claim 1, wherein the group W, if present, is independently selected from: -Me, -Et, -nPr, -iPr, -tBu, -Ph, —CH₂-Ph.

53. A compound according to claim 1, wherein R¹⁴ is independently —H.

54. A compound according to claim 1, wherein the group 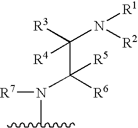
is the following group:

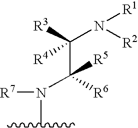

55-56. (canceled)

57. A compound according to claim 1, wherein each of R¹, R², R³, R⁴, R⁵, R⁶, and R⁷ is independently —H or a group G.

58-59. (canceled)

60. A compound according to claim 1, wherein the group: 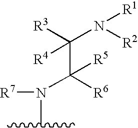
is independently selected from the following groups:

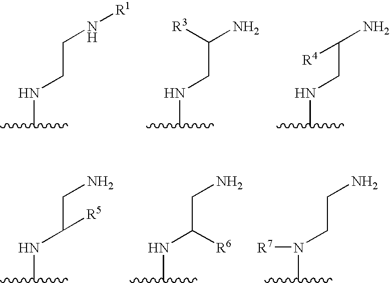

61. (canceled)

62. A compound according to claim 1, wherein the group: 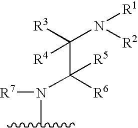
is independently selected from the following groups:

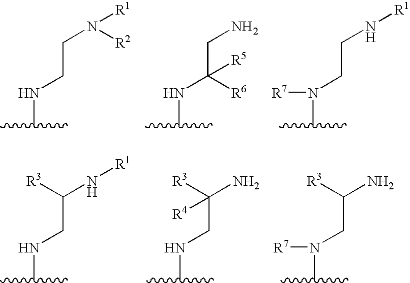

63-64. (canceled)

65. A compound according to claim 1, wherein the group: 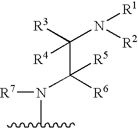
is independently selected from the following groups:

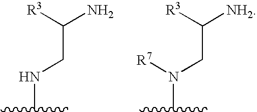

66-70. (canceled)

71. A compound according to claim 1, wherein each group G, if present, is independently C₁₋₇alkyl, and is independently unsubstituted or substituted with one or more substituents selected from: —F, —Cl, —Br, —I, —OH, —OMe, —OCF₃, —SMe, —NH₂, —NHMe, —NMe₂, —C(═O)OH, —C(═O)OMe, —C(═O)NH₂, —OC(═O)Me, —NO₂, -Ph, -Ph-CF₃.

72. A compound according to claim 1, wherein each group G, if present, is independently C₁₋₇alkyl, and is unsubstituted.

73. (canceled)

74. A compound according to claim 1, wherein each group Y, if present, is independently selected from: —C(═O)OH, —C(═O)OMe, —C(═O)OEt, —C(═O)OPh, —C(═O)OCH₂Ph, —C(═O)NH₂, —C(═O)NH Me, —C(═O)NHEt, —C(═O)NMe₂, —C(═O)NEt₂.

75. A compound according to claim 1, wherein each group Z, if present, is independently selected from: —C(═O)Me, —C(═O)Et, —C(═O)OMe, —C(═O)OEt, —C(═O)OPh, —C(═O)OCH₂Ph, —C(═O)NH₂, —C(═O)NHMe, —C(═O)NHEt, —C(═O)NMe₂, —C(═O)NEt₂, —S(═O)₂Me, —S(═O)₂Et, —S(═O)₂Ph, —S(═O)₂Ph-Me.

76. A compound according to claim 1, wherein:
one of R³ and R⁴ is independently C₁₋₆alkyl or C₃₋₆cycloalkyl;
the other of R³ and R⁴ is independently —H;
R⁷ is independently —H or C₁₋₆alkyl; and
each of R¹, R², R⁵, and R⁶ is independently —H.

77. A compound according to claim 1, wherein:
one of R³ and R⁴ is independently C₁₋₄alkyl or C₃₋₄cycloalkyl;
the other of R³ and R⁴ is independently —H;
R⁷ is independently —H or C₁₋₄alkyl; and
each of R¹, R², R⁵, and R⁶ is independently —H.

78. (canceled)

79. A compound according to claim 1, selected from the following compounds and pharmaceutically acceptable salts thereof: compounds XX-001 to XX-099, XX-101 to XX-109, XX-112, XX-114 to XX-344, and YY-001 to YY-003.

80. A pharmaceutical composition comprising a compound according to claim 1, and a pharmaceutically acceptable carrier or diluent.

81. A method of preparing a pharmaceutical composition comprising the step of admixing a compound according to claim 1, and a pharmaceutically acceptable carrier or diluent.

82.-98. (canceled)

99. A method for the treatment of a disease or condition that is mediated by PKD (e.g., PKD1, PKD2, PKD3) or a disease or condition that is ameliorated by the inhibition of PKD (e.g., PKD1, PKD2, PKD3) or a proliferative condition or cancer or a hyperproliferative skin disorder or psoriasis or actinic keratosis or non-melanoma skin cancer or a disease or condition that is characterised by inappropriate, excessive, and/or undesirable angiogenesis or an inflammatory disease or a disease or disorder associated with heart remodelling or myocyte hypertrophy of the heart or impaired contractility of the heart or pump failure of the heart or pathologic cardiac hypertrophy or heart failure comprising administering to a subject in need of treatment a therapeutically-effective amount of a compound as defined in claim 1, without the recited proviso.

100.-106. (canceled)

107. A method of inhibiting PKD (e.g., PKD1, PKD2, PKD3) in a cell, in vitro or in vivo, comprising contacting the cell with an effective amount of a compound as defined in claim 1, without the recited proviso.

108. A method of inhibiting cell proliferation, inhibiting cell cycle progression, promoting apoptosis, or a combination of one or more these, in vitro or in vivo, comprising contacting the cell with an effective amount of a compound as defined in claim 1, without the recited proviso.


SEQUENCE LISTING (7 sequences)

SEQ ID NO: 1 - DNA, 1161 bp, Mus musculus
atgtcgtactaccatcaccatcaccatcacgattacgatatcccaacgaccgaaaacctgtattttcagggcgccatgggatccaactcacacaaagatatttctgtgagcatttccgtttcaaattgccagattcaggaaaatgtggatatcagcacagtctatcaaatatttcctgatgaagttctgggttccggacagtttggaattgtttatggaggtaaacatcgtaaaacaggaagagatgtagctattaagattattgacaaattaagatttcctacaaaacaagaaagtcagcttcgtaatgaggttgcaattttacagaaccttcatcaccctggtgttgtaaatttggagtgtatgtttgagacgcctgaaagagtgtttgttgttatggaaaaactccatggagacatgctggagatgatcttgtcaagtgaaaagggctggttgccagaacacataacgaagtttttaattactcagatactagtggctttgcgacatcttcattttaaaaacatcgttcactgtgacctcaagccagaaaatgtgttgctggcatcagccgaccctttccctcaggtgaagctctgtgattttggttttgcccggatcattggagagaagtcttttaggaggtcagtggtgggtaccccagcatacctggcacctgaagttctgaggaacaagggctataaccgctctctggacatgtggtctgttggggtcatcatctatgtgagcctgagtggtaccttcccttttaatgaagatgaagatatccatgatcagatccagaatgcagccttcatgtatccacccaacccgtggaaggagatttctcatgaagccattgatcttatcaataacttgctacaagtgaaaatgagaaaacgctacagtgtggataaaaccttgagtcacccttggctacaggactatcagacctggttagatttacgagagctggaatgcagaattggagaacgctatattacccacgaaagcgatgactccaggtgggaacagtacgcaggcgagcaggggctgcagtacccggcgcacctgatcagtctgagtgctagccacagcgacagtcctgaggctgaagagagagagatgaaagccctcagtgagcgtgtcagcatcctctga

SEQ ID NO: 2 - protein, 386 aa, Mus musculus
MSYYHHHHHHDYDIPTTENLYFQGAMGSNSHKDISVSISVSNCQIQENVDISTVYQIFPDEVLGSGQFGIVYGGKHRKTGRDVAIKIIDKLRFPTKQESQLRNEVAILQNLHHPGVVNLECMFETPERVFVVMEKLHGDMLEMILSSEKGWLPEHITKFLITQILVALRHLHFKNIVHCDLKPENVLLASADPFPQVKLCDFGFARIIGEKSFRRSVVGTPAYLAPEVLRNKGYNRSLDMWSVGVIIYVSLSGTFPFNEDEDIHDQIQNAAFMYPPNPWKEISHEAIDLINNLLQVKMRKRYSVDKTLSHPWLQDYQTWLDLRELECRIGERYITHESDDSRWEQYAGEQGLQYPAHLISLSASHSDSPEAEEREMKALSERVSIL

SEQ ID NO: 3 - protein, 257 aa, Mus musculus
IFPDEVLGSGQFGIVYGGKHRKTGRDVAIKIIDKLRFPTKQESQLRNEVAILQNLHHPGVVNLECMFETPERVFVVMEKLHGDMLEMILSSEKGWLPEHITKFLITQILVALRHLHFKNIVHCDLKPENVLLASADPFPQVKLCDFGFARIIGEKSFRRSVVGTPAYLAPEVLRNKGYNRSLDMWSVGVIIYVSLSGTFPFNEDEDIHDQIQNAAFMYPPNPWKEISHEAIDLINNLLQVKMRKRYSVDKTLSHPWL

SEQ ID NO: 4 - protein, 257 aa, Homo sapiens
IFPDEVLGSGQFGIVYGGKHRKTGRDVAIKIIDKLRFPTKQESQLRNEVAILQNLHHPGVVNLECMFETPERVFVVMEKLHGDMLEMILSSEKGRLPEHITKFLITQILVALRHLHFKNIVHCDLKPENVLLASADPFPQVKLCDFGFARIIGEKSFRRSVVGTPAYLAPEVLRNKGYNRSLDMWSVGVIIYVSLSGTFPFNEDEDIHDQIQNAAFMYPPNPWKEISHEAIDLINNLLQVKMRKRYSVDKTLSHPWL

SEQ ID NO: 5 - protein, 257 aa, Homo sapiens
IFPDEVLGSGQFGVVYGGKHRKTGRDVAVKVIDKLRFPTKQESQLRNEVAILQSLRHPGIVNLECMFETPEKVFVVMEKLHGDMLEMILSSEKGRLPERLTKFLITQILVALRHLHFKNIVHCDLKPENVLLASADPFPQVKLCDFGFARIIGEKSFRRSVVGTPAYLAPEVLLNQGYNRSLDMWSVGVIMYVSLSGTFPFNEDEDINDQIQNAAFMYPASPWSHISAGAIDLINNLLQVKMRKRYSVDKSLSHPWL

SEQ ID NO: 6 - protein, 257 aa, Homo sapiens
IFADEVLGSGGFGIVYGGKHRKTGRDVAIKVIDKMRFPTKQESQLRNEVAILQNLHHPGIVNLECMFETPERVFVVMEKLHGDMLEMILSSEKSRLPERITKFMVTQILVALRNLHFKNIVHCDLKPENVLLASAEPFPQVKLCDFGFARIIGEKSFRRSVVGTPAYLAPEVLRSKGYNRSLDMWSVGVIIYVSLSGTFPFNEDEDINDQIQNAAFMYPPNPWREISGEAIDLINNLLQVKMRKRYSVDKSLSHPWL

SEQ ID NO: 7 - protein, 10 aa, Artificial sequence
  Synthetic sequence Fluorescein labelled glycogen synthase-derived peptide
KKLNRTLSVA